(19) United States
(12) Patent Application Publication
(10) Pub. No.: US 2025/0179354 A1
(43) Pub. Date: Jun. 5, 2025

(54) HETEROCYCLIC COMPOUND, ORGANIC LIGHT EMITTING DEVICE COMPRISING SAME AND COMPOSITION FOR ORGANIC MATERIAL LAYER OF ORGANIC LIGHT EMITTING DEVICE

(71) Applicant: LT MATERIALS CO.,LTD., Yongin-City (KR)

(72) Inventors: Yu-Jin HEO, Yongin-City (KR); Jun-Tae MO, Yongin-City (KR); Dong-Jun KIM, Yongin-City (KR); Dae-Hyuk CHOI, Yongin-City (KR)

(73) Assignee: LT MATERIALS CO.,LTD., Yongin-City (KR)

(21) Appl. No.: 18/960,031

(22) Filed: Nov. 26, 2024

(30) Foreign Application Priority Data
Nov. 30, 2023 (KR) 10-2023-0171543

(51) Int. Cl. C09K 11/06; H10K 50/11; H10K 85/60

(52) U.S. Cl. CPC C09K 11/06; H10K 50/11; H10K 85/657; C09K 2211/1062; C09K 2211/1066

(57) ABSTRACT

Disclosed are a heterocyclic compound of Chemical Formula 1, an organic light emitting device including the same and a composition for an organic material layer of an organic light emitting device.

### CROSS-REFERENCE TO RELATED APPLICATIONS

This application claims priority to and the benefit of Korean Patent Application No. 10-2023-0171543 filed in the Korean Intellectual Property Office on Nov. 30, 2023, the entire contents of which are incorporated herein by reference.

### TECHNICAL FIELD

The present specification relates to a heterocyclic compound, an organic light emitting device including the same and a composition for an organic material layer of the organic light emitting device.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

### Patent Document

(Patent Document 1) U.S. Pat. No. 4,356,429

### SUMMARY OF THE INVENTION

The present invention has been made in an effort to provide a heterocyclic compound, an organic light emitting device including the same and a composition for an organic material layer of an organic light emitting device.

An exemplary embodiment of the present invention provides a heterocyclic compound of the following Chemical Formula 1.

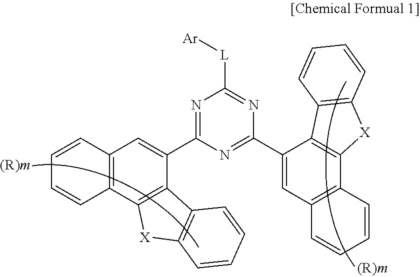

In Chemical Formula 1,
X is O or S,
L is a direct bond; a substituted or unsubstituted C6 to C60 arylene group; or a substituted or unsubstituted C2 to C60 heteroarylene group,
Ar is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R is hydrogen; deuterium; a halogen group; a cyano group; a substituted or unsubstituted C1 to C60 alkyl group; a substituted or unsubstituted C1 to C60 haloalkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
m is an integer from 1 to 9, and when m is 2 or higher, substituents in the parenthesis are the same as or different from each other.

Another exemplary embodiment provides an organic light emitting device including: a first electrode; a second electrode; and an organic material layer having one or more layers provided between the first electrode and the second electrode, in which one or more layers of the organic material layer include one or more of the heterocyclic compounds.

Yet another exemplary embodiment provides a composition for an organic material layer of an organic light emitting device, including the heterocyclic compound.

When used in an organic light emitting device, the heterocyclic compound described in the present specification can lower the driving voltage of the device, improve the light emitting efficiency, and improve the service life characteristics of the device. Specifically, when in the heterocyclic compound of the present invention, positions 6 of the two identical naphthobenzofuran groups/naphthobenzothiophene groups are bonded to triazine as shown in Chemical Formula 1 and the heterocyclic compound is used as a light emitting layer material, the performance of the device can be effectively improved. As described above, triazine is bonded to the specific positions of the two identical naphthobenzofuran groups/naphthobenzothiophene groups to have a symmetric structure, thereby expanding the conjugation of the molecule and having an appropriate band gap and homo level. Accordingly, charge transfer is facilitated, so that there is an effect that the light emitting efficiency is increased.

### BRIEF DESCRIPTION OF THE DRAWINGS

FIGS. 1 to 3 are views each exemplarily illustrating a stacking structure of an organic light emitting device according to an exemplary embodiment of the present specification.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification, “N to N′” means N or more and N′ or less.

In the present specification,

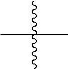

of a chemical formula means a position to which a constituent element is bonded.

The term “substitution” means that a hydrogen atom bonded to a carbon atom or nitrogen atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or a substituent to which two or more substituents selected among the exemplified substituents are linked.

In the present specification, Cn1 to Cn2 (n1 and n2 are integers of 1 or higher) means a range of carbon atoms. For example, a C1 to C60 alkyl group means an alkyl group having 1 to 60 carbon atoms.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the deuterium content may be 0% to 100%, and the deuterium content may be expressed as a deuterium substitution rate.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the substitution rate T % of the specific substituent may be defined as T2/T1×100=T %.

That is, in an example, a deuterium substitution rate of 20% in a phenyl group represented by

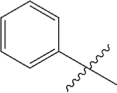 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium substitution rate of 20% in the phenyl group may be represented by the following structural formula.

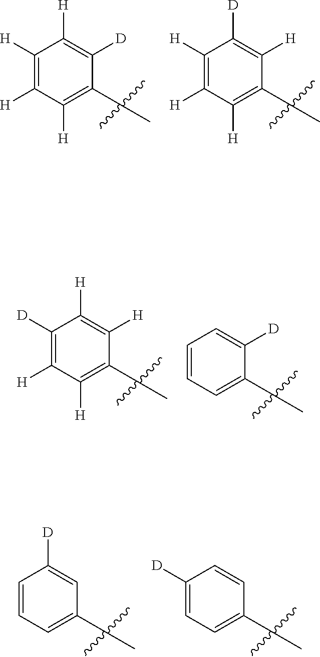 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium substitution rate of 0%” may mean a phenyl group that does not include a deuterium atom as a substituent, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(=0)₂(R105), and the above-described examples of the alkyl group may be applied to R105.

In the present specification, an arylsulfoxy group is represented by —S(=0)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes 0, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

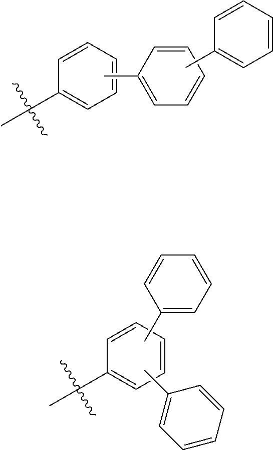

In the present specification, the fluorenyl group may be substituted, and adjacent substituents may be bonded to each other to form a ring.

When the fluorenyl group is substituted, the substituent may be the following structures, but is not limited thereto.

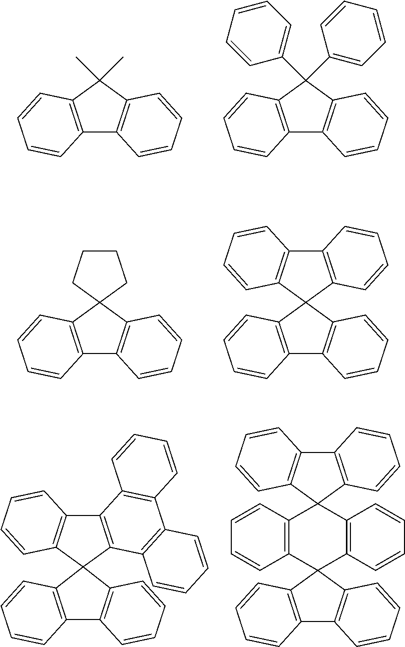

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, a benzocarbazole group may be any one of the following structures.

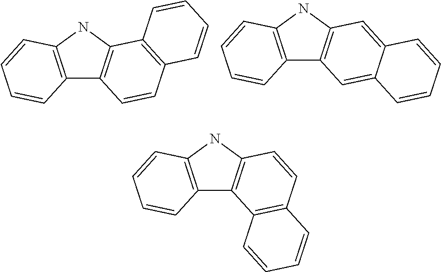

In the present specification, a dibenzocarbazole group may be any one of the following structures.

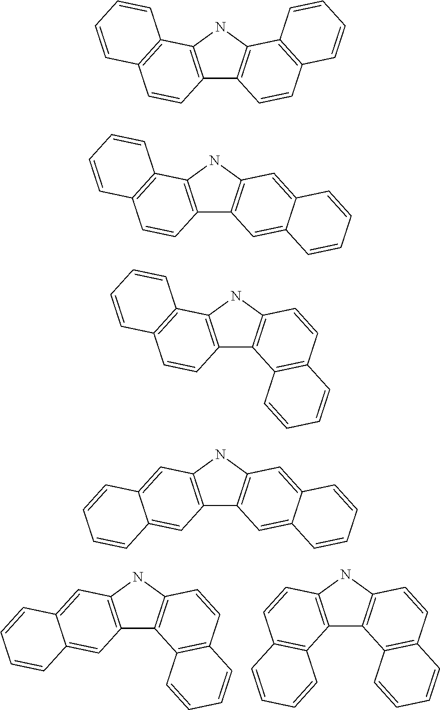

In the present specification, when the substituent is a carbazole group, a benzocarbazole group, or a dibenzocarbazole group, it means being bonded to the nitrogen or carbon of the carbazole group, the benzocarbazole group, or the dibenzocarbazole group.

In the present specification, when a carbazole group, a benzocarbazole group, or a dibenzocarbazole group is substituted, an additional substituent may be substituted at the nitrogen or carbon of the carbazole group, the benzocarbazole group, or the dibenzocarbazole group.

In the present specification, a naphthobenzofuran group may be any one of the following structures.

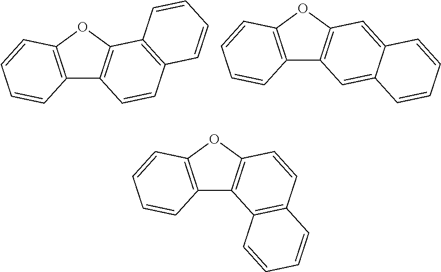

In the present specification, a naphthobenzothiophene group may be any one of the following structures.

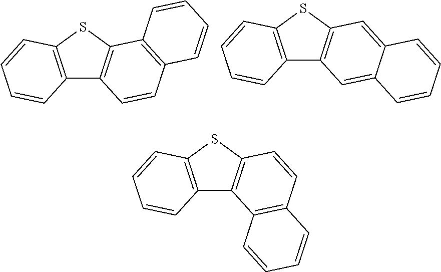

In the present specification, a silyl group includes Si and is a substituent to which the Si atom is directly linked as a radical, and is represented by —Si(R107) (R108) (R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

The silyl group may include an alkylsilyl group, an arylsilyl group, a heteroarylsilyl group, an alkylarylsilyl group, an arylheteroarylsilyl group, and the like, according to the substituent bonded to the Si element. An alkylsilyl group, an arylsilyl group, or a heteroarylsilyl group means that an alkyl group, an aryl group, or a heteroaryl group is substituted with the Si element of a silyl group, respectively, an alkylarylsilyl group means that an alkyl group and an aryl group are substituted with the Si element of a silyl group, and an arylheteroarylsilyl group means that an aryl group and a heteroaryl group are substituted with the Si element of a silyl group.

Specific examples of the silyl group include the following structures, but are not limited thereto.

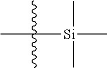 [SMILES: *[Si](C)(C)C]

(A trimethylsilyl group),

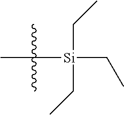 [SMILES: *[Si](CC)(CC)CC]

(a triethylsilyl group),

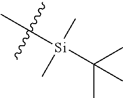 [SMILES: *[Si](C)(C)C(C)(C)C]

(a t-butyldimethylsilyl group),

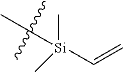 [SMILES: *[Si](C)(C)C=C]

(a vinyldimethylsilyl group),

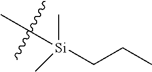 [SMILES: *[Si](C)(C)CCC]

(a propyldimethylsilyl group),

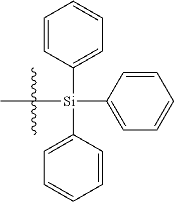 [SMILES: *[Si](c1ccccc1)(c1ccccc1)c1ccccc1]

a triphehylsilyl group),

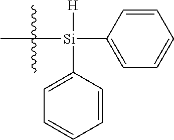 [SMILES: *[SiH](c1ccccc1)c1ccccc1]

(a diphenylsilyl group), and

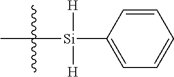 [SMILES: *[SiH2]c1ccccc1]

(a phenylsilyl group)

In the present specification, a phosphine oxide group is represented by —P(═O) (R110) (R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112) (R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described description of the aryl group may be applied to an arylene group except for a divalent arylene group.

In the present specification, the above-described description of the heteroaryl group may be applied to a heteroarylene group except for a divalent heteroarylene group.

An exemplary embodiment of the present specification provides a heterocyclic compound of the following Chemical Formula 1.

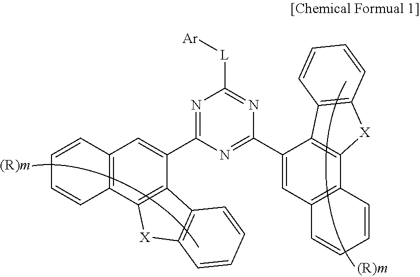

In Chemical Formula 1,
X is O or S,
L is a direct bond; a substituted or unsubstituted C6 to C60 arylene group; or a substituted or unsubstituted C2 to C60 heteroarylene group,
Ar is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R is hydrogen; deuterium; a halogen group; a cyano group; a substituted or unsubstituted C1 to C60 alkyl group; a substituted or unsubstituted C1 to C60 haloalkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
m is an integer from 1 to 9, and when m is 2 or higher, substituents in the parenthesis are the same as or different from each other.

The heterocyclic compound according to an exemplary embodiment of the present specification has two identical naphthobenzofuran groups/naphthobenzothiophene groups as substituents of a triazine group, and the structure and substitution position of the naphthobenzofuran groups/naphthobenzothiophene groups are specified. By having such a structure, the compound helps to improve the performance of an organic light emitting device when used as a material for the organic light emitting device.

That is, in Chemical Formula 1 above, the two

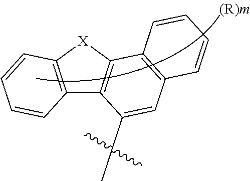

have the same structure. Specifically, the two

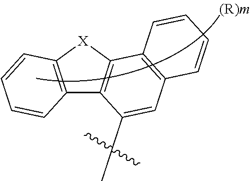

have the same type of substituent R at the same position. In the structure,

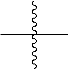

is a position bonded to triazine.
In an exemplary embodiment of the present specification, X may be O.

In an exemplary embodiment of the present specification, X may be S.

In an exemplary embodiment of the present specification, Chemical Formula 1 may be represented by the following Chemical Formula 1-O or 1-S.

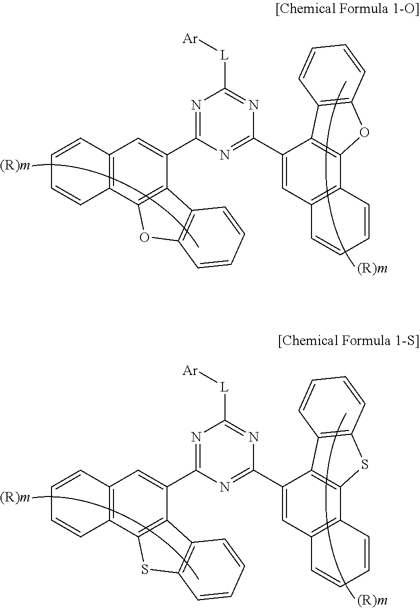

In Chemical Formulae 1-O and 1-S,
the definition of each substituent is the same as the definition in Chemical Formula 1.

In an exemplary embodiment of the present specification, L may be a direct bond; a substituted or unsubstituted C6 to C30 arylene group; or a substituted or unsubstituted C2 to C30 heteroarylene group.

In an exemplary embodiment of the present specification, L may be a direct bond; a substituted or unsubstituted C6 to C20 arylene group; or a substituted or unsubstituted C2 to C20 heteroarylene group.

In an exemplary embodiment of the present specification, L may be a direct bond; a substituted or unsubstituted phenylene group; a substituted or unsubstituted naphthylene group; a substituted or unsubstituted divalent dibenzofuran group; a substituted or unsubstituted divalent dibenzothiophene group; or a substituted or unsubstituted divalent carbazole group.

In an exemplary embodiment of the present specification, L may be a direct bond; a C6 to C60 arylene group unsubstituted or substituted with deuterium; or a C2 to C60 heteroarylene group unsubstituted or substituted with deuterium.

In an exemplary embodiment of the present specification, L may be a direct bond; a C6 to C30 arylene group unsubstituted or substituted with deuterium; or C2 to C30 heteroarylene group unsubstituted or substituted with deuterium.

In an exemplary embodiment of the present specification, L may be a direct bond; a C6 to C20 arylene group unsubstituted or substituted with deuterium; or a C2 to C20 heteroarylene group unsubstituted or substituted with deuterium.

In an exemplary embodiment of the present specification, L may be a direct bond; a phenylene group unsubstituted or substituted with deuterium; a naphthylene group unsubstituted or substituted with deuterium; a divalent dibenzofuran group unsubstituted or substituted with deuterium; a divalent dibenzothiophene group unsubstituted or substituted with deuterium; or a divalent carbazole group unsubstituted or substituted with deuterium.

In an exemplary embodiment of the present specification, L may be a direct bond, or selected from the following structures.

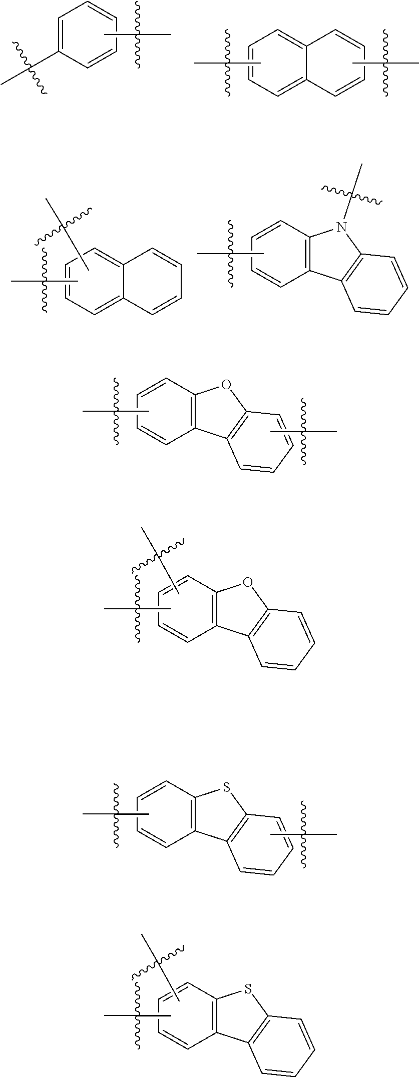

In the structures,

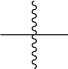

is a position to which a triazine group and Ar are bonded, and the structures may be further substituted with deuterium.

In an exemplary embodiment of the present specification, Ar may be a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In an exemplary embodiment of the present specification, Ar may be a substituted or unsubstituted C6 to C60 aryl group; or a substituted or unsubstituted C2 to C60 heteroaryl group.

In an exemplary embodiment of the present specification, Ar may be a substituted or unsubstituted C6 to C30 aryl group; or a substituted or unsubstituted C2 to C30 heteroaryl group.

In an exemplary embodiment of the present specification, Ar may be a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenylenyl group; a substituted or unsubstituted fluoranthenyl group; a substituted or unsubstituted fluorenyl group; a substituted or unsubstituted dibenzofuran group; a substituted or unsubstituted dibenzothiophene group; a substituted or unsubstituted carbazole group; a substituted or unsubstituted naphthobenzofuran group; or a substituted or unsubstituted naphthobenzothiophene group.

In an exemplary embodiment of the present specification, Ar may be a phenyl group; a biphenyl group; a terphenyl group; a naphthyl group; a phenanthrenyl group; a triphenylenyl group; a fluoranthenyl group; a fluorenyl group; a dibenzofuran group; a dibenzothiophene group; a carbazole group; a naphthobenzofuran group; or a naphthobenzothiophene group, and Ar may be further substituted with one or more substituents or two or more linking groups selected from deuterium, a halogen group, an alkyl group, an aryl group, and a heteroaryl group.

In an exemplary embodiment of the present specification, Ar may be a C6 to C60 aryl group unsubstituted or substituted with one or more substituents or two or more linking groups selected from deuterium, a halogen group, an alkyl group, an aryl group, and a heteroaryl group; or a C2 to C60 heteroaryl group unsubstituted or substituted with one or more substituents or two or more linking groups selected from deuterium, a halogen group, an alkyl group, an aryl group, and a heteroaryl group.

In an exemplary embodiment of the present specification, Ar may be a C6 to C30 aryl group unsubstituted or substituted with one or more substituents or two or more linking groups selected from deuterium, a halogen group, an alkyl group, an aryl group, and a heteroaryl group; or a C2 to C30 heteroaryl group unsubstituted or substituted with one or more substituents or two or more linking groups selected from deuterium, a halogen group, an alkyl group, an aryl group, and a heteroaryl group.

In an exemplary embodiment of the present specification, Ar may be a C6 to C60 aryl group unsubstituted or substituted with one or more substituents or two or more linking groups selected from deuterium, a halogen group, an alkyl group, an aryl group, and a heteroaryl group; or a C2 to C60 heteroaryl group unsubstituted or substituted with one or more substituents or two or more linking groups selected from deuterium and an aryl group.

In an exemplary embodiment of the present specification, Ar may be a C6 to C30 aryl group unsubstituted or substituted with one or more substituents or two or more linking groups selected from deuterium, a halogen group, an alkyl group, an aryl group, and a heteroaryl group; or a C2 to C30 heteroaryl group unsubstituted or substituted with one or more substituents or two or more linkers selected from deuterium and an aryl group.

The linking group means a substituent formed by linking two or more substituents. For example, a linking group of a halogen group and an aryl group may include an aryl group substituted with a halogen group, and a linking group of deuterium, a halogen group and an aryl group may include an aryl group substituted with deuterium and a halogen group.

In an exemplary embodiment of the present specification, R may be hydrogen; deuterium; a halogen group; a cyano group; a substituted or unsubstituted C1 to C40 alkyl group; a substituted or unsubstituted C1 to C40 haloalkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In an exemplary embodiment of the present specification, R may be hydrogen; deuterium; a halogen group; a substituted or unsubstituted C1 to C60 haloalkyl group; or a substituted or unsubstituted C6 to C60 aryl group.

In an exemplary embodiment of the present specification, R may be hydrogen; deuterium; a halogen group; a substituted or unsubstituted C1 to C30 haloalkyl group; or a substituted or unsubstituted C6 to C30 aryl group.

In an exemplary embodiment of the present specification, R may be hydrogen; deuterium; a halogen group; a substituted or unsubstituted C1 to C10 haloalkyl group; or a substituted or unsubstituted C6 to C20 aryl group.

In an exemplary embodiment of the present specification, R may be hydrogen; deuterium; a halogen group; —CF₃; or a substituted or unsubstituted phenyl group.

In an exemplary embodiment of the present specification, R may be hydrogen; deuterium; a halogen group; a C1 to C60 haloalkyl group; or a C6 to C60 aryl group unsubstituted or substituted with deuterium.

In an exemplary embodiment of the present specification, R may be hydrogen; deuterium; a halogen group; a C1 to C30 haloalkyl group; or a C6 to C30 aryl group unsubstituted or substituted with deuterium.

In an exemplary embodiment of the present specification, R may be hydrogen; deuterium; a halogen group; a C1 to C10 haloalkyl group; or a C6 to C20 aryl group unsubstituted or substituted with deuterium.

In an exemplary embodiment of the present specification, R may be hydrogen; deuterium; a halogen group; —CF₃; or a phenyl group unsubstituted or substituted with deuterium.

In an exemplary embodiment of the present specification, Chemical Formula 1 may be represented by any one of the following Chemical Formulae 1-1 to 1-4.

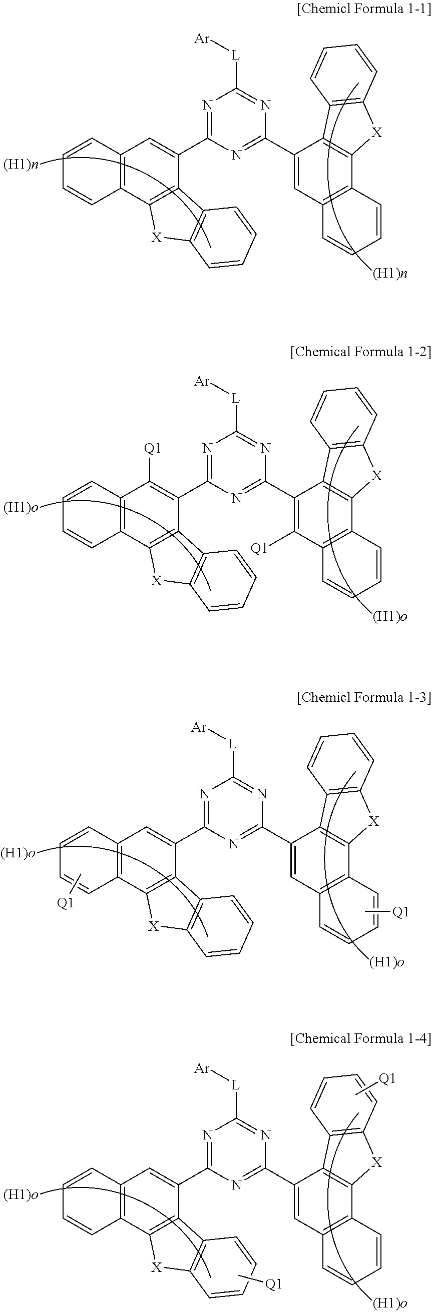

In Chemical Formulae 1-1 to 1-4,
the definitions of L, Ar, and X are the same as the definitions in Chemical Formula 1,
H1 is hydrogen; or deuterium,
Q1 is a halogen group; a substituted or unsubstituted C1 to C60 haloalkyl group; or a substituted or unsubstituted C6 to C60 aryl group,
n is an integer from 1 to 9, and when n is 2 or higher, substituents in the parenthesis are the same as or different from each other, and
o is an integer from 1 to 8, and when o is 2 or higher, substituents in the parenthesis are the same as or different from each other.

In an exemplary embodiment of the present specification, Q1 may be a halogen group; a substituted or unsubstituted C1 to C30 haloalkyl group; or a substituted or unsubstituted C6 to C30 aryl group.

In an exemplary embodiment of the present specification, Q1 may be a halogen group; a substituted or unsubstituted C1 to C10 haloalkyl group; or a substituted or unsubstituted C6 to C20 aryl group.

In an exemplary embodiment of the present specification, Q1 may be a halogen group; a C1 to C30 haloalkyl group; or a C6 to C30 aryl group unsubstituted or substituted with deuterium.

In an exemplary embodiment of the present specification, Q1 may be a halogen group; a C1 to C10 haloalkyl group; or a C6 to C20 aryl group unsubstituted or substituted with deuterium.

In an exemplary embodiment of the present specification, Q1 may be a halogen group; —CF₃; or a phenyl group unsubstituted or substituted with deuterium.

In the two

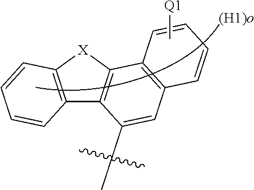

s of Chemical Formula 1-3, the substitution positions of Q1 are the same.

In the two

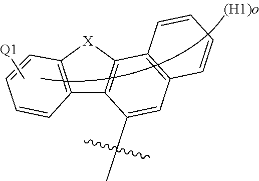

s of Chemical Formula 1-4, the substitution positions of Q1 are the same.

In an exemplary embodiment of the present specification, Chemical Formula 1 may be represented by the following Chemical Formula 1-1-1.

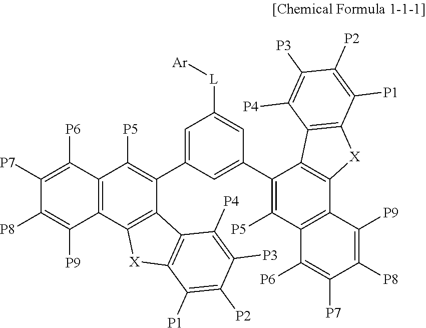

In Chemical Formula 1-1-1,
the definitions of L, Ar, and X are the same as the definitions in Chemical Formula 1, and
P1 to P9 are each independently hydrogen; deuterium; a halogen group; a substituted or unsubstituted C1 to C60 haloalkyl group; or a substituted or unsubstituted C6 to C60 aryl group.

In an exemplary embodiment of the present specification, P1 to P9 may be each independently hydrogen; deuterium; a halogen group; a substituted or unsubstituted C1 to C30 haloalkyl group; or a substituted or unsubstituted C6 to C30 aryl group.

In an exemplary embodiment of the present specification, P1 to P9 may be each independently hydrogen; deuterium; a halogen group; a substituted or unsubstituted C1 to C10 haloalkyl group; or a substituted or unsubstituted C6 to C20 aryl group.

In an exemplary embodiment of the present specification, P1 to P9 may be each independently hydrogen; deuterium; a halogen group; a C1 to C30 haloalkyl group; or a C6 to C30 aryl group unsubstituted or substituted with deuterium.

In an exemplary embodiment of the present specification, P1 to P9 may be each independently hydrogen; deuterium; a halogen group; a C1 to C10 haloalkyl group; or a C6 to C20 aryl group unsubstituted or substituted with deuterium.

In an exemplary embodiment of the present specification, P1 to P9 may be each independently hydrogen; deuterium; a halogen group; —CF₃; or a phenyl group unsubstituted or substituted with deuterium.

In an exemplary embodiment of the present specification, the deuterium substitution rate of Chemical Formula 1 may be 0% to 100%.

In an exemplary embodiment of the present specification, the deuterium substitution rate of Chemical Formula 1 may be 0% or 5% to 100%.

In an exemplary embodiment of the present specification, the deuterium substitution rate of Chemical Formula 1 may be 0% or 10% to 100%.

In an exemplary embodiment of the present specification, the deuterium substitution rate of Chemical Formula 1 may be 0% or 20% to 100%.

In an exemplary embodiment of the present specification, the deuterium substitution rate of Chemical Formula 1 may be 0% or 30% to 100%.

In the present specification, the deuterium substitution rate of Chemical Formula 1 means the substitution rate of deuterium with respect to the total number of hydrogen and deuterium atoms included in Chemical Formula 1 above. For example, when Chemical Formula 1 includes 20 hydrogen atoms and 20 deuterium atoms, the substitution rate of the 20 deuterium atoms with respect to the total of 40 hydrogen and deuterium atoms is 50%.

In an exemplary embodiment of the present specification, the deuterium substitution rate of the heterocyclic compound of Chemical Formula 1 satisfies the above range, the photochemical characteristics of a compound which includes deuterium and a compound which does not include deuterium are almost similar, but when deposited on a thin film, the deuterium-containing material tends to be packed with a narrower intermolecular distance.

Accordingly, when an electron only device (EOD) and a hole only device (HOD) are manufactured and the current density thereof according to voltage is confirmed, it can be confirmed that among the heterocyclic compounds of Chemical Formula 1 of the present invention, a compound including deuterium exhibits much more balanced charge transport characteristics than a compound which does not include deuterium.

Further, when the surface of a thin film is observed using an atomic force microscope (AFM), it can be confirmed that the thin film made of a compound including deuterium is deposited with a more uniform surface without any aggregated portion.

Additionally, since the single bond dissociation energy of carbon and deuterium is higher than the single bond dissociation energy of carbon and hydrogen, among the heterocyclic compounds of Chemical Formula 1 of the present invention, a compound including deuterium has the increased stability of the total molecules, so that there is an effect that the service life of the device is improved.

In an exemplary embodiment of the present specification, Chemical Formula 1 may be represented by any one of the following compounds.

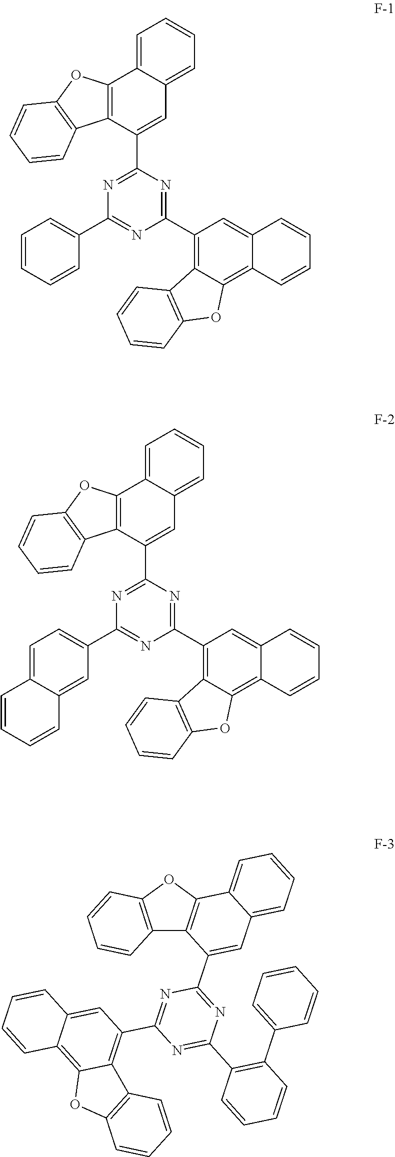 [SMILES: c1ccc(-c2nc(-c3cc4ccccc4c4oc5ccccc5c34)nc(-c3cc4ccccc4c4oc5ccccc5c34)n2)cc1 ; c1ccc2cc(-c3nc(-c4cc5ccccc5c5oc6ccccc6c45)nc(-c4cc5ccccc5c5oc6ccccc6c45)n3)ccc2c1 ; c1ccc(-c2ccccc2-c2nc(-c3cc4ccccc4c4oc5ccccc5c34)nc(-c3cc4ccccc4c4oc5ccccc5c34)n2)cc1]

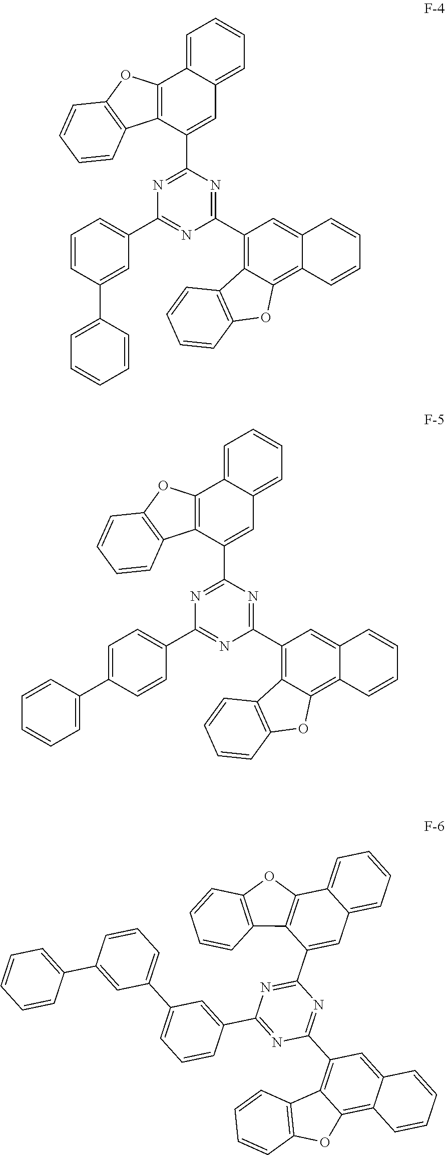 [SMILES: c1ccc(-c2cccc(-c3nc(-c4cc5ccccc5c5oc6ccccc6c45)nc(-c4cc5ccccc5c5oc6ccccc6c45)n3)c2)cc1 ; c1ccc(-c2ccc(-c3nc(-c4cc5ccccc5c5oc6ccccc6c45)nc(-c4cc5ccccc5c5oc6ccccc6c45)n3)cc2)cc1 ; c1ccc(-c2cccc(-c3cccc(-c4nc(-c5cc6ccccc6c6oc7ccccc7c56)nc(-c5cc6ccccc6c6oc7ccccc7c56)n4)c3)c2)cc1]

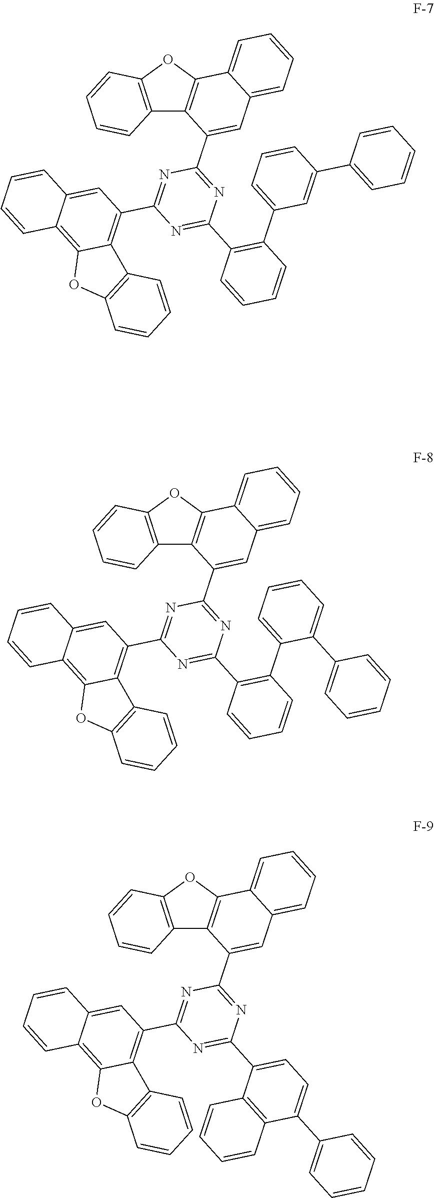 [SMILES: c1ccc(-c2cccc(-c3ccccc3-c3nc(-c4cc5ccccc5c5oc6ccccc6c45)nc(-c4cc5ccccc5c5oc6ccccc6c45)n3)c2)cc1 ; c1ccc(-c2ccccc2-c2ccccc2-c2nc(-c3cc4ccccc4c4oc5ccccc5c34)nc(-c3cc4ccccc4c4oc5ccccc5c34)n2)cc1 ; c1ccc(-c2ccc(-c3nc(-c4cc5ccccc5c5oc6ccccc6c45)nc(-c4cc5ccccc5c5oc6ccccc6c45)n3)c3ccccc23)cc1]

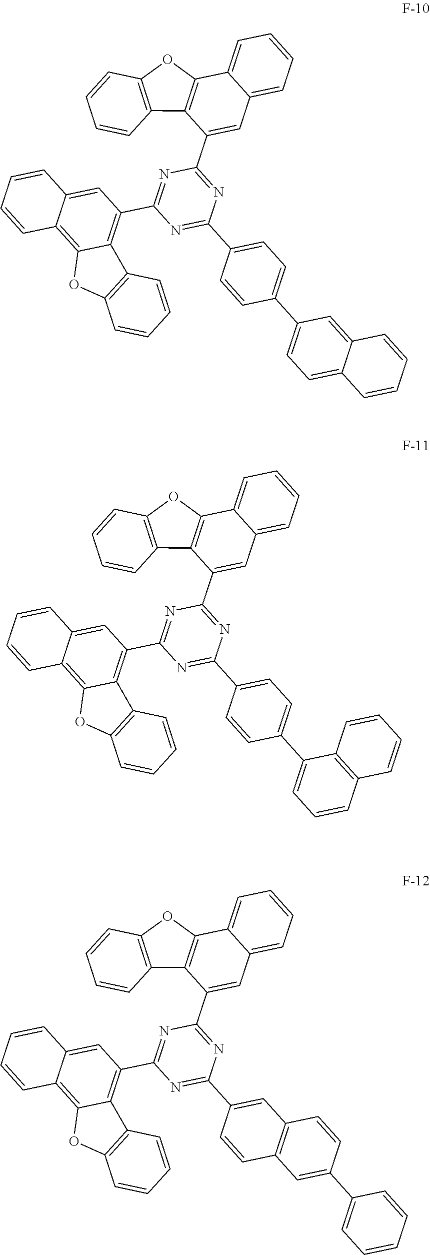 [SMILES: c1ccc2cc(-c3ccc(-c4nc(-c5cc6ccccc6c6oc7ccccc7c56)nc(-c5cc6ccccc6c6oc7ccccc7c56)n4)cc3)ccc2c1 ; c1ccc2c(-c3ccc(-c4nc(-c5cc6ccccc6c6oc7ccccc7c56)nc(-c5cc6ccccc6c6oc7ccccc7c56)n4)cc3)cccc2c1 ; c1ccc(-c2ccc3cc(-c4nc(-c5cc6ccccc6c6oc7ccccc7c56)nc(-c5cc6ccccc6c6oc7ccccc7c56)n4)ccc3c2)cc1]

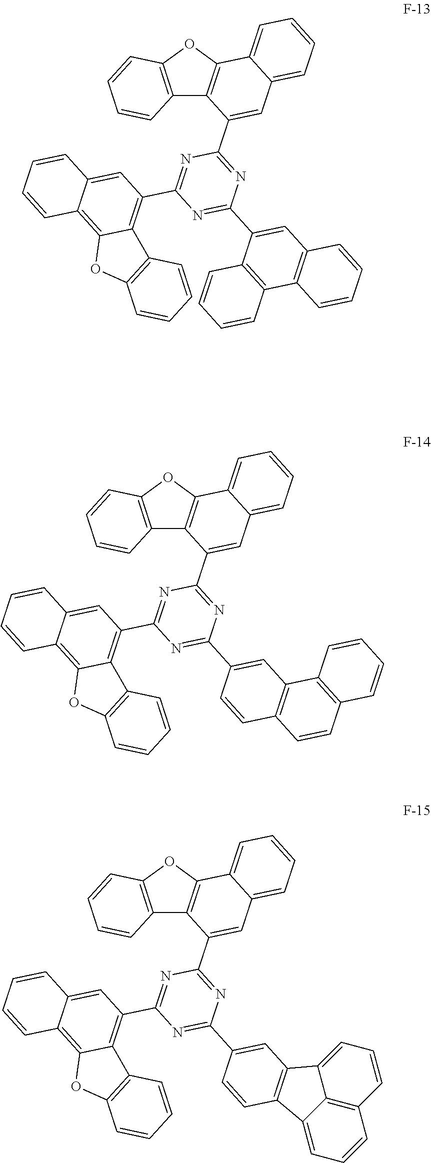 [SMILES: c1ccc2c(c1)cc(-c1nc(-c3cc4ccccc4c4oc5ccccc5c34)nc(-c3cc4ccccc4c4oc5ccccc5c34)n1)c1ccccc12 ; c1ccc2c(c1)ccc1ccc(-c3nc(-c4cc5ccccc5c5oc6ccccc6c45)nc(-c4cc5ccccc5c5oc6ccccc6c45)n3)cc12 ; c1cc2c3c(cccc3c1)-c1cc(-c3nc(-c4cc5ccccc5c5oc6ccccc6c45)nc(-c4cc5ccccc5c5oc6ccccc6c45)n3)ccc1-2]

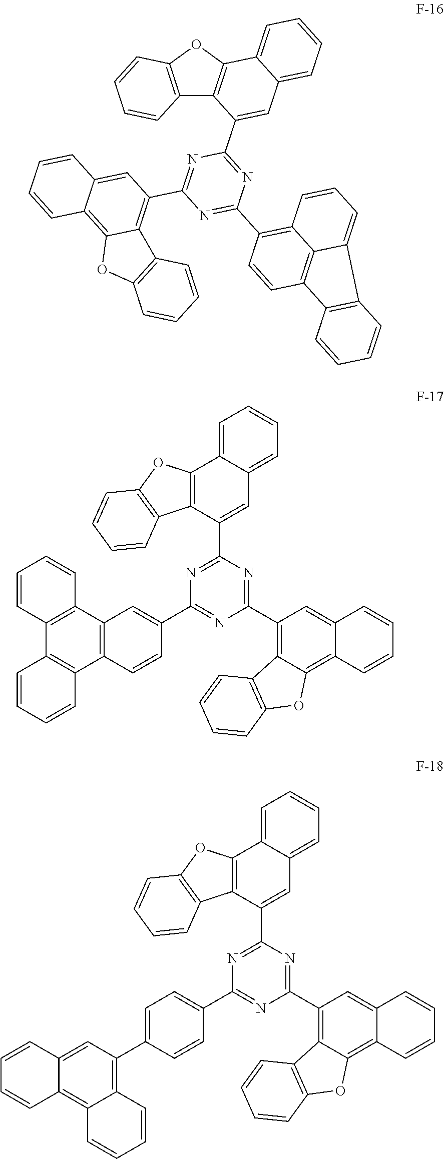 [SMILES: c1ccc2c(c1)-c1cccc3c(-c4nc(-c5cc6ccccc6c6oc7ccccc7c56)nc(-c5cc6ccccc6c6oc7ccccc7c56)n4)ccc-2c13 ; c1ccc2c(c1)cc(-c1nc(-c3ccc4c5ccccc5c5ccccc5c4c3)nc(-c3cc4ccccc4c4oc5ccccc5c34)n1)c1c3ccccc3oc21 ; c1ccc2c(c1)cc(-c1ccc(-c3nc(-c4cc5ccccc5c5oc6ccccc6c45)nc(-c4cc5ccccc5c5oc6ccccc6c45)n3)cc1)c1ccccc12]

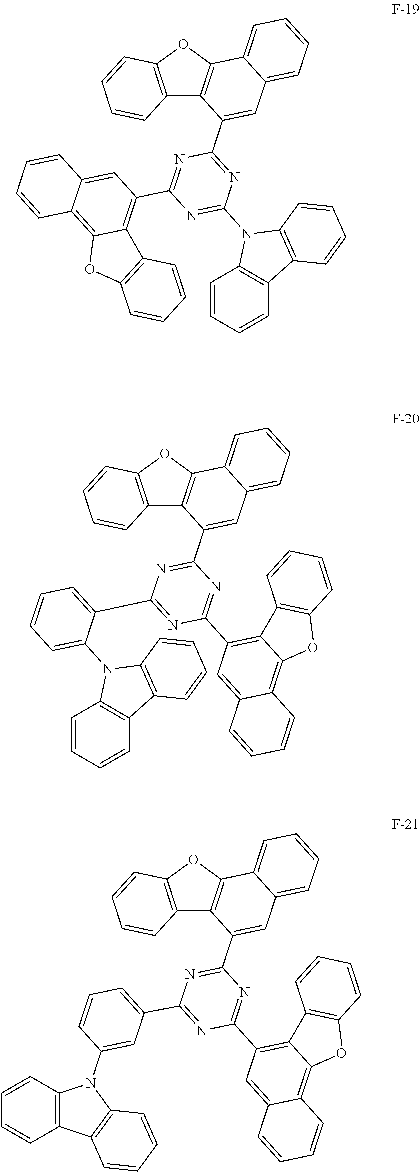 [SMILES: c1ccc2c(c1)cc(-c1nc(-c3cc4ccccc4c4oc5ccccc5c34)nc(-n3c4ccccc4c4ccccc43)n1)c1c3ccccc3oc21 ; c1ccc(-n2c3ccccc3c3ccccc32)c(-c2nc(-c3cc4ccccc4c4oc5ccccc5c34)nc(-c3cc4ccccc4c4oc5ccccc5c34)n2)c1 ; c1cc(-c2nc(-c3cc4ccccc4c4oc5ccccc5c34)nc(-c3cc4ccccc4c4oc5ccccc5c34)n2)cc(-n2c3ccccc3c3ccccc32)c1]

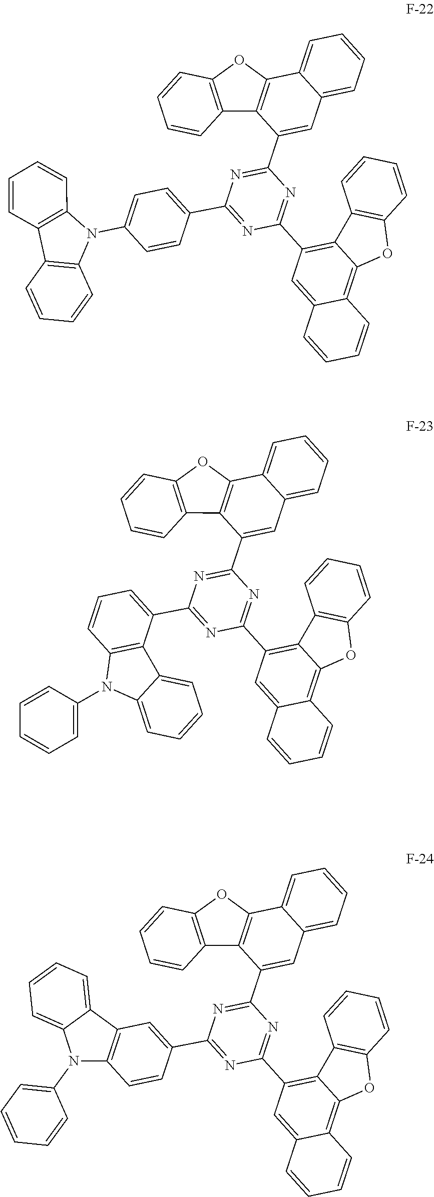 [SMILES: c1ccc2c(c1)cc(-c1nc(-c3ccc(-n4c5ccccc5c5ccccc54)cc3)nc(-c3cc4ccccc4c4oc5ccccc5c34)n1)c1c3ccccc3oc21 ; c1ccc(-n2c3ccccc3c3c(-c4nc(-c5cc6ccccc6c6oc7ccccc7c56)nc(-c5cc6ccccc6c6oc7ccccc7c56)n4)cccc32)cc1 ; c1ccc(-n2c3ccccc3c3cc(-c4nc(-c5cc6ccccc6c6oc7ccccc7c56)nc(-c5cc6ccccc6c6oc7ccccc7c56)n4)ccc32)cc1]

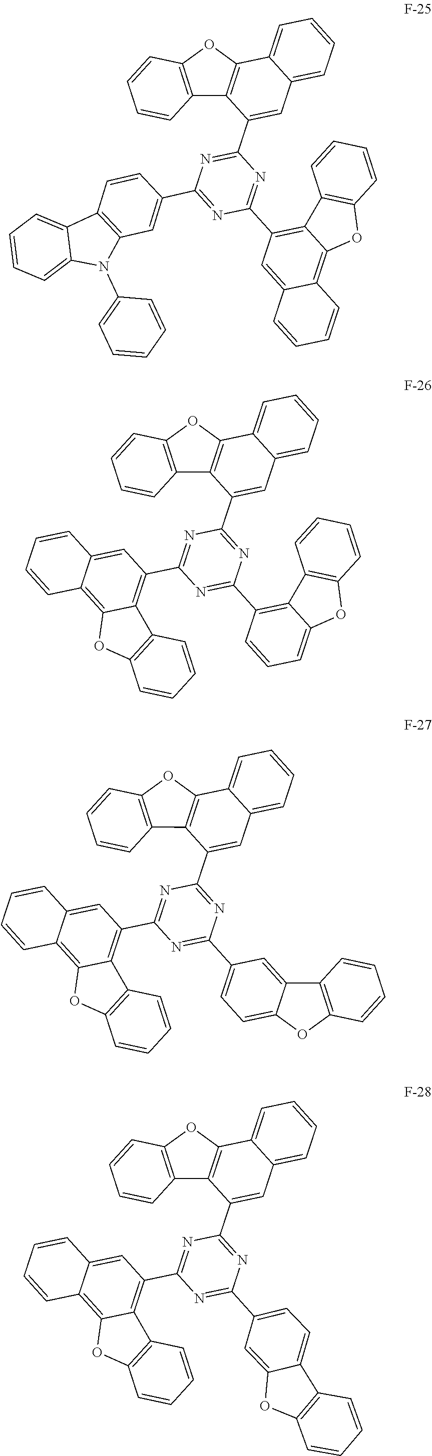 [SMILES: c1ccc(-n2c3ccccc3c3ccc(-c4nc(-c5cc6ccccc6c6oc7ccccc7c56)nc(-c5cc6ccccc6c6oc7ccccc7c56)n4)cc32)cc1 ; c1ccc2c(c1)cc(-c1nc(-c3cccc4oc5ccccc5c34)nc(-c3cc4ccccc4c4oc5ccccc5c34)n1)c1c3ccccc3oc21 ; c1ccc2c(c1)cc(-c1nc(-c3ccc4oc5ccccc5c4c3)nc(-c3cc4ccccc4c4oc5ccccc5c34)n1)c1c3ccccc3oc21 ; c1ccc2c(c1)cc(-c1nc(-c3ccc4c(c3)oc3ccccc34)nc(-c3cc4ccccc4c4oc5ccccc5c34)n1)c1c3ccccc3oc21]

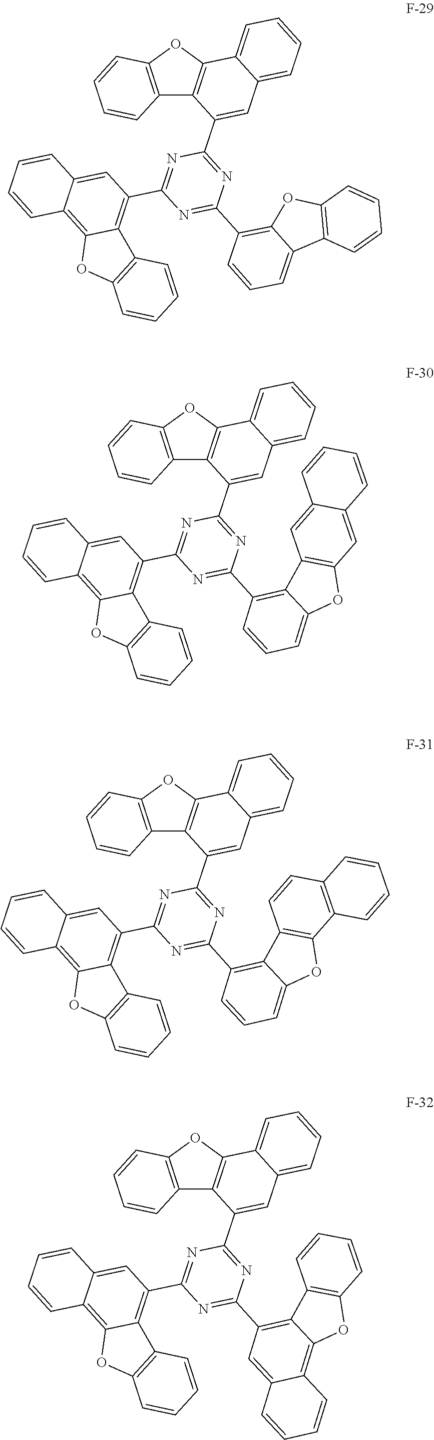 [SMILES: c1ccc2c(c1)cc(-c1nc(-c3cccc4c3oc3ccccc34)nc(-c3cc4ccccc4c4oc5ccccc5c34)n1)c1c3ccccc3oc21 ; c1ccc2cc3c(cc2c1)oc1cccc(-c2nc(-c4cc5ccccc5c5oc6ccccc6c45)nc(-c4cc5ccccc5c5oc6ccccc6c45)n2)c13 ; c1ccc2c(c1)ccc1c2oc2cccc(-c3nc(-c4cc5ccccc5c5oc6ccccc6c45)nc(-c4cc5ccccc5c5oc6ccccc6c45)n3)c21 ; c1ccc2c(c1)cc(-c1nc(-c3cc4ccccc4c4oc5ccccc5c34)nc(-c3cc4ccccc4c4oc5ccccc5c34)n1)c1c3ccccc3oc21]

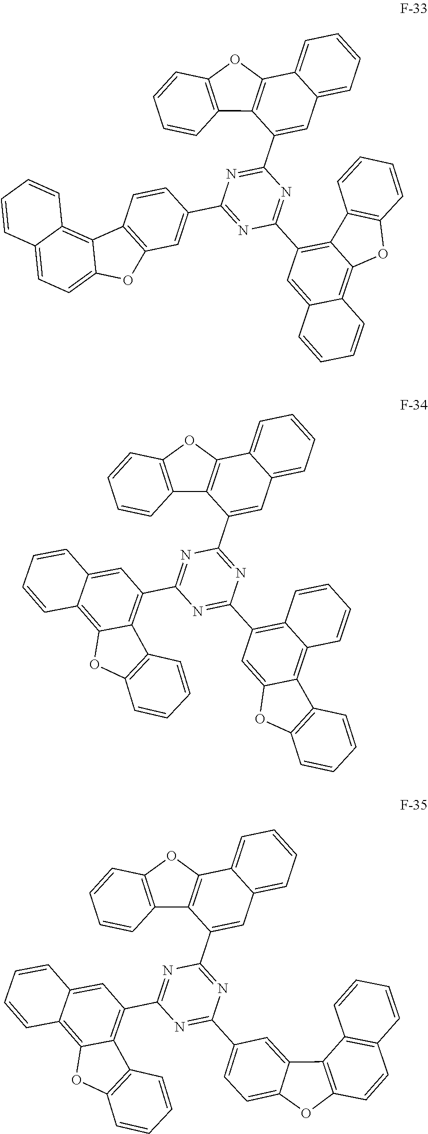 [SMILES: c1ccc2c(c1)cc(-c1nc(-c3ccc4c(c3)oc3ccc5ccccc5c34)nc(-c3cc4ccccc4c4oc5ccccc5c34)n1)c1c3ccccc3oc21 ; c1ccc2c(c1)cc(-c1nc(-c3cc4oc5ccccc5c4c4ccccc34)nc(-c3cc4ccccc4c4oc5ccccc5c34)n1)c1c3ccccc3oc21 ; c1ccc2c(c1)cc(-c1nc(-c3ccc4oc5ccc6ccccc6c5c4c3)nc(-c3cc4ccccc4c4oc5ccccc5c34)n1)c1c3ccccc3oc21]

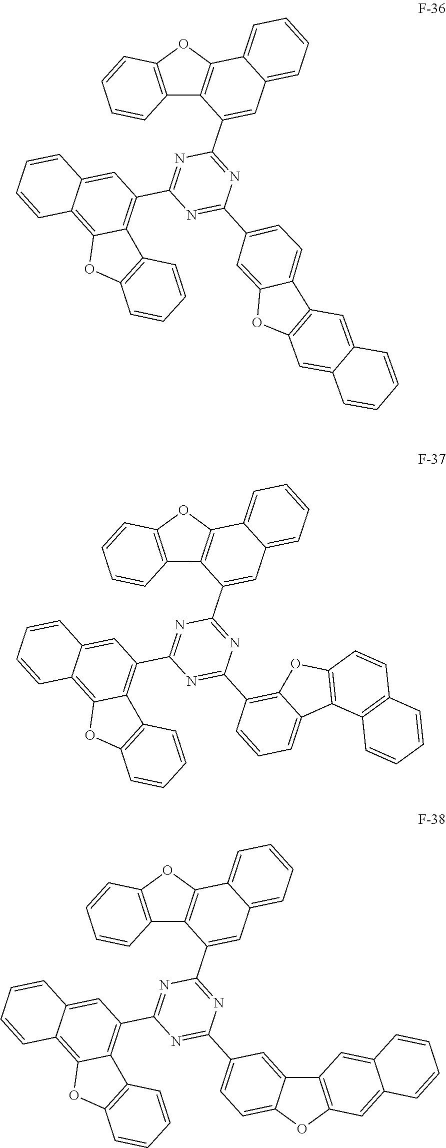 [SMILES: c1ccc2cc3c(cc2c1)oc1cc(-c2nc(-c4cc5ccccc5c5oc6ccccc6c45)nc(-c4cc5ccccc5c5oc6ccccc6c45)n2)ccc13 ; c1ccc2c(c1)cc(-c1nc(-c3cccc4c3oc3ccc5ccccc5c34)nc(-c3cc4ccccc4c4oc5ccccc5c34)n1)c1c3ccccc3oc21 ; c1ccc2cc3c(cc2c1)oc1ccc(-c2nc(-c4cc5ccccc5c5oc6ccccc6c45)nc(-c4cc5ccccc5c5oc6ccccc6c45)n2)cc13]

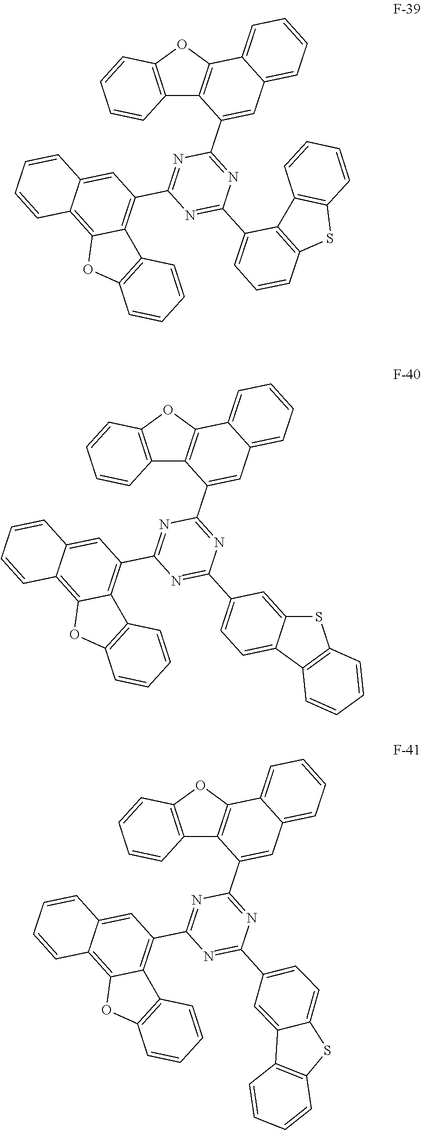 [SMILES: c1ccc2c(c1)cc(-c1nc(-c3cccc4sc5ccccc5c34)nc(-c3cc4ccccc4c4oc5ccccc5c34)n1)c1c3ccccc3oc21 ; c1ccc2c(c1)cc(-c1nc(-c3ccc4c(c3)sc3ccccc34)nc(-c3cc4ccccc4c4oc5ccccc5c34)n1)c1c3ccccc3oc21 ; c1ccc2c(c1)cc(-c1nc(-c3ccc4sc5ccccc5c4c3)nc(-c3cc4ccccc4c4oc5ccccc5c34)n1)c1c3ccccc3oc21]

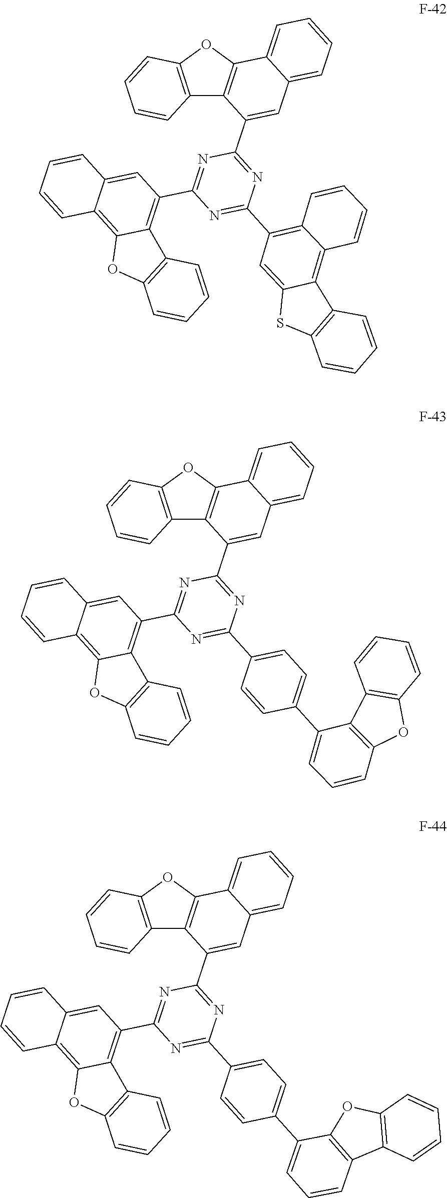 [SMILES: c1ccc2c(c1)cc(-c1nc(-c3cc4sc5ccccc5c4c4ccccc34)nc(-c3cc4ccccc4c4oc5ccccc5c34)n1)c1c3ccccc3oc21 ; c1ccc2c(c1)cc(-c1nc(-c3ccc(-c4cccc5oc6ccccc6c45)cc3)nc(-c3cc4ccccc4c4oc5ccccc5c34)n1)c1c3ccccc3oc21 ; c1ccc2c(c1)cc(-c1nc(-c3ccc(-c4cccc5c4oc4ccccc45)cc3)nc(-c3cc4ccccc4c4oc5ccccc5c34)n1)c1c3ccccc3oc21]

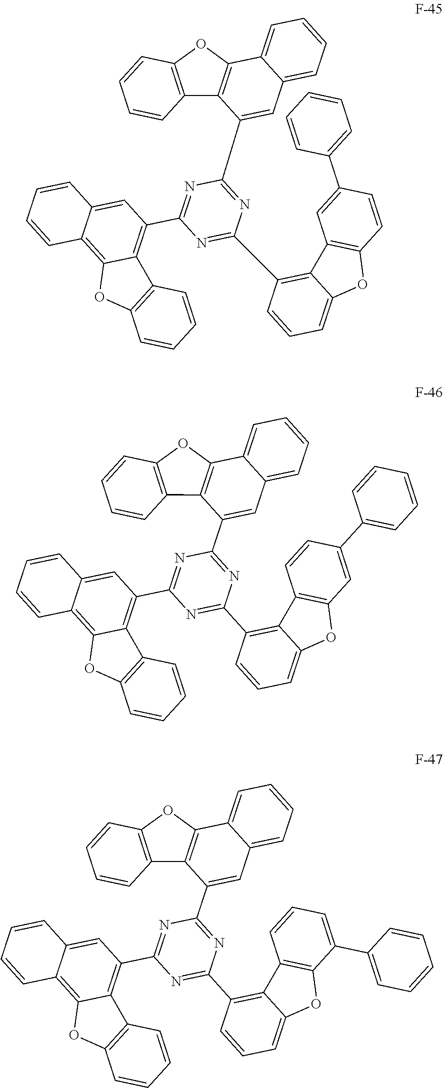 [SMILES: c1ccc(-c2ccc3oc4cccc(-c5nc(-c6cc7ccccc7c7oc8ccccc8c67)nc(-c6cc7ccccc7c7oc8ccccc8c67)n5)c4c3c2)cc1 ; c1ccc(-c2ccc3c(c2)oc2cccc(-c4nc(-c5cc6ccccc6c6oc7ccccc7c56)nc(-c5cc6ccccc6c6oc7ccccc7c56)n4)c23)cc1 ; c1ccc(-c2cccc3c2oc2cccc(-c4nc(-c5cc6ccccc6c6oc7ccccc7c56)nc(-c5cc6ccccc6c6oc7ccccc7c56)n4)c23)cc1]

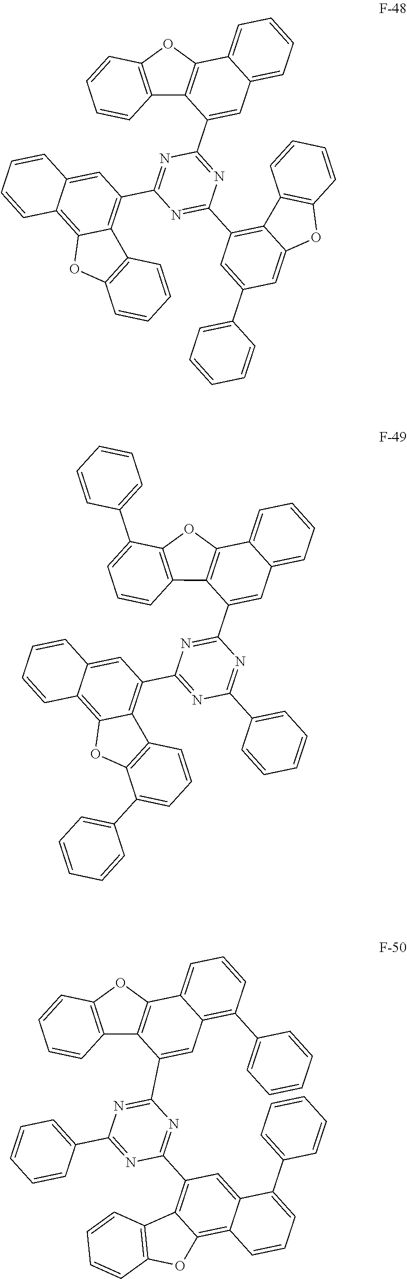 [SMILES: c1ccc(-c2cc(-c3nc(-c4cc5ccccc5c5oc6ccccc6c45)nc(-c4cc5ccccc5c5oc6ccccc6c45)n3)c3c(c2)oc2ccccc23)cc1 ; c1ccc(-c2nc(-c3cc4ccccc4c4oc5c(-c6ccccc6)cccc5c34)nc(-c3cc4ccccc4c4oc5c(-c6ccccc6)cccc5c34)n2)cc1 ; c1ccc(-c2nc(-c3cc4c(-c5ccccc5)cccc4c4oc5ccccc5c34)nc(-c3cc4c(-c5ccccc5)cccc4c4oc5ccccc5c34)n2)cc1]

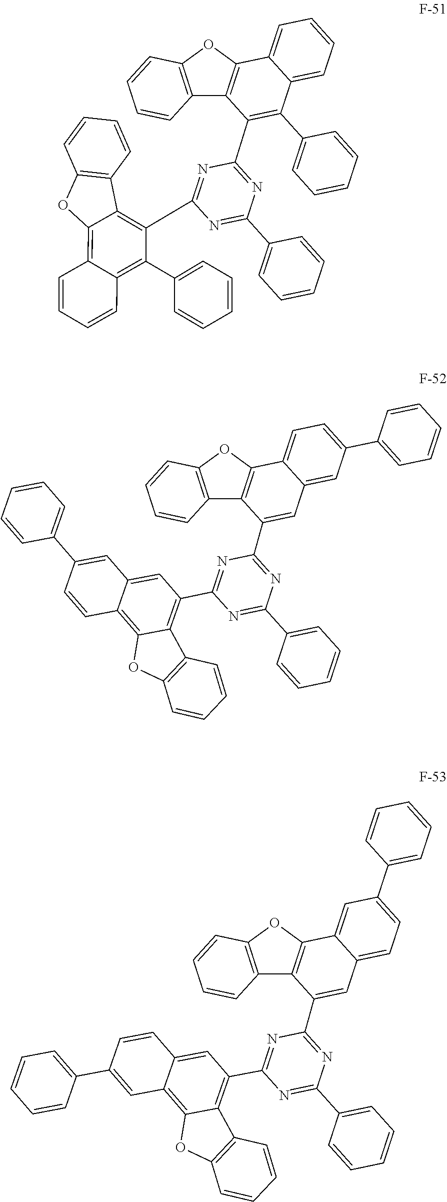 [SMILES: c1ccc(-c2nc(-c3c(-c4ccccc4)c4ccccc4c4oc5ccccc5c34)nc(-c3c(-c4ccccc4)c4ccccc4c4oc5ccccc5c34)n2)cc1 ; c1ccc(-c2ccc3c(c2)cc(-c2nc(-c4ccccc4)nc(-c4cc5cc(-c6ccccc6)ccc5c5oc6ccccc6c45)n2)c2c4ccccc4oc32)cc1 ; c1ccc(-c2ccc3cc(-c4nc(-c5ccccc5)nc(-c5cc6ccc(-c7ccccc7)cc6c6oc7ccccc7c56)n4)c4c5ccccc5oc4c3c2)cc1]

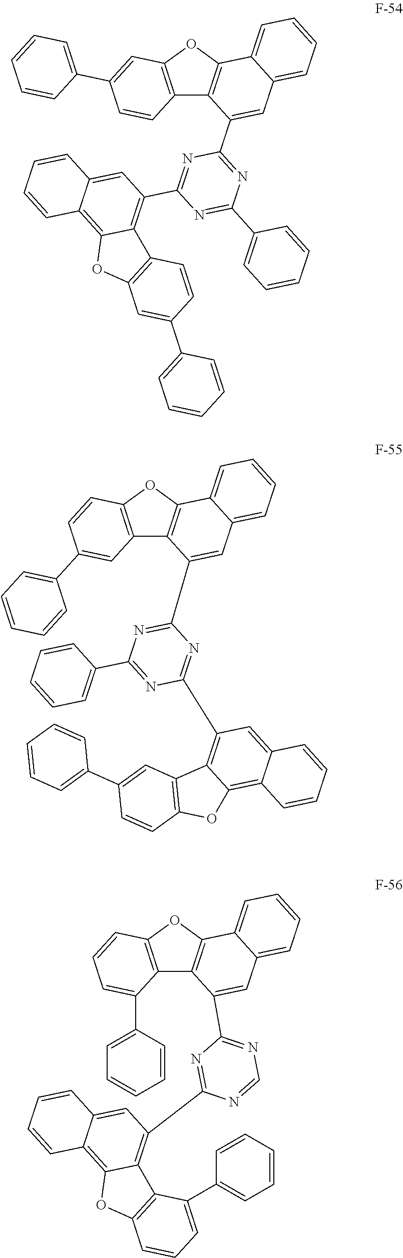 [SMILES: c1ccc(-c2ccc3c(c2)oc2c4ccccc4cc(-c4nc(-c5ccccc5)nc(-c5cc6ccccc6c6oc7cc(-c8ccccc8)ccc7c56)n4)c32)cc1 ; c1ccc(-c2ccc3oc4c5ccccc5cc(-c5nc(-c6ccccc6)nc(-c6cc7ccccc7c7oc8ccc(-c9ccccc9)cc8c67)n5)c4c3c2)cc1 ; c1ccc(-c2cccc3oc4c5ccccc5cc(-c5ncnc(-c6cc7ccccc7c7oc8cccc(-c9ccccc9)c8c67)n5)c4c23)cc1]

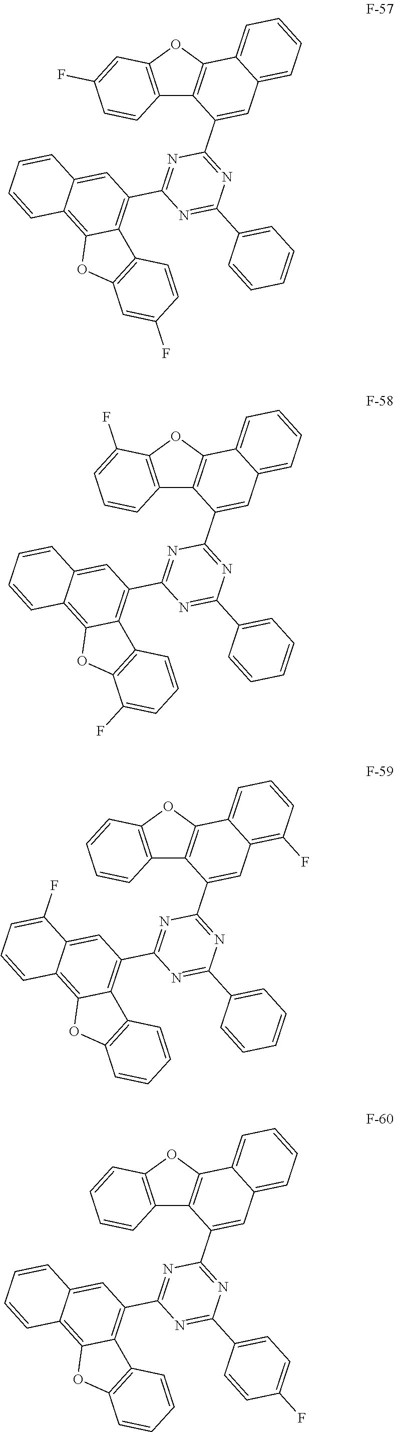 [SMILES: Fc1ccc2c(c1)oc1c3ccccc3cc(-c3nc(-c4ccccc4)nc(-c4cc5ccccc5c5oc6cc(F)ccc6c45)n3)c21 ; Fc1cccc2c1oc1c3ccccc3cc(-c3nc(-c4ccccc4)nc(-c4cc5ccccc5c5oc6c(F)cccc6c45)n3)c21 ; Fc1cccc2c1cc(-c1nc(-c3ccccc3)nc(-c3cc4c(F)cccc4c4oc5ccccc5c34)n1)c1c3ccccc3oc21 ; Fc1ccc(-c2nc(-c3cc4ccccc4c4oc5ccccc5c34)nc(-c3cc4ccccc4c4oc5ccccc5c34)n2)cc1]

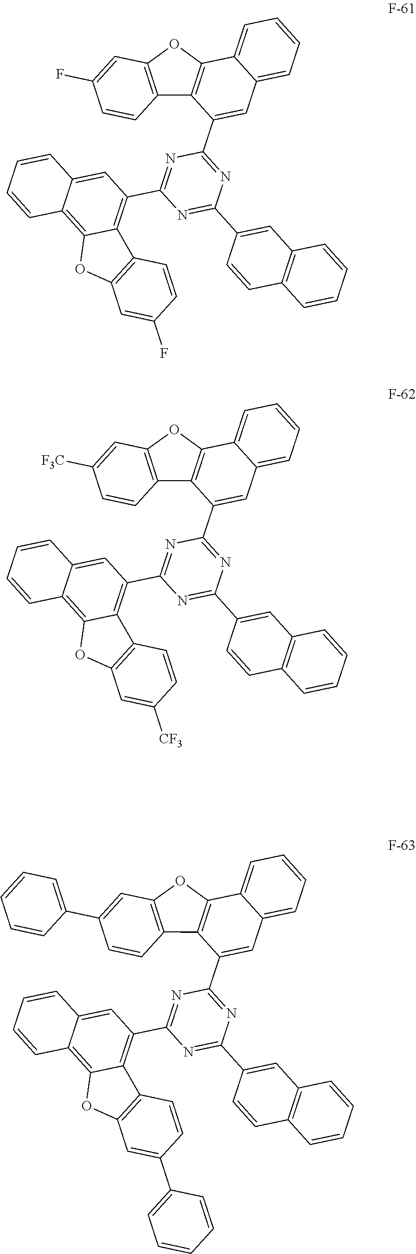 [SMILES: Fc1ccc2c(c1)oc1c3ccccc3cc(-c3nc(-c4ccc5ccccc5c4)nc(-c4cc5ccccc5c5oc6cc(F)ccc6c45)n3)c21 ; FC(F)(F)c1ccc2c(c1)oc1c3ccccc3cc(-c3nc(-c4ccc5ccccc5c4)nc(-c4cc5ccccc5c5oc6cc(C(F)(F)F)ccc6c45)n3)c21 ; c1ccc(-c2ccc3c(c2)oc2c4ccccc4cc(-c4nc(-c5ccc6ccccc6c5)nc(-c5cc6ccccc6c6oc7cc(-c8ccccc8)ccc7c56)n4)c32)cc1]

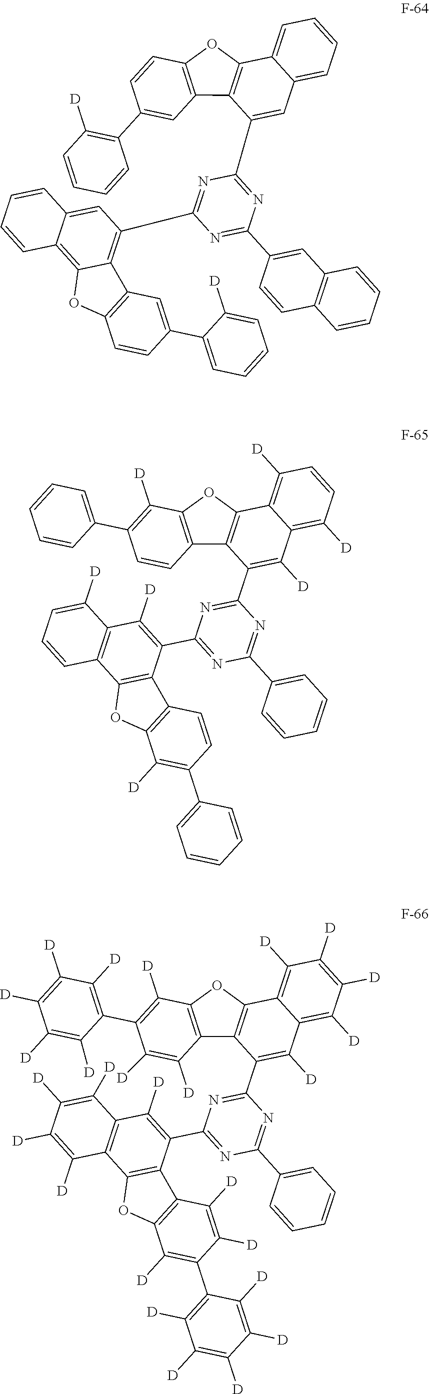 [SMILES: [2H]c1ccccc1-c1ccc2oc3c4ccccc4cc(-c4nc(-c5ccc6ccccc6c5)nc(-c5cc6ccccc6c6oc7ccc(-c8ccccc8[2H])cc7c56)n4)c3c2c1 ; [2H]c1c(-c2ccccc2)ccc2c1oc1c3cccc([2H])c3c([2H])c(-c3nc(-c4ccccc4)nc(-c4c([2H])c5c([2H])ccc([2H])c5c5oc6c([2H])c(-c7ccccc7)ccc6c45)n3)c21 ; [2H]c1c([2H])c([2H])c(-c2c([2H])c([2H])c3c(oc4c5c([2H])c([2H])c([2H])c([2H])c5c([2H])c(-c5nc(-c6ccccc6)nc(-c6c([2H])c7c([2H])c([2H])c([2H])c([2H])c7c7oc8c([2H])c(-c9c([2H])c([2H])c([2H])c([2H])c9[2H])c([2H])c([2H])c8c67)n5)c43)c2[2H])c([2H])c1[2H]]

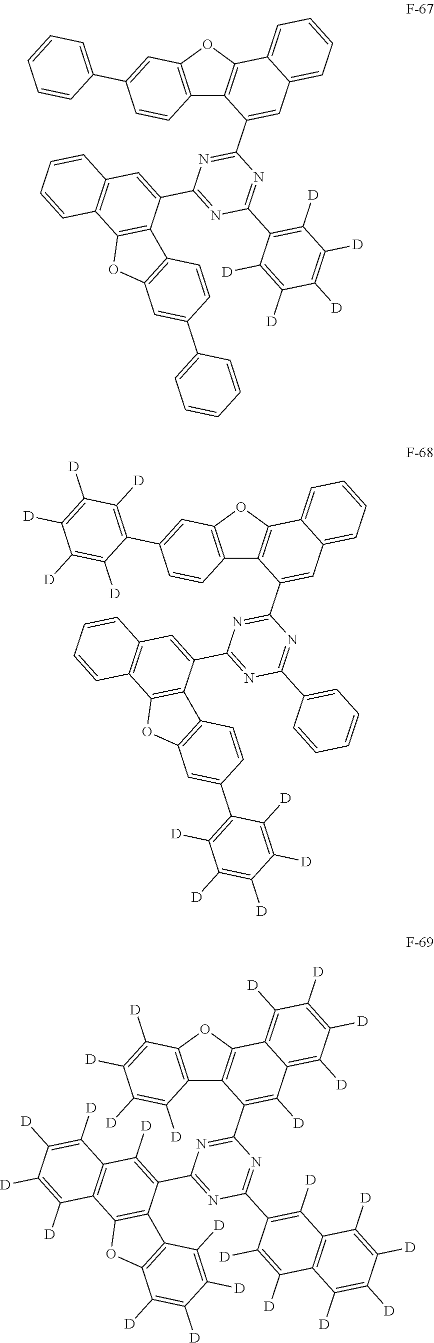

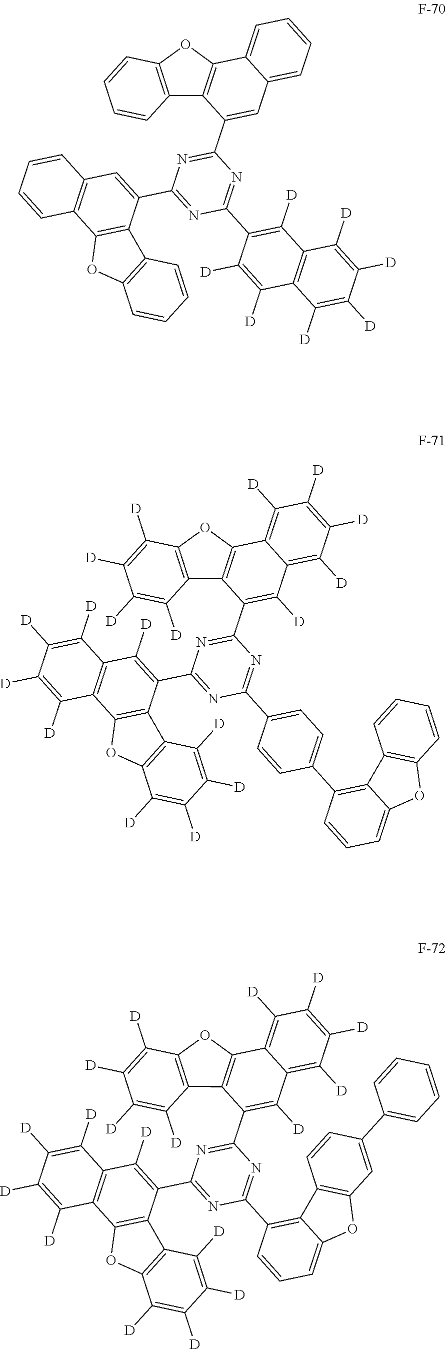

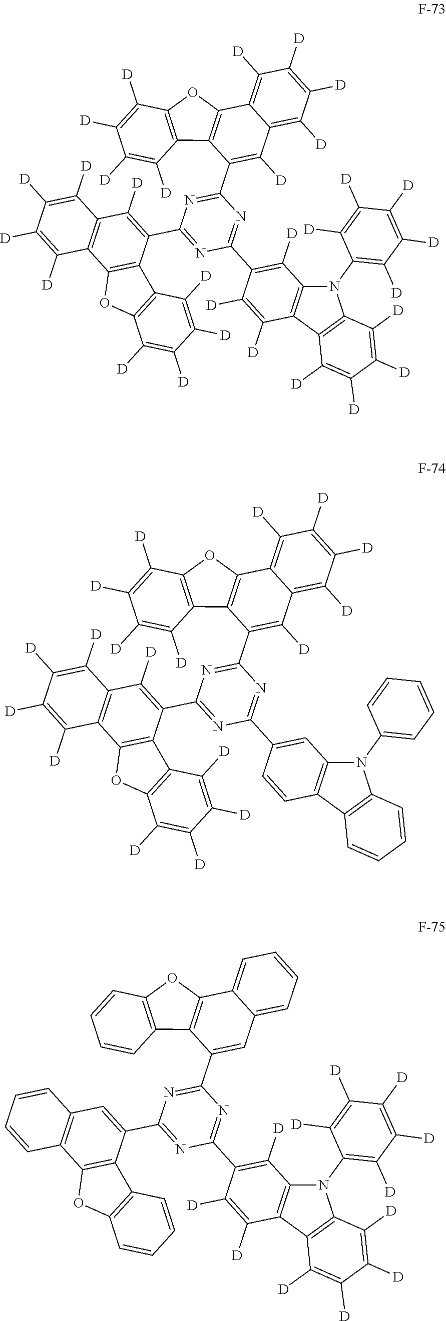

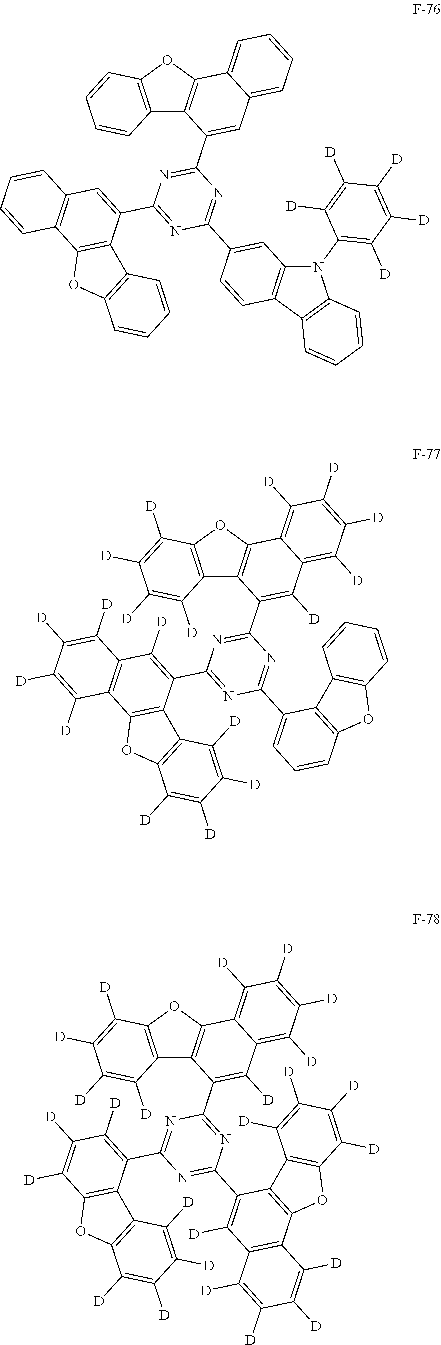

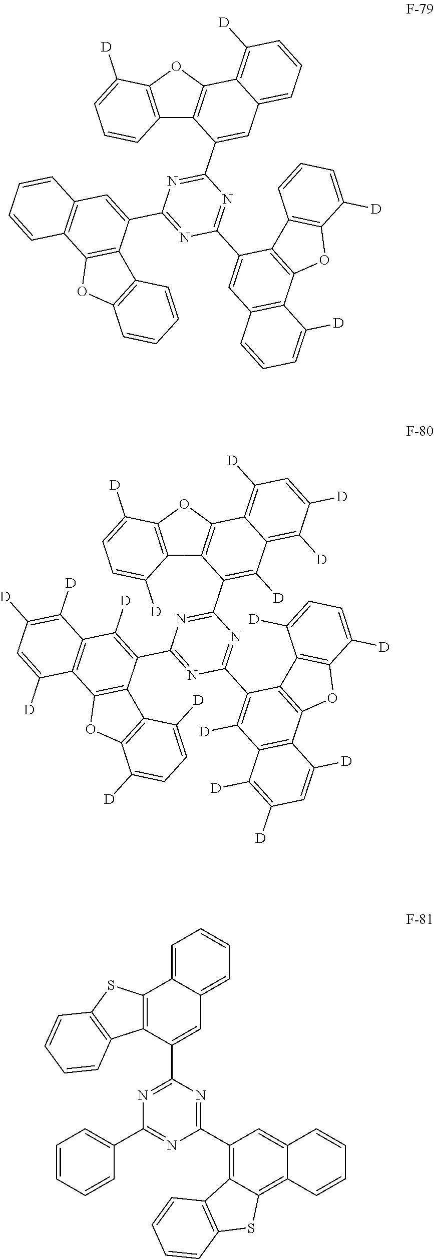 [SMILES: [2H]c1cccc2c1oc1c3c([2H])cccc3cc(-c3nc(-c4cc5ccccc5c5oc6ccccc6c45)nc(-c4cc5cccc([2H])c5c5oc6c([2H])cccc6c45)n3)c21 ; [2H]c1cc([2H])c2c(c1[2H])c([2H])c(-c1nc(-c3c([2H])c4c([2H])c([2H])cc([2H])c4c4oc5c([2H])ccc([2H])c5c34)nc(-c3c([2H])c4c([2H])c([2H])cc([2H])c4c4oc5c([2H])ccc([2H])c5c34)n1)c1c2oc2c([2H])ccc([2H])c21 ; c1ccc(-c2nc(-c3cc4ccccc4c4sc5ccccc5c34)nc(-c3cc4ccccc4c4sc5ccccc5c34)n2)cc1]

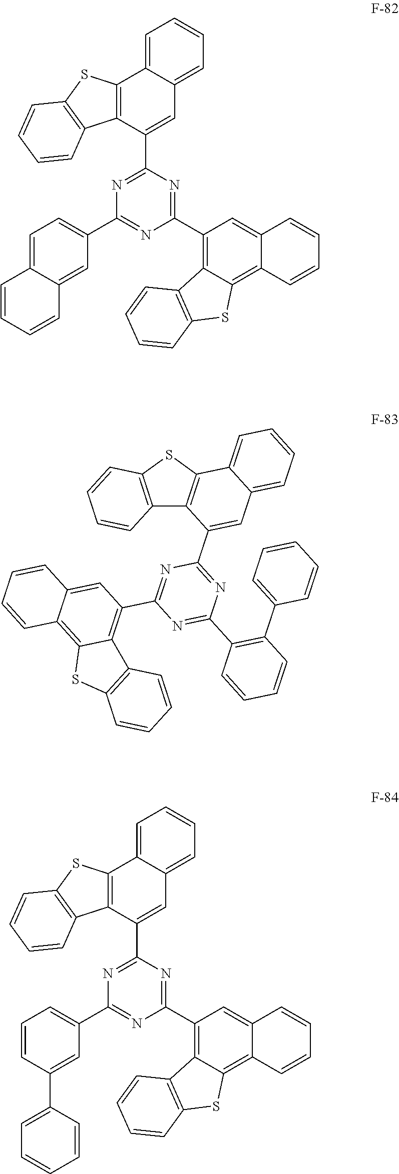 [SMILES: c1ccc2cc(-c3nc(-c4cc5ccccc5c5sc6ccccc6c45)nc(-c4cc5ccccc5c5sc6ccccc6c45)n3)ccc2c1 ; c1ccc(-c2ccccc2-c2nc(-c3cc4ccccc4c4sc5ccccc5c34)nc(-c3cc4ccccc4c4sc5ccccc5c34)n2)cc1 ; c1ccc(-c2cccc(-c3nc(-c4cc5ccccc5c5sc6ccccc6c45)nc(-c4cc5ccccc5c5sc6ccccc6c45)n3)c2)cc1]

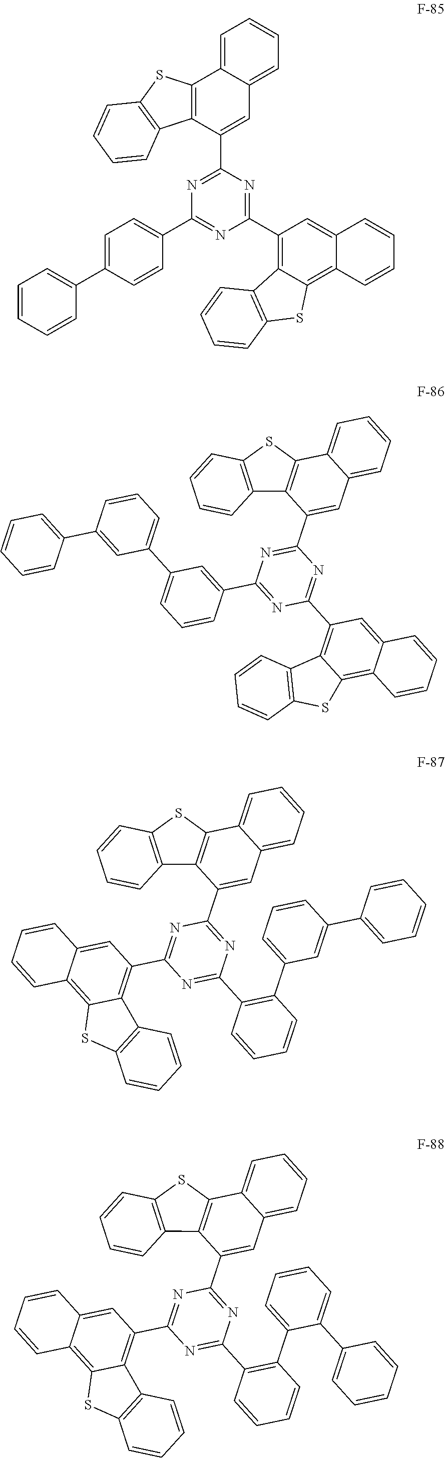 [SMILES: c1ccc(-c2ccc(-c3nc(-c4cc5ccccc5c5sc6ccccc6c45)nc(-c4cc5ccccc5c5sc6ccccc6c45)n3)cc2)cc1 ; c1ccc(-c2cccc(-c3cccc(-c4nc(-c5cc6ccccc6c6sc7ccccc7c56)nc(-c5cc6ccccc6c6sc7ccccc7c56)n4)c3)c2)cc1 ; c1ccc(-c2cccc(-c3ccccc3-c3nc(-c4cc5ccccc5c5sc6ccccc6c45)nc(-c4cc5ccccc5c5sc6ccccc6c45)n3)c2)cc1 ; c1ccc(-c2ccccc2-c2ccccc2-c2nc(-c3cc4ccccc4c4sc5ccccc5c34)nc(-c3cc4ccccc4c4sc5ccccc5c34)n2)cc1]

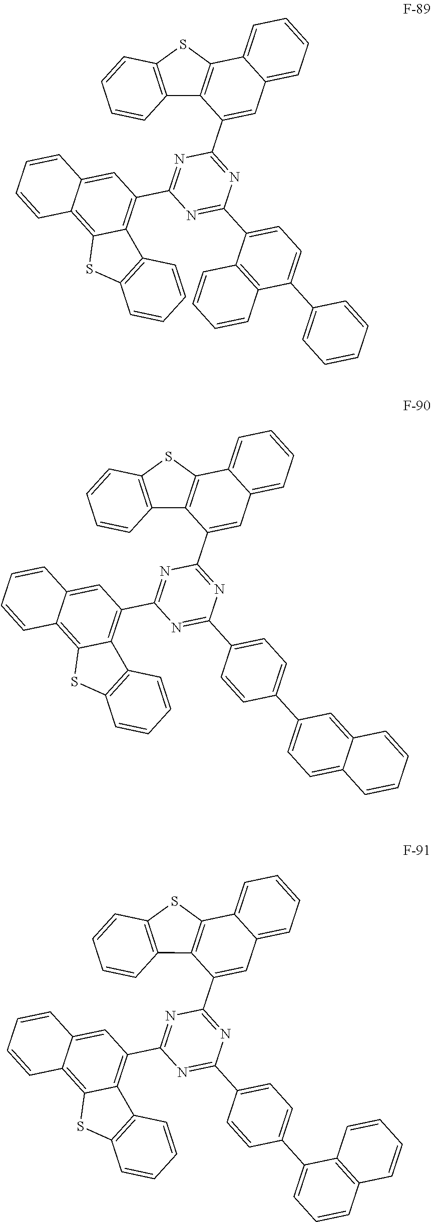 [SMILES: c1ccc(-c2ccc(-c3nc(-c4cc5ccccc5c5sc6ccccc6c45)nc(-c4cc5ccccc5c5sc6ccccc6c45)n3)c3ccccc23)cc1 ; c1ccc2cc(-c3ccc(-c4nc(-c5cc6ccccc6c6sc7ccccc7c56)nc(-c5cc6ccccc6c6sc7ccccc7c56)n4)cc3)ccc2c1 ; c1ccc2c(-c3ccc(-c4nc(-c5cc6ccccc6c6sc7ccccc7c56)nc(-c5cc6ccccc6c6sc7ccccc7c56)n4)cc3)cccc2c1]

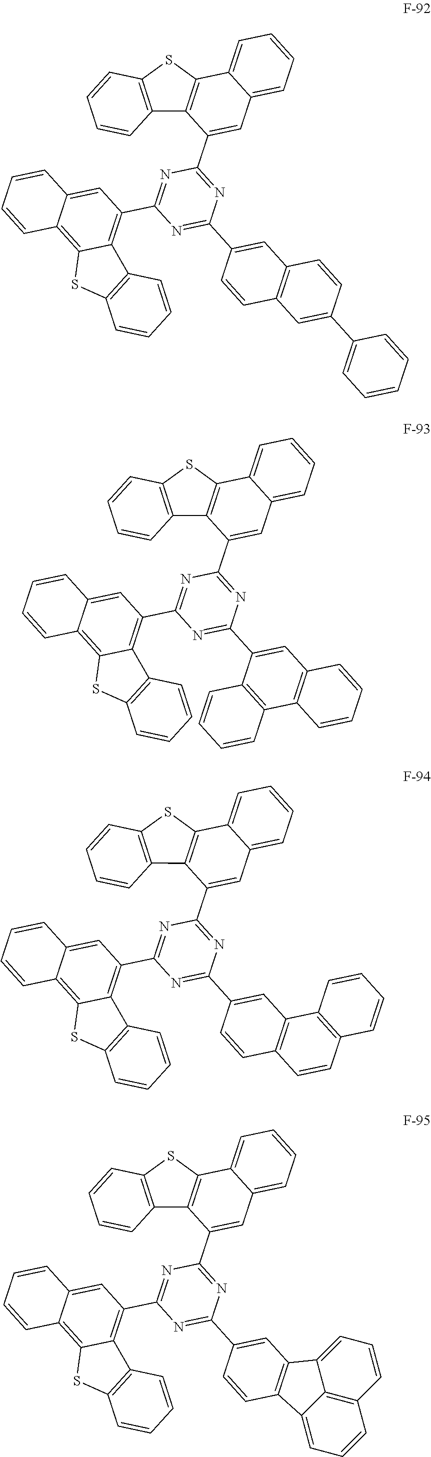 [SMILES: c1ccc(-c2ccc3cc(-c4nc(-c5cc6ccccc6c6sc7ccccc7c56)nc(-c5cc6ccccc6c6sc7ccccc7c56)n4)ccc3c2)cc1 ; c1ccc2c(c1)cc(-c1nc(-c3cc4ccccc4c4sc5ccccc5c34)nc(-c3cc4ccccc4c4sc5ccccc5c34)n1)c1ccccc12 ; c1ccc2c(c1)ccc1ccc(-c3nc(-c4cc5ccccc5c5sc6ccccc6c45)nc(-c4cc5ccccc5c5sc6ccccc6c45)n3)cc12 ; c1cc2c3c(cccc3c1)-c1cc(-c3nc(-c4cc5ccccc5c5sc6ccccc6c45)nc(-c4cc5ccccc5c5sc6ccccc6c45)n3)ccc1-2]

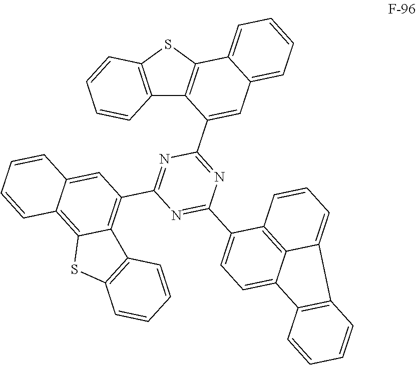 [SMILES: c1ccc2c(c1)-c1cccc3c(-c4nc(-c5cc6ccccc6c6sc7ccccc7c56)nc(-c5cc6ccccc6c6sc7ccccc7c56)n4)ccc-2c13]

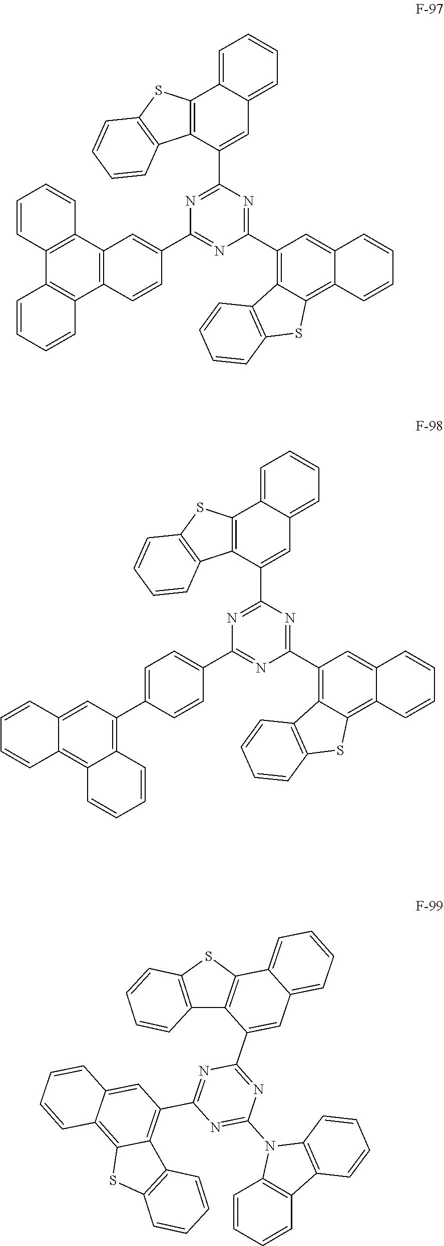 [SMILES: c1ccc2c(c1)cc(-c1nc(-c3ccc4c5ccccc5c5ccccc5c4c3)nc(-c3cc4ccccc4c4sc5ccccc5c34)n1)c1c3ccccc3sc21 ; c1ccc2c(c1)cc(-c1ccc(-c3nc(-c4cc5ccccc5c5sc6ccccc6c45)nc(-c4cc5ccccc5c5sc6ccccc6c45)n3)cc1)c1ccccc12 ; c1ccc2c(c1)cc(-c1nc(-c3cc4ccccc4c4sc5ccccc5c34)nc(-n3c4ccccc4c4ccccc43)n1)c1c3ccccc3sc21]

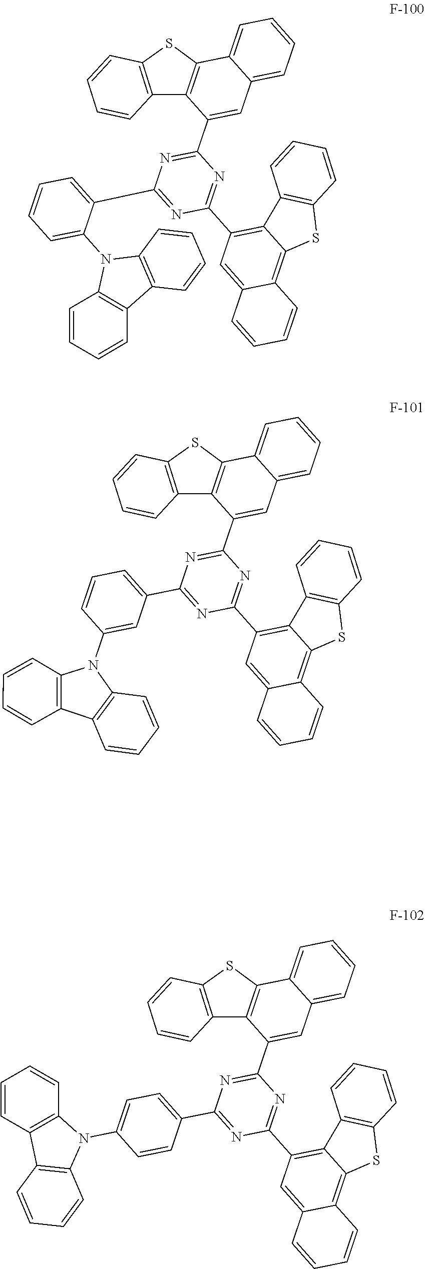 [SMILES: c1ccc(-n2c3ccccc3c3ccccc32)c(-c2nc(-c3cc4ccccc4c4sc5ccccc5c34)nc(-c3cc4ccccc4c4sc5ccccc5c34)n2)c1 ; c1cc(-c2nc(-c3cc4ccccc4c4sc5ccccc5c34)nc(-c3cc4ccccc4c4sc5ccccc5c34)n2)cc(-n2c3ccccc3c3ccccc32)c1 ; c1ccc2c(c1)cc(-c1nc(-c3ccc(-n4c5ccccc5c5ccccc54)cc3)nc(-c3cc4ccccc4c4sc5ccccc5c34)n1)c1c3ccccc3sc21]

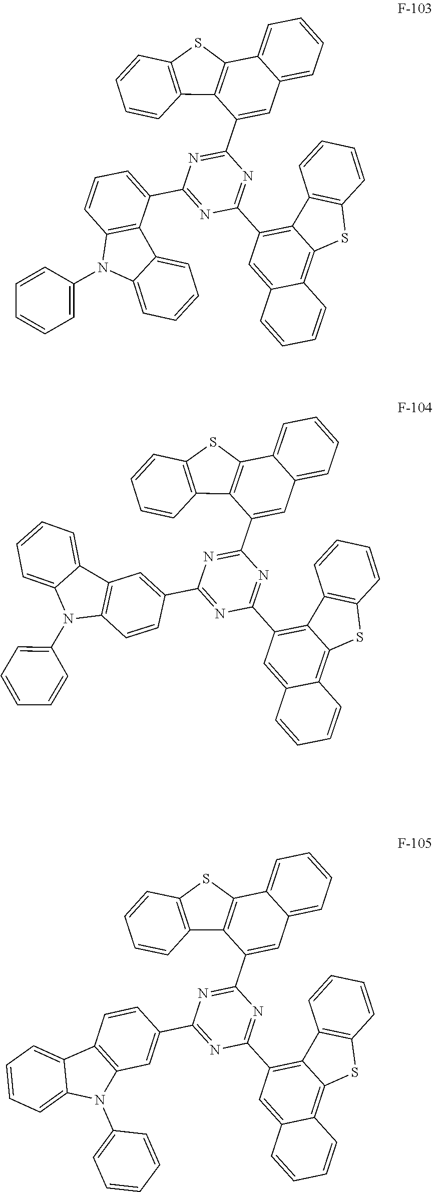 [SMILES: c1ccc(-n2c3ccccc3c3c(-c4nc(-c5cc6ccccc6c6sc7ccccc7c56)nc(-c5cc6ccccc6c6sc7ccccc7c56)n4)cccc32)cc1 ; c1ccc(-n2c3ccccc3c3cc(-c4nc(-c5cc6ccccc6c6sc7ccccc7c56)nc(-c5cc6ccccc6c6sc7ccccc7c56)n4)ccc32)cc1 ; c1ccc(-n2c3ccccc3c3ccc(-c4nc(-c5cc6ccccc6c6sc7ccccc7c56)nc(-c5cc6ccccc6c6sc7ccccc7c56)n4)cc32)cc1]

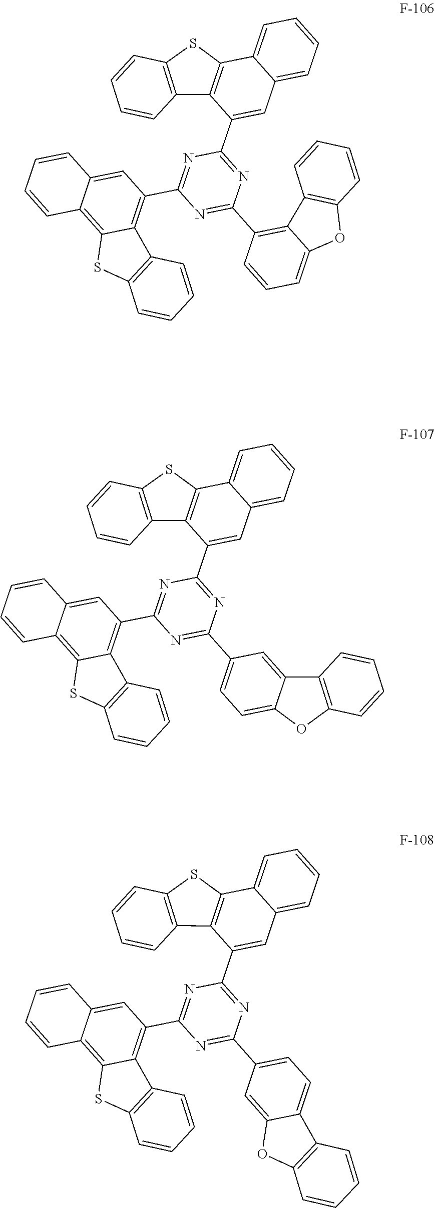 [SMILES: c1ccc2c(c1)cc(-c1nc(-c3cccc4oc5ccccc5c34)nc(-c3cc4ccccc4c4sc5ccccc5c34)n1)c1c3ccccc3sc21 ; c1ccc2c(c1)cc(-c1nc(-c3ccc4oc5ccccc5c4c3)nc(-c3cc4ccccc4c4sc5ccccc5c34)n1)c1c3ccccc3sc21 ; c1ccc2c(c1)cc(-c1nc(-c3ccc4c(c3)oc3ccccc34)nc(-c3cc4ccccc4c4sc5ccccc5c34)n1)c1c3ccccc3sc21]

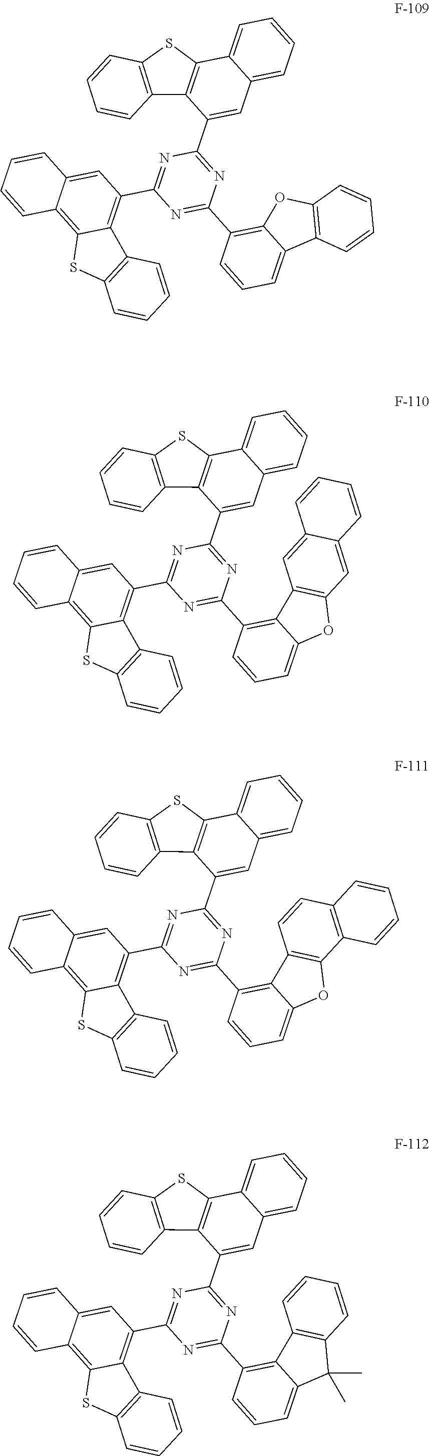 [SMILES: c1ccc2c(c1)cc(-c1nc(-c3cccc4c3oc3ccccc34)nc(-c3cc4ccccc4c4sc5ccccc5c34)n1)c1c3ccccc3sc21 ; c1ccc2cc3c(cc2c1)oc1cccc(-c2nc(-c4cc5ccccc5c5sc6ccccc6c45)nc(-c4cc5ccccc5c5sc6ccccc6c45)n2)c13 ; c1ccc2c(c1)ccc1c2oc2cccc(-c3nc(-c4cc5ccccc5c5sc6ccccc6c45)nc(-c4cc5ccccc5c5sc6ccccc6c45)n3)c21 ; CC1(C)c2ccccc2-c2c(-c3nc(-c4cc5ccccc5c5sc6ccccc6c45)nc(-c4cc5ccccc5c5sc6ccccc6c45)n3)cccc21]

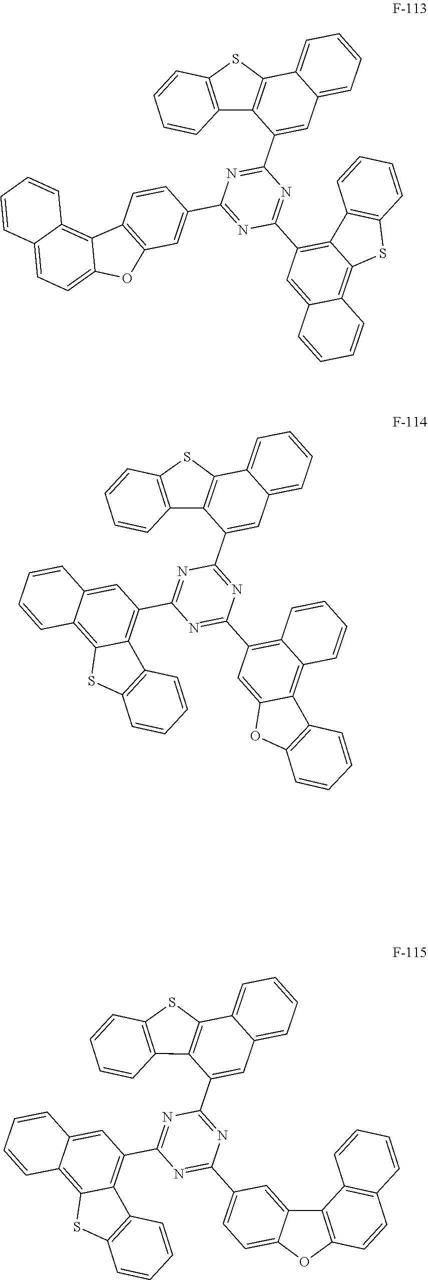 [SMILES: c1ccc2c(c1)cc(-c1nc(-c3ccc4c(c3)oc3ccc5ccccc5c34)nc(-c3cc4ccccc4c4sc5ccccc5c34)n1)c1c3ccccc3sc21 ; c1ccc2c(c1)cc(-c1nc(-c3cc4oc5ccccc5c4c4ccccc34)nc(-c3cc4ccccc4c4sc5ccccc5c34)n1)c1c3ccccc3sc21 ; c1ccc2c(c1)cc(-c1nc(-c3ccc4oc5ccc6ccccc6c5c4c3)nc(-c3cc4ccccc4c4sc5ccccc5c34)n1)c1c3ccccc3sc21]

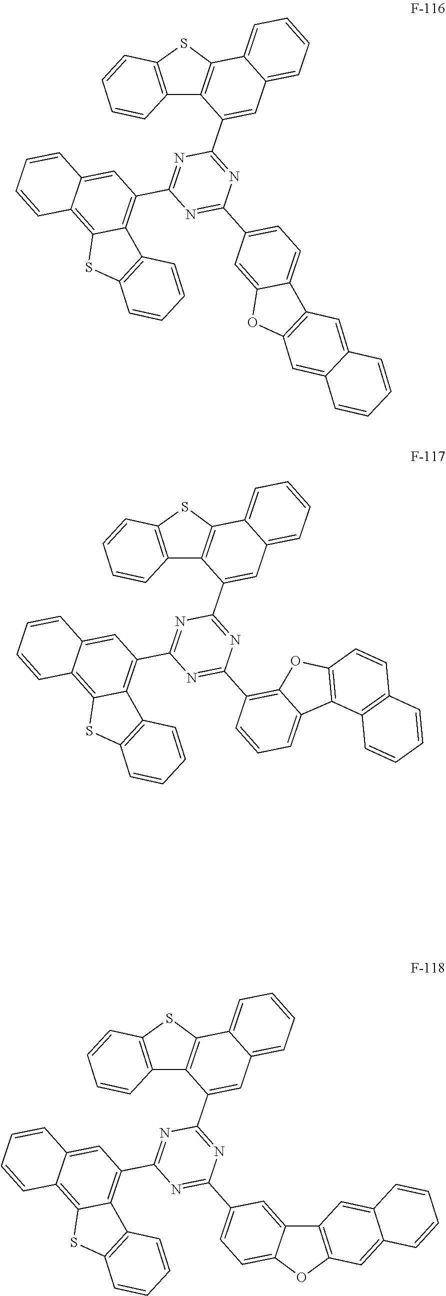 [SMILES: c1ccc2cc3c(cc2c1)oc1cc(-c2nc(-c4cc5ccccc5c5sc6ccccc6c45)nc(-c4cc5ccccc5c5sc6ccccc6c45)n2)ccc13 ; c1ccc2c(c1)cc(-c1nc(-c3cccc4c3oc3ccc5ccccc5c34)nc(-c3cc4ccccc4c4sc5ccccc5c34)n1)c1c3ccccc3sc21 ; c1ccc2cc3c(cc2c1)oc1ccc(-c2nc(-c4cc5ccccc5c5sc6ccccc6c45)nc(-c4cc5ccccc5c5sc6ccccc6c45)n2)cc13]

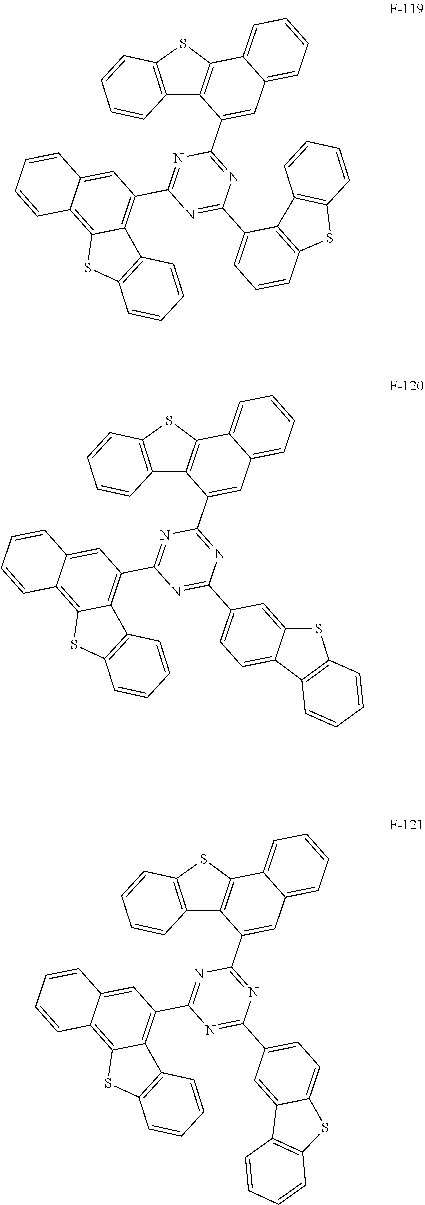 [SMILES: c1ccc2c(c1)cc(-c1nc(-c3cccc4sc5ccccc5c34)nc(-c3cc4ccccc4c4sc5ccccc5c34)n1)c1c3ccccc3sc21 ; c1ccc2c(c1)cc(-c1nc(-c3ccc4c(c3)sc3ccccc34)nc(-c3cc4ccccc4c4sc5ccccc5c34)n1)c1c3ccccc3sc21 ; c1ccc2c(c1)cc(-c1nc(-c3ccc4sc5ccccc5c4c3)nc(-c3cc4ccccc4c4sc5ccccc5c34)n1)c1c3ccccc3sc21]

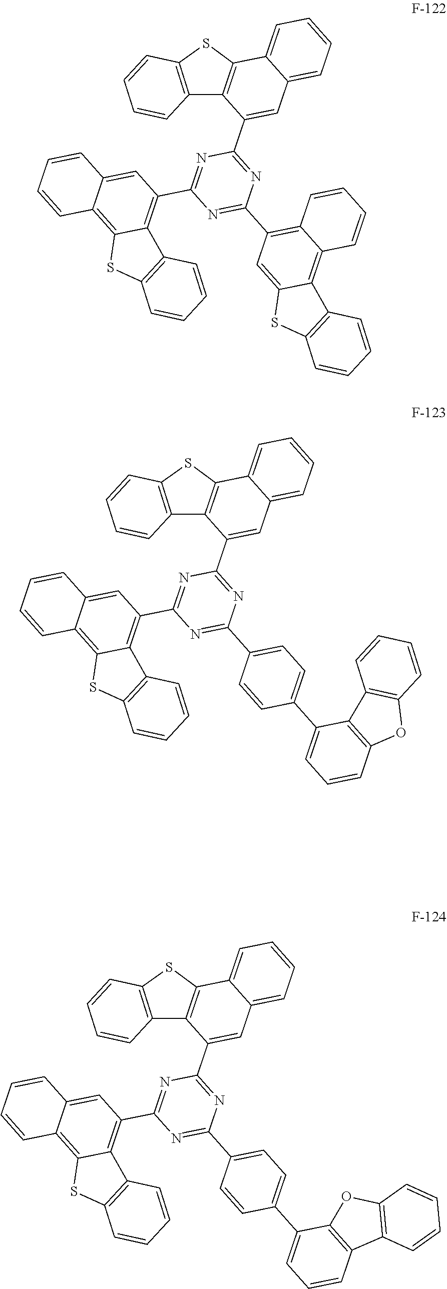 [SMILES: c1ccc2c(c1)cc(-c1nc(-c3cc4sc5ccccc5c4c4ccccc34)nc(-c3cc4ccccc4c4sc5ccccc5c34)n1)c1c3ccccc3sc21 ; c1ccc2c(c1)cc(-c1nc(-c3ccc(-c4cccc5oc6ccccc6c45)cc3)nc(-c3cc4ccccc4c4sc5ccccc5c34)n1)c1c3ccccc3sc21 ; c1ccc2c(c1)cc(-c1nc(-c3ccc(-c4cccc5c4oc4ccccc45)cc3)nc(-c3cc4ccccc4c4sc5ccccc5c34)n1)c1c3ccccc3sc21]

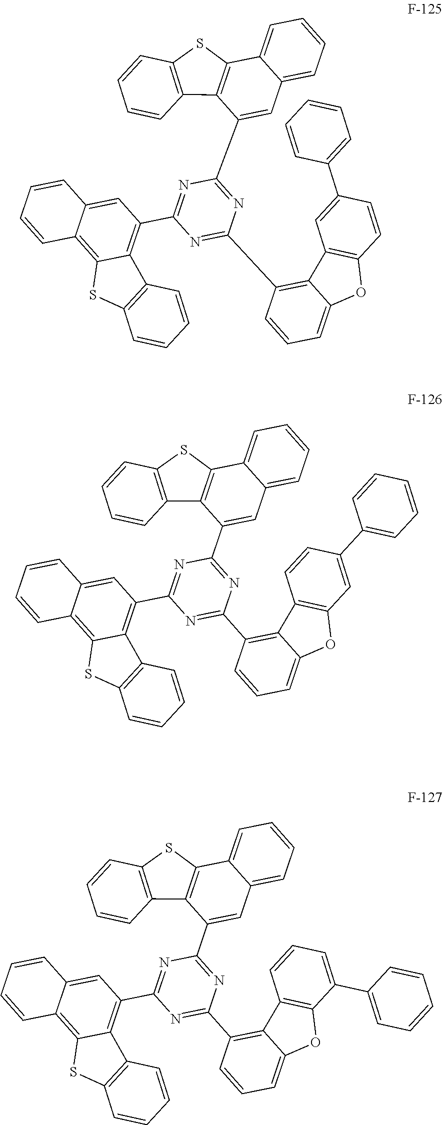 [SMILES: c1ccc(-c2ccc3oc4cccc(-c5nc(-c6cc7ccccc7c7sc8ccccc8c67)nc(-c6cc7ccccc7c7sc8ccccc8c67)n5)c4c3c2)cc1 ; c1ccc(-c2ccc3c(c2)oc2cccc(-c4nc(-c5cc6ccccc6c6sc7ccccc7c56)nc(-c5cc6ccccc6c6sc7ccccc7c56)n4)c23)cc1 ; c1ccc(-c2cccc3c2oc2cccc(-c4nc(-c5cc6ccccc6c6sc7ccccc7c56)nc(-c5cc6ccccc6c6sc7ccccc7c56)n4)c23)cc1]

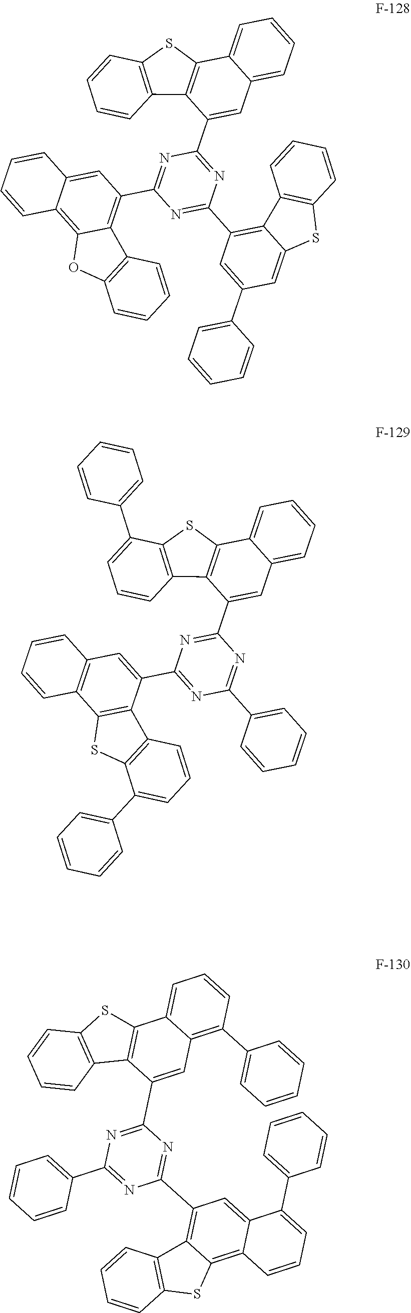 [SMILES: c1ccc(-c2cc(-c3nc(-c4cc5ccccc5c5oc6ccccc6c45)nc(-c4cc5ccccc5c5sc6ccccc6c45)n3)c3c(c2)sc2ccccc23)cc1 ; c1ccc(-c2nc(-c3cc4ccccc4c4sc5c(-c6ccccc6)cccc5c34)nc(-c3cc4ccccc4c4sc5c(-c6ccccc6)cccc5c34)n2)cc1 ; c1ccc(-c2nc(-c3cc4c(-c5ccccc5)cccc4c4sc5ccccc5c34)nc(-c3cc4c(-c5ccccc5)cccc4c4sc5ccccc5c34)n2)cc1]

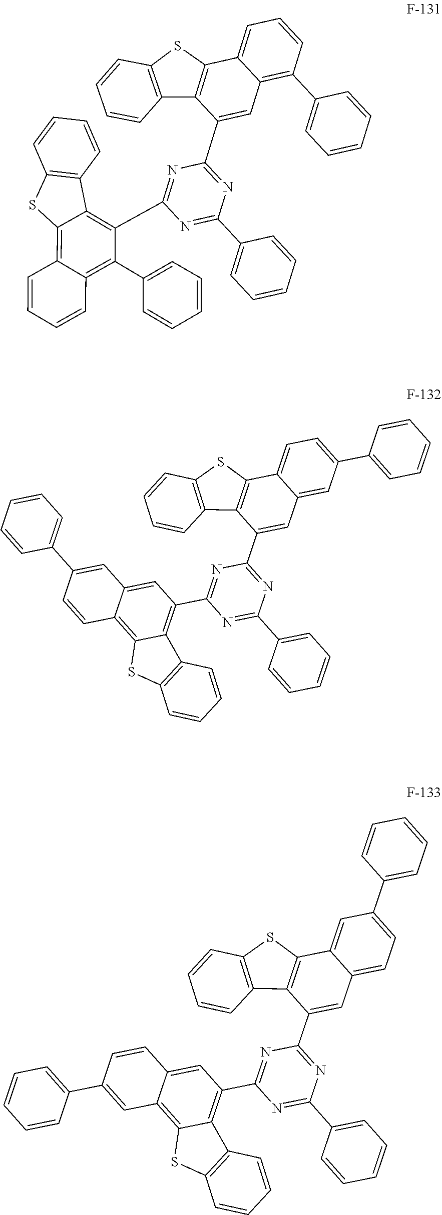 [SMILES: c1ccc(-c2nc(-c3cc4c(-c5ccccc5)cccc4c4sc5ccccc5c34)nc(-c3c(-c4ccccc4)c4ccccc4c4sc5ccccc5c34)n2)cc1 ; c1ccc(-c2ccc3c(c2)cc(-c2nc(-c4ccccc4)nc(-c4cc5cc(-c6ccccc6)ccc5c5sc6ccccc6c45)n2)c2c4ccccc4sc32)cc1 ; c1ccc(-c2ccc3cc(-c4nc(-c5ccccc5)nc(-c5cc6ccc(-c7ccccc7)cc6c6sc7ccccc7c56)n4)c4c5ccccc5sc4c3c2)cc1]

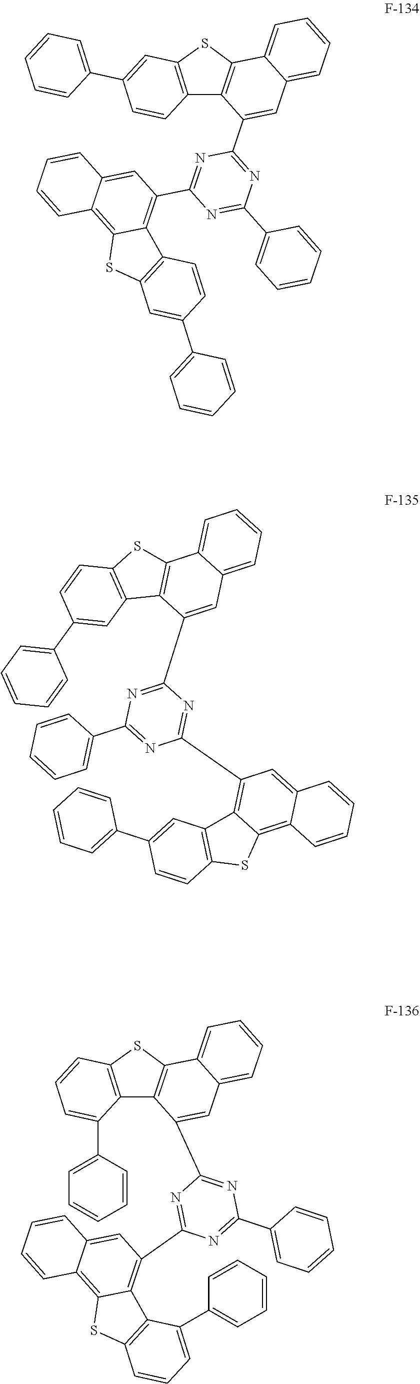 [SMILES: c1ccc(-c2ccc3c(c2)sc2c4ccccc4cc(-c4nc(-c5ccccc5)nc(-c5cc6ccccc6c6sc7cc(-c8ccccc8)ccc7c56)n4)c32)cc1 ; c1ccc(-c2ccc3sc4c5ccccc5cc(-c5nc(-c6ccccc6)nc(-c6cc7ccccc7c7sc8ccc(-c9ccccc9)cc8c67)n5)c4c3c2)cc1 ; c1ccc(-c2nc(-c3cc4ccccc4c4sc5cccc(-c6ccccc6)c5c34)nc(-c3cc4ccccc4c4sc5cccc(-c6ccccc6)c5c34)n2)cc1]

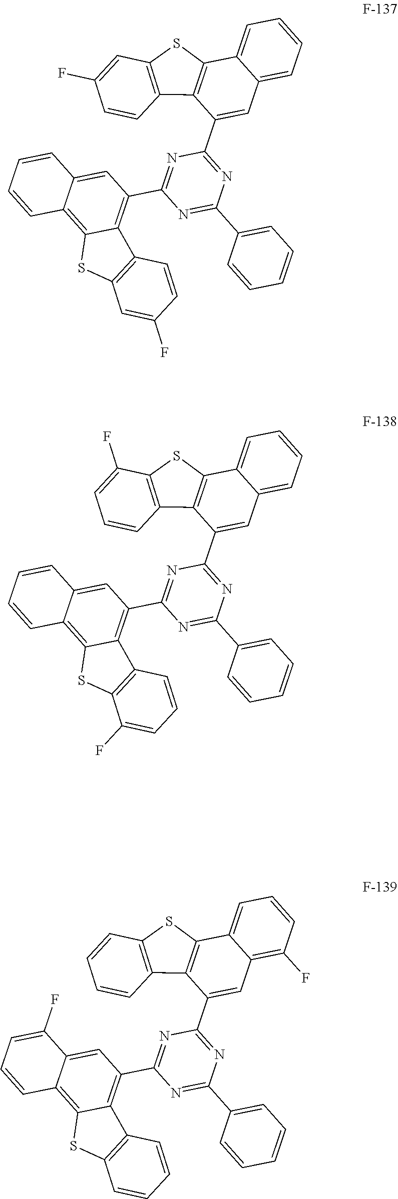 [SMILES: Fc1ccc2c(c1)sc1c3ccccc3cc(-c3nc(-c4ccccc4)nc(-c4cc5ccccc5c5sc6cc(F)ccc6c45)n3)c21 ; Fc1cccc2c1sc1c3ccccc3cc(-c3nc(-c4ccccc4)nc(-c4cc5ccccc5c5sc6c(F)cccc6c45)n3)c21 ; Fc1cccc2c1cc(-c1nc(-c3ccccc3)nc(-c3cc4c(F)cccc4c4sc5ccccc5c34)n1)c1c3ccccc3sc21]

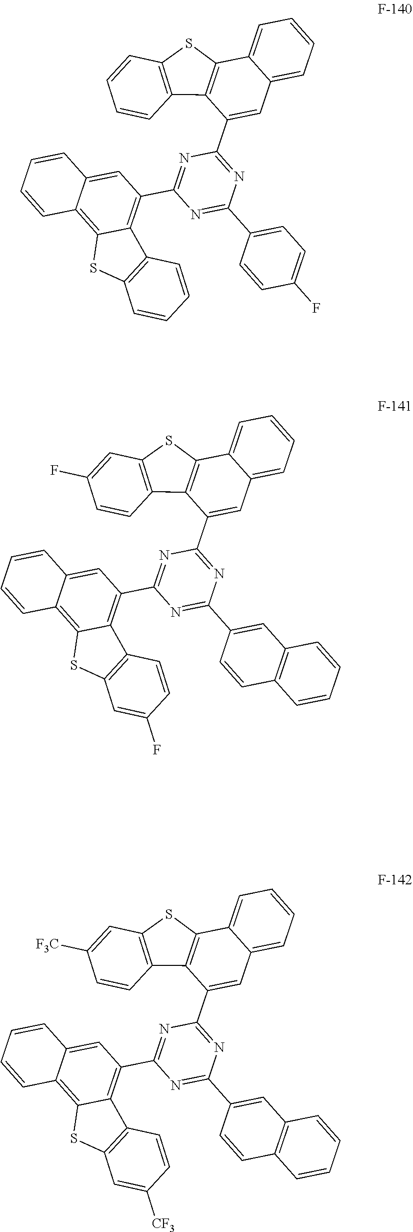 [SMILES: Fc1ccc(-c2nc(-c3cc4ccccc4c4sc5ccccc5c34)nc(-c3cc4ccccc4c4sc5ccccc5c34)n2)cc1 ; Fc1ccc2c(c1)sc1c3ccccc3cc(-c3nc(-c4ccc5ccccc5c4)nc(-c4cc5ccccc5c5sc6cc(F)ccc6c45)n3)c21 ; FC(F)(F)c1ccc2c(c1)sc1c3ccccc3cc(-c3nc(-c4ccc5ccccc5c4)nc(-c4cc5ccccc5c5sc6cc(C(F)(F)F)ccc6c45)n3)c21]

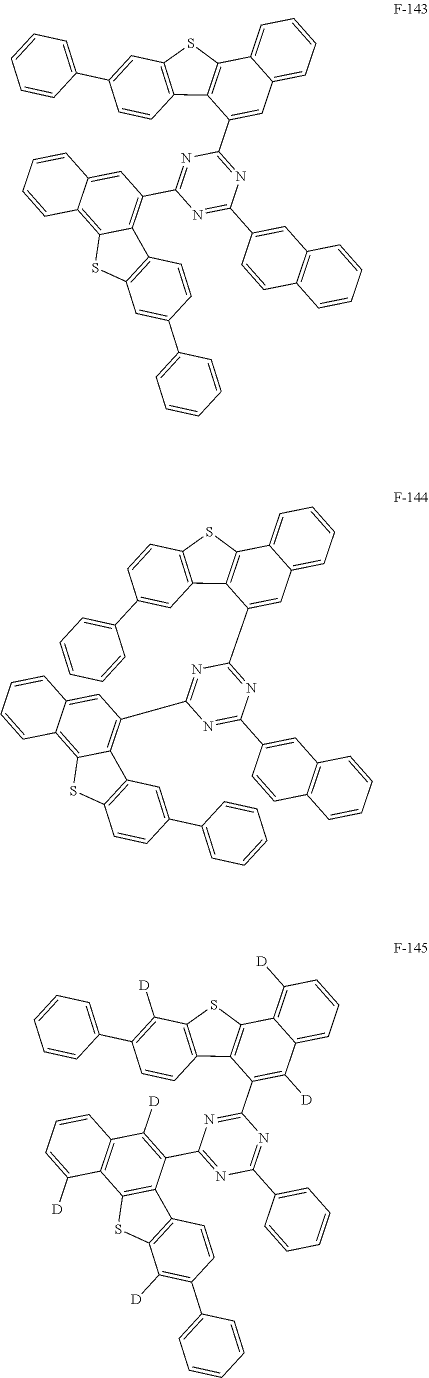 [SMILES: c1ccc(-c2ccc3c(c2)sc2c4ccccc4cc(-c4nc(-c5ccc6ccccc6c5)nc(-c5cc6ccccc6c6sc7cc(-c8ccccc8)ccc7c56)n4)c32)cc1 ; c1ccc(-c2ccc3sc4c5ccccc5cc(-c5nc(-c6ccc7ccccc7c6)nc(-c6cc7ccccc7c7sc8ccc(-c9ccccc9)cc8c67)n5)c4c3c2)cc1 ; [2H]c1c(-c2nc(-c3ccccc3)nc(-c3c([2H])c4cccc([2H])c4c4sc5c([2H])c(-c6ccccc6)ccc5c34)n2)c2c3ccc(-c4ccccc4)c([2H])c3sc2c2c([2H])cccc12]

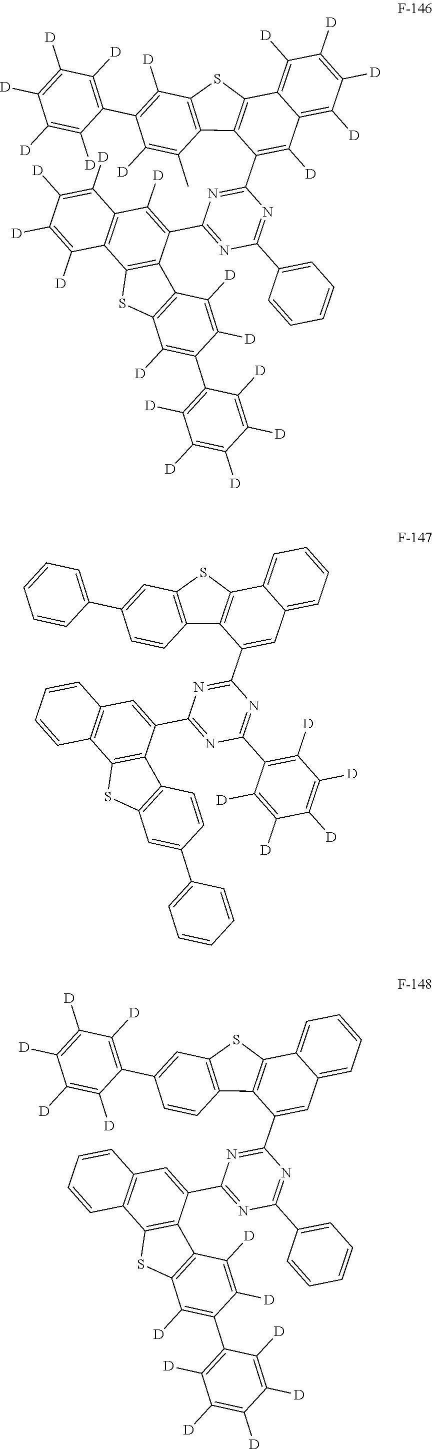

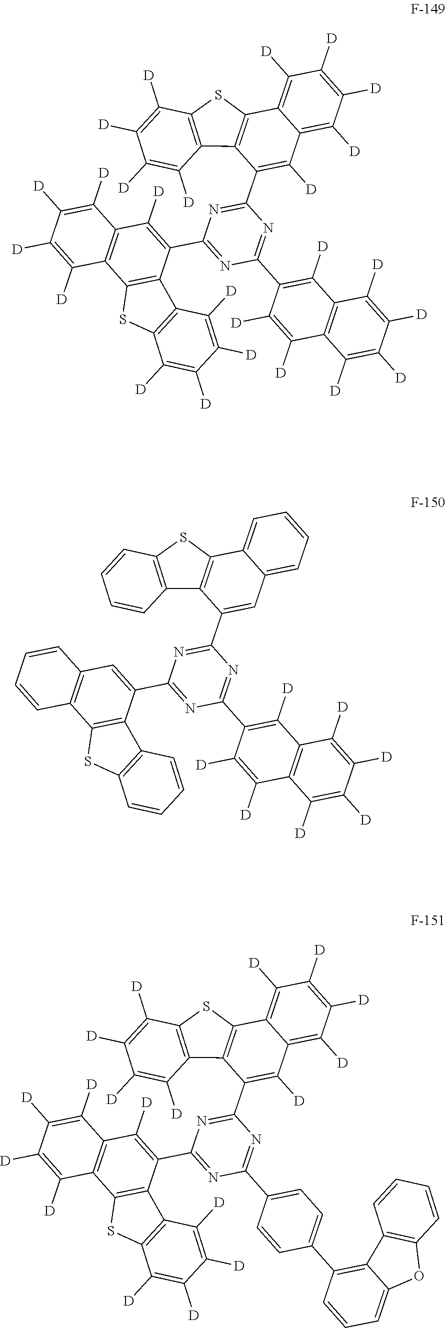

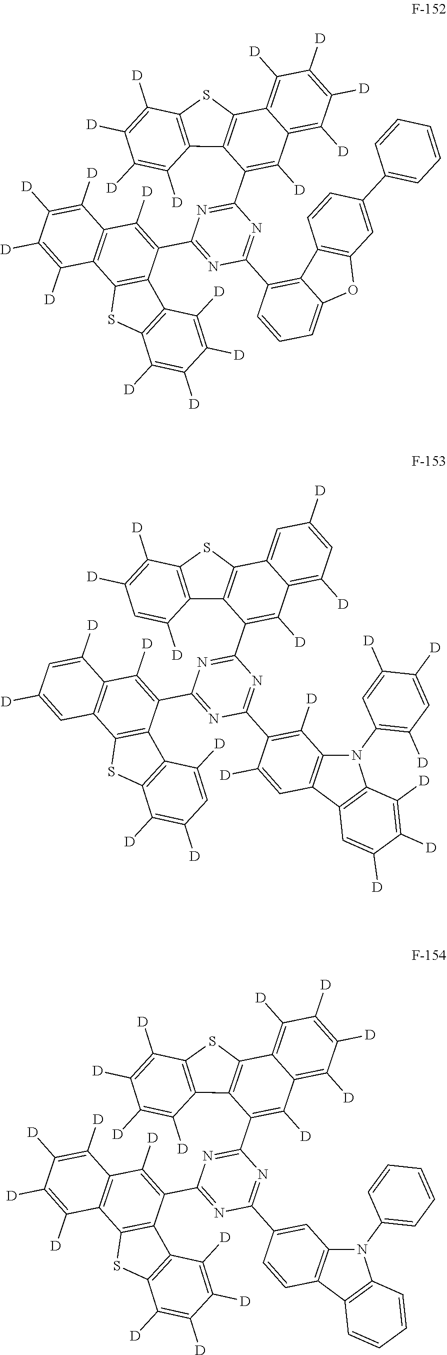

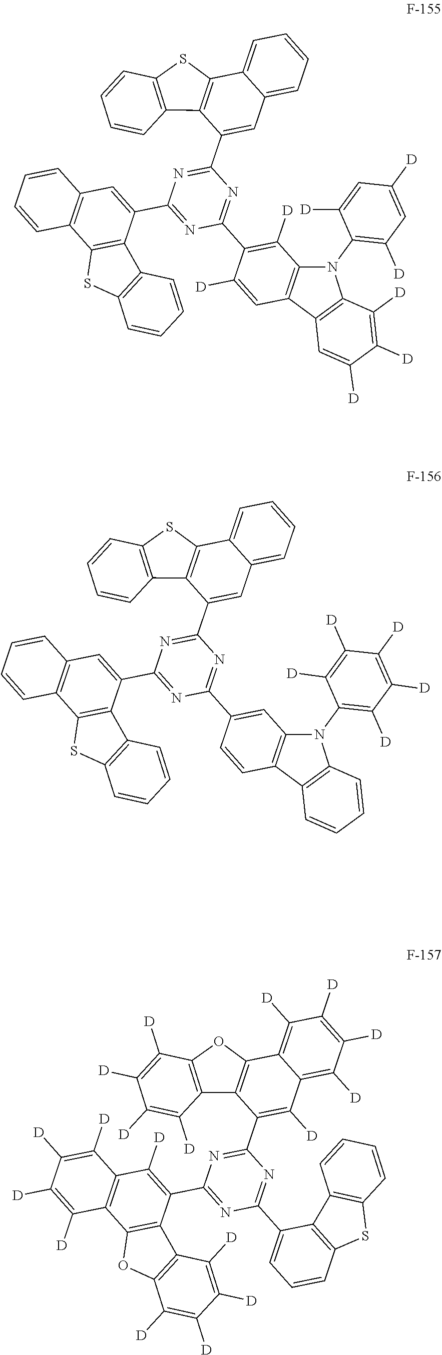 [SMILES: [2H]c1cc([2H])c(-n2c3c([2H])c([2H])c([2H])cc3c3cc([2H])c(-c4nc(-c5cc6ccccc6c6sc7ccccc7c56)nc(-c5cc6ccccc6c6sc7ccccc7c56)n4)c([2H])c32)c([2H])c1 ; [2H]c1c([2H])c([2H])c(-n2c3ccccc3c3ccc(-c4nc(-c5cc6ccccc6c6sc7ccccc7c56)nc(-c5cc6ccccc6c6sc7ccccc7c56)n4)cc32)c([2H])c1[2H] ; [2H]c1c([2H])c([2H])c2c(oc3c4c([2H])c([2H])c([2H])c([2H])c4c([2H])c(-c4nc(-c5cccc6sc7ccccc7c56)nc(-c5c([2H])c6c([2H])c([2H])c([2H])c([2H])c6c6oc7c([2H])c([2H])c([2H])c([2H])c7c56)n4)c32)c1[2H]]

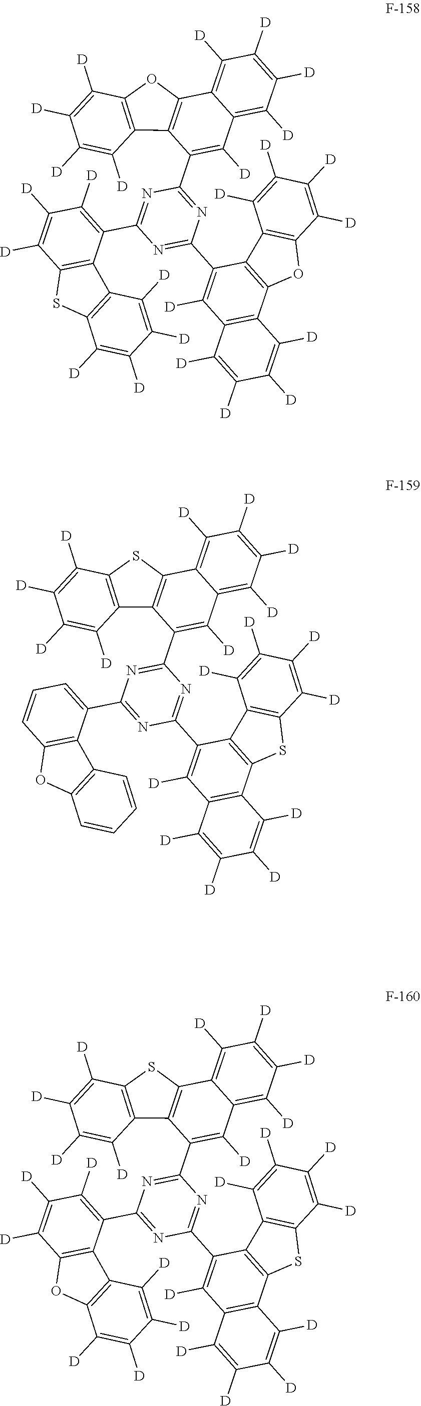

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include the one or more heterocyclic compounds of Chemical Formula 1.

In an exemplary embodiment of the present specification, the organic material layer includes a light emitting layer, and the light emitting layer may include one or more of the heterocyclic compounds.

In an exemplary embodiment of the present specification, the organic material layer includes a light emitting layer, and the light emitting layer may include one of the heterocyclic compounds.

In an exemplary embodiment of the present specification, the organic material layer includes a light emitting layer, the light emitting layer includes a host, and the host may include one or more of the heterocyclic compounds.

In an exemplary embodiment of the present specification, the organic material layer includes a light emitting layer, the light emitting layer includes a host, and the host may include one of the heterocyclic compounds.

In an exemplary embodiment of the present specification, the organic material layer includes a light emitting layer, the light emitting layer includes a host, the host includes a green host, and the green host may include one or more of the heterocyclic compounds.

In an exemplary embodiment of the present specification, the organic material layer includes a light emitting layer, the light emitting layer includes a host, the host includes a red host, and the red host may include one or more of the heterocyclic compounds.

In an exemplary embodiment of the present specification, the organic material layer includes a light emitting layer, the light emitting layer includes a host, the host includes a blue host, and the blue host may include one or more of the heterocyclic compounds.

In an exemplary embodiment of the present specification, the organic material layer includes a light emitting layer, and the light emitting layer may include the heterocyclic compound as an N-type host.

In an exemplary embodiment of the present specification, the organic material layer including the heterocyclic compound may further include a compound of the following Chemical Formula 2.

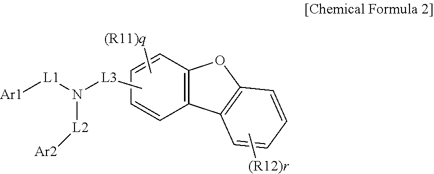

In Chemical Formula 2,
L1 to L3 are each independently a direct bond; a substituted or unsubstituted C6 to C60 arylene group; or a substituted or unsubstituted C2 to C60 heteroarylene group,
Ar1 and Ar2 are each independently a substituted or unsubstituted C6 to C60 aryl group; or a substituted or unsubstituted C2 to C60 heteroaryl group,
R11 and R12 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or are bonded to an adjacent group to form a substituted or unsubstituted C6 to C60 aromatic ring or a substituted or unsubstituted C2 to C60 hetero ring,
q is an integer from 1 to 3,
r is an integer of 1 to 4, and
when q and r are each 2 or higher, substituents in the parenthesis are the same as or different from each other.

In an exemplary embodiment of the present specification, the organic material layer includes a light emitting layer, and the light emitting layer may further include the compound of Chemical Formula 2.

In an exemplary embodiment of the present specification, the organic material layer includes a light emitting layer, the light emitting layer includes a host, and the host may further include the compound of Chemical Formula 2.

In an exemplary embodiment of the present specification, the light emitting layer may further include the compound of Chemical Formula 2 as a P-type host.

In an exemplary embodiment of the present specification, L1 to L3 may be each independently a direct bond; or a substituted or unsubstituted C6 to C60 arylene group.

In an exemplary embodiment of the present specification, L1 to L3 may be each independently a direct bond; or a substituted or unsubstituted C6 to C40 arylene group.

In an exemplary embodiment of the present specification, L1 to L3 may be each independently a direct bond; or a substituted or unsubstituted C6 to C20 arylene group.

In an exemplary embodiment of the present specification, L1 to L3 may be each independently a direct bond; a substituted or unsubstituted phenylene group; or a substituted or unsubstituted biphenylene group.

In an exemplary embodiment of the present specification, L1 to L3 may be each independently a direct bond; or a C6 to C60 arylene group unsubstituted or substituted with deuterium.

In an exemplary embodiment of the present specification, L1 to L3 may be each independently a direct bond; or a C6 to C40 arylene group unsubstituted or substituted with deuterium.

In an exemplary embodiment of the present specification, L1 to L3 may be each independently a direct bond; or a C6 to C20 arylene group unsubstituted or substituted with deuterium.

In an exemplary embodiment of the present specification, L1 to L3 may be each independently a direct bond; a phenylene group unsubstituted or substituted with deuterium; or a biphenylene group unsubstituted or substituted with deuterium.

In an exemplary embodiment of the present specification, Ar1 and Ar2 may be each independently a substituted or unsubstituted C6 to C40 aryl group; or a substituted or unsubstituted C2 to C40 heteroaryl group.

In an exemplary embodiment of the present specification, Ar1 and Ar2 may be each independently a substituted or unsubstituted C6 to C30 aryl group; or a substituted or unsubstituted C2 to C30 heteroaryl group.

In an exemplary embodiment of the present specification, Ar1 and Ar2 may be each independently a substituted or unsubstituted C6 to C30 aryl group; or a C2 to C30 heteroaryl group which is substituted or unsubstituted and includes O or S.

In an exemplary embodiment of the present specification, Ar1 and Ar2 may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted fluorenyl group; a substituted or unsubstituted dibenzofuran group; or a substituted or unsubstituted dibenzothiophene group.

In an exemplary embodiment of the present specification, Ar1 and Ar2 may be each independently a C6 to C60 aryl group unsubstituted or substituted with one or more substituents of deuterium and an alkyl group; or a C2 to C60 heteroaryl group unsubstituted or substituted with deuterium.

In an exemplary embodiment of the present specification, Ar1 and Ar2 may be each independently a C6 to C40 aryl group unsubstituted or substituted with one or more substituents of deuterium and an alkyl group; or a C2 to C40 heteroaryl group unsubstituted or substituted with deuterium.

In an exemplary embodiment of the present specification, Ar1 and Ar2 may be each independently a C6 to C30 aryl group unsubstituted or substituted with one or more substituents of deuterium and an alkyl group; or a C2 to C30 heteroaryl group unsubstituted or substituted with deuterium.

In an exemplary embodiment of the present specification, Ar1 and Ar2 may be each independently a C6 to C30 aryl group unsubstituted or substituted with one or more substituents of deuterium and an alkyl group; or a C2 to C30 heteroaryl group unsubstituted or substituted with deuterium and including O or S.

In an exemplary embodiment of the present specification, Ar1 and Ar2 may be each independently a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a phenanthrenyl group unsubstituted or substituted with deuterium; a dimethylfluorenyl group unsubstituted or substituted with deuterium; a dibenzofuran group unsubstituted or substituted with deuterium; or a dibenzothiophene group unsubstituted or substituted with deuterium.

In an exemplary embodiment of the present specification, R11 and R12 may be each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or may be bonded to an adjacent group to form a substituted or unsubstituted C6 to C60 aromatic ring or a substituted or unsubstituted C2 to C60 hetero ring.

In an exemplary embodiment of the present specification, R11 and R12 may be each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or may be bonded to an adjacent group to form a substituted or unsubstituted C6 to C60 aromatic ring.

In an exemplary embodiment of the present specification, R11 and R12 may be each independently hydrogen; deuterium; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group, or may be bonded to an adjacent group to form a substituted or unsubstituted C6 to C40 aromatic ring or a substituted or unsubstituted C2 to C40 hetero ring.

In an exemplary embodiment of the present specification, R11 and R12 may be each independently hydrogen; deuterium; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group, or may be bonded to an adjacent group to form a substituted or unsubstituted C6 to C20 aromatic ring or a substituted or unsubstituted C2 to C20 hetero ring.

In an exemplary embodiment of the present specification, R11 and R12 may be each independently hydrogen; deuterium; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group, or may be bonded to an adjacent group to form a substituted or unsubstituted C6 to C20 aromatic ring.

In an exemplary embodiment of the present specification, R11 and R12 may be each independently hydrogen; deuterium; a substituted or unsubstituted C6 to C60 aryl group; or a substituted or unsubstituted C2 to C60 heteroaryl group, or may be bonded to an adjacent group to form a substituted or unsubstituted C6 to C60 aromatic ring or a substituted or unsubstituted C2 to C60 hetero ring.

In an exemplary embodiment of the present specification, R11 and R12 may be each independently hydrogen; deuterium; a substituted or unsubstituted C6 to C40 aryl group; or a substituted or unsubstituted C2 to C40 heteroaryl group, or may be bonded to an adjacent group to form a substituted or unsubstituted C6 to C40 aromatic ring or a substituted or unsubstituted C2 to C40 hetero ring.

In an exemplary embodiment of the present specification, R11 and R12 may be each independently hydrogen; deuterium; a substituted or unsubstituted C6 to C20 aryl group; or a substituted or unsubstituted C2 to C20 heteroaryl group, or may be bonded to an adjacent group to form a substituted or unsubstituted C6 to C20 aromatic ring or a substituted or unsubstituted C2 to C20 hetero ring.

In an exemplary embodiment of the present specification, R11 and R12 may be each independently hydrogen; deuterium; a substituted or unsubstituted C6 to C20 aryl group; or a substituted or unsubstituted C2 to C20 heteroaryl group, or may be bonded to an adjacent group to form a substituted or unsubstituted C6 to C20 aromatic ring.

In an exemplary embodiment of the present specification, R11 and R12 may be each independently hydrogen; deuterium; a substituted or unsubstituted C6 to C20 aryl group; or a substituted or unsubstituted C2 to C20 heteroaryl group, or may be bonded to an adjacent group to form a substituted or unsubstituted benzene ring.

In an exemplary embodiment of the present specification, R11 and R12 may be each independently hydrogen; deuterium; a substituted or unsubstituted C6 to C20 aryl group; or a C2 to C20 heteroaryl group which is substituted or unsubstituted and includes N, or may be bonded to an adjacent group to form a substituted or unsubstituted C6 to C20 aromatic ring.

In an exemplary embodiment of the present specification, R11 and R12 may be each independently hydrogen; deuterium; a substituted or unsubstituted C6 to C20 aryl group; or a C2 to C20 heteroaryl group which is substituted or unsubstituted and includes N, or may be bonded to an adjacent group to form a substituted or unsubstituted benzene ring.

In an exemplary embodiment of the present specification, R11 and R12 may be each independently hydrogen; deuterium; a substituted or unsubstituted phenyl group; a substituted or unsubstituted biphenyl group; a substituted or unsubstituted naphthyl group; or a substituted or unsubstituted carbazole group, or may be bonded to an adjacent group to form a substituted or unsubstituted benzene ring.

In an exemplary embodiment of the present specification, R11 and R12 may be each independently hydrogen; deuterium; a C to C20 aryl group unsubstituted or substituted with deuterium or an aryl group; or a C2 to C20 heteroaryl group unsubstituted or substituted with deuterium or an aryl group and including N, or may be bonded to an adjacent group to form a benzene ring unsubstituted or substituted with deuterium.

In an exemplary embodiment of the present specification, R11 and R12 may be each independently hydrogen; deuterium; or a substituted or unsubstituted C6 to C60 aryl group, or may be bonded to an adjacent group to form a substituted or unsubstituted C6 to C60 aromatic ring.

In an exemplary embodiment of the present specification, R11 and R12 may be each independently hydrogen; deuterium; or a substituted or unsubstituted C6 to C40 aryl group, or may be bonded to an adjacent group to form a substituted or unsubstituted C6 to C40 aromatic ring.

In an exemplary embodiment of the present specification, R11 and R12 may be each independently hydrogen; deuterium; or a substituted or unsubstituted C6 to C20 aryl group, or may be bonded to an adjacent group to form a substituted or unsubstituted C6 to C20 aromatic ring.

In an exemplary embodiment of the present specification, R11 and R12 may be each independently hydrogen; deuterium; or a substituted or unsubstituted C6 to C20 aryl group, or may be bonded to an adjacent group to form a substituted or unsubstituted benzene ring.

In an exemplary embodiment of the present specification, R11 and R12 may be each independently hydrogen; deuterium; a substituted or unsubstituted phenyl group; a substituted or unsubstituted biphenyl group; or a substituted or unsubstituted naphthyl group, or may be bonded to an adjacent group to form a substituted or unsubstituted benzene ring.

In an exemplary embodiment of the present specification, Chemical Formula 2 may be represented by any one of the following Chemical Formulae 2-1 to 2-4.

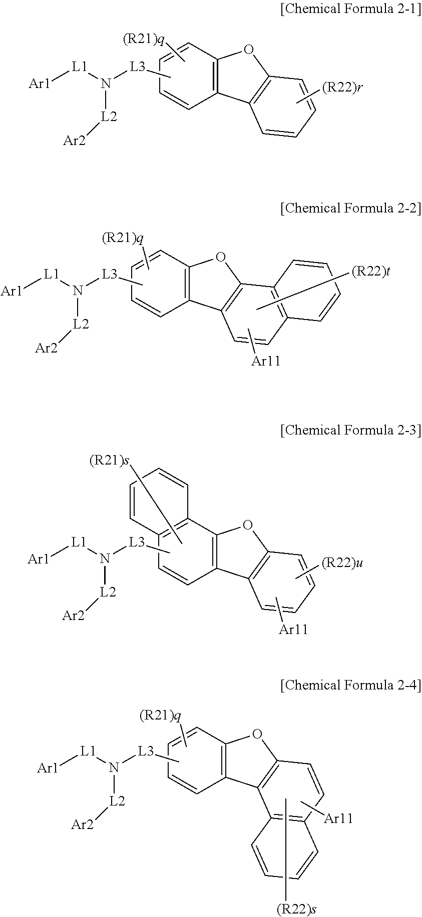

In Chemical Formulae 2-1 to 2-4,
the definitions of L1 to L3, Ar1, Ar2, q, and r are the same as the definitions in Chemical Formula 2,
Ar11 is a substituted or unsubstituted C6 to C60 aryl group; or a substituted or unsubstituted C2 to C60 heteroaryl group,
R21 and R22 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
s and t are each an integer from 1 to 5,
u is an integer of 1 to 3, and
when s, t, and u are each 2 or higher, substituents in the parenthesis are the same as or different from each other.

In an exemplary embodiment of the present specification, Ar11 may be a substituted or unsubstituted C6 to C30 aryl group; or a substituted or unsubstituted C2 to C30 heteroaryl group.

In an exemplary embodiment of the present specification, Ar11 may be a substituted or unsubstituted C6 to C20 aryl group; or a substituted or unsubstituted C2 to C20 heteroaryl group.

In an exemplary embodiment of the present specification, Ar11 may be a substituted or unsubstituted C6 to C20 aryl group; or a C2 to C20 heteroaryl group which is substituted or unsubstituted and includes N.

In an exemplary embodiment of the present specification, Ar11 may be a substituted or unsubstituted phenyl group; a substituted or unsubstituted biphenyl group; a substituted or unsubstituted naphthyl group; or a substituted or unsubstituted carbazole group.

In an exemplary embodiment of the present specification, Ar11 may be a C6 to C30 aryl group unsubstituted or substituted with deuterium or an aryl group; or a C2 to C30 heteroaryl group unsubstituted or substituted with deuterium or an aryl group.

In an exemplary embodiment of the present specification, Ar11 may be a C6 to C20 aryl group unsubstituted or substituted with deuterium or an aryl group; or a C2 to C20 heteroaryl group unsubstituted or substituted with deuterium or an aryl group.

In an exemplary embodiment of the present specification, Ar11 may be a C6 to C20 aryl group unsubstituted or substituted with deuterium or an aryl group; or a C2 to C20 heteroaryl group unsubstituted or substituted with deuterium or an aryl group and including N.

In an exemplary embodiment of the present specification, Ar11 may be a phenyl group unsubstituted or substituted with deuterium or an aryl group; a biphenyl group unsubstituted or substituted with deuterium; a naphthyl group unsubstituted or substituted with deuterium or an aryl group; or a carbazole group unsubstituted or substituted with deuterium or an aryl group.

In an exemplary embodiment of the present specification, Ar11 may be a substituted or unsubstituted C6 to C30 aryl group.

In an exemplary embodiment of the present specification, Ar11 may be a substituted or unsubstituted C6 to C20 aryl group.

In an exemplary embodiment of the present specification, Ar11 may be a substituted or unsubstituted phenyl group; a substituted or unsubstituted biphenyl group; or a substituted or unsubstituted naphthyl group.

In an exemplary embodiment of the present specification, Ar11 may be a C6 to C30 aryl group unsubstituted or substituted with deuterium or an aryl group.

In an exemplary embodiment of the present specification, Ar11 may be a C6 to C20 aryl group unsubstituted or substituted with deuterium or an aryl group.

In an exemplary embodiment of the present specification, Ar11 may be a phenyl group unsubstituted or substituted with deuterium or an aryl group; a biphenyl group unsubstituted or substituted with deuterium; or a naphthyl group unsubstituted or substituted with deuterium or an aryl group.

In an exemplary embodiment of the present specification, R21 and R22 may be each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R21 and R22 may be each independently hydrogen; deuterium; a substituted or unsubstituted C1 to C60 alkyl group; a substituted or unsubstituted C3 to C60 cycloalkyl group; or a substituted or unsubstituted C6 to C60 aryl group.

In an exemplary embodiment of the present specification, R21 and R22 may be each independently hydrogen; or deuterium.

In an exemplary embodiment of the present specification, Chemical Formula 2 may be selected from the following compounds.

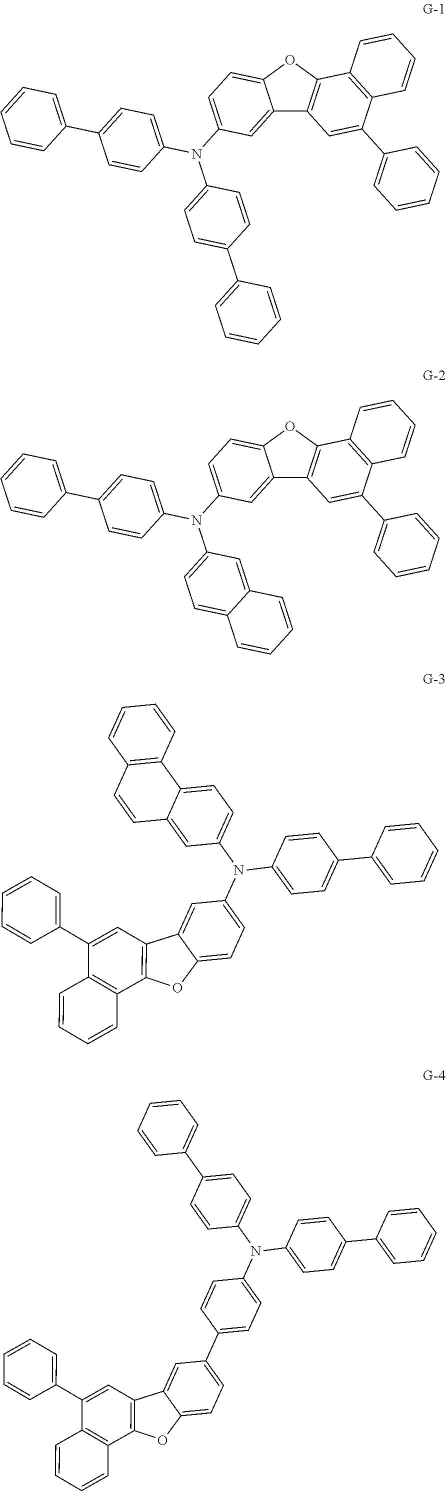 [SMILES: c1ccc(-c2ccc(N(c3ccc(-c4ccccc4)cc3)c3ccc4oc5c6ccccc6c(-c6ccccc6)cc5c4c3)cc2)cc1 ; c1ccc(-c2ccc(N(c3ccc4ccccc4c3)c3ccc4oc5c6ccccc6c(-c6ccccc6)cc5c4c3)cc2)cc1 ; c1ccc(-c2ccc(N(c3ccc4c(ccc5ccccc54)c3)c3ccc4oc5c6ccccc6c(-c6ccccc6)cc5c4c3)cc2)cc1 ; c1ccc(-c2ccc(N(c3ccc(-c4ccccc4)cc3)c3ccc(-c4ccc5oc6c7ccccc7c(-c7ccccc7)cc6c5c4)cc3)cc2)cc1]

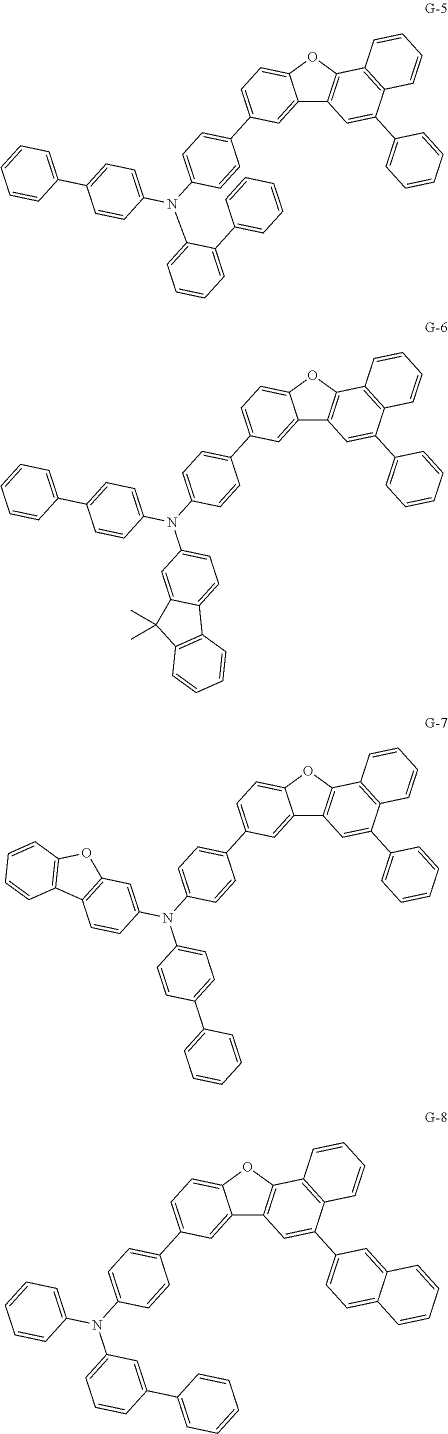 [SMILES: c1ccc(-c2ccc(N(c3ccc(-c4ccc5oc6c7ccccc7c(-c7ccccc7)cc6c5c4)cc3)c3ccccc3-c3ccccc3)cc2)cc1 ; CC1(C)c2ccccc2-c2ccc(N(c3ccc(-c4ccccc4)cc3)c3ccc(-c4ccc5oc6c7ccccc7c(-c7ccccc7)cc6c5c4)cc3)cc21 ; c1ccc(-c2ccc(N(c3ccc(-c4ccc5oc6c7ccccc7c(-c7ccccc7)cc6c5c4)cc3)c3ccc4c(c3)oc3ccccc34)cc2)cc1 ; c1ccc(-c2cccc(N(c3ccccc3)c3ccc(-c4ccc5oc6c7ccccc7c(-c7ccc8ccccc8c7)cc6c5c4)cc3)c2)cc1]

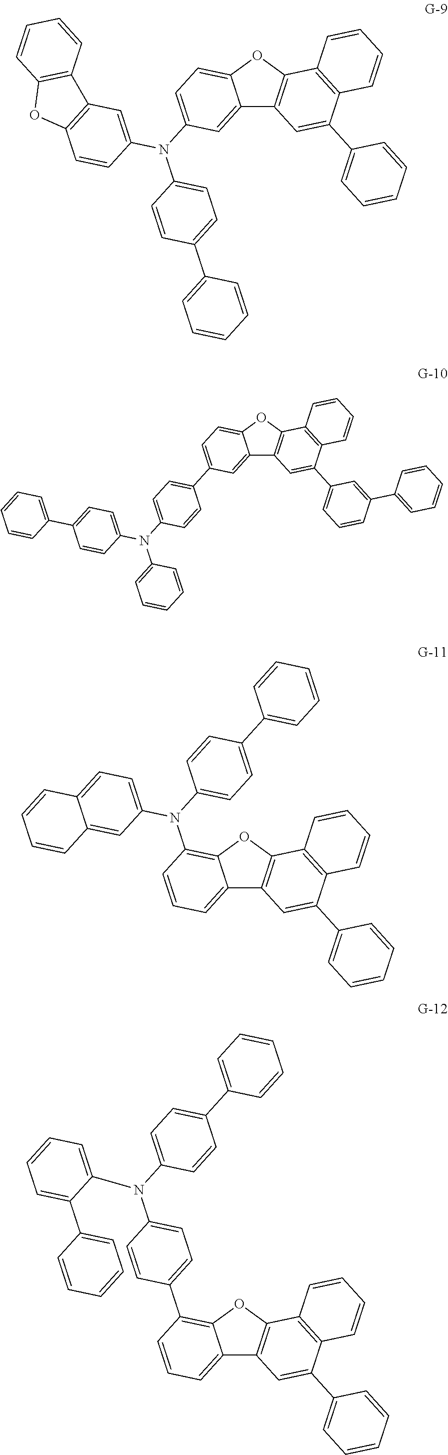 [SMILES: c1ccc(-c2ccc(N(c3ccc4oc5ccccc5c4c3)c3ccc4oc5c6ccccc6c(-c6ccccc6)cc5c4c3)cc2)cc1 ; c1ccc(-c2ccc(N(c3ccccc3)c3ccc(-c4ccc5oc6c7ccccc7c(-c7cccc(-c8ccccc8)c7)cc6c5c4)cc3)cc2)cc1 ; c1ccc(-c2ccc(N(c3ccc4ccccc4c3)c3cccc4c3oc3c5ccccc5c(-c5ccccc5)cc43)cc2)cc1 ; c1ccc(-c2ccc(N(c3ccc(-c4cccc5c4oc4c6ccccc6c(-c6ccccc6)cc54)cc3)c3ccccc3-c3ccccc3)cc2)cc1]

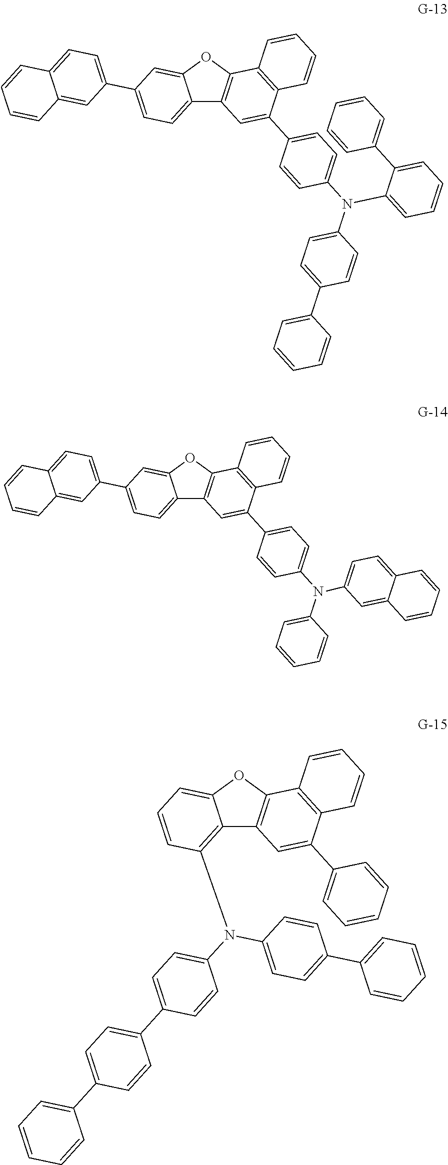 [SMILES: c1ccc(-c2ccc(N(c3ccc(-c4cc5c6ccc(-c7ccc8ccccc8c7)cc6oc5c5ccccc45)cc3)c3ccccc3-c3ccccc3)cc2)cc1 ; c1ccc(N(c2ccc(-c3cc4c5ccc(-c6ccc7ccccc7c6)cc5oc4c4ccccc34)cc2)c2ccc3ccccc3c2)cc1 ; c1ccc(-c2ccc(-c3ccc(N(c4ccc(-c5ccccc5)cc4)c4cccc5oc6c7ccccc7c(-c7ccccc7)cc6c45)cc3)cc2)cc1]

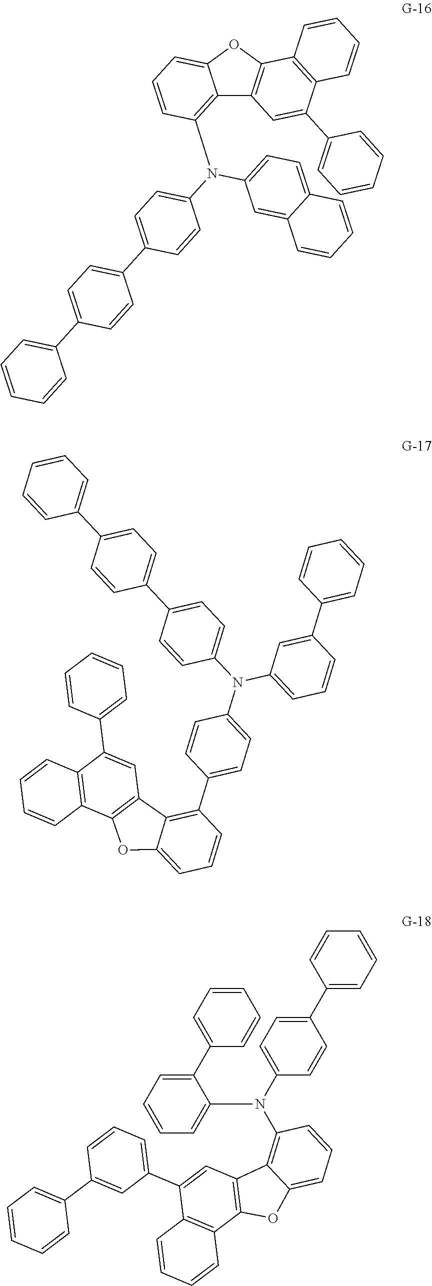 [SMILES: c1ccc(-c2ccc(-c3ccc(N(c4ccc5ccccc5c4)c4cccc5oc6c7ccccc7c(-c7ccccc7)cc6c45)cc3)cc2)cc1 ; c1ccc(-c2ccc(-c3ccc(N(c4ccc(-c5cccc6oc7c8ccccc8c(-c8ccccc8)cc7c56)cc4)c4cccc(-c5ccccc5)c4)cc3)cc2)cc1 ; c1ccc(-c2ccc(N(c3ccccc3-c3ccccc3)c3cccc4oc5c6ccccc6c(-c6cccc(-c7ccccc7)c6)cc5c34)cc2)cc1]

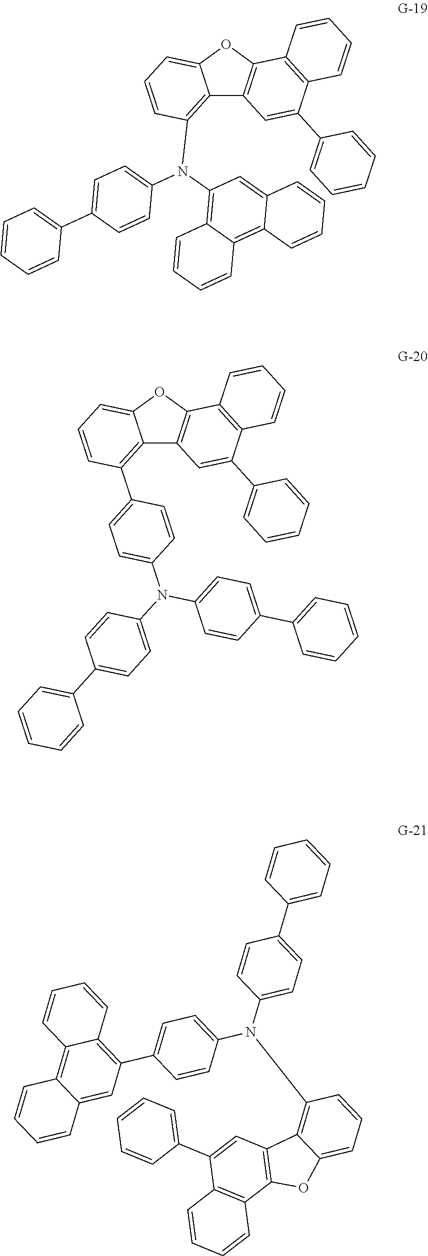 [SMILES: c1ccc(-c2ccc(N(c3cc4ccccc4c4ccccc34)c3cccc4oc5c6ccccc6c(-c6ccccc6)cc5c34)cc2)cc1 ; c1ccc(-c2ccc(N(c3ccc(-c4ccccc4)cc3)c3ccc(-c4cccc5oc6c7ccccc7c(-c7ccccc7)cc6c45)cc3)cc2)cc1 ; c1ccc(-c2ccc(N(c3ccc(-c4cc5ccccc5c5ccccc45)cc3)c3cccc4oc5c6ccccc6c(-c6ccccc6)cc5c34)cc2)cc1]

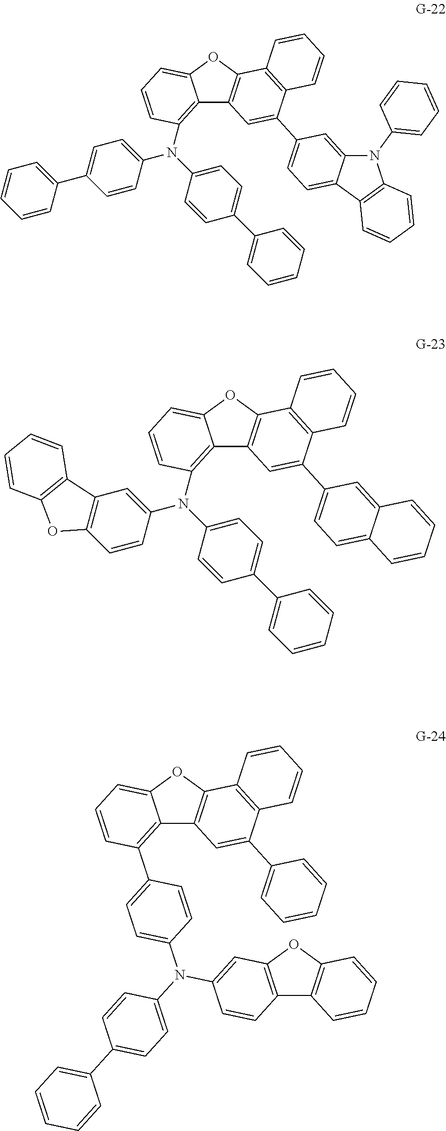 [SMILES: c1ccc(-c2ccc(N(c3ccc(-c4ccccc4)cc3)c3cccc4oc5c6ccccc6c(-c6ccc7c8ccccc8n(-c8ccccc8)c7c6)cc5c34)cc2)cc1 ; c1ccc(-c2ccc(N(c3ccc4oc5ccccc5c4c3)c3cccc4oc5c6ccccc6c(-c6ccc7ccccc7c6)cc5c34)cc2)cc1 ; c1ccc(-c2ccc(N(c3ccc(-c4cccc5oc6c7ccccc7c(-c7ccccc7)cc6c45)cc3)c3ccc4c(c3)oc3ccccc34)cc2)cc1]

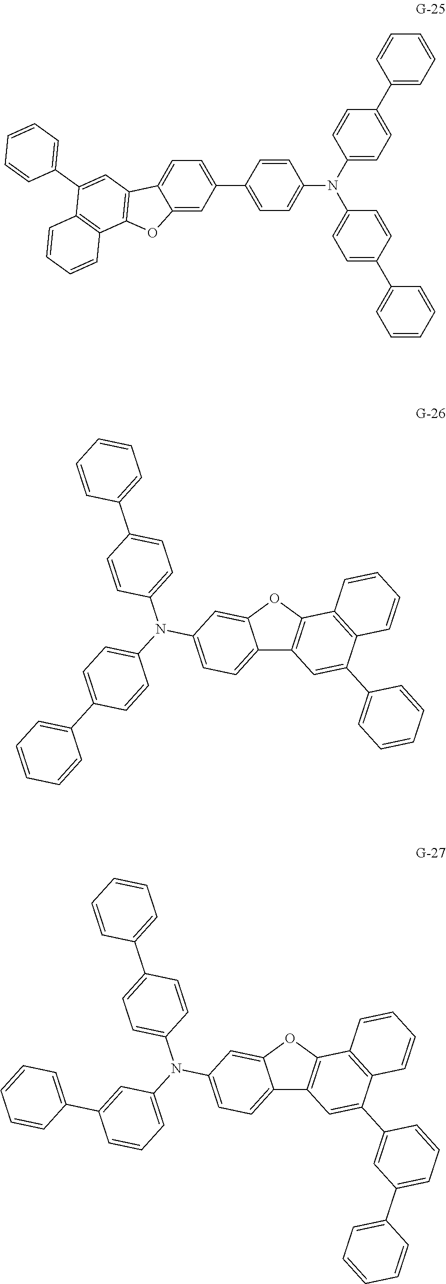 [SMILES: c1ccc(-c2ccc(N(c3ccc(-c4ccccc4)cc3)c3ccc(-c4ccc5c(c4)oc4c6ccccc6c(-c6ccccc6)cc54)cc3)cc2)cc1 ; c1ccc(-c2ccc(N(c3ccc(-c4ccccc4)cc3)c3ccc4c(c3)oc3c5ccccc5c(-c5ccccc5)cc43)cc2)cc1 ; c1ccc(-c2ccc(N(c3cccc(-c4ccccc4)c3)c3ccc4c(c3)oc3c5ccccc5c(-c5cccc(-c6ccccc6)c5)cc43)cc2)cc1]

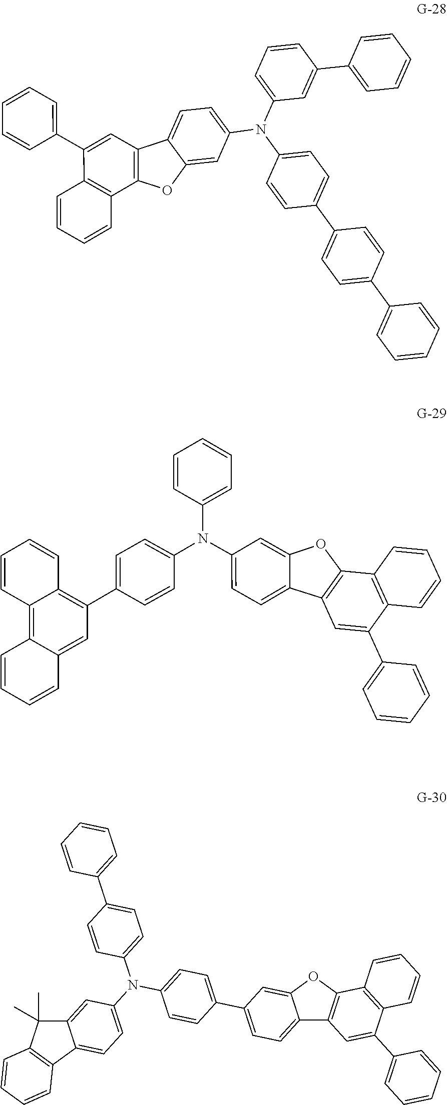 [SMILES: c1ccc(-c2ccc(-c3ccc(N(c4cccc(-c5ccccc5)c4)c4ccc5c(c4)oc4c6ccccc6c(-c6ccccc6)cc54)cc3)cc2)cc1 ; c1ccc(-c2cc3c4ccc(N(c5ccccc5)c5ccc(-c6cc7ccccc7c7ccccc67)cc5)cc4oc3c3ccccc23)cc1 ; CC1(C)c2ccccc2-c2ccc(N(c3ccc(-c4ccccc4)cc3)c3ccc(-c4ccc5c(c4)oc4c6ccccc6c(-c6ccccc6)cc54)cc3)cc21]

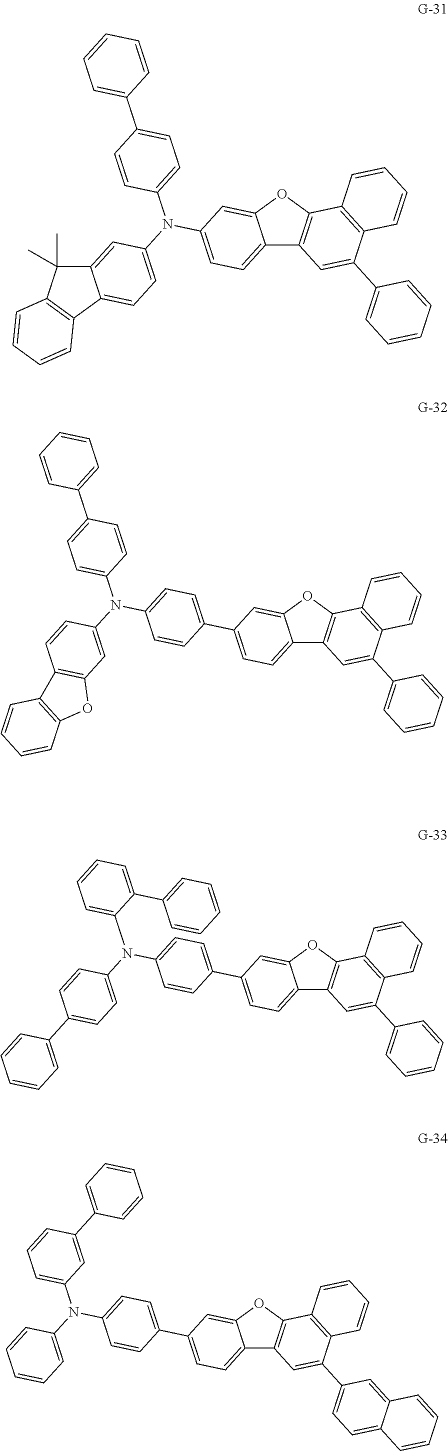 [SMILES: CC1(C)c2ccccc2-c2ccc(N(c3ccc(-c4ccccc4)cc3)c3ccc4c(c3)oc3c5ccccc5c(-c5ccccc5)cc43)cc21 ; c1ccc(-c2ccc(N(c3ccc(-c4ccc5c(c4)oc4c6ccccc6c(-c6ccccc6)cc54)cc3)c3ccc4c(c3)oc3ccccc34)cc2)cc1 ; c1ccc(-c2ccc(N(c3ccc(-c4ccc5c(c4)oc4c6ccccc6c(-c6ccccc6)cc54)cc3)c3ccccc3-c3ccccc3)cc2)cc1 ; c1ccc(-c2cccc(N(c3ccccc3)c3ccc(-c4ccc5c(c4)oc4c6ccccc6c(-c6ccc7ccccc7c6)cc54)cc3)c2)cc1]

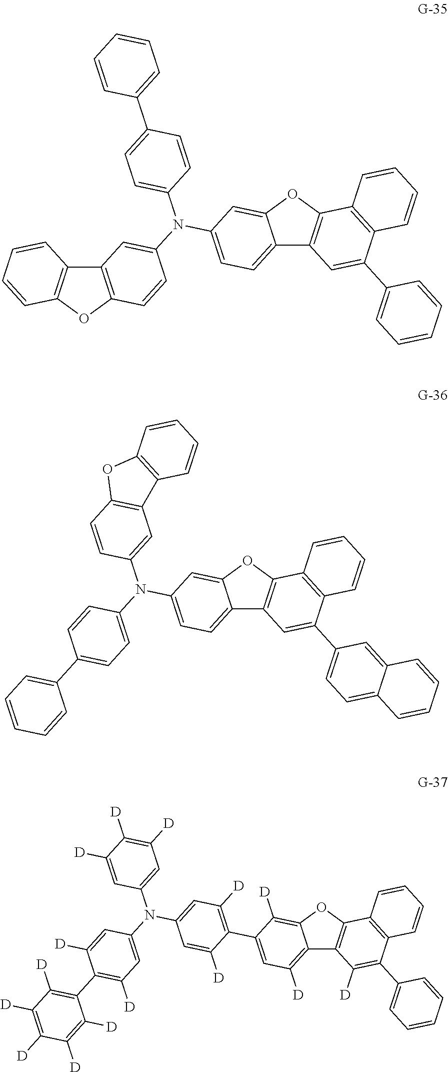 [SMILES: c1ccc(-c2ccc(N(c3ccc4c(c3)oc3c5ccccc5c(-c5ccccc5)cc43)c3ccc4oc5ccccc5c4c3)cc2)cc1 ; c1ccc(-c2ccc(N(c3ccc4c(c3)oc3c5ccccc5c(-c5ccc6ccccc6c5)cc43)c3ccc4oc5ccccc5c4c3)cc2)cc1 ; [2H]c1cc(N(c2cc([2H])c(-c3cc([2H])c4c(oc5c6ccccc6c(-c6ccccc6)c([2H])c54)c3[2H])c([2H])c2)c2cc([2H])c(-c3c([2H])c([2H])c([2H])c([2H])c3[2H])c([2H])c2)cc([2H])c1[2H]]

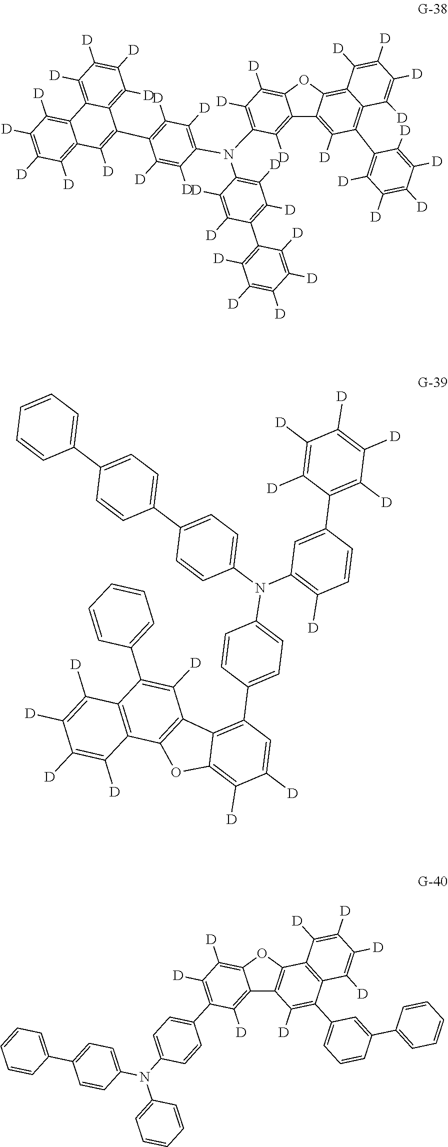

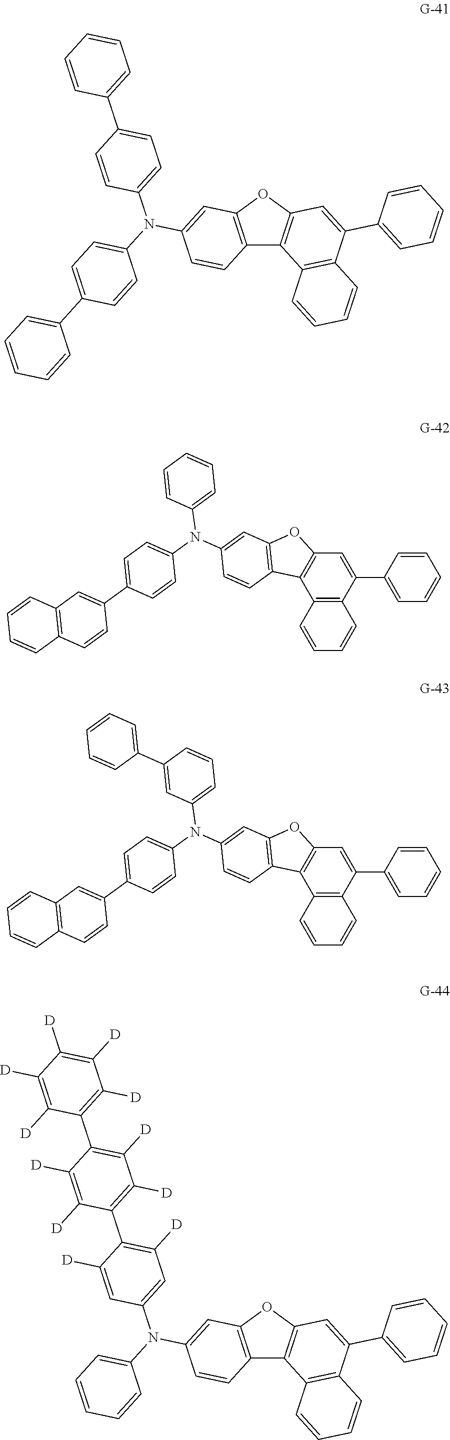 [SMILES: c1ccc(-c2ccc(N(c3ccc(-c4ccccc4)cc3)c3ccc4c(c3)oc3cc(-c5ccccc5)c5ccccc5c34)cc2)cc1 ; c1ccc(-c2cc3oc4cc(N(c5ccccc5)c5ccc(-c6ccc7ccccc7c6)cc5)ccc4c3c3ccccc23)cc1 ; c1ccc(-c2cccc(N(c3ccc(-c4ccc5ccccc5c4)cc3)c3ccc4c(c3)oc3cc(-c5ccccc5)c5ccccc5c34)c2)cc1 ; [2H]c1cc(N(c2ccccc2)c2ccc3c(c2)oc2cc(-c4ccccc4)c4ccccc4c23)cc([2H])c1-c1c([2H])c([2H])c(-c2c([2H])c([2H])c([2H])c([2H])c2[2H])c([2H])c1[2H]]

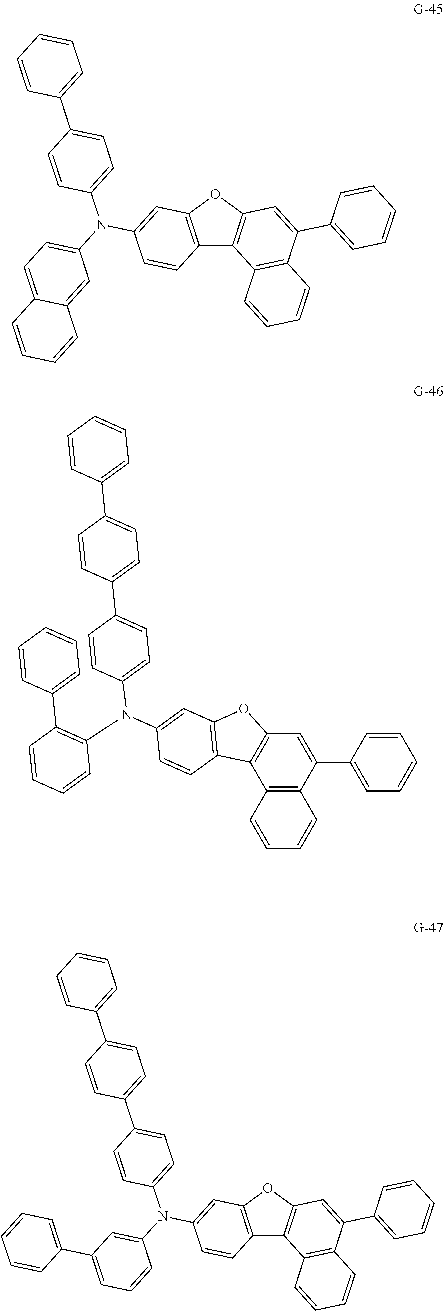 [SMILES: c1ccc(-c2ccc(N(c3ccc4ccccc4c3)c3ccc4c(c3)oc3cc(-c5ccccc5)c5ccccc5c34)cc2)cc1 ; c1ccc(-c2ccc(-c3ccc(N(c4ccc5c(c4)oc4cc(-c6ccccc6)c6ccccc6c45)c4ccccc4-c4ccccc4)cc3)cc2)cc1 ; c1ccc(-c2ccc(-c3ccc(N(c4cccc(-c5ccccc5)c4)c4ccc5c(c4)oc4cc(-c6ccccc6)c6ccccc6c45)cc3)cc2)cc1]

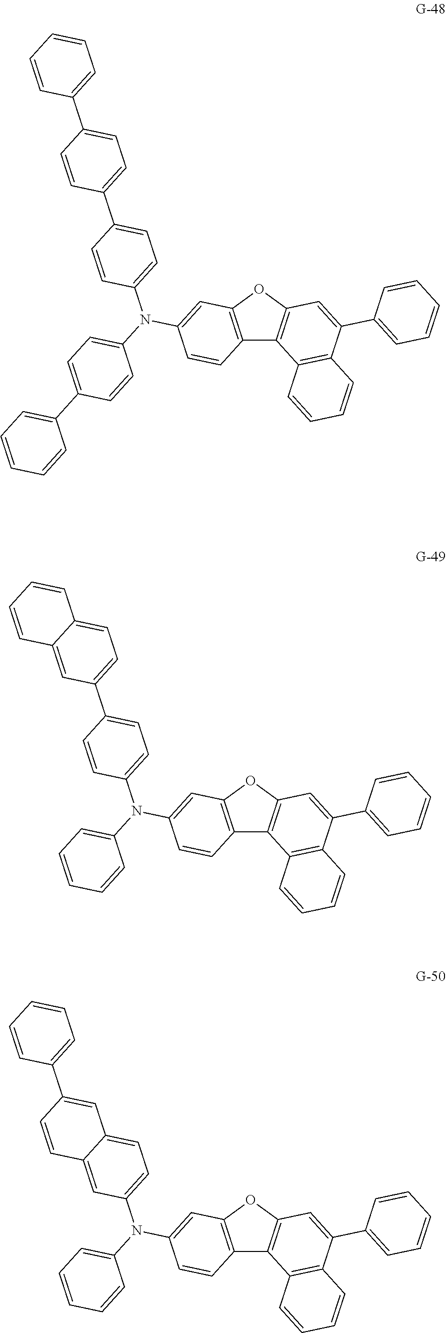 [SMILES: c1ccc(-c2ccc(-c3ccc(N(c4ccc(-c5ccccc5)cc4)c4ccc5c(c4)oc4cc(-c6ccccc6)c6ccccc6c45)cc3)cc2)cc1 ; c1ccc(-c2cc3oc4cc(N(c5ccccc5)c5ccc(-c6ccc7ccccc7c6)cc5)ccc4c3c3ccccc23)cc1 ; c1ccc(-c2ccc3cc(N(c4ccccc4)c4ccc5c(c4)oc4cc(-c6ccccc6)c6ccccc6c45)ccc3c2)cc1]

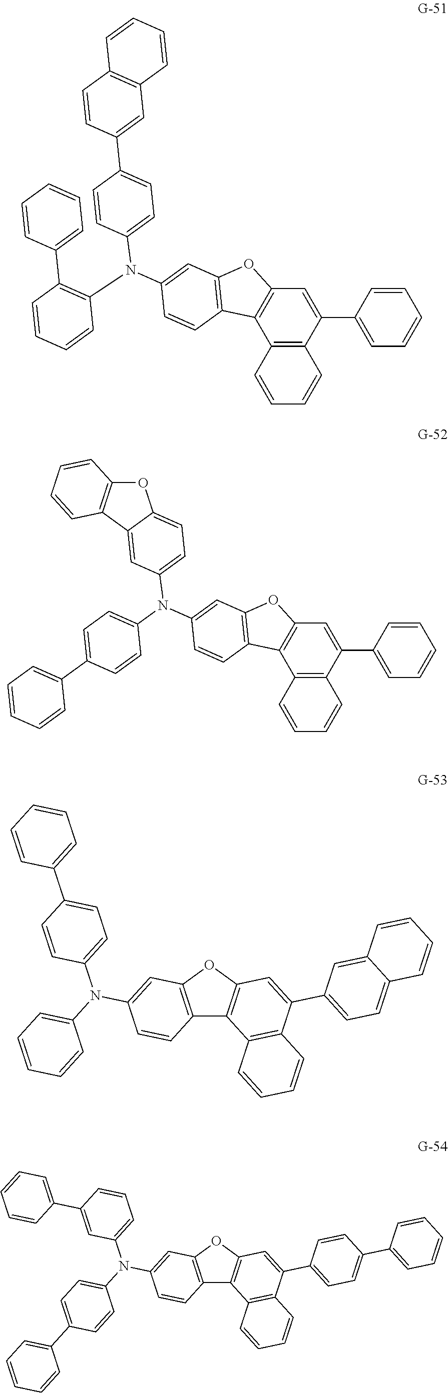 [SMILES: c1ccc(-c2ccccc2N(c2ccc(-c3ccc4ccccc4c3)cc2)c2ccc3c(c2)oc2cc(-c4ccccc4)c4ccccc4c23)cc1 ; c1ccc(-c2ccc(N(c3ccc4c(c3)oc3cc(-c5ccccc5)c5ccccc5c34)c3ccc4oc5ccccc5c4c3)cc2)cc1 ; c1ccc(-c2ccc(N(c3ccccc3)c3ccc4c(c3)oc3cc(-c5ccc6ccccc6c5)c5ccccc5c34)cc2)cc1 ; c1ccc(-c2ccc(-c3cc4oc5cc(N(c6ccc(-c7ccccc7)cc6)c6cccc(-c7ccccc7)c6)ccc5c4c4ccccc34)cc2)cc1]

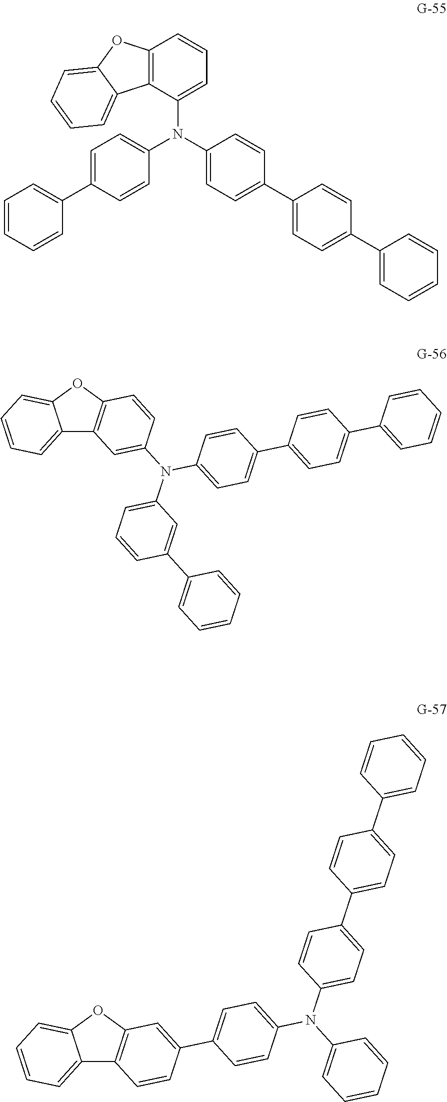 [SMILES: c1ccc(-c2ccc(-c3ccc(N(c4ccc(-c5ccccc5)cc4)c4cccc5oc6ccccc6c45)cc3)cc2)cc1 ; c1ccc(-c2ccc(-c3ccc(N(c4cccc(-c5ccccc5)c4)c4ccc5oc6ccccc6c5c4)cc3)cc2)cc1 ; c1ccc(-c2ccc(-c3ccc(N(c4ccccc4)c4ccc(-c5ccc6c(c5)oc5ccccc56)cc4)cc3)cc2)cc1]

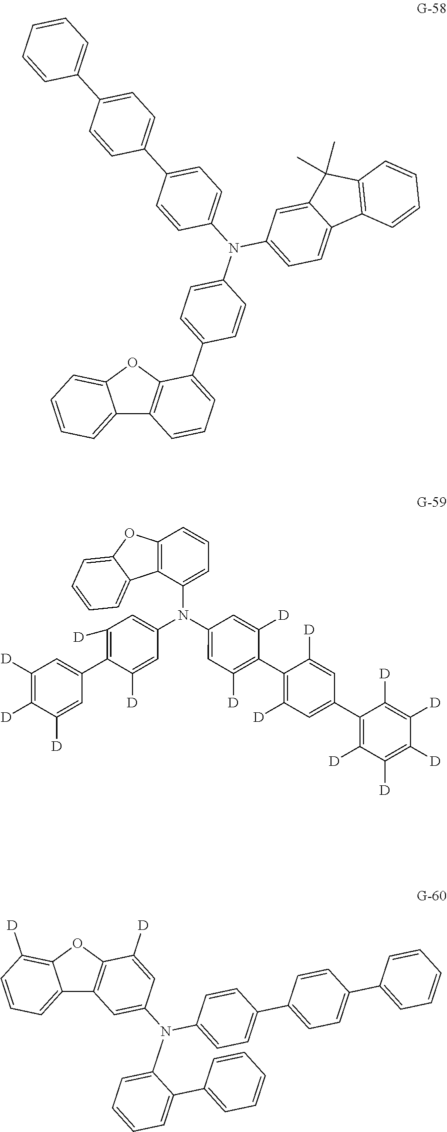 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; [2H]c1cc(-c2c([2H])cc(N(c3cc([2H])c(-c4c([2H])cc(-c5c([2H])c([2H])c([2H])c([2H])c5[2H])cc4[2H])c([2H])c3)c3cccc4oc5ccccc5c34)cc2[2H])cc([2H])c1[2H] ; [2H]c1cccc2c1oc1c([2H])cc(N(c3ccc(-c4ccc(-c5ccccc5)cc4)cc3)c3ccccc3-c3ccccc3)cc12]

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical methods and materials for manufacturing an organic light emitting device, except that an organic material layer having one or more layers is formed by using the heterocyclic compound of the above-described Chemical Formula 1.

The heterocyclic compound of Chemical Formula 1 may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a light emitting layer of a blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a light emitting layer of a green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a light emitting layer of a red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

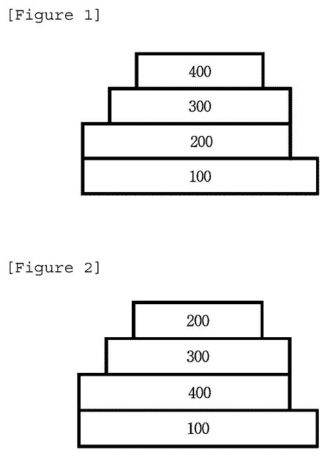

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented.

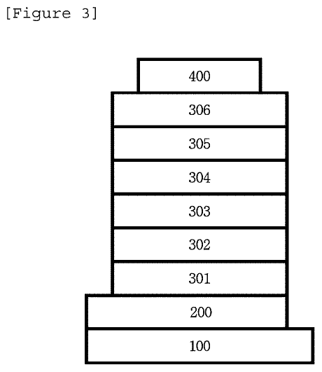

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

Another exemplary embodiment of the present specification provides a composition for an organic material layer including the heterocyclic compound.

In an exemplary embodiment of the present specification, the composition for an organic material layer may further include the compound of Chemical Formula 2.

In an exemplary embodiment of the present specification, the composition for an organic material layer may include the heterocyclic compound and the compound of Chemical Formula 2 at a weight ratio of 1:10 to 10:1.

In an exemplary embodiment of the present specification, the composition for an organic material layer may include the heterocyclic compound and the compound of Chemical Formula 2 at a weight ratio of 1:8 to 8:1, 1:5 to 5:1, or 1:3 to 3:1.

In an exemplary embodiment of the present specification, the composition for an organic material layer may include the heterocyclic compound and the compound of Chemical Formula 2 at a weight ratio of 1:1 to 5:1, or 1:1 to 3:1.

Still another exemplary embodiment of the present specification provides a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the above-described composition for an organic material layer.

In an exemplary embodiment of the present specification, the forming of the organic material layer may include pre-mixing the composition for an organic layer of the organic light emitting device to deposit the pre-mixed composition onto a single supply source.

The pre-mixing means that before the heterocyclic compound of Chemical Formula 1 and the compound of Chemical Formula 2 are deposited onto an organic material layer, the materials are first mixed and the mixture is contained in one common container and mixed. Since one deposition source is used instead of using two or more deposition sources during the pre-mixing, there is an advantage in that the process is more simplified.

When the composition for an organic material layer is pre-mixed, the deposition conditions, such as the deposition rate, may be significantly affected by the inherent thermal characteristics of the material during the deposition of the pre-mixed material, so that the inherent thermal characteristics of each pre-mixed material need to be confirmed. When the thermal properties of the materials are not similar, the deposition process cannot be repeated or reproduced, and an uniform OLED device cannot be manufactured.

In order to overcome this problem, the electrical characteristics of the material may be controlled by utilizing the appropriate combination of the basic structure of each material and the substituent, and simultaneously, the thermal characteristics may also be adjusted according to the form of the molecular structure. The thermal characteristics of each material may be adjusted to secure the diversity of various pre-mixing deposition processes between a host and a host. Through this, it is possible to secure the diversity of the pre-mixing deposition process utilizing not only two compounds as hosts but also 3 or more types of host materials.

In an exemplary embodiment of the present specification, the composition for an organic material layer may include other hosts in addition to the compound of Chemical Formula 2.

In an exemplary embodiment of the present specification, the composition for an organic material layer includes the compound of Chemical Formula 2, and may further include another host.

Hereinafter, the present specification will be described in more detail through Examples, but these Examples are provided only for exemplifying the present application, and are not intended to limit the scope of the present application.

### Preparation Examples

### <Preparation Example 1> Preparation of Compound D-1

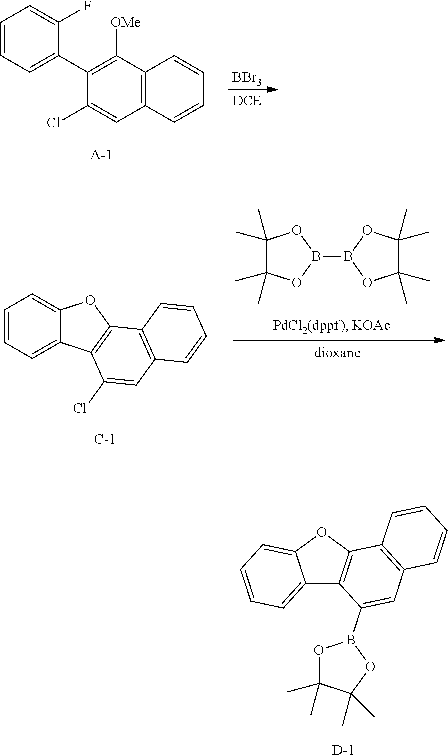

### 1) Synthesis of Compound C-1

3-chloro-2-(2-fluorophenyl)-1-methoxynaphthalene (A-1) (57.4 g, 200 mmol) was put into a 1000 mL round-bottom flask, a nitrogen atmosphere was created, then DCE (350 mL) was added thereto, and BBr₃ (100 g, 38 mL, 400 mmol) was slowly added dropwise thereto at 0° C. for 30 minutes. After stirring at room temperature for 4 hours, the reaction was terminated by adding ice-cold water (200 mL) to the reactant, and then extraction was performed with MC. The extracted organic solvent was dried over Mg₂SO₄, and then concentrated. After purification using a silica-gel column and methanol, Compound C-1 (37.9 g, 150 mmol, yield 75%) was obtained as a white solid.

### 2) Synthesis of Compound D-1

Compound C-1 (37.9 g, 150 mmol), [1,1″-bis(diphenylphosphino)ferrocene]dichloropalladium(II) (PdCl₂ (dppf)) (8.6 g, 11.75 mmol), and potassium acetate (KOAc) (44 g, 450 mmol) were put into a 1000 mL round-bottom flask, a nitrogen atmosphere was created, then 1,4-dioxane (350 mL) was added thereto, and the resulting mixture was stirred at 100° C. for 6 hours. After the reaction temperature was lowered to room temperature, the reaction was terminated by adding water (100 mL) thereto, and then extraction was performed with methylene chloride (MC). The extracted organic solvent was dried over Mg₂SO₄, and then concentrated. After re-crystallization using a silica-gel column and methanol, Compound D-1 (48 g, 140 mmol, 93%) was obtained as a white solid.

### <Preparation Example 2> Preparation of Compound D-3

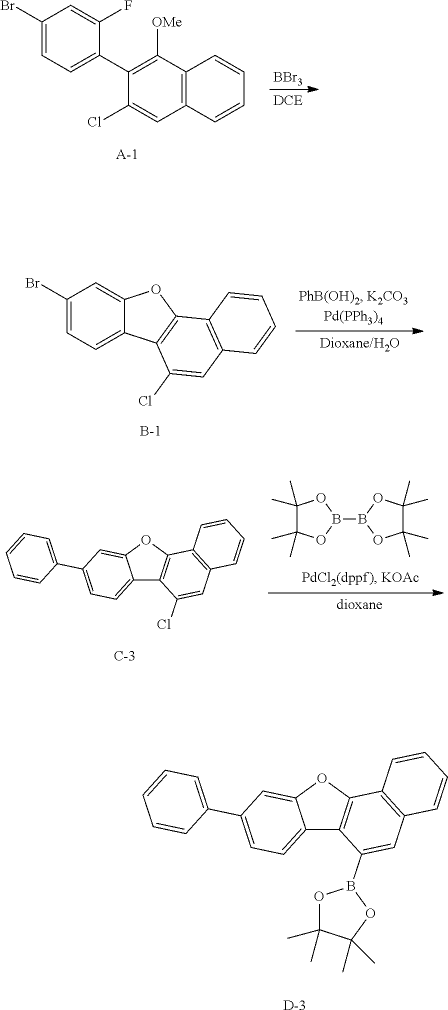

### 2) Synthesis of Compound C-3

Compound B-1 (50 g, 150 mmol, yield 73%) as a white solid was obtained by performing synthesis in the same manner as in the method of synthesizing Compound C-1 in Preparation Example 1, except that Compound A-2 was used instead of Compound A-1.

### 2) Synthesis of Compound C-3

Compound B-1 (50 g, 150 mmol), phenylboronic acid (X) (222 g, 200 mmol), tetrakis(triphenylphosphine)palladium(0) (Pd(PPh₃)₄) (18.5 g, 7 mmol), and K₂CO₃ (41 g, 300 mmol) were put into a 1000 mL round-bottom flask, dioxane (300 mL)/H₂O (60 mL) were added thereto, and the resulting mixture was stirred at 120° C. for 6 hours. After the reaction was completed, the reaction temperature was lowered to room temperature, and the resulting product was washed with water, and then extracted with MC. The extracted organic solvent was dried over Mg₂SO₄, and then concentrated. Silica-gel column chromatography and re-crystallization were performed to obtain Compound C-3 (47 g, 143 mmol, 96%) as a white solid.

### 2) Synthesis of Compound D-3

Compound C-3 (47 g, 143 mmol), PdCl₂(dppf) (5.23 g, 7.15 mmol), and KOAc (28.06 g, 286 mmol) were put into a 1000 mL round-bottom flask, a nitrogen atmosphere was created, then 1,4-dioxane (500 mL) was added thereto, and the resulting mixture was stirred at 100° C. for 3 hours. After the reaction temperature was lowered to room temperature, the reaction was terminated by adding water (100 mL) thereto, and then extraction was performed with MC. The extracted organic solvent was dried over Mg₂SO₄, and then concentrated. After re-crystallization using a silica-gel column and methanol, Compound D-3 (54 g, 129 mmol, 90%) was obtained as a white solid.

### <Preparation Example 3> Preparation of Compound C-2

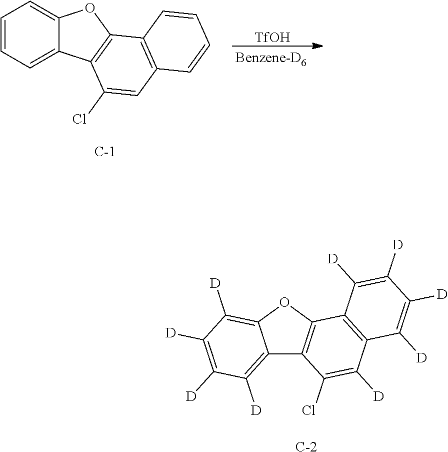

Compound C-1 (20 g, 79 mmol) was put into a 1000 mL round-bottom flask and dissolved in benzene-D6 (200 mL) solvent, and then an ice bath was set up. After trifluoromethanesulfonic acid (TfOH) (49.8 mL, 554 mmol) was slowly added dropwise thereto, the resulting mixture was stirred at 60° C. for 12 hours. The reaction temperature was lowered to room temperature, an ice bath was set up, a neutralization reaction was performed using distilled water, and then extraction was performed with MC. The extracted organic solvent was dried over Mg₂SO₄, and then concentrated. After purification using methanol, Compound C-2 (20 g, 76.4 mmol, 97%) was obtained as a white solid.

### <Preparation Example 4> Preparation of Compounds in Table 1

In the method of Preparation Example 1 or 2, Compound A in the following Table 1 was used instead of Compound A-1 or A-2, and in Preparation Example 2, Compound X was used instead of phenylboronic acid, and when the core structure includes deuterium, the method of Preparation Example 3 was additionally carried out (indicated as +3) in the step prior to the preparation of Compound D to synthesize Compounds B to D in the following Table 1.

TABLE 1

| Preparation | | Compound B | | Compound C | Compound D |
|---|---|---|---|---|---|
| Example | Compound A | (Yield %) | Compound X | (Yield %) | (Yield %) |
| 1 | 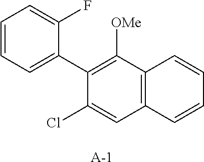 | — | — | 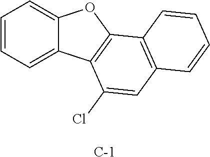 (75%) | 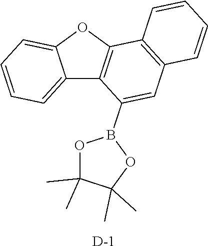 (93%) |
| 1 + 3 | 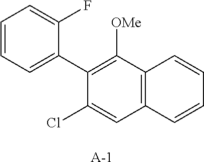 | — | — | 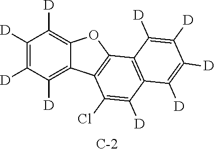 (97%) | 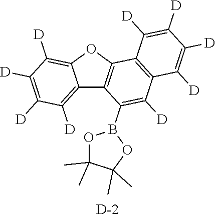 (93%) |
| 2 | 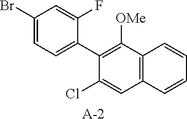 | 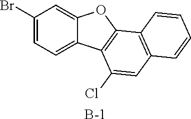 (73%) | 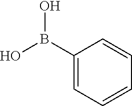 | 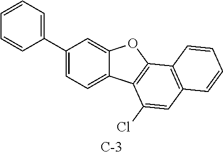 (96%) | 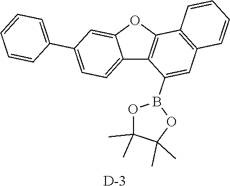 (90%) |
| 2 + 3 | 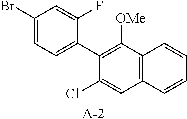 | 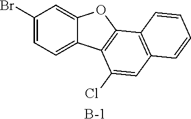 (73%) | 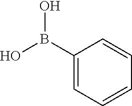 | 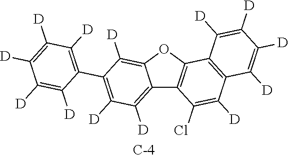 (79%) | 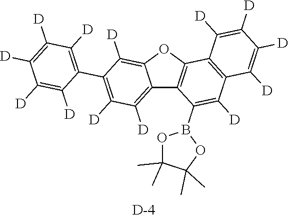 (92%) |
| 2 | 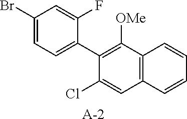 | 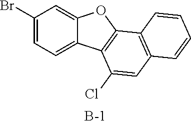 (73%) | 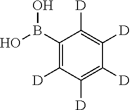 [SMILES: [2H]c1c([2H])c([2H])c(B(O)O)c([2H])c1[2H]] | 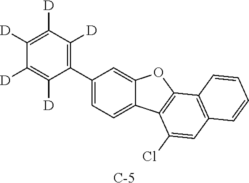 (77%) | 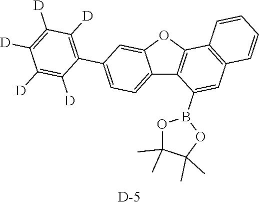 (90%) |
| 2 | 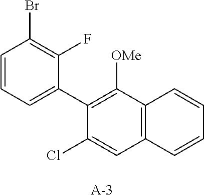 | 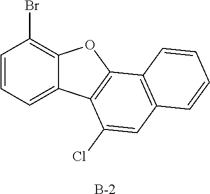 (77%) | 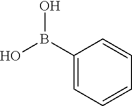 | 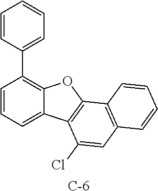 (90%) | 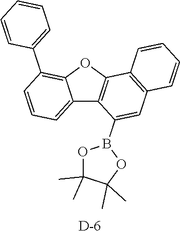 (91%) |
| 1 | 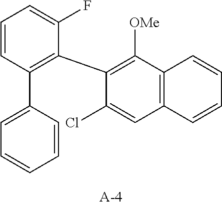 [SMILES: COc1c(-c2c(F)cccc2-c2ccccc2)c(Cl)cc2ccccc12 ; "A-4"] | — | 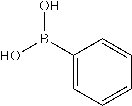 | 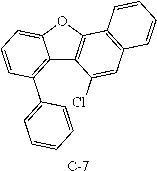 (69%) | 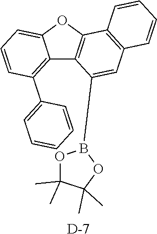 (88%) |
| 1 | 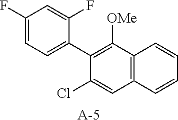 | — | — | 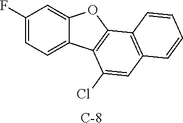 (66%) | 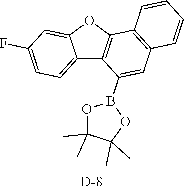 (84%) |
| 1 | 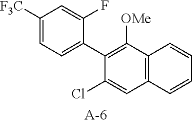 | — | — | 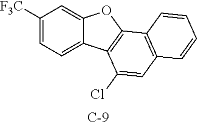 (65%) | 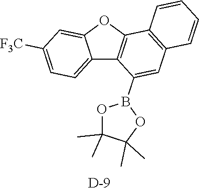 (90%) |
| 1 | 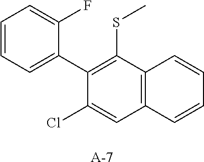 | — | — | 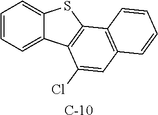 (72%) | 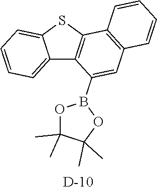 (92%) |
| 1 + 3 | 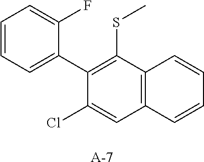 | — | — | 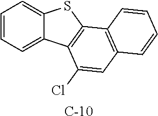 (72%) | 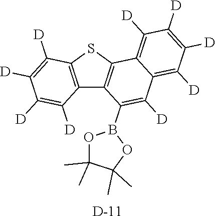 (87%) |
| 2 | 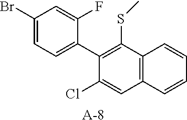 | 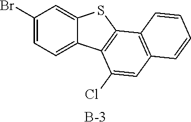 (70%) | 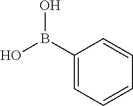 | 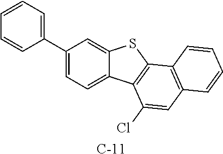 (90%) | 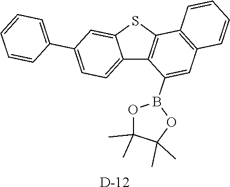 (90%) |
| 1 | 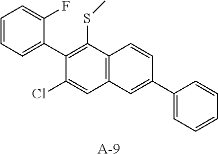 [SMILES: CSc1c(-c2ccccc2F)c(Cl)cc2cc(-c3ccccc3)ccc12 ; "A-9"] | — | — | 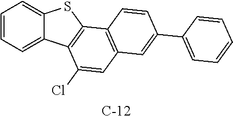 (68%) | 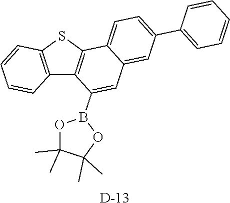 (91%) |
| 1 | 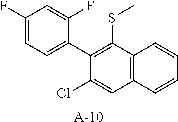 | — | — | 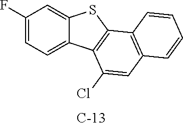 (59%) | 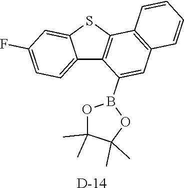 (87%) |
| 2 + 3 | 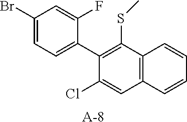 | 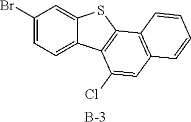 (70%) | 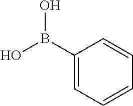 | 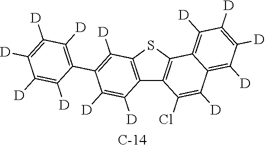 (70%) | 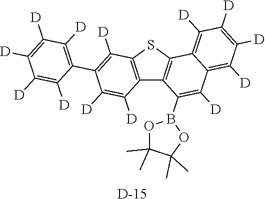 (84%) |
| 2 + 3 | 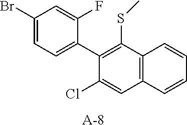 | 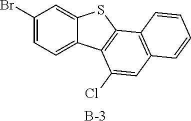 (70%) | 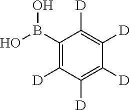 [SMILES: [2H]c1c([2H])c([2H])c(B(O)O)c([2H])c1[2H]] | 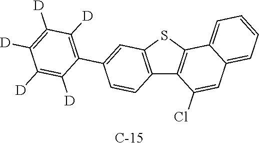 (82%) | 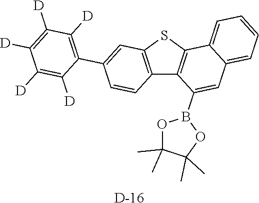 (90%) |

### <Preparation Example 5> Preparation of Compound F-1

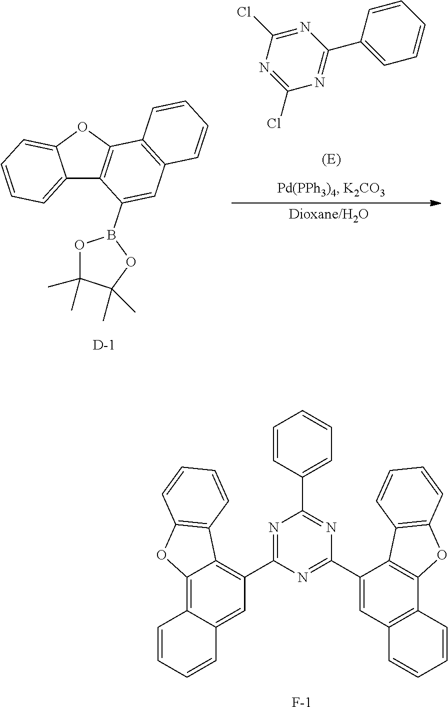

Compound D-1 (7.3 g, 21 mmol), 2,4-dichloro-6-phenyl-1,3,5-triazine (E) (2.3 g, 10 mmol), Pd(PPh₃)₄ (0.59 g, 0.51 mmol), and K₂CO₃ (2.81 g, 20.35 mmol) were put into a 250 mL round-bottom flask, dioxine (70 mL)/H₂O (10 mL) were added thereto, and the resulting mixture was stirred at 120° C. for 12 hours. After the reaction was completed, the reaction temperature was lowered to room temperature and a precipitated solid was filtered. The filtered solid was washed with water, dissolved in chloroform, and then purified using a silica-gel column and acetone to obtain Compound F-1 (5.2 g, 8.8 mmol, 87%) as a white solid.

The following Target Compound F was synthesized by performing synthesis in the same manner as in Preparation Example 5, except that Compound D in the following Table 2 was used instead of Compound D-1 and Compound F in the following Table 2 was used instead of Compound (F).

TABLE 2

| Compound D | Compound E | Compound F (Yield) |
|---|---|---|
| 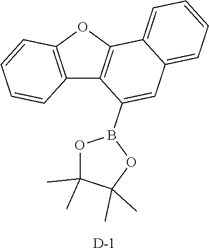 | 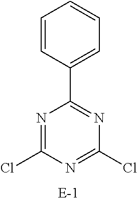 [SMILES: Clc1nc(Cl)nc(-c2ccccc2)n1] | 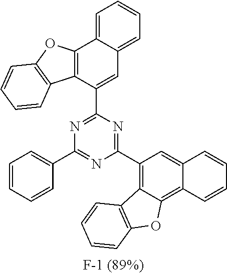 [SMILES: c1ccc(-c2nc(-c3cc4ccccc4c4oc5ccccc5c34)nc(-c3cc4ccccc4c4oc5ccccc5c34)n2)cc1] |
| 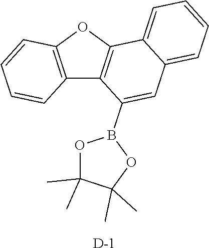 | 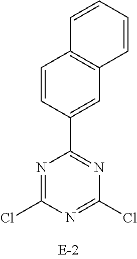 [SMILES: Clc1nc(Cl)nc(-c2ccc3ccccc3c2)n1] | 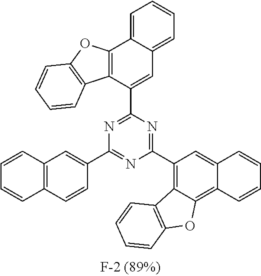 [SMILES: c1ccc2cc(-c3nc(-c4cc5ccccc5c5oc6ccccc6c45)nc(-c4cc5ccccc5c5oc6ccccc6c45)n3)ccc2c1] |
| 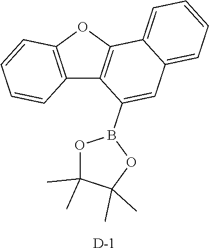 | 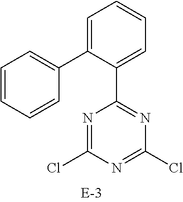 [SMILES: Clc1nc(Cl)nc(-c2ccccc2-c2ccccc2)n1] | 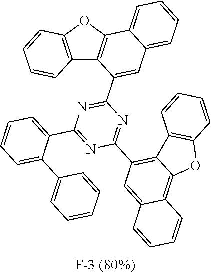 [SMILES: c1ccc(-c2ccccc2-c2nc(-c3cc4ccccc4c4oc5ccccc5c34)nc(-c3cc4ccccc4c4oc5ccccc5c34)n2)cc1] |
| 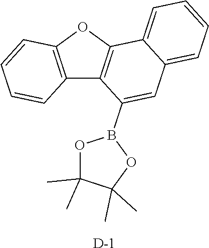 | 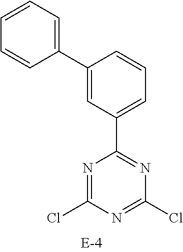 [SMILES: Clc1nc(Cl)nc(-c2cccc(-c3ccccc3)c2)n1] | 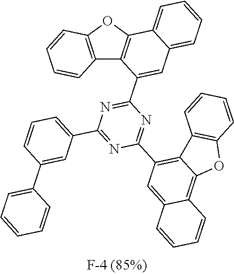 [SMILES: c1ccc(-c2cccc(-c3nc(-c4cc5ccccc5c5oc6ccccc6c45)nc(-c4cc5ccccc5c5oc6ccccc6c45)n3)c2)cc1] |
| 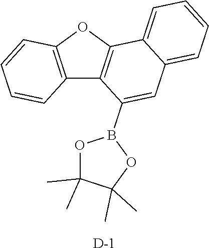 | 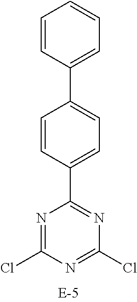 [SMILES: Clc1nc(Cl)nc(-c2ccc(-c3ccccc3)cc2)n1] | 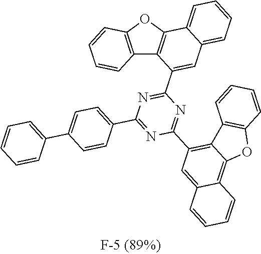 [SMILES: c1ccc(-c2ccc(-c3nc(-c4cc5ccccc5c5oc6ccccc6c45)nc(-c4cc5ccccc5c5oc6ccccc6c45)n3)cc2)cc1] |
| 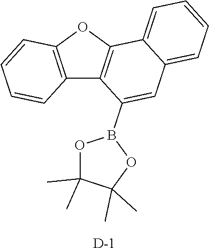 | 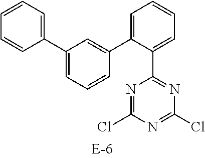 [SMILES: Clc1nc(Cl)nc(-c2ccccc2-c2cccc(-c3ccccc3)c2)n1] | 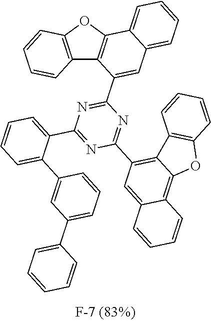 [SMILES: c1ccc(-c2cccc(-c3ccccc3-c3nc(-c4cc5ccccc5c5oc6ccccc6c45)nc(-c4cc5ccccc5c5oc6ccccc6c45)n3)c2)cc1] |
| 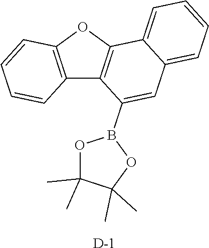 | 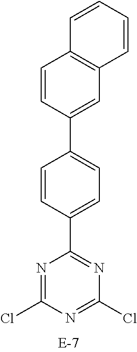 [SMILES: Clc1nc(Cl)nc(-c2ccc(-c3ccc4ccccc4c3)cc2)n1] | 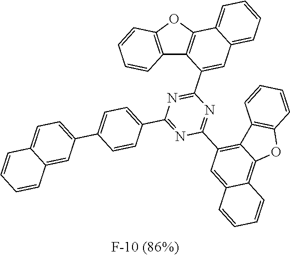 [SMILES: c1ccc2cc(-c3ccc(-c4nc(-c5cc6ccccc6c6oc7ccccc7c56)nc(-c5cc6ccccc6c6oc7ccccc7c56)n4)cc3)ccc2c1] |
| 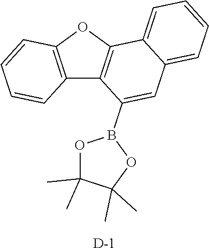 | 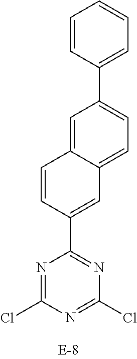 [SMILES: Clc1nc(Cl)nc(-c2ccc3cc(-c4ccccc4)ccc3c2)n1] | 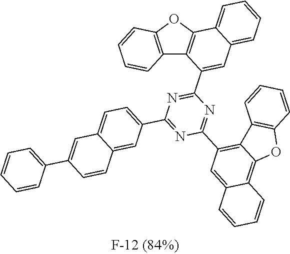 [SMILES: c1ccc(-c2ccc3cc(-c4nc(-c5cc6ccccc6c6oc7ccccc7c56)nc(-c5cc6ccccc6c6oc7ccccc7c56)n4)ccc3c2)cc1] |
| 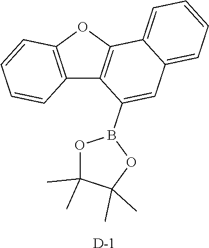 | 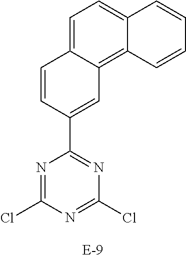 [SMILES: Clc1nc(Cl)nc(-c2ccc3ccc4ccccc4c3c2)n1] | 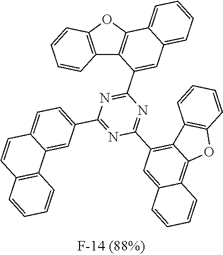 [SMILES: c1ccc2c(c1)ccc1ccc(-c3nc(-c4cc5ccccc5c5oc6ccccc6c45)nc(-c4cc5ccccc5c5oc6ccccc6c45)n3)cc12] |
| 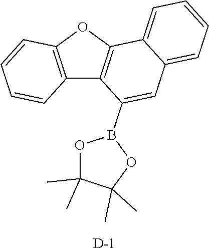 | 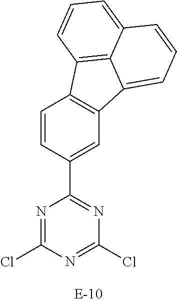 [SMILES: Clc1nc(Cl)nc(-c2ccc3c(c2)-c2cccc4cccc-3c24)n1] | 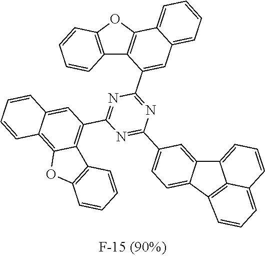 [SMILES: c1cc2c3c(cccc3c1)-c1cc(-c3nc(-c4cc5ccccc5c5oc6ccccc6c45)nc(-c4cc5ccccc5c5oc6ccccc6c45)n3)ccc1-2] |
| 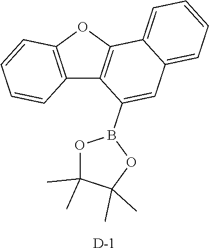 | 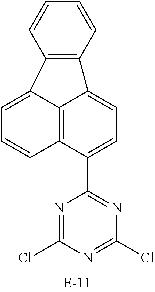 [SMILES: Clc1nc(Cl)nc(-c2ccc3c4c(cccc24)-c2ccccc2-3)n1] | 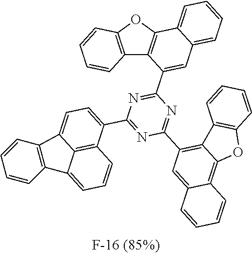 [SMILES: c1ccc2c(c1)-c1cccc3c(-c4nc(-c5cc6ccccc6c6oc7ccccc7c56)nc(-c5cc6ccccc6c6oc7ccccc7c56)n4)ccc-2c13] |
| 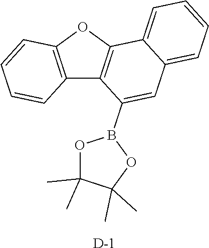 | 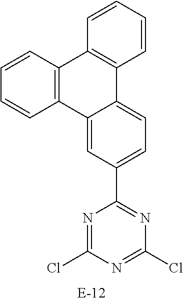 [SMILES: Clc1nc(Cl)nc(-c2ccc3c4ccccc4c4ccccc4c3c2)n1] | 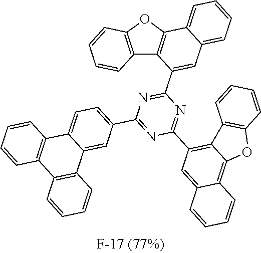 [SMILES: c1ccc2c(c1)cc(-c1nc(-c3ccc4c5ccccc5c5ccccc5c4c3)nc(-c3cc4ccccc4c4oc5ccccc5c34)n1)c1c3ccccc3oc21] |
| 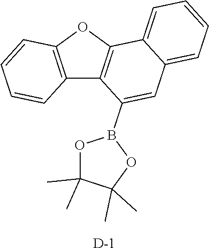 | 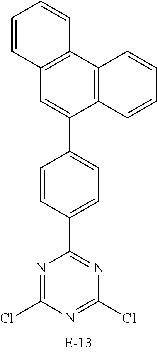 [SMILES: Clc1nc(Cl)nc(-c2ccc(-c3cc4ccccc4c4ccccc34)cc2)n1] | 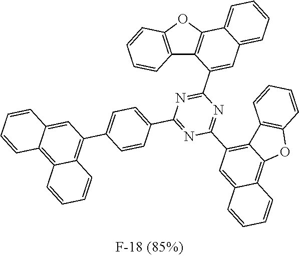 [SMILES: c1ccc2c(c1)cc(-c1ccc(-c3nc(-c4cc5ccccc5c5oc6ccccc6c45)nc(-c4cc5ccccc5c5oc6ccccc6c45)n3)cc1)c1ccccc12] |
| 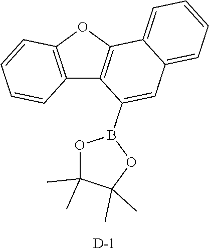 | 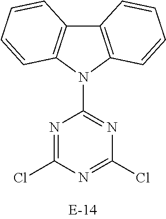 [SMILES: Clc1nc(Cl)nc(-n2c3ccccc3c3ccccc32)n1] | 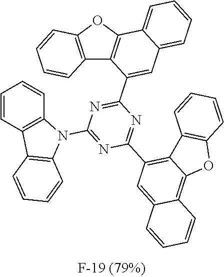 [SMILES: c1ccc2c(c1)cc(-c1nc(-c3cc4ccccc4c4oc5ccccc5c34)nc(-n3c4ccccc4c4ccccc43)n1)c1c3ccccc3oc21] |
| 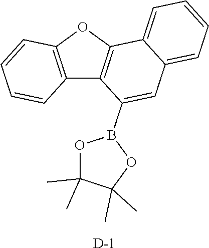 | 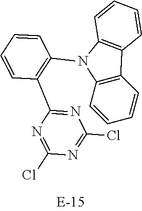 [SMILES: Clc1nc(Cl)nc(-c2ccccc2-n2c3ccccc3c3ccccc32)n1] | 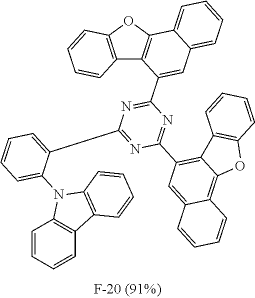 [SMILES: c1ccc(-n2c3ccccc3c3ccccc32)c(-c2nc(-c3cc4ccccc4c4oc5ccccc5c34)nc(-c3cc4ccccc4c4oc5ccccc5c34)n2)c1] |
| 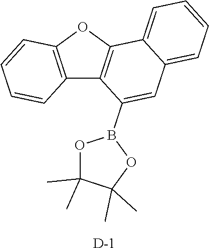 | 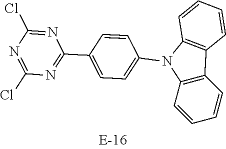 [SMILES: Clc1nc(Cl)nc(-c2ccc(-n3c4ccccc4c4ccccc43)cc2)n1] | 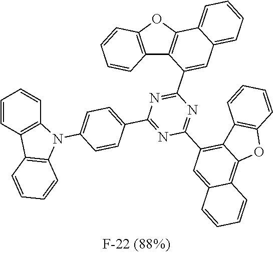 [SMILES: c1ccc2c(c1)cc(-c1nc(-c3ccc(-n4c5ccccc5c5ccccc54)cc3)nc(-c3cc4ccccc4c4oc5ccccc5c34)n1)c1c3ccccc3oc21] |
| 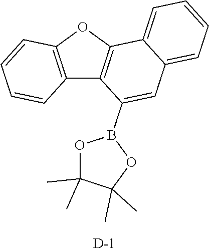 | 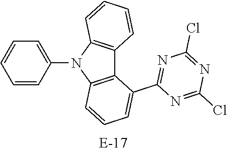 [SMILES: Clc1nc(Cl)nc(-c2cccc3c2c2ccccc2n3-c2ccccc2)n1] | 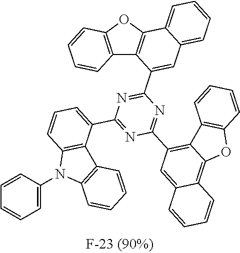 [SMILES: c1ccc(-n2c3ccccc3c3c(-c4nc(-c5cc6ccccc6c6oc7ccccc7c56)nc(-c5cc6ccccc6c6oc7ccccc7c56)n4)cccc32)cc1] |
| 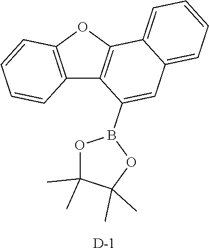 | 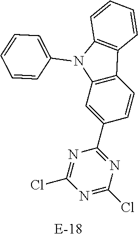 [SMILES: Clc1nc(Cl)nc(-c2ccc3c4ccccc4n(-c4ccccc4)c3c2)n1] | 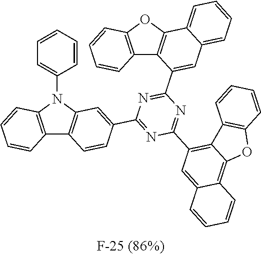 [SMILES: c1ccc(-n2c3ccccc3c3ccc(-c4nc(-c5cc6ccccc6c6oc7ccccc7c56)nc(-c5cc6ccccc6c6oc7ccccc7c56)n4)cc32)cc1] |
| 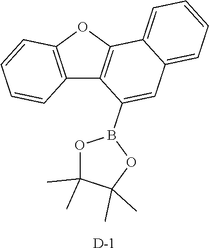 | 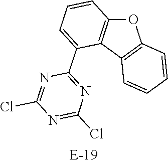 [SMILES: Clc1nc(Cl)nc(-c2cccc3oc4ccccc4c23)n1] | 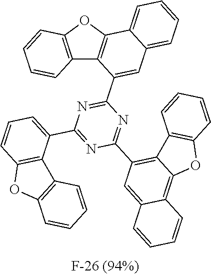 [SMILES: c1ccc2c(c1)cc(-c1nc(-c3cccc4oc5ccccc5c34)nc(-c3cc4ccccc4c4oc5ccccc5c34)n1)c1c3ccccc3oc21] |
| 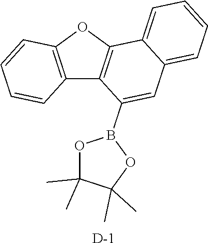 | 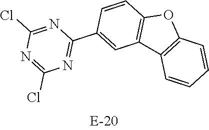 [SMILES: Clc1nc(Cl)nc(-c2ccc3oc4ccccc4c3c2)n1] | 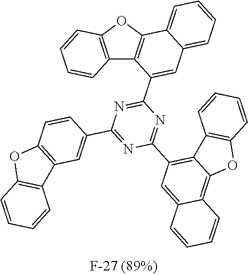 [SMILES: c1ccc2c(c1)cc(-c1nc(-c3ccc4oc5ccccc5c4c3)nc(-c3cc4ccccc4c4oc5ccccc5c34)n1)c1c3ccccc3oc21] |
| 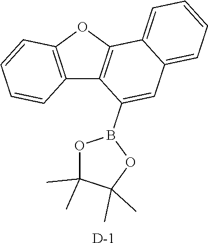 | 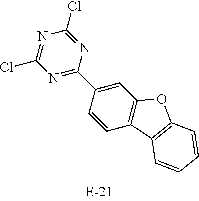 [SMILES: Clc1nc(Cl)nc(-c2ccc3c(c2)oc2ccccc23)n1] | 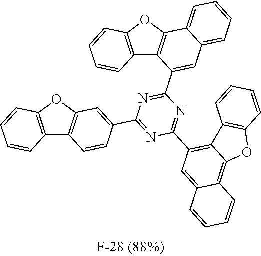 [SMILES: c1ccc2c(c1)cc(-c1nc(-c3ccc4c(c3)oc3ccccc34)nc(-c3cc4ccccc4c4oc5ccccc5c34)n1)c1c3ccccc3oc21] |
| 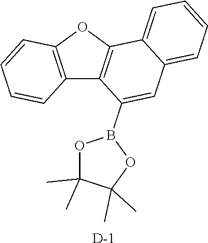 | 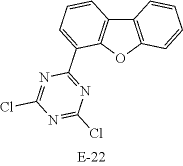 [SMILES: Clc1nc(Cl)nc(-c2cccc3c2oc2ccccc23)n1] | 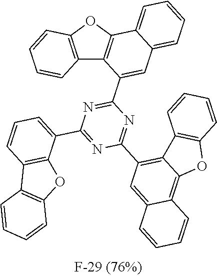 [SMILES: c1ccc2c(c1)cc(-c1nc(-c3cccc4c3oc3ccccc34)nc(-c3cc4ccccc4c4oc5ccccc5c34)n1)c1c3ccccc3oc21] |
| 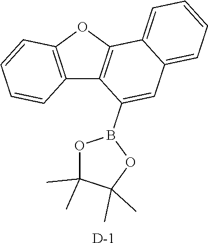 | 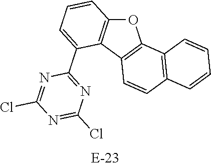 [SMILES: Clc1nc(Cl)nc(-c2cccc3oc4c5ccccc5ccc4c23)n1] | 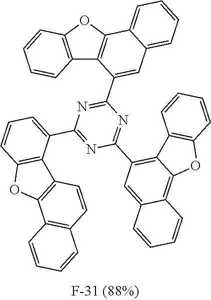 [SMILES: c1ccc2c(c1)ccc1c2oc2cccc(-c3nc(-c4cc5ccccc5c5oc6ccccc6c45)nc(-c4cc5ccccc5c5oc6ccccc6c45)n3)c21] |
| 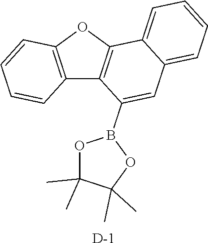 | 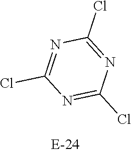 | 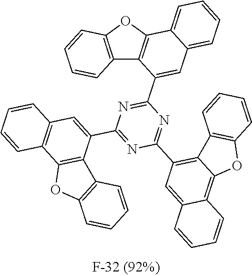 [SMILES: c1ccc2c(c1)cc(-c1nc(-c3cc4ccccc4c4oc5ccccc5c34)nc(-c3cc4ccccc4c4oc5ccccc5c34)n1)c1c3ccccc3oc21] |
| 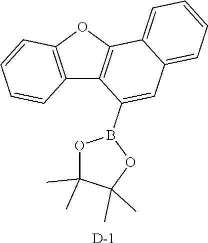 | 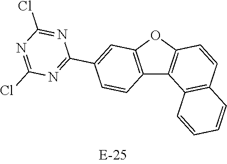 [SMILES: Clc1nc(Cl)nc(-c2ccc3c(c2)oc2ccc4ccccc4c23)n1] | 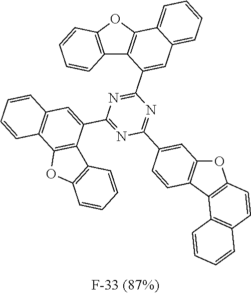 [SMILES: c1ccc2c(c1)cc(-c1nc(-c3ccc4c(c3)oc3ccc5ccccc5c34)nc(-c3cc4ccccc4c4oc5ccccc5c34)n1)c1c3ccccc3oc21] |
| 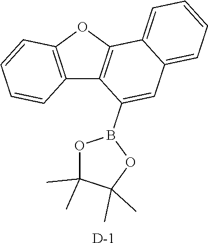 | 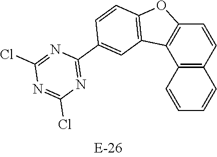 [SMILES: Clc1nc(Cl)nc(-c2ccc3oc4ccc5ccccc5c4c3c2)n1] | 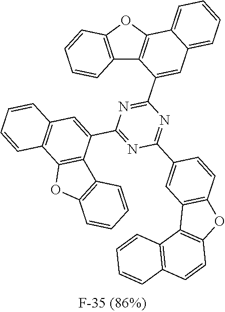 [SMILES: c1ccc2c(c1)cc(-c1nc(-c3ccc4oc5ccc6ccccc6c5c4c3)nc(-c3cc4ccccc4c4oc5ccccc5c34)n1)c1c3ccccc3oc21] |
| 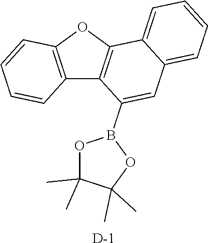 | 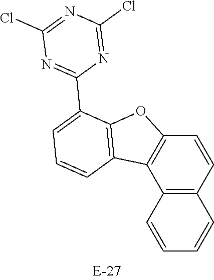 [SMILES: Clc1nc(Cl)nc(-c2cccc3c2oc2ccc4ccccc4c23)n1] | 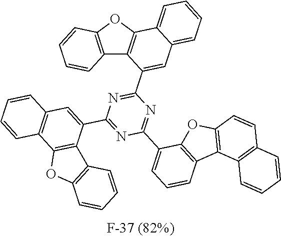 [SMILES: c1ccc2c(c1)cc(-c1nc(-c3cccc4c3oc3ccc5ccccc5c34)nc(-c3cc4ccccc4c4oc5ccccc5c34)n1)c1c3ccccc3oc21] |
| 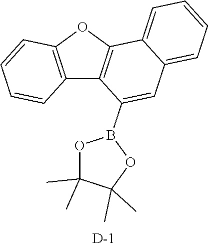 | 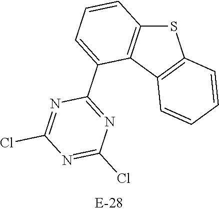 [SMILES: Clc1nc(Cl)nc(-c2cccc3sc4ccccc4c23)n1] | 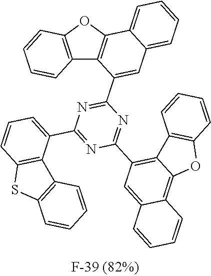 [SMILES: c1ccc2c(c1)cc(-c1nc(-c3cccc4sc5ccccc5c34)nc(-c3cc4ccccc4c4oc5ccccc5c34)n1)c1c3ccccc3oc21] |
| 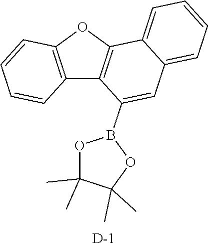 | 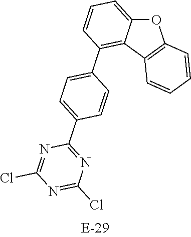 [SMILES: Clc1nc(Cl)nc(-c2ccc(-c3cccc4oc5ccccc5c34)cc2)n1] | 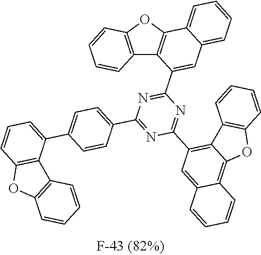 [SMILES: c1ccc2c(c1)cc(-c1nc(-c3ccc(-c4cccc5oc6ccccc6c45)cc3)nc(-c3cc4ccccc4c4oc5ccccc5c34)n1)c1c3ccccc3oc21] |
| 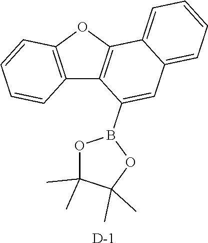 | 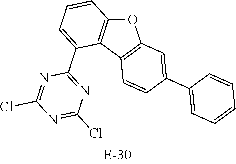 [SMILES: Clc1nc(Cl)nc(-c2cccc3oc4cc(-c5ccccc5)ccc4c23)n1] | 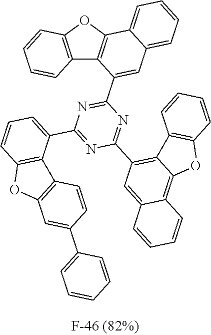 [SMILES: c1ccc(-c2ccc3c(c2)oc2cccc(-c4nc(-c5cc6ccccc6c6oc7ccccc7c56)nc(-c5cc6ccccc6c6oc7ccccc7c56)n4)c23)cc1] |
| 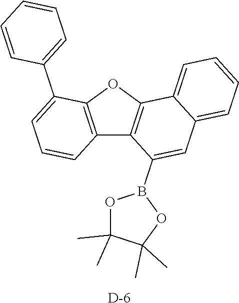 | 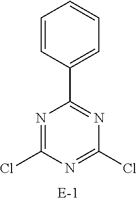 [SMILES: Clc1nc(Cl)nc(-c2ccccc2)n1] | 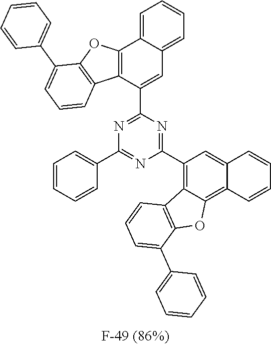 [SMILES: c1ccc(-c2nc(-c3cc4ccccc4c4oc5c(-c6ccccc6)cccc5c34)nc(-c3cc4ccccc4c4oc5c(-c6ccccc6)cccc5c34)n2)cc1] |
| 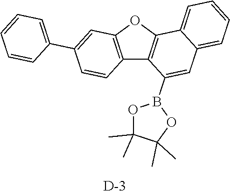 | | 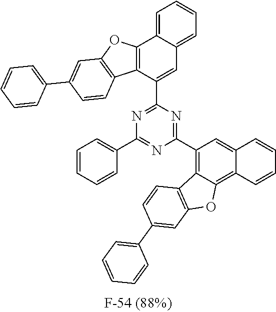 [SMILES: c1ccc(-c2ccc3c(c2)oc2c4ccccc4cc(-c4nc(-c5ccccc5)nc(-c5cc6ccccc6c6oc7cc(-c8ccccc8)ccc7c56)n4)c32)cc1] |
| 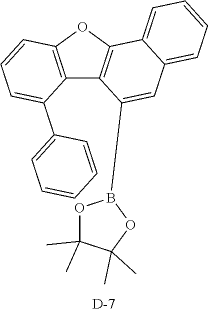 | | 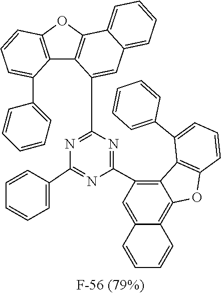 [SMILES: c1ccc(-c2nc(-c3cc4ccccc4c4oc5cccc(-c6ccccc6)c5c34)nc(-c3cc4ccccc4c4oc5cccc(-c6ccccc6)c5c34)n2)cc1] |
| 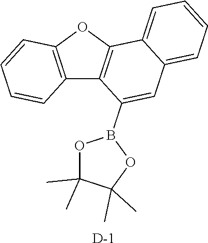 | 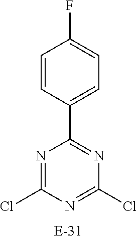 [SMILES: Fc1ccc(-c2nc(Cl)nc(Cl)n2)cc1] | 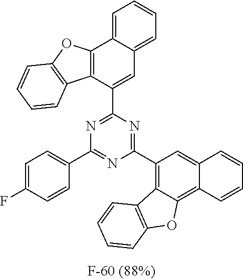 [SMILES: Fc1ccc(-c2nc(-c3cc4ccccc4c4oc5ccccc5c34)nc(-c3cc4ccccc4c4oc5ccccc5c34)n2)cc1] |
| 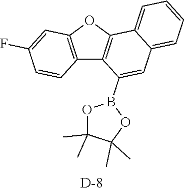 | 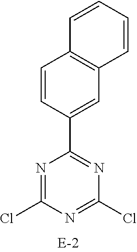 [SMILES: Clc1nc(Cl)nc(-c2ccc3ccccc3c2)n1] | 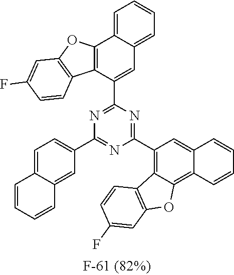 [SMILES: Fc1ccc2c(c1)oc1c3ccccc3cc(-c3nc(-c4ccc5ccccc5c4)nc(-c4cc5ccccc5c5oc6cc(F)ccc6c45)n3)c21] |
| 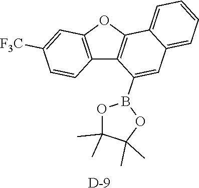 | | 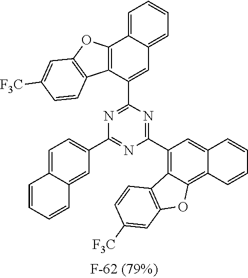 [SMILES: FC(F)(F)c1ccc2c(c1)oc1c3ccccc3cc(-c3nc(-c4ccc5ccccc5c4)nc(-c4cc5ccccc5c5oc6cc(C(F)(F)F)ccc6c45)n3)c21] |
| 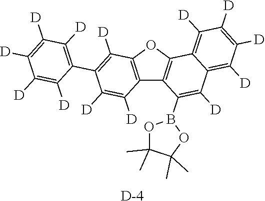 | 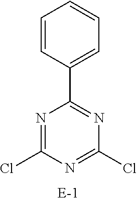 [SMILES: Clc1nc(Cl)nc(-c2ccccc2)n1] | 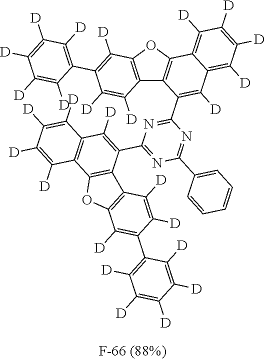 [SMILES: [2H]c1c([2H])c([2H])c(-c2c([2H])c([2H])c3c(oc4c5c([2H])c([2H])c([2H])c([2H])c5c([2H])c(-c5nc(-c6ccccc6)nc(-c6c([2H])c7c([2H])c([2H])c([2H])c([2H])c7c7oc8c([2H])c(-c9c([2H])c([2H])c([2H])c([2H])c9[2H])c([2H])c([2H])c8c67)n5)c43)c2[2H])c([2H])c1[2H]] |
| 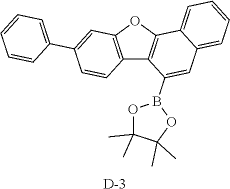 | 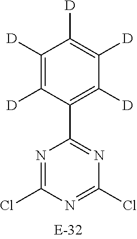 [SMILES: [2H]c1c([2H])c([2H])c(-c2nc(Cl)nc(Cl)n2)c([2H])c1[2H]] | 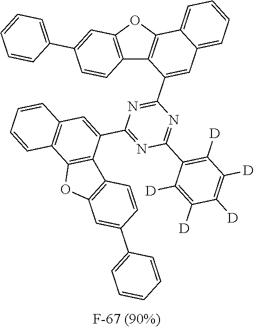 [SMILES: [2H]c1c([2H])c([2H])c(-c2nc(-c3cc4ccccc4c4oc5cc(-c6ccccc6)ccc5c34)nc(-c3cc4ccccc4c4oc5cc(-c6ccccc6)ccc5c34)n2)c([2H])c1[2H]] |
| 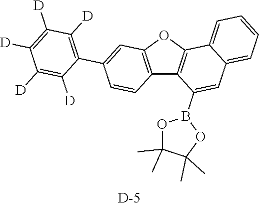 | 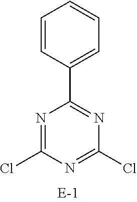 [SMILES: Clc1nc(Cl)nc(-c2ccccc2)n1] | 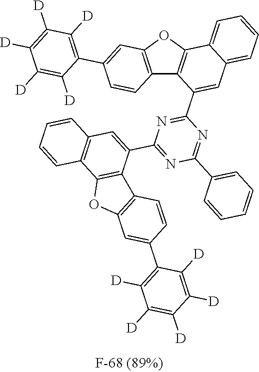 [SMILES: [2H]c1c([2H])c([2H])c(-c2ccc3c(c2)oc2c4ccccc4cc(-c4nc(-c5ccccc5)nc(-c5cc6ccccc6c6oc7cc(-c8c([2H])c([2H])c([2H])c([2H])c8[2H])ccc7c56)n4)c32)c([2H])c1[2H]] |
| 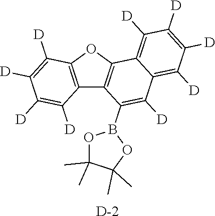 | 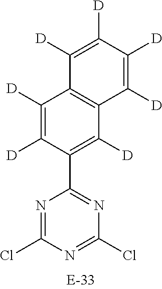 [SMILES: [2H]c1c([2H])c([2H])c2c([2H])c(-c3nc(Cl)nc(Cl)n3)c([2H])c([2H])c2c1[2H]] | 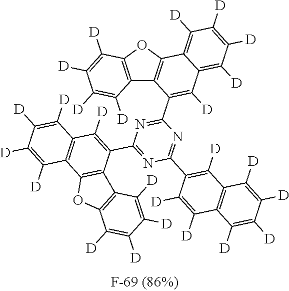 [SMILES: [2H]c1c([2H])c([2H])c2c(oc3c4c([2H])c([2H])c([2H])c([2H])c4c([2H])c(-c4nc(-c5c([2H])c([2H])c6c([2H])c([2H])c([2H])c([2H])c6c5[2H])nc(-c5c([2H])c6c([2H])c([2H])c([2H])c([2H])c6c6oc7c([2H])c([2H])c([2H])c([2H])c7c56)n4)c32)c1[2H]] |
| 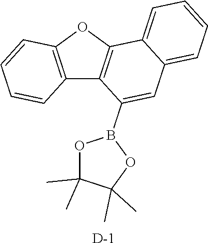 | 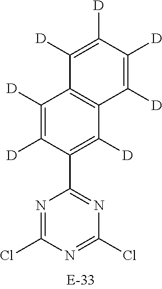 [SMILES: [2H]c1c([2H])c([2H])c2c([2H])c(-c3nc(Cl)nc(Cl)n3)c([2H])c([2H])c2c1[2H]] | 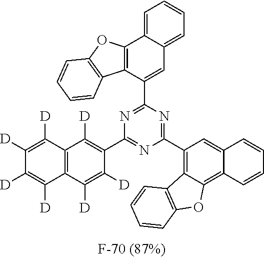 [SMILES: [2H]c1c([2H])c([2H])c2c([2H])c(-c3nc(-c4cc5ccccc5c5oc6ccccc6c45)nc(-c4cc5ccccc5c5oc6ccccc6c45)n3)c([2H])c([2H])c2c1[2H]] |
| 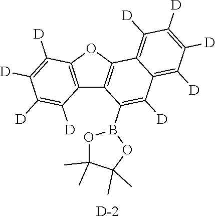 | 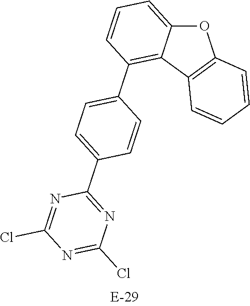 [SMILES: Clc1nc(Cl)nc(-c2ccc(-c3cccc4oc5ccccc5c34)cc2)n1] | 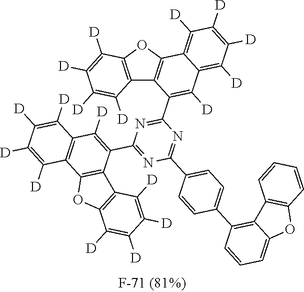 [SMILES: [2H]c1c([2H])c([2H])c2c(oc3c4c([2H])c([2H])c([2H])c([2H])c4c([2H])c(-c4nc(-c5ccc(-c6cccc7oc8ccccc8c67)cc5)nc(-c5c([2H])c6c([2H])c([2H])c([2H])c([2H])c6c6oc7c([2H])c([2H])c([2H])c([2H])c7c56)n4)c32)c1[2H]] |
| | 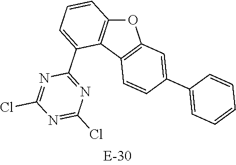 [SMILES: Clc1nc(Cl)nc(-c2cccc3oc4cc(-c5ccccc5)ccc4c23)n1] | 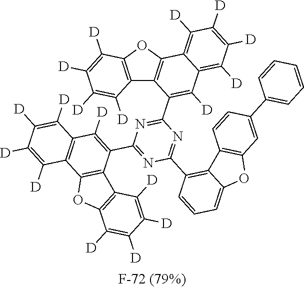 [SMILES: [2H]c1c([2H])c([2H])c2c(oc3c4c([2H])c([2H])c([2H])c([2H])c4c([2H])c(-c4nc(-c5cccc6oc7cc(-c8ccccc8)ccc7c56)nc(-c5c([2H])c6c([2H])c([2H])c([2H])c([2H])c6c6oc7c([2H])c([2H])c([2H])c([2H])c7c56)n4)c32)c1[2H]] |
| | 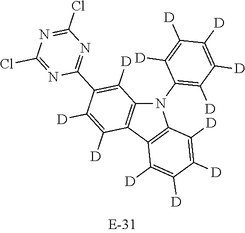 [SMILES: [2H]c1c([2H])c([2H])c(-n2c3c([2H])c([2H])c([2H])c([2H])c3c3c([2H])c([2H])c(-c4nc(Cl)nc(Cl)n4)c([2H])c32)c([2H])c1[2H]] | 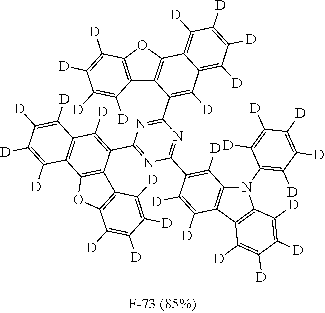 [SMILES: [2H]c1c([2H])c([2H])c(-n2c3c([2H])c([2H])c([2H])c([2H])c3c3c([2H])c([2H])c(-c4nc(-c5c([2H])c6c([2H])c([2H])c([2H])c([2H])c6c6oc7c([2H])c([2H])c([2H])c([2H])c7c56)nc(-c5c([2H])c6c([2H])c([2H])c([2H])c([2H])c6c6oc7c([2H])c([2H])c([2H])c([2H])c7c56)n4)c([2H])c32)c([2H])c1[2H]] |
| | 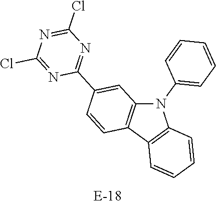 [SMILES: Clc1nc(Cl)nc(-c2ccc3c4ccccc4n(-c4ccccc4)c3c2)n1] | 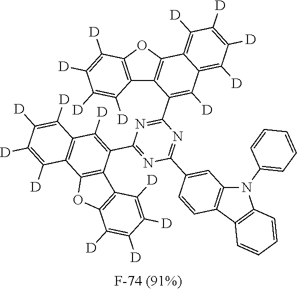 [SMILES: [2H]c1c([2H])c([2H])c2c(oc3c4c([2H])c([2H])c([2H])c([2H])c4c([2H])c(-c4nc(-c5ccc6c7ccccc7n(-c7ccccc7)c6c5)nc(-c5c([2H])c6c([2H])c([2H])c([2H])c([2H])c6c6oc7c([2H])c([2H])c([2H])c([2H])c7c56)n4)c32)c1[2H]] |
| 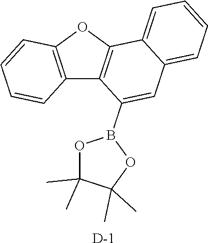 | 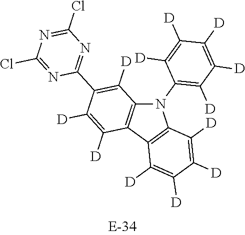 [SMILES: [2H]c1c([2H])c([2H])c(-n2c3c([2H])c([2H])c([2H])c([2H])c3c3c([2H])c([2H])c(-c4nc(Cl)nc(Cl)n4)c([2H])c32)c([2H])c1[2H]] | 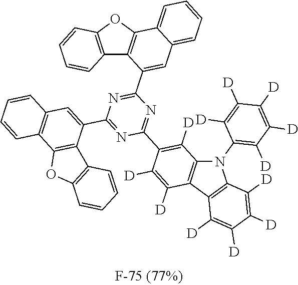 [SMILES: [2H]c1c([2H])c([2H])c(-n2c3c([2H])c([2H])c([2H])c([2H])c3c3c([2H])c([2H])c(-c4nc(-c5cc6ccccc6c6oc7ccccc7c56)nc(-c5cc6ccccc6c6oc7ccccc7c56)n4)c([2H])c32)c([2H])c1[2H]] |
| 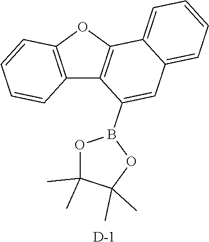 | 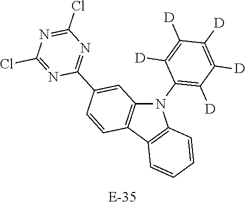 [SMILES: [2H]c1c([2H])c([2H])c(-n2c3ccccc3c3ccc(-c4nc(Cl)nc(Cl)n4)cc32)c([2H])c1[2H]] | 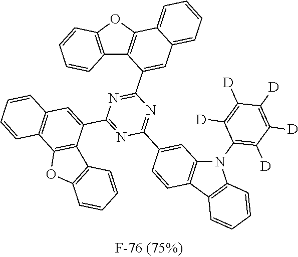 [SMILES: [2H]c1c([2H])c([2H])c(-n2c3ccccc3c3ccc(-c4nc(-c5cc6ccccc6c6oc7ccccc7c56)nc(-c5cc6ccccc6c6oc7ccccc7c56)n4)cc32)c([2H])c1[2H]] |
| 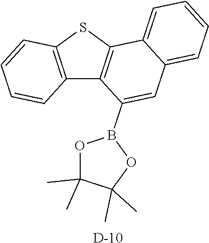 | 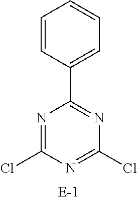 [SMILES: Clc1nc(Cl)nc(-c2ccccc2)n1] | 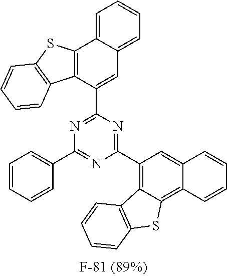 [SMILES: c1ccc(-c2nc(-c3cc4ccccc4c4sc5ccccc5c34)nc(-c3cc4ccccc4c4sc5ccccc5c34)n2)cc1] |
| | 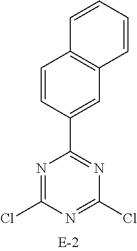 [SMILES: Clc1nc(Cl)nc(-c2ccc3ccccc3c2)n1] | 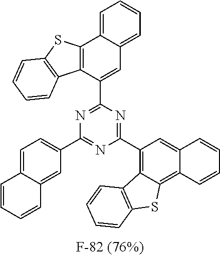 [SMILES: c1ccc2cc(-c3nc(-c4cc5ccccc5c5sc6ccccc6c45)nc(-c4cc5ccccc5c5sc6ccccc6c45)n3)ccc2c1] |
| | 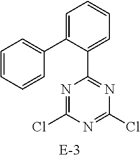 [SMILES: Clc1nc(Cl)nc(-c2ccccc2-c2ccccc2)n1] | 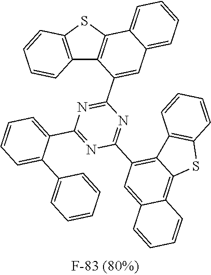 [SMILES: c1ccc(-c2ccccc2-c2nc(-c3cc4ccccc4c4sc5ccccc5c34)nc(-c3cc4ccccc4c4sc5ccccc5c34)n2)cc1] |
| | 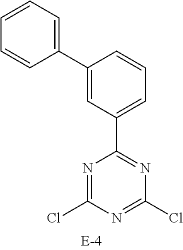 [SMILES: Clc1nc(Cl)nc(-c2cccc(-c3ccccc3)c2)n1] | 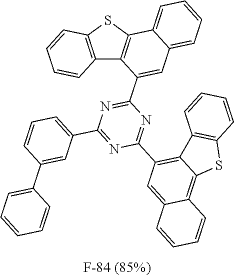 [SMILES: c1ccc(-c2cccc(-c3nc(-c4cc5ccccc5c5sc6ccccc6c45)nc(-c4cc5ccccc5c5sc6ccccc6c45)n3)c2)cc1] |
| | 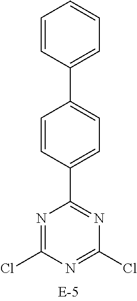 [SMILES: Clc1nc(Cl)nc(-c2ccc(-c3ccccc3)cc2)n1] | 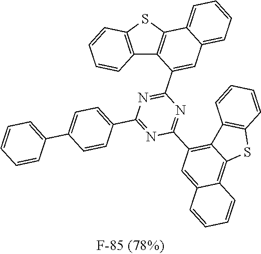 [SMILES: c1ccc(-c2ccc(-c3nc(-c4cc5ccccc5c5sc6ccccc6c45)nc(-c4cc5ccccc5c5sc6ccccc6c45)n3)cc2)cc1] |
| | 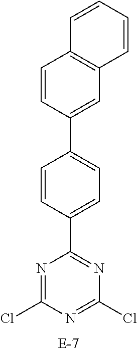 [SMILES: Clc1nc(Cl)nc(-c2ccc(-c3ccc4ccccc4c3)cc2)n1] | 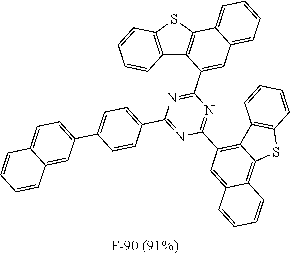 [SMILES: c1ccc2cc(-c3ccc(-c4nc(-c5cc6ccccc6c6sc7ccccc7c56)nc(-c5cc6ccccc6c6sc7ccccc7c56)n4)cc3)ccc2c1] |
| 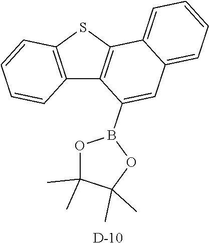 | 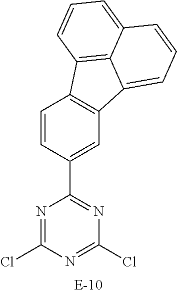 [SMILES: Clc1nc(Cl)nc(-c2ccc3c(c2)-c2cccc4cccc-3c24)n1] | 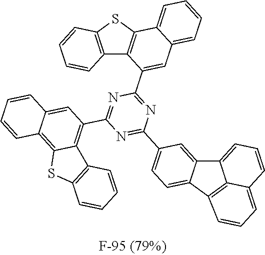 [SMILES: c1cc2c3c(cccc3c1)-c1cc(-c3nc(-c4cc5ccccc5c5sc6ccccc6c45)nc(-c4cc5ccccc5c5sc6ccccc6c45)n3)ccc1-2] |
| | 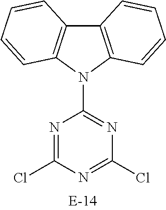 [SMILES: Clc1nc(Cl)nc(-n2c3ccccc3c3ccccc32)n1] | 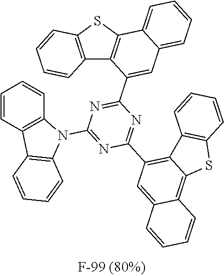 [SMILES: c1ccc2c(c1)cc(-c1nc(-c3cc4ccccc4c4sc5ccccc5c34)nc(-n3c4ccccc4c4ccccc43)n1)c1c3ccccc3sc21] |
| | 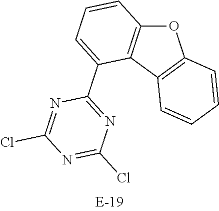 [SMILES: Clc1nc(Cl)nc(-c2cccc3oc4ccccc4c23)n1] | 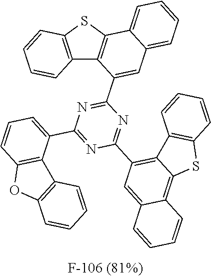 [SMILES: c1ccc2c(c1)cc(-c1nc(-c3cccc4oc5ccccc5c34)nc(-c3cc4ccccc4c4sc5ccccc5c34)n1)c1c3ccccc3sc21] |
| | 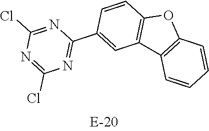 [SMILES: Clc1nc(Cl)nc(-c2ccc3oc4ccccc4c3c2)n1] | 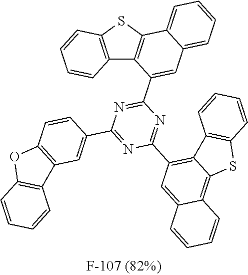 [SMILES: c1ccc2c(c1)cc(-c1nc(-c3ccc4oc5ccccc5c4c3)nc(-c3cc4ccccc4c4sc5ccccc5c34)n1)c1c3ccccc3sc21] |
| | 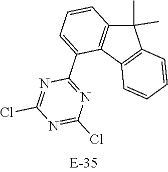 [SMILES: CC1(C)c2ccccc2-c2c(-c3nc(Cl)nc(Cl)n3)cccc21] | 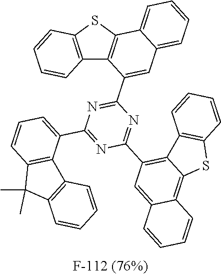 [SMILES: CC1(C)c2ccccc2-c2c(-c3nc(-c4cc5ccccc5c5sc6ccccc6c45)nc(-c4cc5ccccc5c5sc6ccccc6c45)n3)cccc21] |
| | 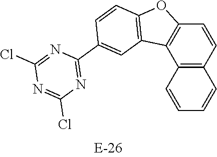 [SMILES: Clc1nc(Cl)nc(-c2ccc3oc4ccc5ccccc5c4c3c2)n1] | 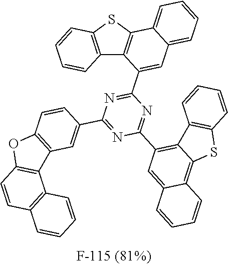 [SMILES: c1ccc2c(c1)cc(-c1nc(-c3ccc4oc5ccc6ccccc6c5c4c3)nc(-c3cc4ccccc4c4sc5ccccc5c34)n1)c1c3ccccc3sc21] |
| | 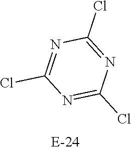 | 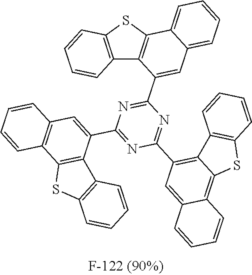 [SMILES: c1ccc2c(c1)cc(-c1nc(-c3cc4ccccc4c4sc5ccccc5c34)nc(-c3cc4ccccc4c4sc5ccccc5c34)n1)c1c3ccccc3sc21] |
| 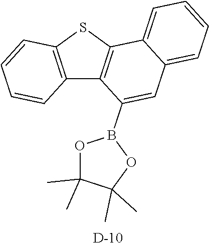 | 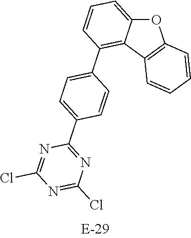 [SMILES: Clc1nc(Cl)nc(-c2ccc(-c3cccc4oc5ccccc5c34)cc2)n1] | 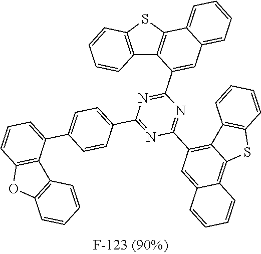 [SMILES: c1ccc2c(c1)cc(-c1nc(-c3ccc(-c4cccc5oc6ccccc6c45)cc3)nc(-c3cc4ccccc4c4sc5ccccc5c34)n1)c1c3ccccc3sc21] |
| 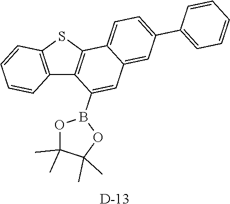 | 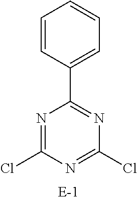 [SMILES: Clc1nc(Cl)nc(-c2ccccc2)n1] | 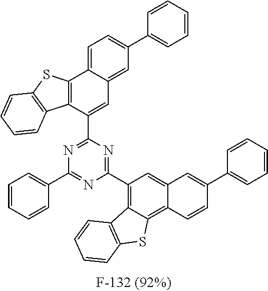 [SMILES: c1ccc(-c2ccc3c(c2)cc(-c2nc(-c4ccccc4)nc(-c4cc5cc(-c6ccccc6)ccc5c5sc6ccccc6c45)n2)c2c4ccccc4sc32)cc1] |
| 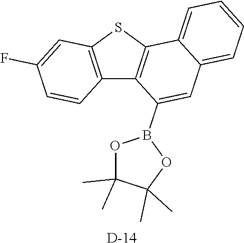 | 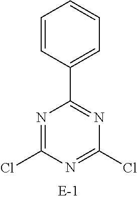 [SMILES: Clc1nc(Cl)nc(-c2ccccc2)n1] | 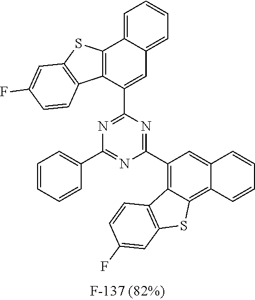 [SMILES: Fc1ccc2c(c1)sc1c3ccccc3cc(-c3nc(-c4ccccc4)nc(-c4cc5ccccc5c5sc6cc(F)ccc6c45)n3)c21] |
| 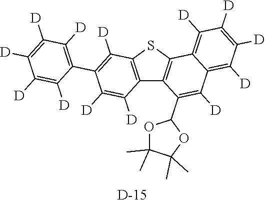 | 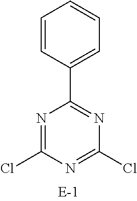 [SMILES: Clc1nc(Cl)nc(-c2ccccc2)n1] | 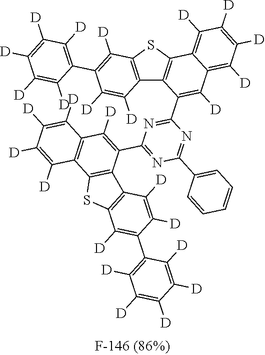 [SMILES: [2H]c1c([2H])c([2H])c(-c2c([2H])c([2H])c3c(sc4c5c([2H])c([2H])c([2H])c([2H])c5c([2H])c(-c5nc(-c6ccccc6)nc(-c6c([2H])c7c([2H])c([2H])c([2H])c([2H])c7c7sc8c([2H])c(-c9c([2H])c([2H])c([2H])c([2H])c9[2H])c([2H])c([2H])c8c67)n5)c43)c2[2H])c([2H])c1[2H]] |
| 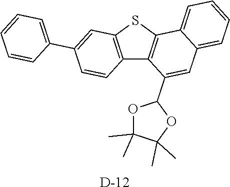 | 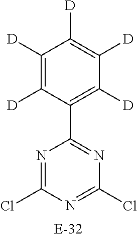 [SMILES: [2H]c1c([2H])c([2H])c(-c2nc(Cl)nc(Cl)n2)c([2H])c1[2H]] | 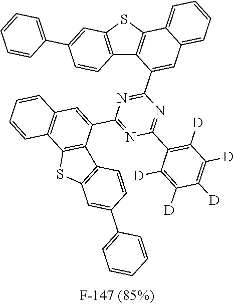 [SMILES: [2H]c1c([2H])c([2H])c(-c2nc(-c3cc4ccccc4c4sc5cc(-c6ccccc6)ccc5c34)nc(-c3cc4ccccc4c4sc5cc(-c6ccccc6)ccc5c34)n2)c([2H])c1[2H]] |
| 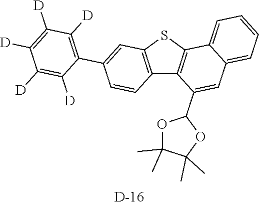 | 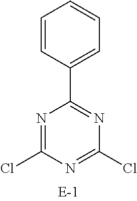 [SMILES: Clc1nc(Cl)nc(-c2ccccc2)n1] | 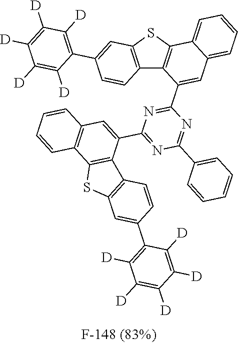 [SMILES: [2H]c1c([2H])c([2H])c(-c2ccc3c(c2)sc2c4ccccc4cc(-c4nc(-c5ccccc5)nc(-c5cc6ccccc6c6sc7cc(-c8c([2H])c([2H])c([2H])c([2H])c8[2H])ccc7c56)n4)c32)c([2H])c1[2H]] |
| 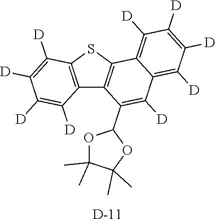 | 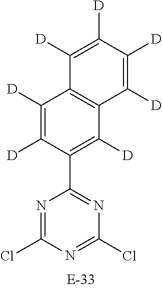 [SMILES: [2H]c1c([2H])c([2H])c2c([2H])c(-c3nc(Cl)nc(Cl)n3)c([2H])c([2H])c2c1[2H]] | 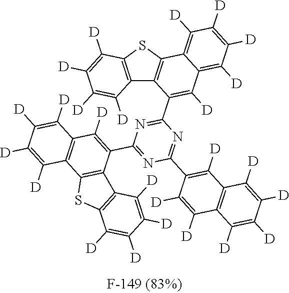 [SMILES: [2H]c1c([2H])c([2H])c2c(sc3c4c([2H])c([2H])c([2H])c([2H])c4c([2H])c(-c4nc(-c5c([2H])c([2H])c6c([2H])c([2H])c([2H])c([2H])c6c5[2H])nc(-c5c([2H])c6c([2H])c([2H])c([2H])c([2H])c6c6sc7c([2H])c([2H])c([2H])c([2H])c7c56)n4)c32)c1[2H]] |
| 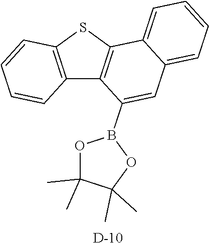 | | 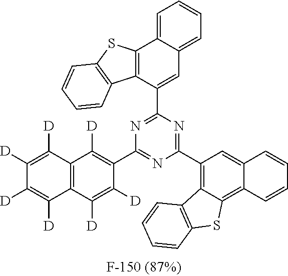 [SMILES: [2H]c1c([2H])c([2H])c2c([2H])c(-c3nc(-c4cc5ccccc5c5sc6ccccc6c45)nc(-c4cc5ccccc5c5sc6ccccc6c45)n3)c([2H])c([2H])c2c1[2H]] |
| 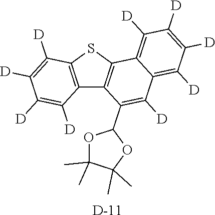 | 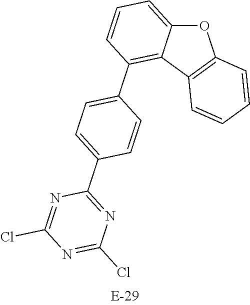 [SMILES: Clc1nc(Cl)nc(-c2ccc(-c3cccc4oc5ccccc5c34)cc2)n1] | 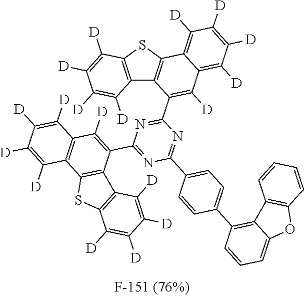 [SMILES: [2H]c1c([2H])c([2H])c2c(sc3c4c([2H])c([2H])c([2H])c([2H])c4c([2H])c(-c4nc(-c5ccc(-c6cccc7oc8ccccc8c67)cc5)nc(-c5c([2H])c6c([2H])c([2H])c([2H])c([2H])c6c6sc7c([2H])c([2H])c([2H])c([2H])c7c56)n4)c32)c1[2H]] |
| | 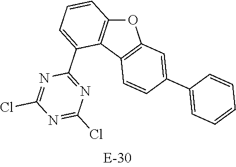 [SMILES: Clc1nc(Cl)nc(-c2cccc3oc4cc(-c5ccccc5)ccc4c23)n1] | 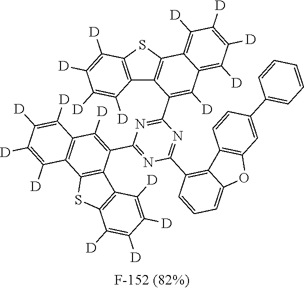 [SMILES: [2H]c1c([2H])c([2H])c2c(sc3c4c([2H])c([2H])c([2H])c([2H])c4c([2H])c(-c4nc(-c5cccc6oc7cc(-c8ccccc8)ccc7c56)nc(-c5c([2H])c6c([2H])c([2H])c([2H])c([2H])c6c6sc7c([2H])c([2H])c([2H])c([2H])c7c56)n4)c32)c1[2H]] |
| | 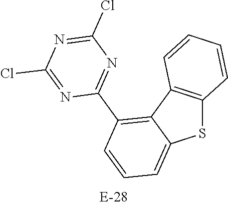 [SMILES: Clc1nc(Cl)nc(-c2cccc3sc4ccccc4c23)n1] | 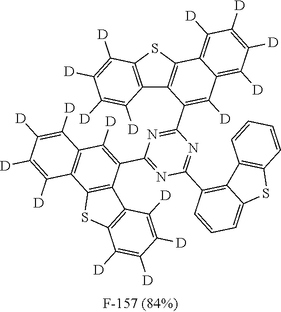 [SMILES: [2H]c1c([2H])c([2H])c2c(sc3c4c([2H])c([2H])c([2H])c([2H])c4c([2H])c(-c4nc(-c5cccc6sc7ccccc7c56)nc(-c5c([2H])c6c([2H])c([2H])c([2H])c([2H])c6c6sc7c([2H])c([2H])c([2H])c([2H])c7c56)n4)c32)c1[2H]] |
| | 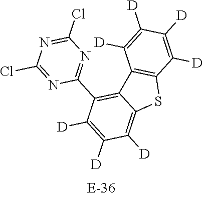 [SMILES: [2H]c1c([2H])c([2H])c2c(sc3c([2H])c([2H])c([2H])c(-c4nc(Cl)nc(Cl)n4)c32)c1[2H]] | 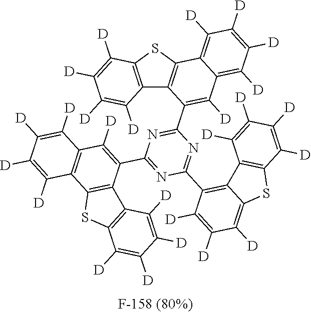 [SMILES: [2H]c1c([2H])c([2H])c2c(sc3c([2H])c([2H])c([2H])c(-c4nc(-c5c([2H])c6c([2H])c([2H])c([2H])c([2H])c6c6sc7c([2H])c([2H])c([2H])c([2H])c7c56)nc(-c5c([2H])c6c([2H])c([2H])c([2H])c([2H])c6c6sc7c([2H])c([2H])c([2H])c([2H])c7c56)n4)c32)c1[2H]] |
| 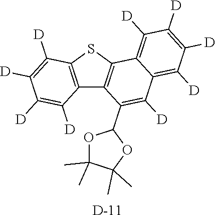 | 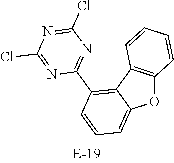 [SMILES: Clc1nc(Cl)nc(-c2cccc3oc4ccccc4c23)n1] | 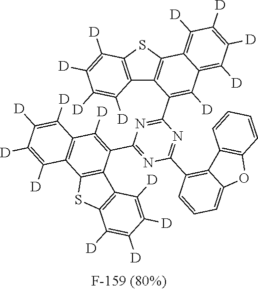 [SMILES: [2H]c1c([2H])c([2H])c2c(sc3c4c([2H])c([2H])c([2H])c([2H])c4c([2H])c(-c4nc(-c5cccc6oc7ccccc7c56)nc(-c5c([2H])c6c([2H])c([2H])c([2H])c([2H])c6c6sc7c([2H])c([2H])c([2H])c([2H])c7c56)n4)c32)c1[2H]] |
| | 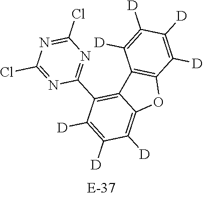 [SMILES: [2H]c1c([2H])c([2H])c2c(oc3c([2H])c([2H])c([2H])c(-c4nc(Cl)nc(Cl)n4)c32)c1[2H]] | 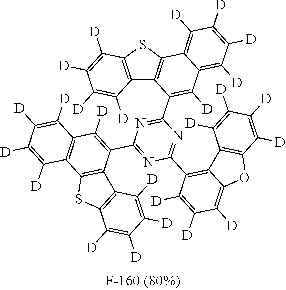 [SMILES: [2H]c1c([2H])c([2H])c2c(oc3c([2H])c([2H])c([2H])c(-c4nc(-c5c([2H])c6c([2H])c([2H])c([2H])c([2H])c6c6sc7c([2H])c([2H])c([2H])c([2H])c7c56)nc(-c5c([2H])c6c([2H])c([2H])c([2H])c([2H])c6c6sc7c([2H])c([2H])c([2H])c([2H])c7c56)n4)c32)c1[2H]] |

### <Preparation Example 6> Preparation of Compound G-15

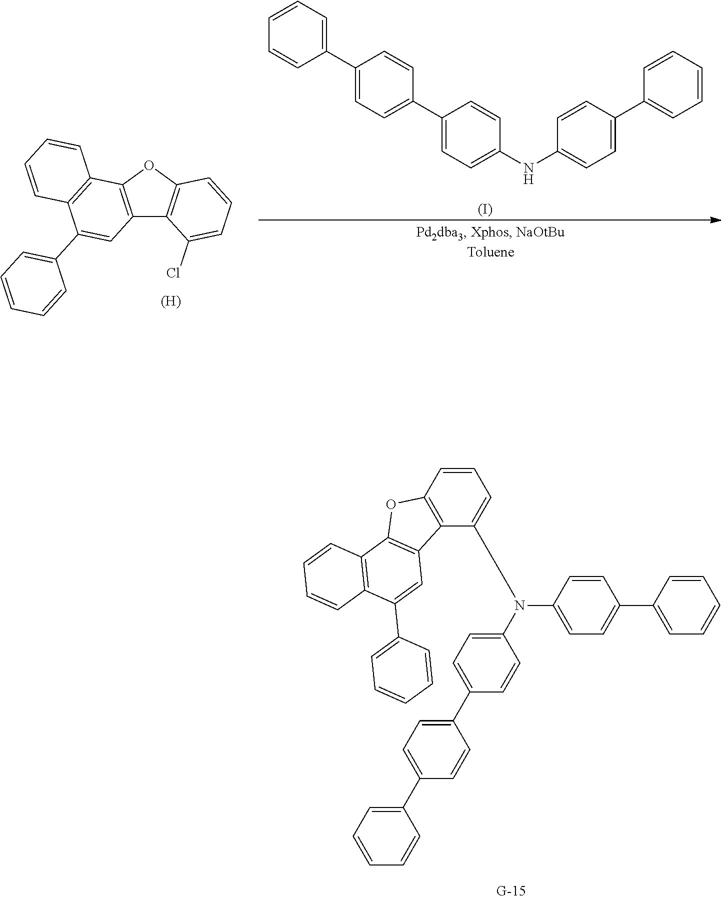

After 7-chloro-5-phenylnaphtho[1,2-b]benzofuran (H) (7 g, 21 mmol), N-([1,1′-biphenyl]-4-yl)-[1,1′:4′,1″-terphenyl]-4-amine (I) (9 g, 22 mmol), Pd₂dba₃ (0.97 g, 1 mmol), Xphos (1 g, 2.11 mmol), and 250 ml (4 g, 42 mmol) of NaOtBu were put into a 500 mL round-bottom flask and dissolved in 150 ml of toluene, the resulting solution was refluxed for 1 hour. After the reaction was completed, extraction was performed using MC and distilled water. After the solvent in the organic layer was concentrated, the residue was dissolved in MC and purified with silica, and then re-crystallized using MC/hexane to obtain 12 g (17.4 mmol, 87%) of Compound G-15 as a white solid.

The following Target Compound G was synthesized by performing synthesis in the same manner as in Preparation Example 6, except that Compound H and Compound I in the following Table 3 were used instead of Compound (H) and Compound (I), respectively, in Preparation Example 6.

TABLE 3

| Compound H | Compound I | Compound G (Yield) |
|---|---|---|
| 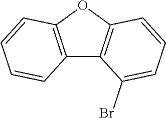 [SMILES: Brc1cccc2oc3ccccc3c12] | 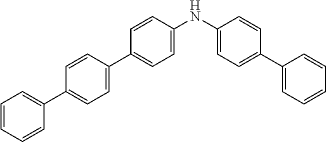 [SMILES: c1ccc(-c2ccc(Nc3ccc(-c4ccc(-c5ccccc5)cc4)cc3)cc2)cc1] | 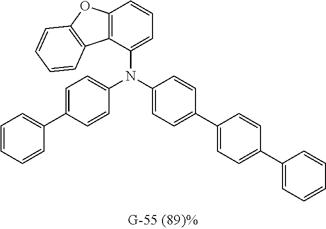 [SMILES: c1ccc(-c2ccc(-c3ccc(N(c4ccc(-c5ccccc5)cc4)c4cccc5oc6ccccc6c45)cc3)cc2)cc1] |
| 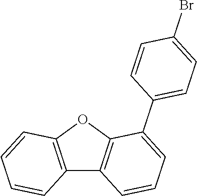 [SMILES: Brc1ccc(-c2cccc3c2oc2ccccc23)cc1] | 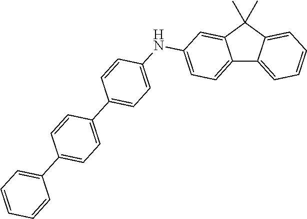 [SMILES: CC1(C)c2ccccc2-c2ccc(Nc3ccc(-c4ccc(-c5ccccc5)cc4)cc3)cc21] | 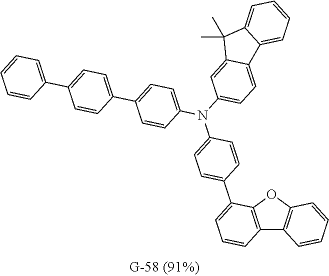 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21] |

It was confirmed by FD-mass spectrometry and ¹H-NMR that the compounds synthesized in Preparation Examples 1 to 6 were synthesized as the desired compounds. The measured values by field desorption mass spectrometry (FD-Mass) are shown in the following Table 4, and the measured values by ¹HNMR (CDCl₃, 200 MHz) are shown in the following Table 5.

TABLE 4

| com- | |
|---|---|
| pound | |
| No. | FD-Mass |
| F-1 | m/z = 589.65 |
| | (C41H23N3O2, 589.18) |
| F-2 | m/z = 639.71 |
| | (C45H25N3O2, 639.19) |
| F-3 | m/z = 665.75 |
| | (C47H27N3O2, 665.21) |
| F-4 | m/z = 665.75 |
| | (C47H27N3O2, 665.21) |
| F-5 | m/z = 665.75 |
| | (C47H27N3O2, 665.21) |
| F-7 | m/z = 741.85 |
| | (C53H31N3O2, 741.24) |
| F-10 | m/z = 715.81 |
| | (C51H29N3O2, 715.23) |
| F-12 | m/z = 715.81 |
| | (C51H29N3O2, 715.23) |
| F-14 | m/z = 689.77 |
| | (C49H27N3O2, 689.21) |
| F-15 | m/z = 713.80 |
| | (C51H27N3O2, 713.21) |
| F-16 | m/z = 713.80 |
| | (C51H27N3O2, 713.21) |
| F-17 | m/z = 739.83 |
| | (C53H29N3O2, 739.23) |
| F-18 | m/z = 765.87 |
| | (C55H31N3O2, 765.24) |
| F-19 | m/z = 678.75 |
| | (C47H26N4O2, 768.21) |
| F-20 | m/z = 754.85 |
| | (C53H30N4O2, 754.24) |
| F-22 | m/z = 754.85 |
| | (C53H30N4O2, 754.24) |
| F-23 | m/z = 754.85 |
| | (C53H30N4O2, 754.24) |
| F-25 | m/z = 754.85 |
| | (C53H30N4O2, 754.24) |
| F-26 | m/z = 679.74 |
| | (C47H25N3O3, 679.19) |
| F-27 | m/z = 679.74 |
| | (C47H25N3O3, 679.19) |
| F-28 | m/z = 679.74 |
| | (C47H25N3O3, 679.19) |
| F-29 | m/z = 679.74 |
| | (C47H25N3O3, 679.19) |
| F-30 | m/z = 729.80 |
| | (C51H27N3O3, 729.21) |
| F-31 | m/z = 729.80 |
| | (C51H27N3O3, 729.21) |
| F-32 | m/z = 729.80 |
| | (C51H27N3O3, 729.21) |
| F-33 | m/z = 729.80 |
| | (C51H27N3O3, 729.21) |
| F-35 | m/z = 729.80 |
| | (C51H27N3O3, 729.21) |
| F-37 | m/z = 729.80 |
| | (C51H27N3O3, 729.21) |
| F-39 | m/z = 695.80 |
| | (C47H25N3O2S, 695.17) |
| F-43 | m/z = 755.83 |
| | (C53H29N3O3, 755.83) |
| F-46 | m/z = 755.83 |
| | (C53H29N3O3, 755.83) |
| F-49 | m/z = 741.85 |
| | (C53H31N3O2, 741.24) |
| F-54 | m/z = 741.85 |
| | (C53H31N3O2, 741.24) |
| F-56 | m/z = 741.85 |
| | (C53H31N3O2, 741.24) |
| F-60 | m/z = 607.64 |
| | (C41H22FN3O2, 607.17) |
| F-61 | m/z = 625.63 |
| | (C41H21F2N3O2, 625.16) |
| F-62 | m/z = 775.71 |
| | (C47H23F6N3O2, 775.17) |
| F-65 | m/z = 754.93 |
| | (C53H18D13N3O2, 754.32) |
| F-66 | m/z = 768.01 |
| | (C53H5D26N3O2, 767.40) |
| F-67 | m/z = 746.88 |
| | (C53H26D5N3O2, 746.27) |
| F-68 | m/z = 751.91 |
| | (C53H21D10N3O2, 751.30) |
| F-69 | m/z = 664.87 |
| | (C45D25N3O2, 664.35) |
| F-70 | m/z = 646.76 |
| | (C45H18D7N3O2, 646.24) |
| F-71 | m/z = 773.94 |
| | (C53H11D18N3O3, 773.33) |
| F-72 | m/z = 773.94 |
| | (C53H11D18N3O3, 773.33) |
| F-73 | m/z = 785.03 (C53D30N4O2, |
| | 784.43) |
| F-74 | m/z = 772.96 |
| | (C53H12D18N4O2, 772.35) |
| F-75 | m/z = 766.92 |
| | (C53H18D12N4O2, 766.31) |
| F-76 | m/z =759.88 |
| | (C53H25D5N4O2, 759.27) |
| F-81 | m/z = 621.78 (C41H23N3S2, |
| | 621.78) |
| F-82 | m/z = 671.84 (C45H25N3S2, |
| | 671.15) |
| F-83 | m/z = 697.87 (C47H27N3S2, |
| | 697.16) |
| F-84 | m/z = 697.87 (C47H27N3S2, |
| | 697.16) |
| F-85 | m/z = 697.87 (C47H27N3S2, |
| | 697.16) |
| F-90 | m/z = 747.93 (C51H29N3S2, |
| | 747.18) |
| F-95 | m/z = 745.92 (C51H27N3S2, |
| | 745.16) |
| F-99 | m/z = 710.87 (C47H26N4S2, |
| | 710.16) |
| F-106 | m/z = 711.86 |
| | (C47H25N3OS2, 711.14) |
| F-107 | m/z = 711.86 |
| | (C47H25N3OS2, 711.14) |
| F-112 | m/z = 737.94 (C50H31N3S2, |
| | 737.20) |
| F-115 | m/z = 761.92 |
| | (C51H27N3OS2, 761.16) |
| F-122 | m/z = 777.98 (C51H27N3S3, |
| | 777.14) |
| F-123 | m/z = 787.96 |
| | (C53H29N3OS2, 787.18) |
| F-132 | m/z = 773.97 (C53H31N3S2, |
| | 773.20) |
| F-137 | m/z = 657.76 |
| | (C41H21F2N3S2, 657.11) |
| F-145 | m/z = 787.05 |
| | (C53H18D13N3S2, 786.28) |
| F-146 | m/z = 800.13 |
| | (C53H5D26N3S2, 799.36) |
| F-147 | m/z = 779.00 |
| | (C53H26D5N3S2, 778.23) |
| F-148 | m/z = 784.03 |
| | (C53H21D10N3S2, 783.26) |
| F-149 | m/z = 696.99 (C45D25N3S2, |
| | 696.31) |
| F-150 | m/z = 678.88 |
| | (C45H18D7N3S2, 678.19) |
| F-151 | m/z = 806.06 |
| | (C53H11D18N3OS2, 805.29) |
| F-152 | m/z = 806.06 |
| | (C53H11D18N3OS2, 805.29) |
| F-157 | m/z = 713.91 |
| | (C47H7D18N3O2S, 713.28) |
| F-158 | m/z = 720.95 |
| | (C47D25N3O2S, 720.32) |
| F-159 | m/z = 729.97 |
| | (C47H7D18N3OS2, 729.26) |
| F-160 | m/z = 737.01 |
| | (C47D25N3OS2, 736.30) |
| G-15 | m/z = 689.27 (C52H35NO, |
| | 789.86) |
| G-55 | m/z = 563.22 (C42H29NO, |
| | 563.70) |
| G-58 | m/z = 679.29 (C51H37NO, |
| | 679.86) |

TABLE 5

| com- | |
|---|---|
| pound | |
| No. | ¹H NMR (CDCl₃, 200 MHz) |
| F-1 | δ = 8.36 (2H, s), 8.02 (2H, d), 7.98 (4H, d), 7.59 (4H, d), |
| | 7.54 (2H, d), 7.5(3H, d), 7.47 (2H, d), 7.39 (2H, d), |
| | 7.31 (2H, d) |
| F-2 | δ = 9.09 (1H, s), 8.49 (1H, d), 8.16 (1H, d), 8.08 (1H, d), |
| | 8.02 (2H, d), 8 (1H, d), 7.98 (4H, d), 7.61 (1H, d), |
| | 7.59 (5H, d), 7.54 (2H, dd), 7.47 (2H, d), 7.39(2H, d), |
| | 7.31 (2H, dd) |
| F-3 | δ = 8.02 (2H, s), 7.98 (4H, d), 7.96 (2H, d), 7.79(2H, d), |
| | 7.6(2H, d), 7.59 (4H, d), 7.54 (2H, d), 7.47 (2H, d), |
| | 7.46(2H, d), 7.41 (1H, dd), 7.39 (2H, d), 7.31 (2H, d) |
| F-4 | δ = 8.38 (1H, s), 8.02 (2H, d), 7.98 (4H, d), 7.94 (1H, d), |
| | 7.75 (2H, d), 7.73 (1H, d), 7.61 (1H, d), 7.59 (4H, d), |
| | 7.54 (2H, d), 7.49 (2H, dd), 7.47 (2H, d), 7.41 (1H, d), |
| | 7.39 (2H, dd), 7.31 (2H, dd) |
| F-5 | δ = 8.02 (2H, s), 7.98 (4H, d), 7.96 (2H, d), 7.75 (2H, d), |
| | 7.59 (4H, d), 7.54 (2H, d), 7.49 (2H, d), 7.47 (2H, d), |
| | 7.41 (1H, d), 7.39(2H, dd), 7.31 (2H, d), 7.25 (2H, d) |
| F-7 | δ = 8.02 (2H, s), 7.98 (4H, d), 7.96 (2H, d), 7.94 (1H, d), |
| | 7.75(2H, d), 7.73(1H, d), 7.61 (2H, d), 7.6(2H, d), |
| | 7.59 (4H, d), 7.54 (2H, dd), 7.49 (2H, d), 7.47 (2H, d), |
| | 7.39 (2H, dd), 7.31 (2H, dd) |
| F-10 | δ = 8.09 (1H, s), 8.06 (1H, d), 8.02 (2H, d), 7.99 (1H, d), |
| | 7.98 (4H, d), 7.96 (2H, d), 7.63 (1H, d), 7.6(1H, d), |
| | 7.59 (4H, d), 7.55 (1H, dd), 7.54 (2H, d), 7.47 (2H, d), |
| | 7.39 (2H, dd), 7.38 (1H, dd), 7.31 (3H, s), 7.25 (2H, t) |
| F-12 | δ = 9.15 (1H, s), 8.51 (1H, d), 8.02 (2H, d), 7.99 (1H, d), |
| | 7.98 (4H, d), 7.91 (1H, d), 7.75(2H, d), 7.59 (4H, d), |
| | 7.55 (1H, d), 7.54 (2H, dd), 7.49(2H, d), 7.47 (2H, d), |
| | 7.41 (2H, dd), 7.39 (2H, dd), 7.38 (1H, s), 7.31 (2H, t) |
| F-14 | δ = 9.27 (1H, s), 8.85 (1H, d), 8.52 (1H, d), 8.15 (1H, d), |
| | 8.02 (2H, d), 7.98 (4H, d), 7.9 (1H, d), 7.75 (2H, d), |
| | 7.68 (1H, d), 7.63 (1H, dd), 7.59 (4H, d), 7.54 (2H, d), |
| | 7.47 (2H, dd), 7.39 (2H, dd), 7.31 (4H, s) |
| F-15 | δ = 8.02 (2H, s), 7.98 (4H, d), 7.8 (2H, d), 7.77 (1H, d), |
| | 7.69 (2H, d), 7.62 (1H, d), 7.6(1H, d), 7.59 (4H, d), |
| | 7.55 (2H, d), 7.54 (2H, dd), 7.47 (2H, d), 7.39 (2H, d), |
| | 7.31 (4H, dd) |
| F-16 | δ = 8.42 (2H, s), 8.1 (2H, d), 8.02 (2H, d), 7.99 (1H, d), |
| | 7.98 (4H, d), 7.82 (1H, d), 7.8 (1H, d), 7.69 (1H, d), |
| | 7.59 (4H, d), 7.55 (1H, dd), 7.54 (2H, d), 7.47 (2H, d), |
| | 7.39 (2H, dd), 7.31 (4H, dd) |
| F-17 | δ = 9.6 (1H, s), 9.27 (1H, d), 8.33 (1H, d), 8.3 (1H, d), |
| | 8.15 (1H, d), 8.02 (2H, d), 7.98 (4H, d), 7.7(2H, d), |
| | 7.64 (2H, d), 7.59 (4H, dd), 7.54 (2H, d), 7.52 (2H, d), |
| | 7.47 (2H, dd), 7.39 (2H, dd), 7.31 (2H, s) |
| F-18 | δ = 9.08 (1H, s), 8.84 (1H, d), 8.27 (1H, d), 8.05 (1H, d), |
| | 8.02 (2H, d), 7.98 (4H, d), 7.96 (2H, d), 7.9 (1H, d), |
| | 7.7 (1H, d), 7.68 (1H, dd), 7.64 (1H, d), 7.63 (1H, d), |
| | 7.59 (4H, dd), 7.54 (2H, dd), 7.47 (2H, s), 7.39 (2H, t), |
| | 7.31 (2H, t), 7.25 (2H, dd) |
| F-19 | δ = 8.55 (1H, s), 8.19 (1H, d), 8.02 (2H, d), 7.98 (4H, d), |
| | 7.94 (1H, d), 7.59 (5H, d), 7.58 (1H, d), 7.54 (2H, d), |
| | 7.5 (1H, d), 7.47 (2H, dd), 7.39 (2H, d), 7.35 (1H, d), |
| | 7.31 (4H, dd) |
| F-20 | δ = 8.55 (1H, s), 8.19 (1H, d), 8.02 (2H, d), 7.98 (4H, d), |
| | 7.94 (1H, d), 7.92 (1H, d), 7.91 (1H, d), 7.8 (1H, d), |
| | 7.59 (5H, d), 7.58 (1H, dd), 7.54 (2H, d), 7.5 (1H, d), |
| | 7.47 (2H, dd), 7.46 (1H, dd), 7.39 (2H, s), 7.35 (1H, t), |
| | 7.31 (2H, t), 7.2 (1H, dd), 7.16 (1H, dd) |
| F-22 | δ = 8.55 (1H, s), 8.19 (1H, d), 8.02 (2H, d), 7.98 (4H, d), |
| | 7.94 (1H, d), 7.92 (2H, d), 7.91 (2H, d), 7.59 (5H, d), |
| | 7.58 (1H, d), 7.54 (2H, dd), 7.5 (1H, d), 7.47 (2H, d), |
| | 7.39 (2H, dd), 7.35 (1H, dd), 7.31 (2H, s), 7.2 (1H, t), |
| | 7.16 (1H, t) |
| F-23 | δ = 8.55 (1H, s), 8.02 (2H, d), 7.98 (4H, d), 7.94 (1H, d), |
| | 7.91 (1H, d), 7.68 (1H, d), 7.62 (2H, d), 7.59 (5H, d), |
| | 7.58 (1H, d), 7.56 (1H, dd), 7.54 (2H, d), 7.5 (2H, d), |
| | 7.47 (2H, dd), 7.39 (2H, dd), 7.35 (1H, s), 7.31 (2H, t), |
| | 7.16 (1H, t) |
| F-25 | δ = 8.55 (1H, s), 8.31 (1H, d), 8.02 (2H, d), 7.98 (4H, d), |
| | 7.94 (1H, d), 7.91 (1H, d), 7.74 (1H, d), 7.62 (2H, d), |
| | 7.59 (5H, d), 7.58 (1H, dd), 7.54 (2H, d), 7.5 (2H, d), |
| | 7.47 (2H, dd), 7.39 (2H, dd), 7.35 (1H, s), 7.31 (2H, t), |
| | 7.16 (1H, t) |
| F-26 | δ = 8.02 (2H, s), 7.98 (5H, d), 7.82 (1H, d), 7.69 (1H, d), |
| | 7.59 (5H, d), 7.57 (1H, d), 7.54 (3H, d), 7.47 (2H, d), |
| | 7.39 (4H, d), 7.35 (1H, dd), 7.31 (5H, d) |
| F-27 | δ = 8.02 (2H, s), 7.98 (5H, d), 7.88 (1H, d), 7.83 (1H, d), |
| | 7.79 (1H, d), 7.59 (5H, d), 7.54 (3H, d), 7.47 (2H, d), |
| | 7.39 (3H, d), 7.31 (7H, dd) |
| F-28 | δ = 8.03 (1H, s), 8.02 (2H, d), 7.98 (5H, d), 7.82 (1H, d), |
| | 7.76 (1H, d), 7.59 (5H, d), 7.54 (3H, d), 7.47 (2H, d), |
| | 7.39 (3H, d), 7.31 (7H, dd) |
| F-29 | δ = 8.08 (1H, s), 8.02 (2H, d), 7.98 (5H, d), 7.88 (1H, d), |
| | 7.59 (5H, d), 7.54 (3H, d), 7.51 (1H, d), 7.47 (2H, d), |
| | 7.39 (3H, d), 7.31 (7H, dd) |
| F-30 | δ = 8.28 (1H, s), 8.11 (1H, d), 8.02 (2H, d), 7.98 (4H, d), |
| | 7.82 (1H, d), 7.75 (1H, d), 7.69 (2H, d), 7.59 (5H, d), |
| | 7.57 (1H, d), 7.54 (2H, dd), 7.49 (1H, d), 7.47 (2H, d), |
| | 7.42 (1H, dd), 7.39 (2H, dd), 7.31 (4H, s) |
| F-31 | δ = 8.16 (1H, s), 8.11 (1H, d), 8.02 (2H, d), 7.98 (4H, d), |
| | 7.84 (1H, d), 7.82 (1H, d), 7.69 (2H, d), 7.67 (1H, d), |
| | 7.59 (5H, d), 7.57 (1H, dd), 7.54 (2H, d), 7.48 (1H, d), |
| | 7.47 (2H, dd), 7.39 (2H, dd), 7.31 (4H, s) |
| F-32 | δ = 8.02 (3H, s), 7.98 (6H, d), 7.59 (7H, d), 7.54 (3H, d), |
| | 7.47 (3H, d), 7.39 (3H, d), 7.31 (5H, d) |
| F-33 | δ = 8.54 (1H, s), 8.03 (1H, d), 8.02 (2H, d), 7.99 (1H, d), |
| | 7.98 (4H, d), 7.82 (1H, d), 7.76 (1H, d), 7.61 (1H, d), |
| | 7.6(1H, d), 7.59 (5H, dd), 7.54 (3H, d), 7.53 (1H, d), |
| | 7.47 (2H, dd), 7.39 (2H, dd), 7.31 (4H, s) |
| F-35 | δ = 8.54 (1H, s), 8.02 (2H, d), 7.99 (1H, d), 7.98 (4H, d), |
| | 7.88 (1H, d), 7.83(1H, d), 7.79 (1H, d), 7.61 (1H, d), |
| | 7.6(1H, d), 7.59(5H, dd), 7.54 (3H, d), 7.53 (1H, d), |
| | 7.47 (2H, dd), 7.39 (2H, dd), 7.31 (4H, s) |
| F-37 | δ = 8.54 (1H, s), 8.08 (1H, d), 8.02 (2H, d), 7.99 (1H, d), |
| | 7.98 (4H, d), 7.88 (1H, d), 7.61 (1H, d), 7.6 (1H, d), |
| | 7.59 (5H, d), 7.54 (3H, dd), 7.53 (1H, d), 7.51 (1H, d), |
| | 7.47 (2H, dd), 7.39 (2H, dd), 7.31 (4H, s) |
| F-39 | δ = 8.45 (1H, s), 8.03 (1H, d), 8.02 (2H, d), 7.98 (4H, d), |
| | 7.94 (1H, d), 7.93 (1H, d), 7.68 (1H, d), 7.59 (5H, d), |
| | 7.56 (1H, d), 7.54 (2H, dd), 7.49 (1H, d), 7.47 (2H, d), |
| | 7.39 (2H, dd), 7.31 (6H, dd) |
| F-43 | δ = 8.02 (2H, s), 7.98 (5H, d), 7.96 (2H, d), 7.82 (1H, d), |
| | 7.69 (1H, d), 7.59 (5H, d), 7.57 (1H, d), 7.54 (3H, d), |
| | 7.47 (2H, d), 7.39 (3H, dd), 7.31 (3H, d), 7.25 (2H, d) |
| F-46 | δ = 8.03 (1H, s), 8.02 (2H, d), 7.98 (4H, d), 7.82 (2H, d), |
| | 7.76 (1H, d), 7.75 (2H, d), 7.69 (1H, d), 7.59 (5H, d), |
| | 7.57 (1H, d), 7.54 (2H, dd), 7.49 (2H, d), 7.47 (2H, d), |
| | 7.41 (1H, dd), 7.39 (2H, dd), 7.31 (2H, s) |
| F-49 | δ = 8.36 (2H, s), 8.08 (2H, d), 8.02 (4H, d), 7.98 (2H, d), |
| | 7.59 (4H, d), 7.51 (6H, d), 7.5 (3H, d), 7.47 (2H, d), |
| | 7.46 (4H, d), 7.41 (2H, dd) |
| F-54 | δ = 8.36 (2H, s), 8.03 (2H, d), 8.02 (2H, d), 7.98 (2H, d), |
| | 7.82 (2H, d), 7.76 (2H, d), 7.75 (4H, d), 7.59 (4H, d), |
| | 7.5 (3H, d), 7.49 (4H, dd), 7.47 (2H, d), 7.41 (2H, d) |
| F-56 | δ = 8.36 (2H, s), 8.02 (2H, d), 7.98 (2H, d), 7.82 (2H, d), |
| | 7.79 (4H, d), 7.69 (2H, d), 7.59 (4H, d), 7.57 (2H, d), |
| | 7.5 (3H, d), 7.47 (2H, dd), 7.46 (4H, d), 7.41 (2H, d) |
| F-60 | δ = 8.02 (2H, s), 7.98 (4H, d), 7.75 (2H, d), 7.59 (4H, d), |
| | 7.54 (2H, d), 7.5 (1H, d), 7.47 (4H, d), 7.46 (4H, d) |
| F-61 | δ = 9.09 (1H, s), 8.49 (1H, d), 8.16 (1H, d), 8.08 (1H, d), |
| | 8.02 (2H, d), 8 (1H, d), 7.98 (2H, d), 7.82 (2H, d), |
| | 7.61 (1H, d), 7.59 (5H, dd), 7.47 (2H, d), 7.41 (2H, d), |
| | 7.39 (2H, dd), 7.31 (4H, dd) |
| F-62 | δ = 9.09 (1H, s), 8.49 (1H, d), 8.16 (1H, d), 8.08 (1H, d), |
| | 8.02 (2H, d), 8 (1H, d), 7.98 (2H, d), 7.77 (2H, d), |
| | 7.61 (1H, d), 7.59 (5H, dd), 7.56 (2H, d), 7.47 (2H, d), |
| | 7.32 (2H, dd) |
| F-65 | δ = 8.36 (2H, s), 8.03 (2H, d), 7.82 (2H, d), 7.73 (4H, d), |
| | 7.51 (4H, d), 7.5 (3H, d), 7.46 (4H, d), 7.41 (3H, d) |
| F-66 | δ = 8.36 (2H, s), 7.51 (2H, d), 7.5 (7H, d), 7.46 (8H, d) |
| F-67 | δ = 8.03 (2H, s), 8.02 (2H, d), 7.98 (2H, d), 7.82 (2H, d), |
| | 7.76 (2H, d), 7.75 (4H, d), 7.59 (4H, d), 7.49 (4H, d), |
| | 7.47 (2H, d), 7.41 (3H, dd) |
| F-68 | δ = 8.36 (2H, s), 8.03 (2H, d), 8.02 (2H, d), 7.98 (2H, d), |
| | 7.82 (2H, d), 7.76 (2H, d), 7.59 (4H, d), 7.5 (3H, d), |
| | 7.49 (1H, d), 7.47 (4H, dd) |
| F-70 | δ = 8.02 (2H, s), 7.98 (4H, d), 7.59 (4H, d), 7.54 (2H, d), |
| | 7.5 (1H, d), 7.49 (1H, d), 7.47 (6H, d) |
| F-71 | δ = 7.98 (1H, s), 7.96 (2H, d), 7.82 (1H, d), 7.69 (1H, d), |
| | 7.57 (1H, d), 7.54 (2H, d), 7.5 (1H, d), 7.49 (1H, d), |
| | 7.47 (6H, d) |
| F-72 | δ = 8.03 (1H, s), 7.82 (2H, d), 7.76 (1H, d), 7.75 (2H, d), |
| | 7.69 (1H, d), 7.57 (1H, d), 7.49 (2H, d), 7.47 (5H, d) |
| F-74 | δ = 8.55 (1H, s), 8.31 (1H, d), 7.94 (1H, d), 7.91 (1H, d), |
| | 7.74 (1H, d), 7.62 (2H, d), 7.58 (1H, d), 7.5 (2H, d), |
| | 7.47 (3H, d) |
| F-75 | δ = 8.02 (2H, s), 7.98 (4H, d), 7.59 (4H, d), 7.54 (2H, d), |
| | 7.47 (2H, d), 7.39 (3H, d), 7.35 (1H, d) |
| F-76 | δ = 8.55 (1H, s), 8.31 (1H, d), 8.02 (2H, d), 7.98 (4H, d), |
| | 7.94 (1H, d), 7.91 (1H, d), 7.74 (1H, d), 7.59 (4H, d), |
| | 7.54 (2H, d), 7.47 (2H, dd), 7.39 (2H, d), 7.35 (1H, d), |
| | 7.31 (2H, dd), 7.16 (2H, dd) |
| F-81 | δ = 8.45 (2H, s), 8.36 (2H, d), 8.02 (2H, d), 7.98 (2H, d), |
| | 7.93 (2H, d), 7.67 (2H, d), 7.59 (4H, d), 7.56 (2H, d), |
| | 7.5 (3H, d), 7.49 (2H, dd) |
| F-82 | δ = 9.09 (1H, s), 8.49 (1H, d), 8.45 (2H, d), 8.16 (1H, d), |
| | 8.08 (1H, d), 8.02 (2H, d), 8 (1H, d), 7.98 (2H, d), |
| | 7.93 (2H, d), 7.67 (2H, dd), 7.61 (1H, d), 7.59 (5H, d), |
| | 7.56 (2H, dd), 7.49 (2H, dd) |
| F-83 | δ = 8.45 (2H, s), 8.02 (2H, d), 7.98 (2H, d), 7.96(2H, d), |
| | 7.93 (2H, d), 7.79 (2H, d), 7.67 (2H, d), 7.6 (2H, d), |
| | 7.59 (4H, d), 7.56 (2H, dd), 7.49 (2H, d), 7.46 (2H, d), |
| | 7.41 (2H, dd) |
| F-84 | δ = 8.45 (2H, s), 8.38 (1H, d), 8.02 (2H, d), 7.98 (2H, d), |
| | 7.94 (1H, d), 7.93 (2H, d), 7.75 (2H, d), 7.73 (1H, d), |
| | 7.67 (2H, d), 7.61 (1H, dd), 7.59 (4H, d), 7.56 (2H, d), |
| | 7.49 (4H, dd), 7.41 (2H, dd) |
| F-85 | δ = 8.45 (2H, s), 8.02 (2H, d), 7.98 (2H, d), 7.96 (2H, d), |
| | 7.93 (2H, d), 7.75 (2H, d), 7.67 (2H, d), 7.59 (4H, d), |
| | 7.56 (2H, d), 7.49 (4H, dd), 7.41 (2H, d), 7.25 (2H, d) |
| F-90 | δ = 8.45 (2H, s), 8.09 (1H, d), 8.06 (1H, d), 8.02 (2H, d), |
| | 7.99 (1H, d), 7.98 (2H, d), 7.96 (2H, d), 7.93 (2H, d), |
| | 7.67 (2H, d), 7.63 (1H, dd), 7.6 (1H, d), 7.59 (4H, d), |
| | 7.56 (2H, dd), 7.55 (1H, dd), 7.49 (2H, s), 7.38 (1H, t), |
| | 7.25 (2H, t) |
| F-95 | δ = 8.45 (2H, s), 8.02 (2H, d), 7.98 (2H, d), 7.93 (2H, d), |
| | 7.8 (2H, d), 7.77 (1H, d), 7.69 (2H, d), 7.67 (2H, d), |
| | 7.62 (1H, d), 7.6 (1H, dd), 7.59 (4H, d), 7.56 (2H, d), |
| | 7.55 (2H, dd), 7.49 (2H, dd) |
| F-99 | δ = 8.55 (1H, s), 8.45 (2H, d), 8.19 (1H, d), 8.02 (2H, d), |
| | 7.98 (2H, d), 7.94 (1H, d), 7.93 (2H, d), 7.67 (2H, d), |
| | 7.59 (4H, d), 7.58 (1H, dd), 7.56 (2H, d), 7.5 (1H, d), |
| | 7.49 (3H, dd), 7.35 (1H, dd), 7.25 (2H, s) |
| F-106 | δ = 8.45 (2H, s), 8.02 (2H, d), 7.98 (3H, d), 7.93 (2H, d), |
| | 7.82 (1H, d), 7.69 (1H, d), 7.67 (2H, d), 7.59 (4H, d), |
| | 7.57 (1H, d), 7.56 (2H, dd), 7.54 (1H, d), 7.49 (2H, d), |
| | 7.39 (1H, dd), 7.31 (1H, dd) |
| F-107 | δ = 8.45 (2H, s), 8.02 (2H, d), 7.98 (3H, d), 7.93 (2H, d), |
| | 7.88 (1H, d), 7.83 (1H, d), 7.79 (1H, d), 7. 67 (2H, d), |
| | 7.59 (4H, d), 7.56 (2H, dd), 7.54 (1H, d), 7.49 (2H, d), |
| | 7.39 (1H, dd), 7.31 (1H, dd) |
| F-112 | δ = 8.45 (2H, s), 8.02 (2H, d), 7.98 (2H, d), 7.93 (2H, d), |
| | 7.9 (1H, d), 7.78 (1H, d), 7.67 (2H, d), 7.65 (1H, d), |
| | 7.59 (4H, d), 7.56 (2H, dd), 7.55 (1H, d), 7.49 (2H, d), |
| | 7.47 (1H, dd), 7.38 (1H, dd), 7.28 (1H, s), 7.2 (1H, t), |
| | 7.16 (1H, t) |
| F-115 | δ = 8.54 (1H, s), 8.45 (2H, d), 8.02 (2H, d), 7.99 (1H, d), |
| | 7.98 (2H, d), 7.93 (2H, d), 7.88 (1H, d), 7.83 (1H, d), |
| | 7.79 (1H, d), 7.67 (2H, dd), 7.61 (1H, d), 7.6 (1H, d), |
| | 7.59 (4H, dd), 7.56 (2H, dd), 7.54 (1H, s), 7.53 (1H, t), |
| | 7.49 (2H, t) |
| F-122 | δ = 8.97 (2H, s), 8.45 (3H, d), 8.2 (1H, d), 8.02 (2H, d), |
| | 7.98 (2H, d), 7.93 (3H, d), 7.67 (2H, d), 7.59 (6H, d), |
| | 7.56 (3H, d), 7.49 (3H, dd) |
| F-123 | δ = 8.45 (2H, s), 8.02 (2H, d), 7.98 (3H, d), 7.96 (2H, d), |
| | 7.93 (2H, d), 7.82 (1H, d), 7.69 (1H, d), 7.67 (2H, d), |
| | 7.59 (4H, d), 7.57 (1H, dd), 7.56 (2H, d), 7.54 (1H, d), |
| | 7.49 (2H, dd), 7.39 (1H, dd), 7.31 (1H, s), 7.25 (2H, t) |
| F-132 | δ = 8.45 (2H, s), 8.36 (2H, d), 8.23 (2H, d), 7.93(2H, d), |
| | 7.75 (4H, d), 7.67 (2H, d), 7.56 (2H, d), 7.55 (2H, d), |
| | 7.5 (3H, d), 7.49 (6H, dd), 7.41 (2H, d), 7.38 (2H, d) |
| F-137 | δ = 8.36 (2H, s), 8.03 (2H, d), 8.02 (2H, d), 7.98 (2H, d), |
| | 7.67 (2H, d), 7.59 (4H, d), 7.57 (2H, d), 7.5 (3H, d), |
| | 7.49 (6H, d) |
| F-145 | δ = 8.36 (2H, s), 8.24 (2H, d), 8.12 (2H, d), 7.94 (2H, d), |
| | 7.73 (2H, d), 7.65 (2H, d), 7.51 (4H, d), 7.5 (3H, d), |
| | 7.46 (4H, d), 7.41 (4H, dd) |
| F-146 | δ = 8.36 (3H, s), 7.65 (2H, d), 7.51 (2H, d), 7.5 (6H, d), |
| | 7.46 (8H, d) |
| F-147 | δ = 8.24 (2H, s), 8.2 (2H, d), 8.17 (2H, d), 8.02 (2H, d), |
| | 7.98 (2H, d), 7.75 (4H, d), 7.67 (2H, d), 7.59 (4H, d), |
| | 7.49 (4H, d), 7.41 (3H, dd) |
| F-148 | δ = 8.36 (2H, s), 8.24 (2H, d), 8.2 (2H, d), 8.17 (2H, d), |
| | 8.02 (2H, d), 7.98 (2H, d), 7.67 (2H, d), 7.59 (4H, d), |
| | 7.5(3H, d) |
| F-150 | δ = 8.45 (2H, s), 8.02 (2H, d), 7.98 (2H, d), 7.93 (2H, d), |
| | 7.67 (2H, d), 7.59 (4H, d), 7.56 (2H, d), 7.5 (3H, d) |
| F-151 | δ = 7.98 (1H, s), 7.96 (2H, d), 7.82 (1H, d), 7.69 (1H, d), |
| | 7.59 (3H, d), 7.57 (1H, d), 7.56 (2H, d) |
| F-152 | δ = 8.03 (1H, s), 7.82 (2H, d), 7.76 (1H, d), 7.75 (2H, d), |
| | 7.69 (1H, d), 7.57 (1H, d), 7.54 (1H, d), 7.5 (3H, d) |
| F-157 | δ = 8.45 (1H, s), 8.03 (1H, d), 7.94 (1H, d), 7.93 (1H, d), |
| | 7.68 (1H, d), 7.57 (1H, d), 7.56 (1H, d), 7.5(3H, d) |
| F-159 | δ = 7.98 (1H, s), 7.91 (1H, d), 7.89 (1H, d), 7.82 (1H, d), |
| | 7.69 (1H, d), 7.57 (1H, d), 7.54 (1H, d), 7.5 (1H, d), |
| | 7.46 (1H, d), 7.44 (1H, dd), 7.41 (4H, d), 7.39 (2H, d), |
| | 7.38 (2H, dd), 7.31 (2H, dd), (5H, s), (5H, t), (5H, t), |
| | (5H, dd), (5H, dd), (5H, t), (5H, d), (5H, d), (5H, d) |
| G-15 | δ = 8.97 (d, 1H), 8.18 (d, 1H), 7.79 (d, 2H), 7.75 (d, 4H), |
| | 7.64~7.25 (m, 26H), 6.91 (d, 1H) |
| G-55 | δ = 7.98 (d, 1H), 7.75 (d, 4H), 7.55~7.25 (m, 23H), |
| | 6.91 (d, 1H) |
| G-58 | δ = 8.08 (d, 1H), 8.02 (d, 1H), 7.98 (d, 1H), 7.86 (d, 1H), |
| | 7.75 (d, 2H), 7.55~7.25 (m, 23H), 1.69 (s, 6H) |

### Experimental Examples

### Experimental Example 1

### (1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′, 4″-tris[2-naphthyl(phenyl)amino]triphenylamine (2-TNATA) and a hole transport layer N,N′-di(1-naphthyl)-N,N′-diphenyl-(1,1′-biphenyl)-4,4′-diamine (NPB), which are common layers, were formed on the ITO transparent electrode (positive electrode).

A light emitting layer was thermally vacuum deposited thereon as follows. The light emitting layer was deposited to have a thickness of 500 Å by using one type of compound described in the following Table 6 as a red host and (piq)₂(Ir) (acac) as a red phosphorescent dopant to dope the host with (piq)₂(Ir) (acac) in an amount of 3%. Thereafter, BCP as a hole blocking layer was deposited to have a thickness of 60 Å, and Alq₃ as an electron transport layer was deposited to have a thickness of 200 Å thereon. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Compounds X1 to X15 used in the following Table 6 are as follows.

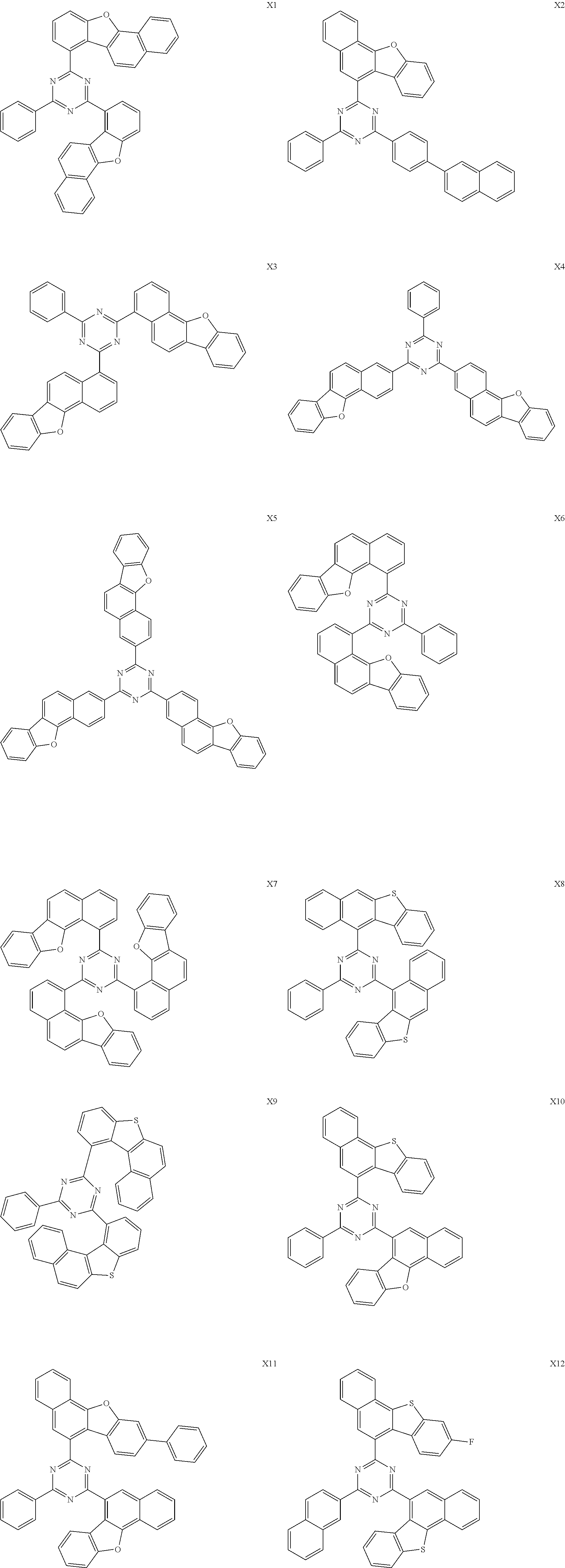

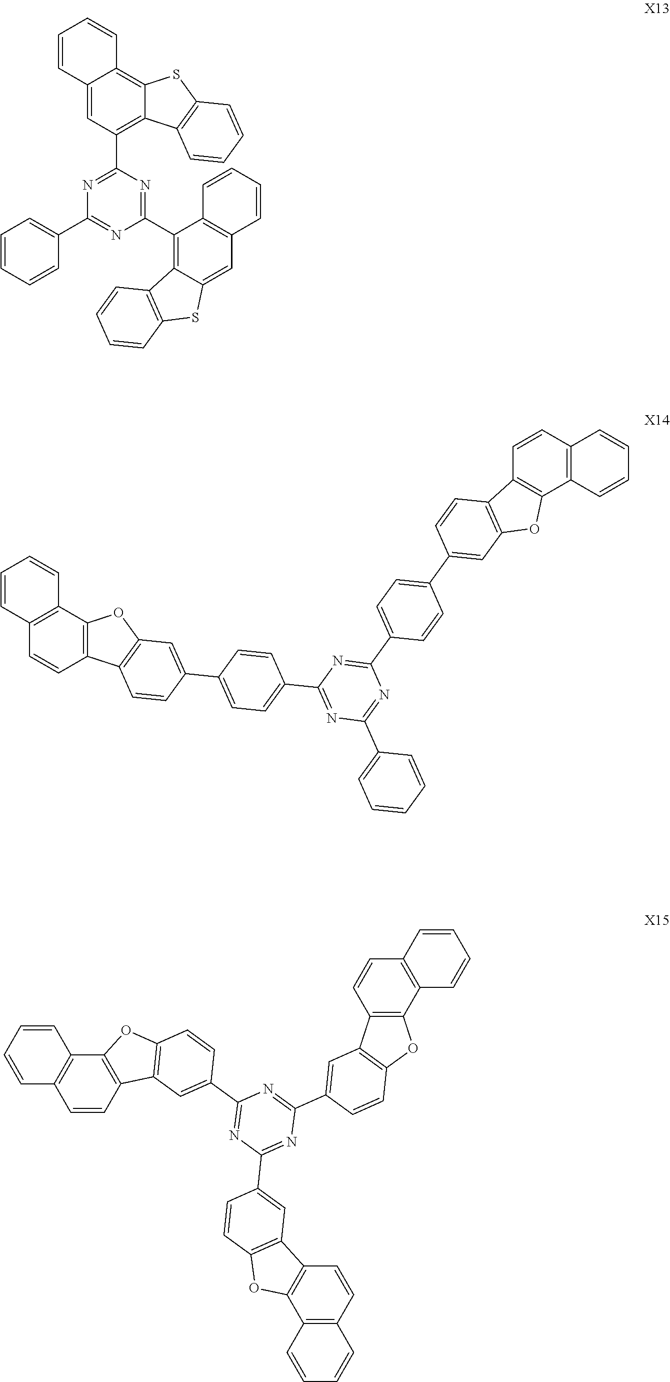 [SMILES: c1ccc(-c2nc(-c3c4ccccc4cc4sc5ccccc5c34)nc(-c3cc4ccccc4c4sc5ccccc5c34)n2)cc1 ; c1ccc(-c2nc(-c3ccc(-c4ccc5c(c4)oc4c6ccccc6ccc54)cc3)nc(-c3ccc(-c4ccc5c(c4)oc4c6ccccc6ccc54)cc3)n2)cc1 ; c1ccc2c(c1)ccc1c3cc(-c4nc(-c5ccc6oc7c8ccccc8ccc7c6c5)nc(-c5ccc6oc7c8ccccc8ccc7c6c5)n4)ccc3oc21]

Meanwhile, all the organic compounds required for manufacturing an OLED device were subjected to vacuum sublimed purification under 10⁻⁸ to 10⁻⁶ torr for each material, and used for the manufacture of OLED.

### (2) Measurement of Driving Voltage, Light Emitting Efficiency, and Service Life of Organic Light Emitting Device

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m². T₉₀ means the service life (unit: hour) that is the time it takes for the luminance to reach 90% relative to the initial luminance.

The characteristics of the organic electroluminescence device manufactured above are as shown in the following Table 6.

TABLE 6

| | | | | | Ser- |
|---|---|---|---|---|---|
| | | Driving | Effic- | Color | vice |
| | Com- | Voltage | iency | coordinate | life |
| | pound | (V) | (cd/A) | (x, y) | (T₉₀) |
| Com- | X1 | 4.26 | 27.3 | (0.686, | 39 |
| parative | | | | 0.314) | |
| Example 1 | | | | | |
| Com- | X2 | 4.37 | 22.8 | (0.685, | 54 |
| parative | | | | 0.315) | |
| Example 2 | | | | | |
| Com- | X3 | 4.39 | 26.1 | (0.685, | 49 |
| parative | | | | 0.315) | |
| Example 3 | | | | | |
| Com- | X4 | 3.89 | 20.1 | (0.682, | 43 |
| parative | | | | 0.318) | |
| Example 4 | | | | | |
| Com- | X5 | 4.59 | 23.4 | (0.684, | 16 |
| parative | | | | 0.316) | |
| Example 5 | | | | | |
| Com- | X6 | 4.59 | 23.1 | (0.685, | 31 |
| parative | | | | 0.315) | |
| Example 6 | | | | | |
| Com- | X7 | 4.42 | 23.5 | (0.685, | 48 |
| parative | | | | 0.315) | |
| Example 7 | | | | | |
| Com- | X8 | 4.79 | 22.6 | (0.682, | 40 |
| parative | | | | 0.318) | |
| Example 8 | | | | | |
| Com- | X9 | 4.18 | 26.8 | (0.682, | 41 |
| parative | | | | 0.318) | |
| Example 9 | | | | | |
| Com- | X10 | 4.01 | 19.9 | (0.682, | 69 |
| parative | | | | 0.318) | |
| Example | | | | | |
| 10 | | | | | |
| Com- | X11 | 3.99 | 21.6 | (0.681, | 73 |
| parative | | | | 0.319) | |
| Example | | | | | |
| 11 | | | | | |
| Com- | X12 | 4.08 | 20.2 | (0.682, | 68 |
| parative | | | | 0.318) | |
| Example | | | | | |
| 12 | | | | | |
| Com- | X13 | 4.27 | 19.5 | (0.682, | 47 |
| parative | | | | 0.318) | |
| Example | | | | | |
| 13 | | | | | |
| Com- | X14 | 4.78 | 18.4 | (0.682, | 30 |
| parative | | | | 0.318) | |
| Example | | | | | |
| 14 | | | | | |
| Com- | X15 | 4.21 | 20.2 | (0.681, | 16 |
| parative | | | | 0.319) | |
| Example | | | | | |
| 15 | | | | | |
| Example 1 | F1 | 3.92 | 38.2 | (0.682, | 164 |
| | | | | 0.318) | |
| Example 2 | F2 | 3.86 | 37.9 | (0.682, | 180 |
| | | | | 0.318) | |
| Example 3 | F3 | 4.02 | 34.3 | (0.682, | 140 |
| | | | | 0.318) | |
| Example 4 | F4 | 3.90 | 37.9 | (0.682, | 158 |
| | | | | 0.318) | |
| Example 5 | F5 | 3.84 | 38.3 | (0.682, | 175 |
| | | | | 0.318) | |
| Example 6 | F7 | 3.86 | 33.9 | (0.682, | 139 |
| | | | | 0.318) | |
| Example 7 | F10 | 3.85 | 38.8 | (0.682, | 180 |
| | | | | 0.318) | |
| Example 8 | F12 | 3.83 | 38.7 | (0.682, | 182 |
| | | | | 0.318) | |
| Example 9 | F14 | 3.87 | 37.0 | (0.682, | 146 |
| | | | | 0.318) | |
| Example | F15 | 4.04 | 29.9 | (0.682, | 128 |
| 10 | | | | 0.318) | |
| Example | F16 | 4.14 | 30.2 | (0.681, | 119 |
| 11 | | | | 0.319) | |
| Example | F17 | 4.01 | 38.1 | (0.682, | 138 |
| 12 | | | | 0.318) | |
| Example | F18 | 3.89 | 35.9 | (0.682, | 130 |
| 13 | | | | 0.318) | |
| Example | F19 | 3.88 | 37.5 | (0.682, | 138 |
| 14 | | | | 0.318) | |
| Example | F20 | 3.98 | 35.1 | (0.682, | 105 |
| 15 | | | | 0.318) | |
| Example | F22 | 3.88 | 38.2 | (0.682, | 118 |
| 16 | | | | 0.318) | |
| Example | F23 | 3.82 | 37.8 | (0.683, | 132 |
| 17 | | | | 0.317) | |
| Example | F25 | 3.91 | 36.2 | (0.682, | 149 |
| 18 | | | | 0.318) | |
| Example | F26 | 3.94 | 38.3 | (0.682, | 169 |
| 19 | | | | 0.318) | |
| Example | F27 | 3.78 | 38.9 | (0.682, | 150 |
| 20 | | | | 0.318) | |
| Example | F28 | 3.69 | 38.6 | (0.682, | 180 |
| 21 | | | | 0.318) | |
| Example | F29 | 3.69 | 40.6 | (0.683, | 78 |
| 22 | | | | 0.317) | |
| Example | F31 | 3.75 | 39.2 | (0.682, | 196 |
| 23 | | | | 0.318) | |
| Example | F32 | 3.79 | 39.9 | (0.682, | 180 |
| 24 | | | | 0.318) | |
| Example | F33 | 3.69 | 38.6 | (0.682, | 176 |
| 25 | | | | 0.318) | |
| Example | F35 | 3.68 | 36.7 | (0.682, | 179 |
| 26 | | | | 0.318) | |
| Example | F37 | 3.71 | 41.8 | (0.682, | 84 |
| 27 | | | | 0.318) | |
| Example | F39 | 3.76 | 34.1 | (0.682, | 166 |
| 28 | | | | 0.318) | |
| Example | F43 | 3.79 | 37.1 | (0.682, | 166 |
| 29 | | | | 0.318) | |
| Example | F46 | 3.77 | 38.9 | (0.682, | 129 |
| 30 | | | | 0.318) | |
| Example | F49 | 3.88 | 38.1 | (0.682, | 188 |
| 31 | | | | 0.318) | |
| Example | F54 | 3.78 | 38.9 | (0.682, | 190 |
| 32 | | | | 0.318) | |
| Example | F56 | 3.84 | 39.1 | (0.682, | 159 |
| 33 | | | | 0.318) | |
| Example | F60 | 3.66 | 38.6 | (0.679, | 88 |
| 34 | | | | 0.321) | |
| Example | F61 | 3.78 | 38.9 | (0.680, | 76 |
| 35 | | | | 0.320) | |
| Example | F62 | 3.61 | 37.9 | (0.679, | 69 |
| 36 | | | | 0.321) | |
| Example | F65 | 3.78 | 38.9 | (0.682, | 200 |
| 37 | | | | 0.318) | |
| Example | F66 | 3.79 | 38.7 | (0.682, | 222 |
| 38 | | | | 0.318) | |
| Example | F67 | 3.78 | 38.9 | (0.682, | 179 |
| 39 | | | | 0.318) | |
| Example | F68 | 3.78 | 38.9 | (0.682, | 189 |
| 40 | | | | 0.318) | |
| Example | F69 | 3.87 | 37.9 | (0.682, | 229 |
| 41 | | | | 0.318) | |
| Example | F70 | 3.86 | 37.9 | (0.682, | 186 |
| 42 | | | | 0.318) | |
| Example | F71 | 3.79 | 37.1 | (0.682, | 190 |
| 43 | | | | 0.318) | |
| Example | F72 | 3.77 | 38.9 | (0.682, | 156 |
| 44 | | | | 0.318) | |
| Example | F73 | 3.91 | 36.2 | (0.682, | 200 |
| 45 | | | | 0.318) | |
| Example | F74 | 3.93 | 35.9 | (0.682, | 206 |
| 46 | | | | 0.318) | |
| Example | F75 | 3.91 | 36.2 | (0.682, | 151 |
| 47 | | | | 0.318) | |
| Example | F76 | 3.91 | 36.2 | (0.682, | 145 |
| 48 | | | | 0.318) | |
| Example | F81 | 3.69 | 31.2 | (0.682, | 139 |
| 49 | | | | 0.318) | |
| Example | F82 | 3.68 | 31.6 | (0.682, | 144 |
| 50 | | | | 0.318) | |
| Example | F83 | 3.76 | 32.1 | (0.682, | 138 |
| 51 | | | | 0.318) | |
| Example | F84 | 3.75 | 32.9 | (0.681, | 150 |
| 52 | | | | 0.319) | |
| Example | F85 | 3.69 | 33.2 | (0.681, | 161 |
| 53 | | | | 0.319) | |
| Example | F90 | 3.69 | 32.4 | (0.682, | 166 |
| 54 | | | | 0.318) | |
| Example | F95 | 3.75 | 31.1 | (0.682, | 139 |
| 55 | | | | 0.318) | |
| Example | F99 | 3.69 | 29.7 | (0.682, | 158 |
| 56 | | | | 0.318) | |
| Example | F106 | 3.77 | 32.7 | (0.682, | 173 |
| 57 | | | | 0.318) | |
| Example | F107 | 3.65 | 30.9 | (0.682, | 169 |
| 58 | | | | 0.318) | |
| Example | F112 | 3.70 | 33.8 | (0.682, | 108 |
| 59 | | | | 0.318) | |
| Example | F115 | 3.82 | 35.8 | (0.682, | 158 |
| 60 | | | | 0.318) | |
| Example | F122 | 3.69 | 35.6 | (0.682, | 189 |
| 61 | | | | 0.318) | |
| Example | F123 | 3.70 | 34.9 | (0.682, | 176 |
| 62 | | | | 0.318) | |
| Example | F132 | 3.74 | 33.9 | (0.682, | 186 |
| 63 | | | | 0.318) | |
| Example | F137 | 3.39 | 27.8 | (0.682, | 102 |
| 64 | | | | 0.318) | |
| Example | F145 | 3.70 | 34.1 | (0.682, | 188 |
| 65 | | | | 0.318) | |
| Example | F146 | 3.76 | 33.8 | (0.682, | 212 |
| 66 | | | | 0.318) | |
| Example | F147 | 3.74 | 33.8 | (0.682, | 186 |
| 67 | | | | 0.318) | |
| Example | F148 | 3.73 | 33.9 | (0.682, | 184 |
| 68 | | | | 0.318) | |
| Example | F149 | 3.70 | 31.0 | (0.682, | 168 |
| 69 | | | | 0.318) | |
| Example | F150 | 3.69 | 31.3 | (0.682, | 149 |
| 70 | | | | 0.318) | |
| Example | F151 | 3.74 | 34.7 | (0.682, | 189 |
| 71 | | | | 0.318) | |
| Example | F152 | 3.76 | 32.1 | (0.682, | 194 |
| 72 | | | | 0.318) | |
| Example | F157 | 3.99 | 37.9 | (0.682, | 212 |
| 73 | | | | 0.318) | |
| Example | F158 | 3.97 | 37.2 | (0.682, | 212 |
| 74 | | | | 0.318) | |
| Example | F159 | 3.69 | 32.8 | (0.682, | 195 |
| 75 | | | | 0.318) | |
| Example | F160 | 3.70 | 32.6 | (0.682, | 194 |
| 76 | | | | 0.318) | |

The heterocyclic compound of Chemical Formula 1 of the present invention has high thermal stability and appropriate molecular weight and band-gap. An appropriate band-gap of the light emitting layer prevents the loss of electrons and holes to help the effective formation of a recombination zone.

As can be seen from the results in Table 6, it could be confirmed that the organic light emitting device using the heterocyclic compound represented by Chemical Formula 1 of the present invention exhibits improved performance compared to the organic light emitting devices using Comparative Compounds X1 to X15.

Specifically, compared to Comparative Examples 1 to 9, 14, and 15, the Example groups of the present invention showed excellent results, particularly in terms of light emitting efficiency and service life, which are believed to be due to the results that when a substituent is substituted at position 6 of a naphtho[1,2-b]benzofuran core as in Chemical Formula 1 of the present invention, the Example groups of the present invention have a higher hole mobility due to a deeper HOMO value than compounds substituted at other positions. In addition, Comparative Examples 10 to 13 have asymmetric structures, which are believed to reduce the conjugation region of the molecule to lower the electron mobility, thereby resulting in lower efficiency than the chemical formulae of the present invention.

### Experimental Example 2

### (1) Manufacture of Organic Light Emitting Device

An organic light emitting device was manufactured in the same manner as in Experimental Example 1, except that two compounds shown in the following Table 7 were used as the red host of the light emitting layer instead of one compound shown in Table 6 above.

### (2) Measurement of Driving Voltage, Light Emitting Efficiency, and Service Life of Organic Light Emitting Device

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m². T₉₀ means the service life (unit: hour) that is the time it takes for the luminance to reach 90% relative to the initial luminance.

The characteristics of the organic electroluminescence device manufactured above are as shown in the following Table 7.

TABLE 7

| | | | | Color | Ser- |
|---|---|---|---|---|---|
| | Compound | Driving | | co- | vice |
| | (weight | Voltage | Efficiency | ordinate | life |
| | ratio) | (V) | (cd/A) | (x, y) | (T₉₀) |
| Compara | G-15:X3 | 4.36 | 35.9 | (0.684, | 92 |
| tive | (1:1) | | | 0.316) | |
| Example | | | | | |
| 16 | | | | | |
| Compara | G-15:X3 | 4.52 | 35.1 | (0.684, | 87 |
| tive | (1.5:1) | | | 0.316) | |
| Example | | | | | |
| 17 | | | | | |
| Compara | G-15:X3 | 4.30 | 36.4 | (0.684, | 99 |
| tive | (1:1.5) | | | 0.316) | |
| Example | | | | | |
| 18 | | | | | |
| Compara | G-55:X3 | 4.38 | 33.9 | (0.684, | 88 |
| tive | (1:1.5) | | | 0.316) | |
| Example | | | | | |
| 19 | | | | | |
| Compara | G-58:X3 | 4.29 | 34.6 | (0.684, | 90 |
| tive | (1:1.5) | | | 0.316) | |
| Example | | | | | |
| 20 | | | | | |
| Compara | G-15:X2 | 4.35 | 32.8 | (0.683, | 112 |
| tive | (1:1.5) | | | 0.37) | |
| Example | | | | | |
| 21 | | | | | |
| Compara | G-58:X2 | 4.36 | 31.9 | (0.683, | 108 |
| tive | (1:1.5) | | | 0.317) | |
| Example | | | | | |
| 22 | | | | | |
| Example | G-55:F-2 | 3.49 | 48.9 | (0.682, | 287 |
| 77 | (1:1.5) | | | 0.318) | |
| Example | G-15:F-2 | 3.52 | 49.2 | (0.682, | 259 |
| 78 | (1:1.5) | | | 0.318) | |
| Example | G-58:F-2 | 3.58 | 48.8 | (0.682, | 280 |
| 79 | (1:1.5) | | | 0.318) | |
| Example | G-55:F-12 | 3.48 | 49.6 | (0.682, | 301 |
| 80 | (1:1.5) | | | 0.318) | |
| Example | G-55:F-12 | 3.62 | 46.8 | (0.682, | 256 |
| 81 | (1.5:1) | | | 0.318) | |
| Example | G-55:F-28 | 3.44 | 49.2 | (0.682, | 270 |
| 82 | (1:1.5) | | | 0.318) | |
| Example | G-55:F-31 | 3.51 | 49.8 | (0.683, | 318 |
| 83 | (1:1.5) | | | 0.317) | |
| Example | G-55:F-29 | 3.46 | 47.2 | (0.683, | 229 |
| 84 | (1:1.5) | | | 0.317) | |
| Example | G-55:F-31 | 3.51 | 48.8 | (0.683, | 300 |
| 85 | (1:1.5) | | | 0.317) | |
| Example | G-15:F-31 | 3.48 | 52.6 | (0.683, | 305 |
| 86 | (1:1.5) | | | 0.317) | |
| Example | G-55:F-69 | 3.48 | 52.7 | (0.683, | 318 |
| 87 | (1:1.5) | | | 0.317) | |
| Example | G-15:F-69 | 3.43 | 52.9 | (0.683, | 346 |
| 88 | (1:1.5) | | | 0.317) | |
| Example | G-58:F-69 | 3.46 | 53.9 | (0.683, | 325 |
| 89 | (1:1.5) | | | 0.317) | |
| Example | G-15:F-73 | 3.76 | 44.8 | (0.682, | 226 |
| 90 | (1:1.5) | | | 0.318) | |
| Example | G-15:F-74 | 3.79 | 44.6 | (0.682, | 219 |
| 91 | (1:1.5) | | | 0.318) | |
| Example | G-15:F-146 | 3.66 | 43.8 | (0.682, | 232 |
| 92 | (1:1) | | | 0.318) | |
| Example | G-15:F-146 | 3.59 | 42.1 | (0.682, | 233 |
| 93 | (1:1.5) | | | 0.318) | |
| Example | G-58:F-146 | 3.49 | 41.8 | (0.682, | 216 |
| 94 | (1:1.5) | | | 0.318) | |
| Example | G-15:F-158 | 3.46 | 48.6 | (0.682, | 277 |
| 95 | (1:1.5) | | | 0.318) | |
| Example | G-58:F-158 | 3.44 | 47.9 | (0.682, | 279 |
| 96 | (1:1.5) | | | 0.318) | |
| Example | G-15:F-160 | 3.69 | 38.9 | (0.683, | 218 |
| 97 | (1.5:1) | | | 0.317) | |
| Example | G-15:F-160 | 3.57 | 40.2 | (0.682, | 220 |
| 98 | (1:1) | | | 0.318) | |
| Example | G-15:F-160 | 3.46 | 42.9 | (0.682, | 228 |
| 99 | (1:1.5) | | | 0.318) | |

As can be seen from the results of Tables 6 and 7 above, when the heterocyclic compound and the compound of Chemical Formula 2 of the present invention are used in combination in the light emitting layer of the organic light emitting device, the improved driving voltage, efficiency and service life characteristics were shown compared to when the compounds are used as a single compound. This corresponds to a result in which by using both the heterocyclic compound of Chemical Formula 1 of the present invention and the compound of Chemical Formula 2 of the present invention for the light emitting layer of the organic light emitting device, an acceptor with a good electron transport ability (n-host) and a donor with a good hole transport ability (p-host) can be used as hosts of the light emitting layer to lower the driving voltage at which electrons and holes are injected, and the efficiency and service life are improved through the effective formation of a recombination zone.

1. A heterocyclic compound of the following Chemical Formula 1: 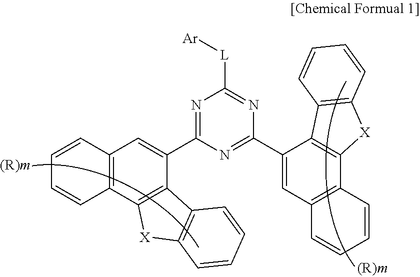
wherein, in Chemical Formula 1,
X is O or S,
L is a direct bond; a substituted or unsubstituted C6 to C60 arylene group; or a substituted or unsubstituted C2 to C60 heteroarylene group,
Ar is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R is hydrogen; deuterium; a halogen group; a cyano group; a substituted or unsubstituted C1 to C60 alkyl group; a substituted or unsubstituted C1 to C60 haloalkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
m is an integer from 1 to 9, and when m is 2 or higher, substituents in the parenthesis are the same as or different from each other.

2. The heterocyclic compound of claim 1, wherein Compound 1 is represented by the following Chemical Formula 1-O or 1-S: 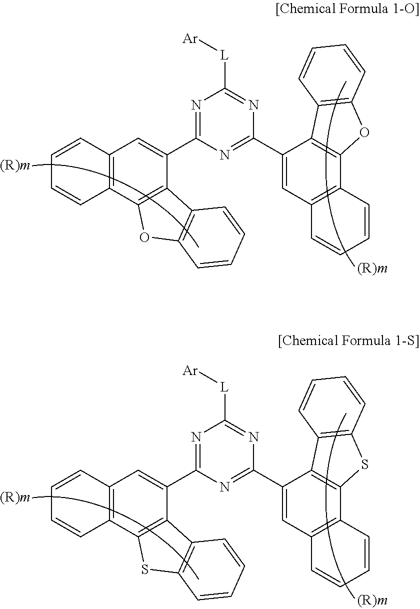
in Chemical Formulae 1-O and 1-S,
the definition of each substituent is the same as the definition in Chemical Formula 1.

3. The heterocyclic compound of claim 1, wherein Ar is a substituted or unsubstituted C6 to C30 aryl group; or a substituted or unsubstituted C2 to C30 heteroaryl group.

4. The heterocyclic compound of claim 1, wherein R is hydrogen; deuterium; a halogen group; a substituted or unsubstituted C1 to C30 haloalkyl group; or a substituted or unsubstituted C6 to C30 aryl group.

5. The heterocyclic compound of claim 1, wherein a deuterium substitution rate of Chemical Formula 1 is 0%, or 5% to 100%.

6. The heterocyclic compound of claim 1, wherein Chemical Formula 1 is represented by any one of the following compounds:

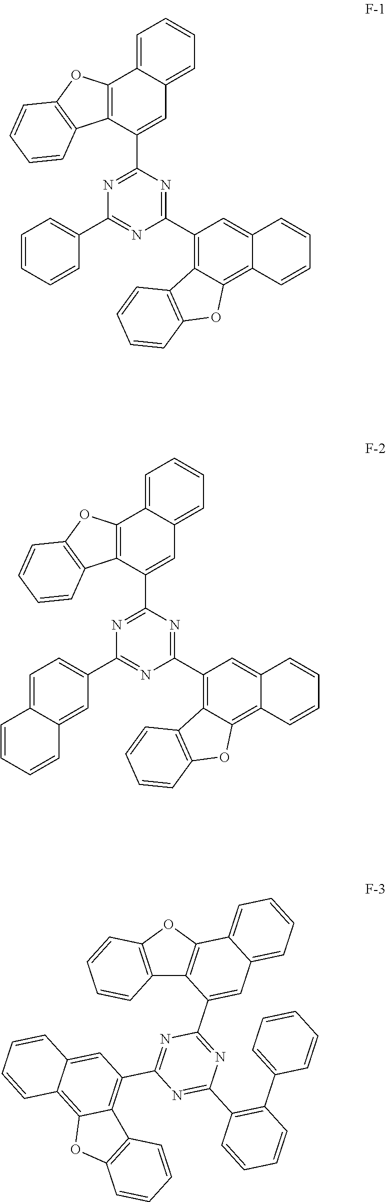 [SMILES: c1ccc(-c2nc(-c3cc4ccccc4c4oc5ccccc5c34)nc(-c3cc4ccccc4c4oc5ccccc5c34)n2)cc1 ; c1ccc2cc(-c3nc(-c4cc5ccccc5c5oc6ccccc6c45)nc(-c4cc5ccccc5c5oc6ccccc6c45)n3)ccc2c1 ; c1ccc(-c2ccccc2-c2nc(-c3cc4ccccc4c4oc5ccccc5c34)nc(-c3cc4ccccc4c4oc5ccccc5c34)n2)cc1]

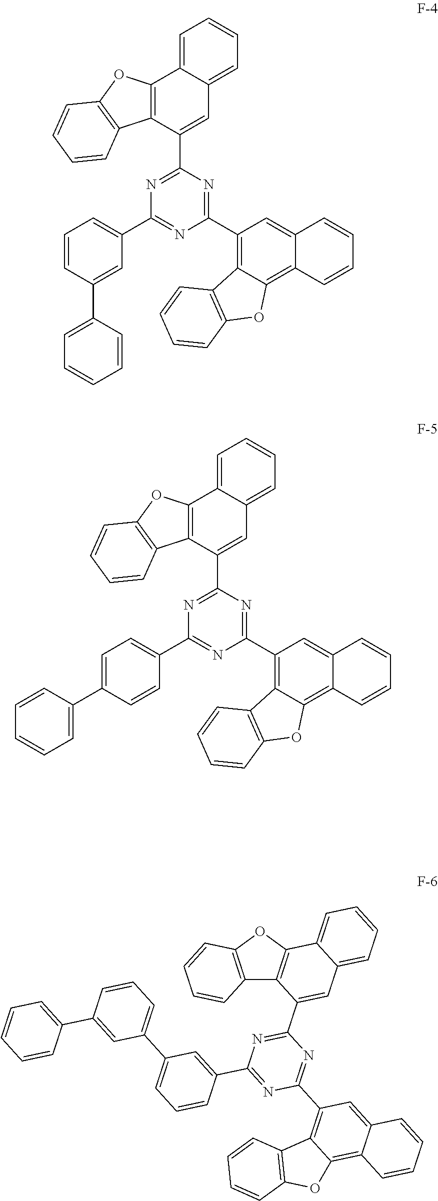 [SMILES: c1ccc(-c2cccc(-c3nc(-c4cc5ccccc5c5oc6ccccc6c45)nc(-c4cc5ccccc5c5oc6ccccc6c45)n3)c2)cc1 ; c1ccc(-c2ccc(-c3nc(-c4cc5ccccc5c5oc6ccccc6c45)nc(-c4cc5ccccc5c5oc6ccccc6c45)n3)cc2)cc1 ; c1ccc(-c2cccc(-c3cccc(-c4nc(-c5cc6ccccc6c6oc7ccccc7c56)nc(-c5cc6ccccc6c6oc7ccccc7c56)n4)c3)c2)cc1]

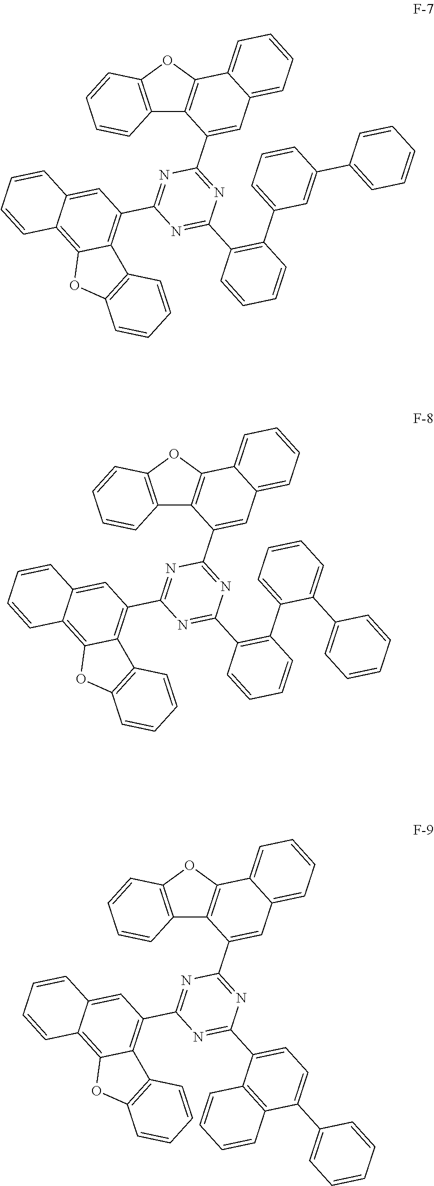 [SMILES: c1ccc(-c2cccc(-c3ccccc3-c3nc(-c4cc5ccccc5c5oc6ccccc6c45)nc(-c4cc5ccccc5c5oc6ccccc6c45)n3)c2)cc1 ; c1ccc(-c2ccccc2-c2ccccc2-c2nc(-c3cc4ccccc4c4oc5ccccc5c34)nc(-c3cc4ccccc4c4oc5ccccc5c34)n2)cc1 ; c1ccc(-c2ccc(-c3nc(-c4cc5ccccc5c5oc6ccccc6c45)nc(-c4cc5ccccc5c5oc6ccccc6c45)n3)c3ccccc23)cc1]

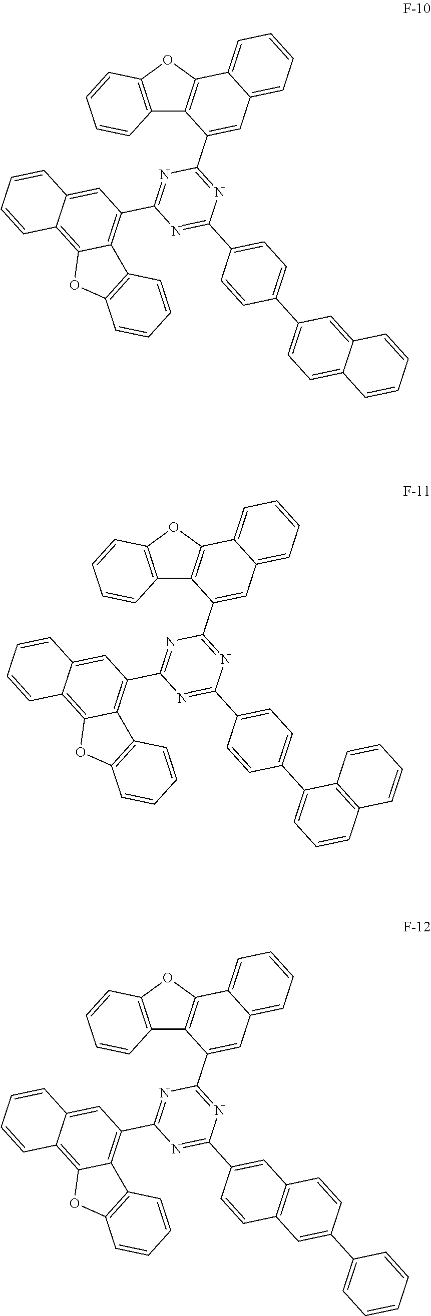 [SMILES: c1ccc2cc(-c3ccc(-c4nc(-c5cc6ccccc6c6oc7ccccc7c56)nc(-c5cc6ccccc6c6oc7ccccc7c56)n4)cc3)ccc2c1 ; c1ccc2c(-c3ccc(-c4nc(-c5cc6ccccc6c6oc7ccccc7c56)nc(-c5cc6ccccc6c6oc7ccccc7c56)n4)cc3)cccc2c1 ; c1ccc(-c2ccc3cc(-c4nc(-c5cc6ccccc6c6oc7ccccc7c56)nc(-c5cc6ccccc6c6oc7ccccc7c56)n4)ccc3c2)cc1]

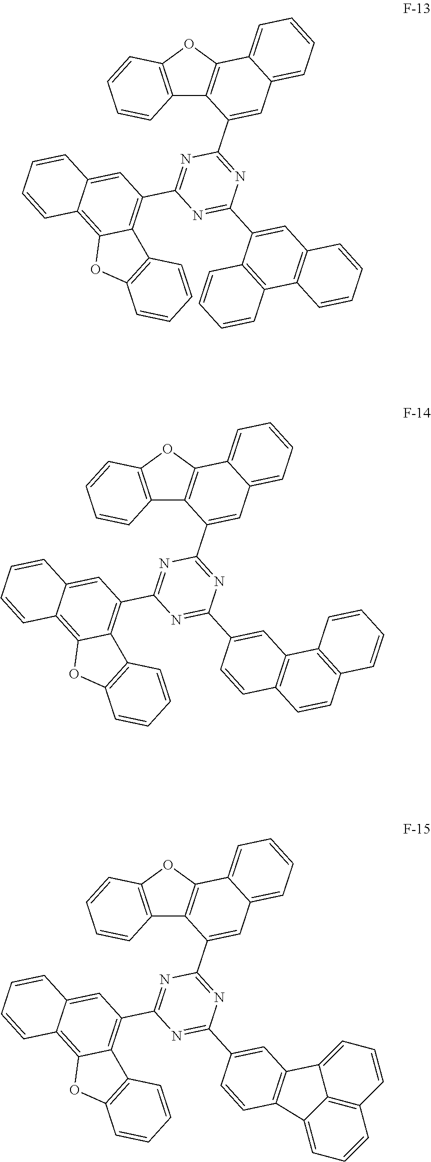 [SMILES: c1ccc2c(c1)cc(-c1nc(-c3cc4ccccc4c4oc5ccccc5c34)nc(-c3cc4ccccc4c4oc5ccccc5c34)n1)c1ccccc12 ; c1ccc2c(c1)ccc1ccc(-c3nc(-c4cc5ccccc5c5oc6ccccc6c45)nc(-c4cc5ccccc5c5oc6ccccc6c45)n3)cc12 ; c1cc2c3c(cccc3c1)-c1cc(-c3nc(-c4cc5ccccc5c5oc6ccccc6c45)nc(-c4cc5ccccc5c5oc6ccccc6c45)n3)ccc1-2]

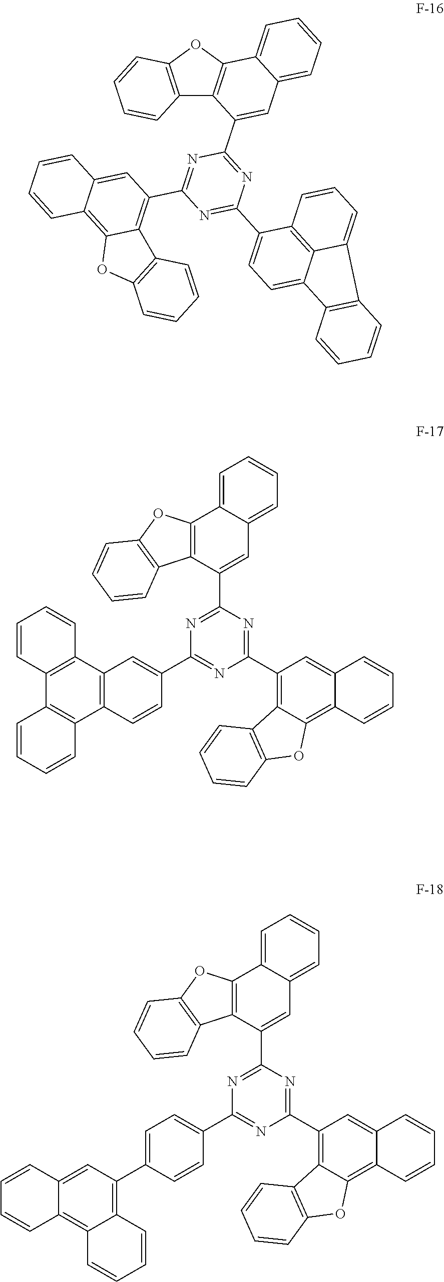 [SMILES: c1ccc2c(c1)-c1cccc3c(-c4nc(-c5cc6ccccc6c6oc7ccccc7c56)nc(-c5cc6ccccc6c6oc7ccccc7c56)n4)ccc-2c13 ; c1ccc2c(c1)cc(-c1nc(-c3ccc4c5ccccc5c5ccccc5c4c3)nc(-c3cc4ccccc4c4oc5ccccc5c34)n1)c1c3ccccc3oc21 ; c1ccc2c(c1)cc(-c1ccc(-c3nc(-c4cc5ccccc5c5oc6ccccc6c45)nc(-c4cc5ccccc5c5oc6ccccc6c45)n3)cc1)c1ccccc12]

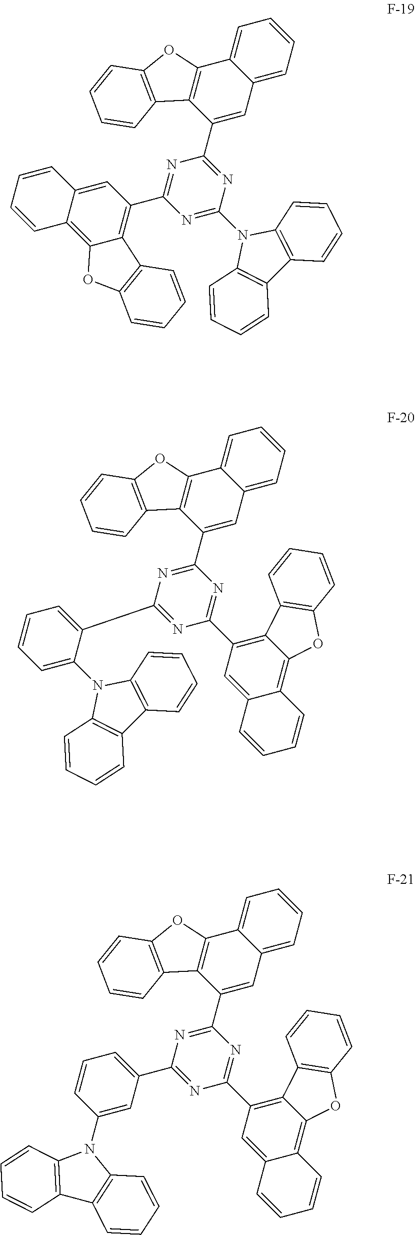 [SMILES: c1ccc2c(c1)cc(-c1nc(-c3cc4ccccc4c4oc5ccccc5c34)nc(-n3c4ccccc4c4ccccc43)n1)c1c3ccccc3oc21 ; c1ccc(-n2c3ccccc3c3ccccc32)c(-c2nc(-c3cc4ccccc4c4oc5ccccc5c34)nc(-c3cc4ccccc4c4oc5ccccc5c34)n2)c1 ; c1cc(-c2nc(-c3cc4ccccc4c4oc5ccccc5c34)nc(-c3cc4ccccc4c4oc5ccccc5c34)n2)cc(-n2c3ccccc3c3ccccc32)c1]

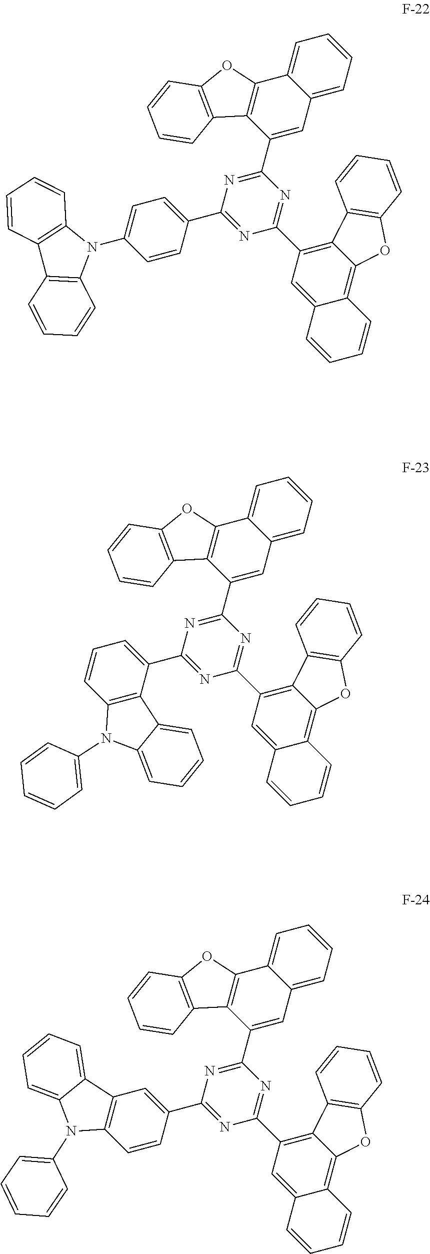 [SMILES: c1ccc2c(c1)cc(-c1nc(-c3ccc(-n4c5ccccc5c5ccccc54)cc3)nc(-c3cc4ccccc4c4oc5ccccc5c34)n1)c1c3ccccc3oc21 ; c1ccc(-n2c3ccccc3c3c(-c4nc(-c5cc6ccccc6c6oc7ccccc7c56)nc(-c5cc6ccccc6c6oc7ccccc7c56)n4)cccc32)cc1 ; c1ccc(-n2c3ccccc3c3cc(-c4nc(-c5cc6ccccc6c6oc7ccccc7c56)nc(-c5cc6ccccc6c6oc7ccccc7c56)n4)ccc32)cc1]

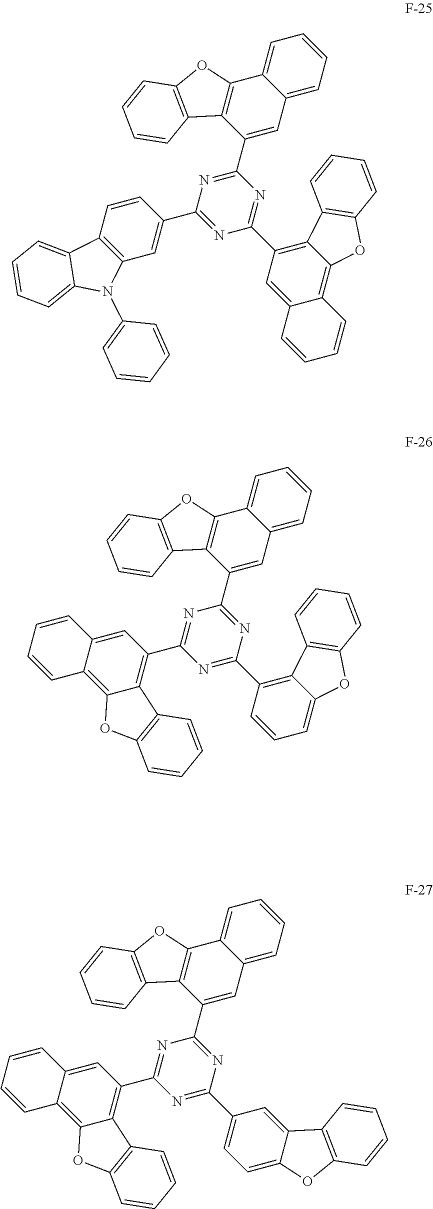 [SMILES: c1ccc(-n2c3ccccc3c3ccc(-c4nc(-c5cc6ccccc6c6oc7ccccc7c56)nc(-c5cc6ccccc6c6oc7ccccc7c56)n4)cc32)cc1 ; c1ccc2c(c1)cc(-c1nc(-c3cccc4oc5ccccc5c34)nc(-c3cc4ccccc4c4oc5ccccc5c34)n1)c1c3ccccc3oc21 ; c1ccc2c(c1)cc(-c1nc(-c3ccc4oc5ccccc5c4c3)nc(-c3cc4ccccc4c4oc5ccccc5c34)n1)c1c3ccccc3oc21]

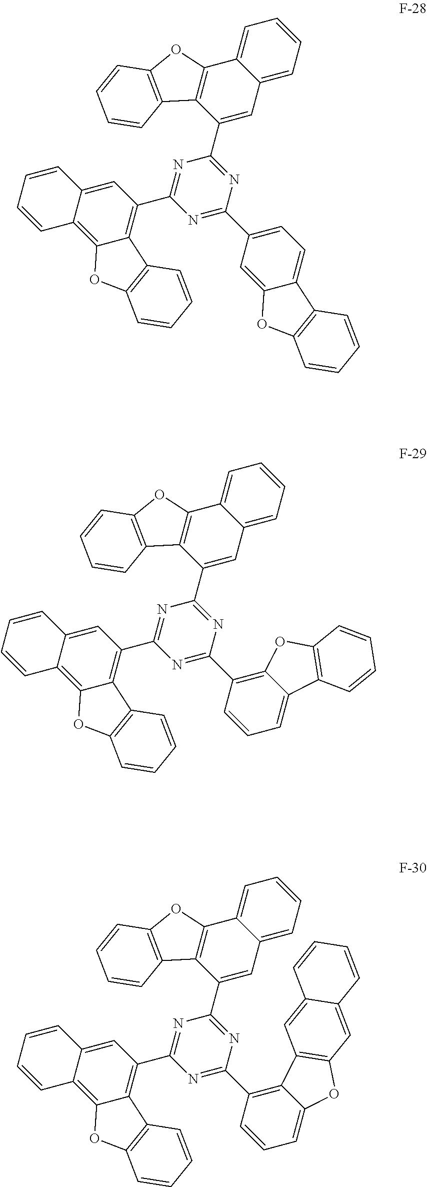 [SMILES: c1ccc2c(c1)cc(-c1nc(-c3ccc4c(c3)oc3ccccc34)nc(-c3cc4ccccc4c4oc5ccccc5c34)n1)c1c3ccccc3oc21 ; c1ccc2c(c1)cc(-c1nc(-c3cccc4c3oc3ccccc34)nc(-c3cc4ccccc4c4oc5ccccc5c34)n1)c1c3ccccc3oc21 ; c1ccc2cc3c(cc2c1)oc1cccc(-c2nc(-c4cc5ccccc5c5oc6ccccc6c45)nc(-c4cc5ccccc5c5oc6ccccc6c45)n2)c13]

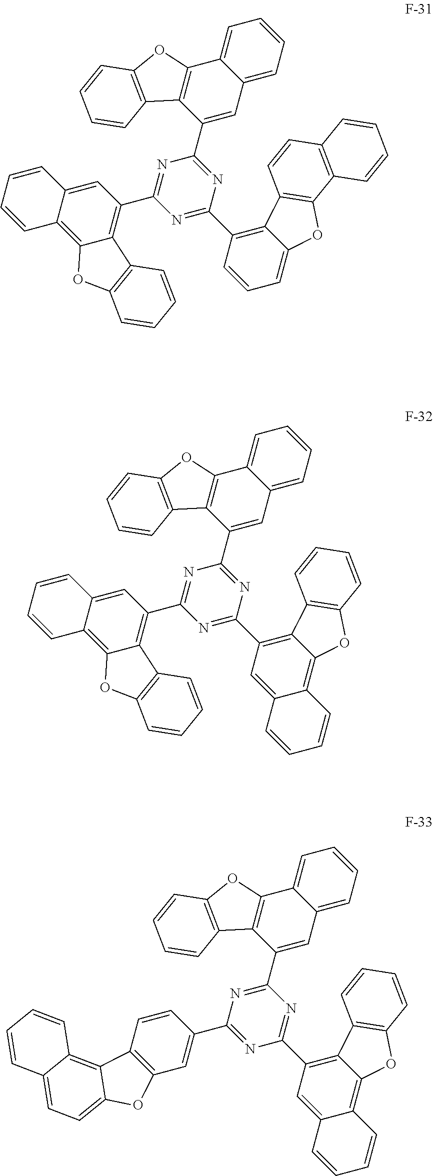 [SMILES: c1ccc2c(c1)ccc1c2oc2cccc(-c3nc(-c4cc5ccccc5c5oc6ccccc6c45)nc(-c4cc5ccccc5c5oc6ccccc6c45)n3)c21 ; c1ccc2c(c1)cc(-c1nc(-c3cc4ccccc4c4oc5ccccc5c34)nc(-c3cc4ccccc4c4oc5ccccc5c34)n1)c1c3ccccc3oc21 ; c1ccc2c(c1)cc(-c1nc(-c3ccc4c(c3)oc3ccc5ccccc5c34)nc(-c3cc4ccccc4c4oc5ccccc5c34)n1)c1c3ccccc3oc21]

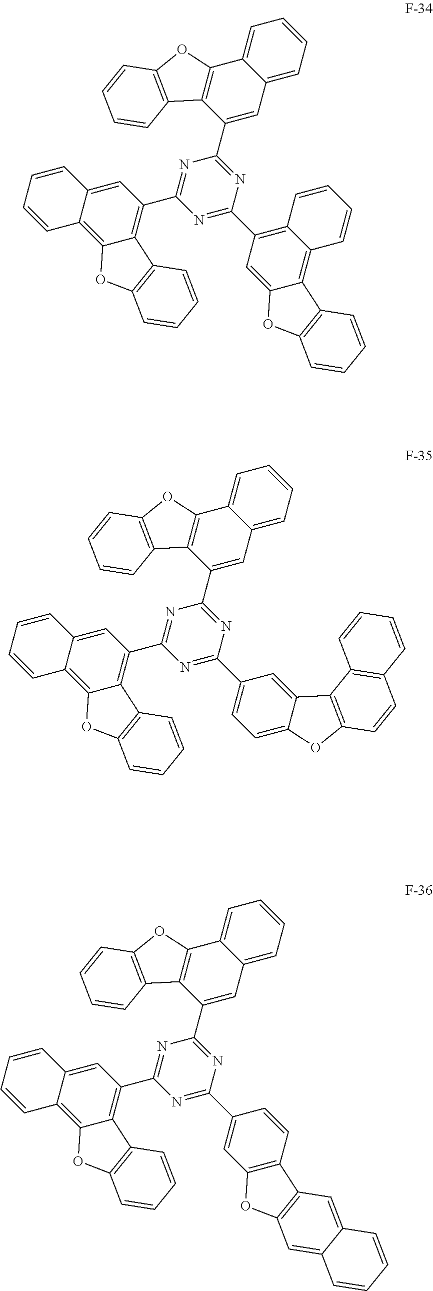 [SMILES: c1ccc2c(c1)cc(-c1nc(-c3cc4oc5ccccc5c4c4ccccc34)nc(-c3cc4ccccc4c4oc5ccccc5c34)n1)c1c3ccccc3oc21 ; c1ccc2c(c1)cc(-c1nc(-c3ccc4oc5ccc6ccccc6c5c4c3)nc(-c3cc4ccccc4c4oc5ccccc5c34)n1)c1c3ccccc3oc21 ; c1ccc2cc3c(cc2c1)oc1cc(-c2nc(-c4cc5ccccc5c5oc6ccccc6c45)nc(-c4cc5ccccc5c5oc6ccccc6c45)n2)ccc13]

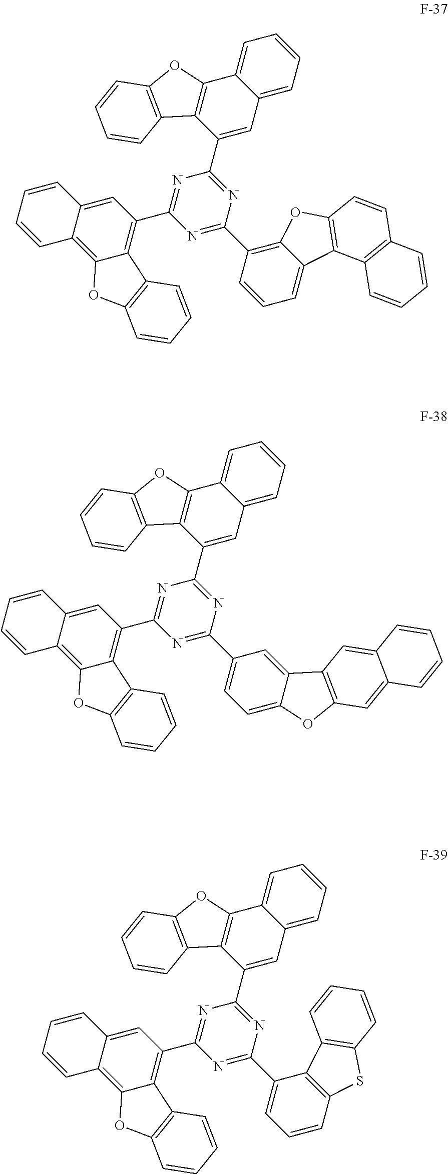 [SMILES: c1ccc2c(c1)cc(-c1nc(-c3cccc4c3oc3ccc5ccccc5c34)nc(-c3cc4ccccc4c4oc5ccccc5c34)n1)c1c3ccccc3oc21 ; c1ccc2cc3c(cc2c1)oc1ccc(-c2nc(-c4cc5ccccc5c5oc6ccccc6c45)nc(-c4cc5ccccc5c5oc6ccccc6c45)n2)cc13 ; c1ccc2c(c1)cc(-c1nc(-c3cccc4sc5ccccc5c34)nc(-c3cc4ccccc4c4oc5ccccc5c34)n1)c1c3ccccc3oc21]

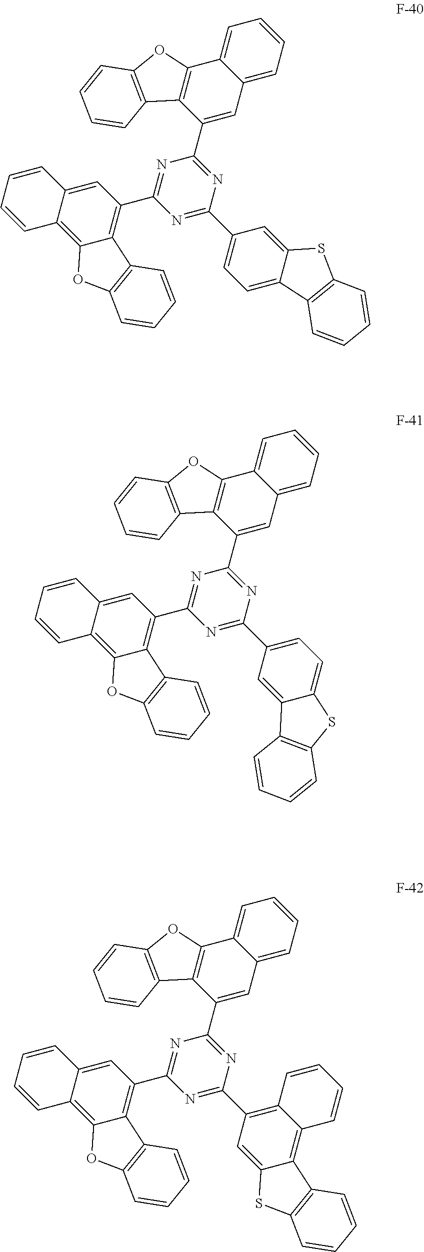 [SMILES: c1ccc2c(c1)cc(-c1nc(-c3ccc4c(c3)sc3ccccc34)nc(-c3cc4ccccc4c4oc5ccccc5c34)n1)c1c3ccccc3oc21 ; c1ccc2c(c1)cc(-c1nc(-c3ccc4sc5ccccc5c4c3)nc(-c3cc4ccccc4c4oc5ccccc5c34)n1)c1c3ccccc3oc21 ; c1ccc2c(c1)cc(-c1nc(-c3cc4sc5ccccc5c4c4ccccc34)nc(-c3cc4ccccc4c4oc5ccccc5c34)n1)c1c3ccccc3oc21]

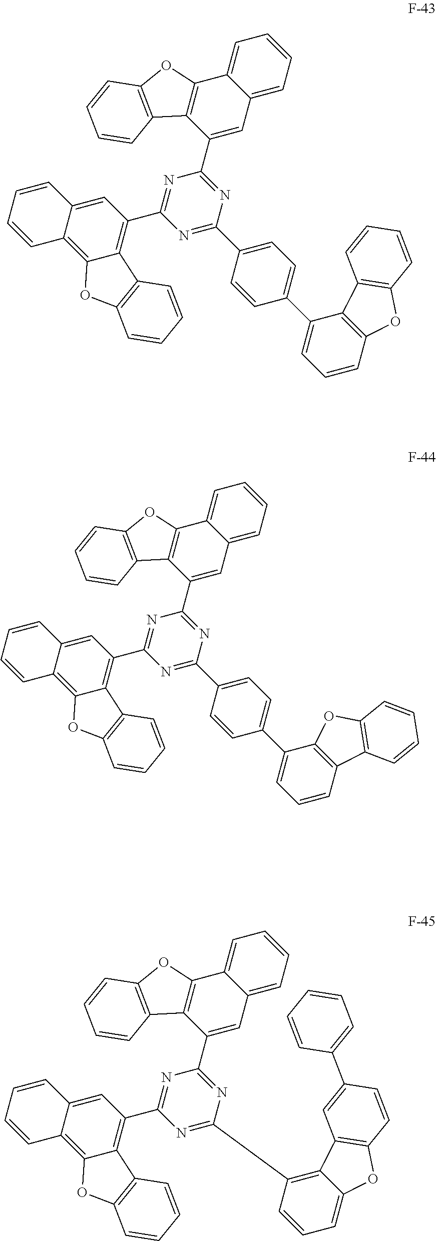 [SMILES: c1ccc2c(c1)cc(-c1nc(-c3ccc(-c4cccc5oc6ccccc6c45)cc3)nc(-c3cc4ccccc4c4oc5ccccc5c34)n1)c1c3ccccc3oc21 ; c1ccc2c(c1)cc(-c1nc(-c3ccc(-c4cccc5c4oc4ccccc45)cc3)nc(-c3cc4ccccc4c4oc5ccccc5c34)n1)c1c3ccccc3oc21 ; c1ccc(-c2ccc3oc4cccc(-c5nc(-c6cc7ccccc7c7oc8ccccc8c67)nc(-c6cc7ccccc7c7oc8ccccc8c67)n5)c4c3c2)cc1]

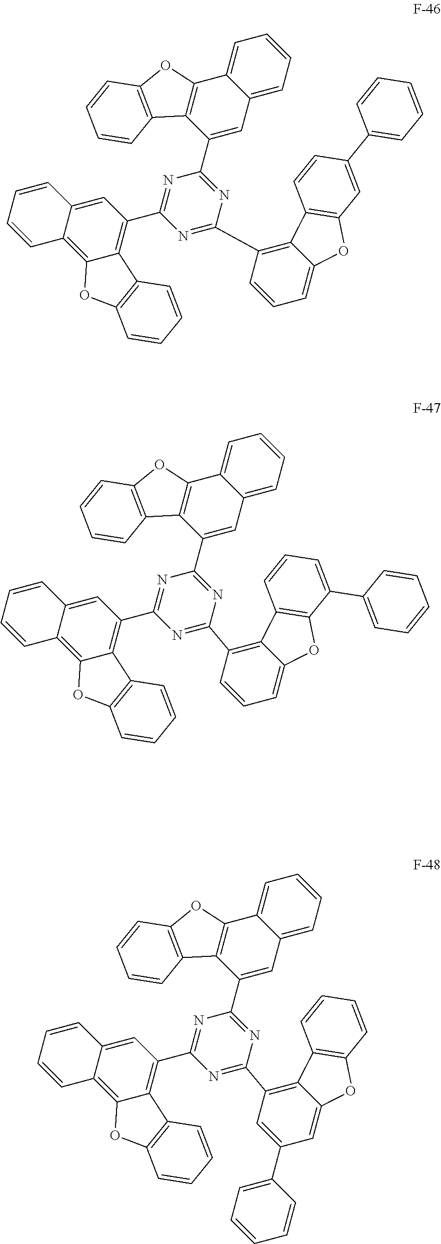 [SMILES: c1ccc(-c2ccc3c(c2)oc2cccc(-c4nc(-c5cc6ccccc6c6oc7ccccc7c56)nc(-c5cc6ccccc6c6oc7ccccc7c56)n4)c23)cc1 ; c1ccc(-c2cccc3c2oc2cccc(-c4nc(-c5cc6ccccc6c6oc7ccccc7c56)nc(-c5cc6ccccc6c6oc7ccccc7c56)n4)c23)cc1 ; c1ccc(-c2cc(-c3nc(-c4cc5ccccc5c5oc6ccccc6c45)nc(-c4cc5ccccc5c5oc6ccccc6c45)n3)c3c(c2)oc2ccccc23)cc1]

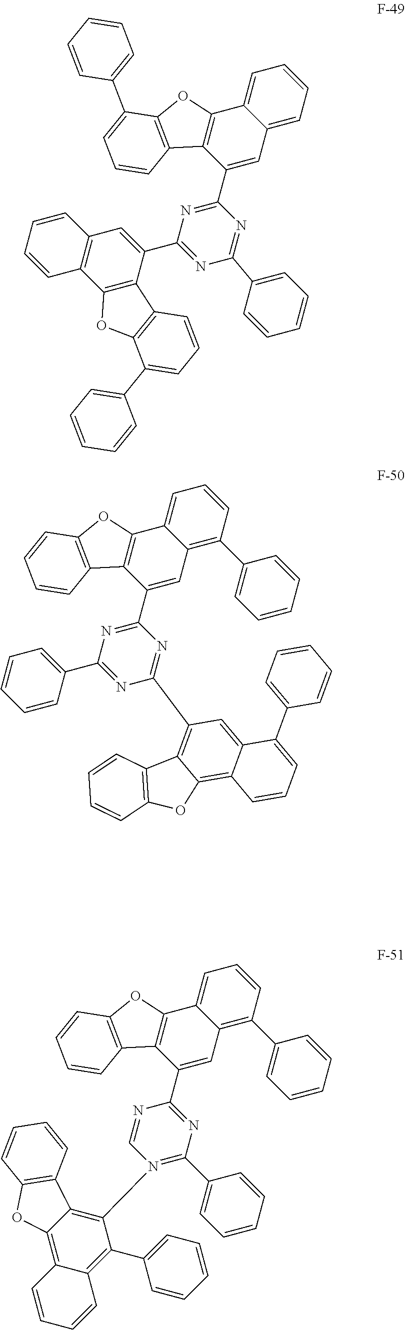

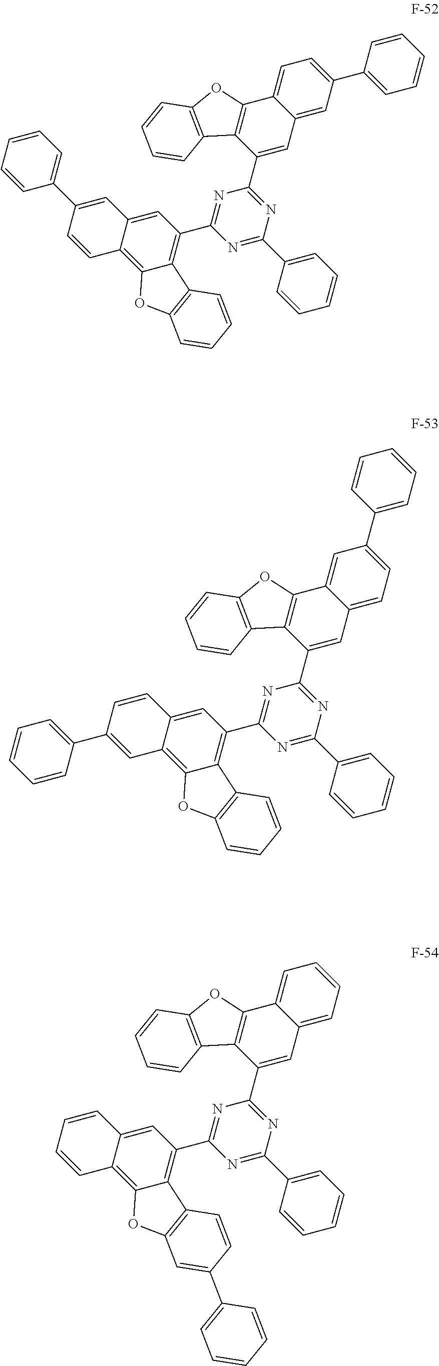 [SMILES: c1ccc(-c2ccc3c(c2)cc(-c2nc(-c4ccccc4)nc(-c4cc5cc(-c6ccccc6)ccc5c5oc6ccccc6c45)n2)c2c4ccccc4oc32)cc1 ; c1ccc(-c2ccc3cc(-c4nc(-c5ccccc5)nc(-c5cc6ccc(-c7ccccc7)cc6c6oc7ccccc7c56)n4)c4c5ccccc5oc4c3c2)cc1 ; c1ccc(-c2ccc3c(c2)oc2c4ccccc4cc(-c4nc(-c5ccccc5)nc(-c5cc6ccccc6c6oc7ccccc7c56)n4)c32)cc1]

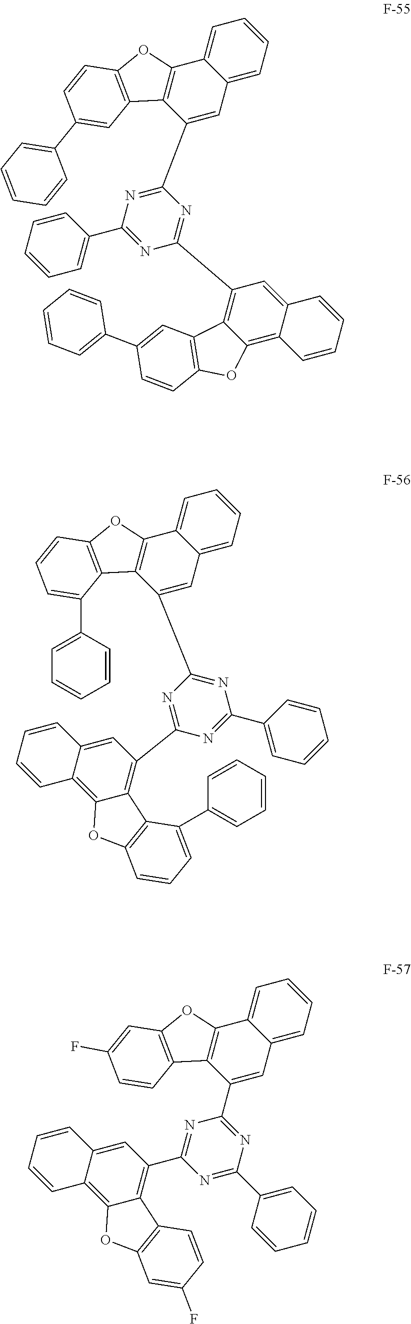 [SMILES: c1ccc(-c2ccc3oc4c5ccccc5cc(-c5nc(-c6ccccc6)nc(-c6cc7ccccc7c7oc8ccc(-c9ccccc9)cc8c67)n5)c4c3c2)cc1 ; c1ccc(-c2nc(-c3cc4ccccc4c4oc5cccc(-c6ccccc6)c5c34)nc(-c3cc4ccccc4c4oc5cccc(-c6ccccc6)c5c34)n2)cc1 ; Fc1ccc2c(c1)oc1c3ccccc3cc(-c3nc(-c4ccccc4)nc(-c4cc5ccccc5c5oc6cc(F)ccc6c45)n3)c21]

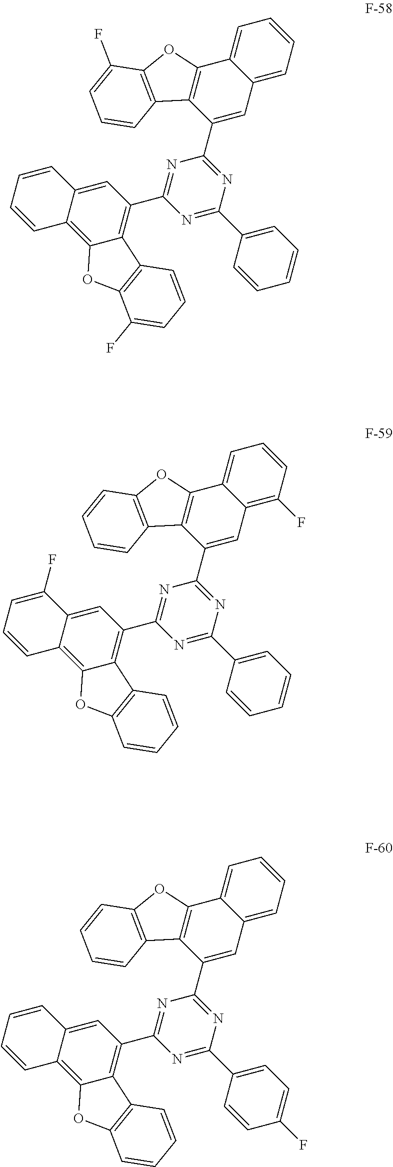 [SMILES: Fc1cccc2c1oc1c3ccccc3cc(-c3nc(-c4ccccc4)nc(-c4cc5ccccc5c5oc6c(F)cccc6c45)n3)c21 ; Fc1cccc2c1cc(-c1nc(-c3ccccc3)nc(-c3cc4c(F)cccc4c4oc5ccccc5c34)n1)c1c3ccccc3oc21 ; Fc1ccc(-c2nc(-c3cc4ccccc4c4oc5ccccc5c34)nc(-c3cc4ccccc4c4oc5ccccc5c34)n2)cc1]

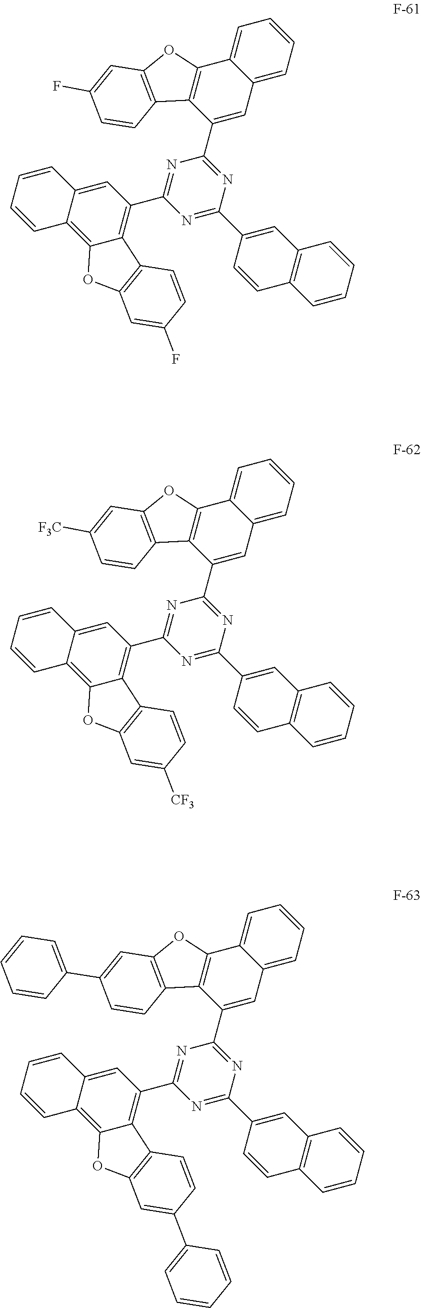 [SMILES: Fc1ccc2c(c1)oc1c3ccccc3cc(-c3nc(-c4ccc5ccccc5c4)nc(-c4cc5ccccc5c5oc6cc(F)ccc6c45)n3)c21 ; FC(F)(F)c1ccc2c(c1)oc1c3ccccc3cc(-c3nc(-c4ccc5ccccc5c4)nc(-c4cc5ccccc5c5oc6cc(C(F)(F)F)ccc6c45)n3)c21 ; c1ccc(-c2ccc3c(c2)oc2c4ccccc4cc(-c4nc(-c5ccc6ccccc6c5)nc(-c5cc6ccccc6c6oc7cc(-c8ccccc8)ccc7c56)n4)c32)cc1]

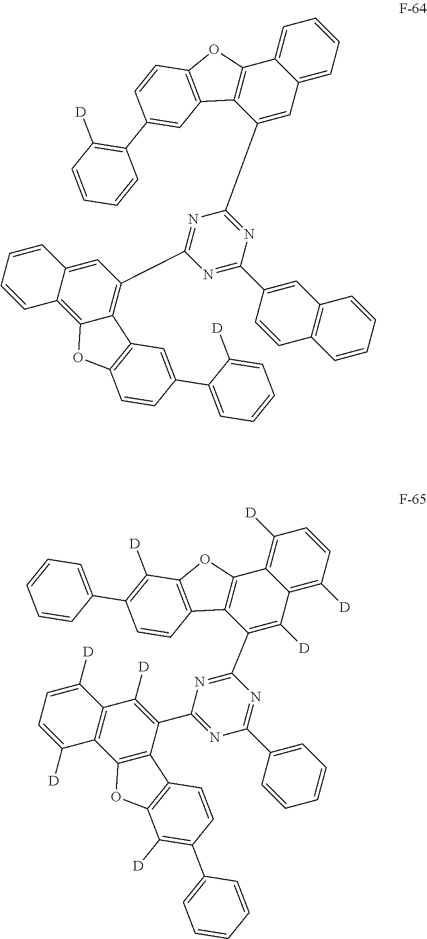 [SMILES: [2H]c1ccccc1-c1ccc2oc3c4ccccc4cc(-c4nc(-c5ccc6ccccc6c5)nc(-c5cc6ccccc6c6oc7ccc(-c8ccccc8[2H])cc7c56)n4)c3c2c1 ; [2H]c1c(-c2ccccc2)ccc2c1oc1c3c([2H])ccc([2H])c3c([2H])c(-c3nc(-c4ccccc4)nc(-c4c([2H])c5c([2H])ccc([2H])c5c5oc6c([2H])c(-c7ccccc7)ccc6c45)n3)c21]

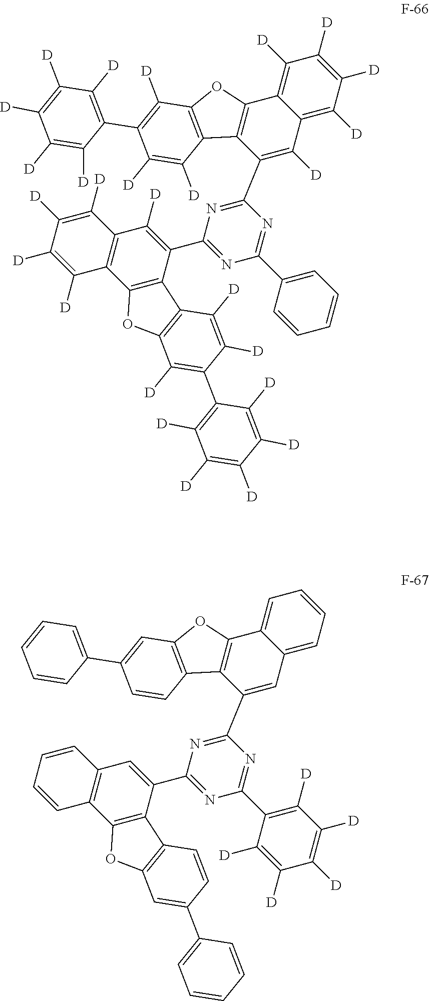 [SMILES: [2H]c1c([2H])c([2H])c(-c2c([2H])c([2H])c3c(oc4c5c([2H])c([2H])c([2H])c([2H])c5c([2H])c(-c5nc(-c6ccccc6)nc(-c6c([2H])c7c([2H])c([2H])c([2H])c([2H])c7c7oc8c([2H])c(-c9c([2H])c([2H])c([2H])c([2H])c9[2H])c([2H])c([2H])c8c67)n5)c43)c2[2H])c([2H])c1[2H] ; [2H]c1c([2H])c([2H])c(-c2nc(-c3cc4ccccc4c4oc5cc(-c6ccccc6)ccc5c34)nc(-c3cc4ccccc4c4oc5cc(-c6ccccc6)ccc5c34)n2)c([2H])c1[2H]]

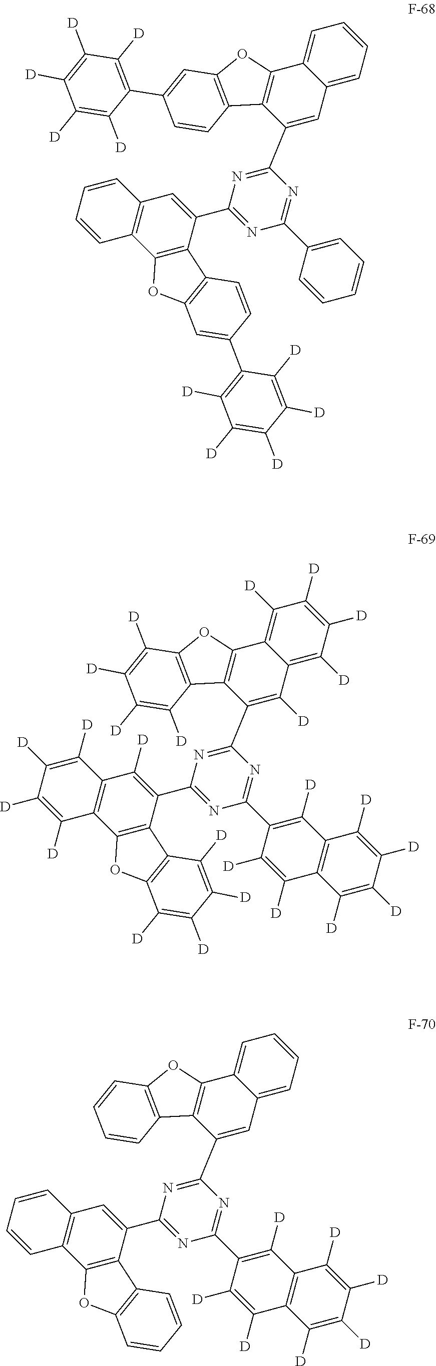

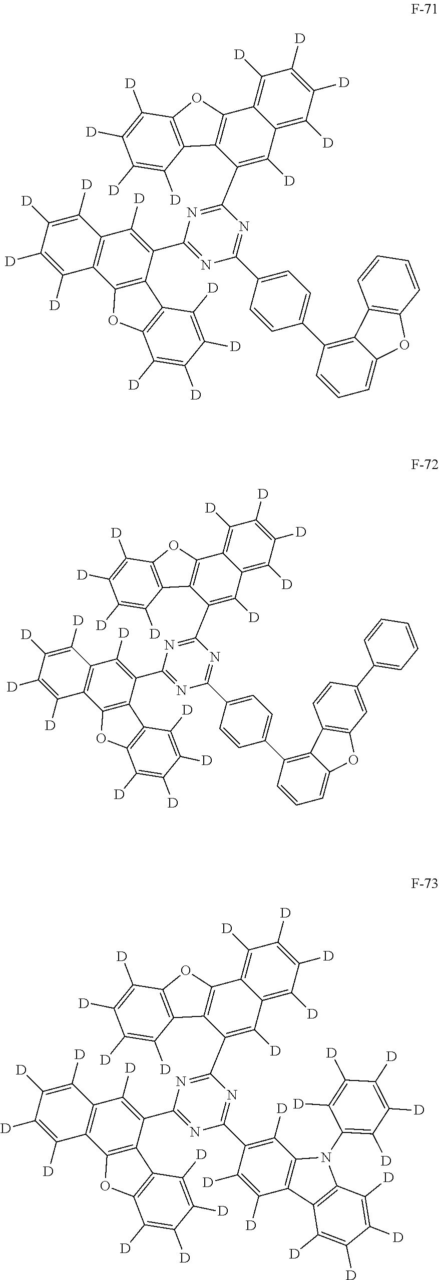

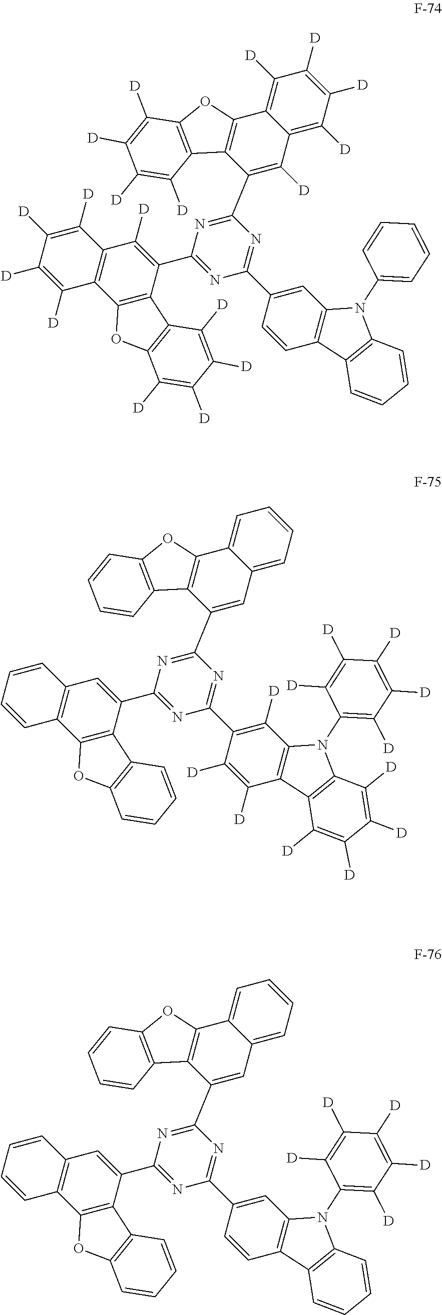 [SMILES: [2H]c1c([2H])c([2H])c2c(oc3c4c([2H])c([2H])c([2H])c([2H])c4c([2H])c(-c4nc(-c5ccc6c7ccccc7n(-c7ccccc7)c6c5)nc(-c5c([2H])c6c([2H])c([2H])c([2H])c([2H])c6c6oc7c([2H])c([2H])c([2H])c([2H])c7c56)n4)c32)c1[2H] ; [2H]c1c([2H])c([2H])c(-n2c3c([2H])c([2H])c([2H])c([2H])c3c3c([2H])c([2H])c(-c4nc(-c5cc6ccccc6c6oc7ccccc7c56)nc(-c5cc6ccccc6c6oc7ccccc7c56)n4)c([2H])c32)c([2H])c1[2H] ; [2H]c1c([2H])c([2H])c(-n2c3ccccc3c3ccc(-c4nc(-c5cc6ccccc6c6oc7ccccc7c56)nc(-c5cc6ccccc6c6oc7ccccc7c56)n4)cc32)c([2H])c1[2H]]

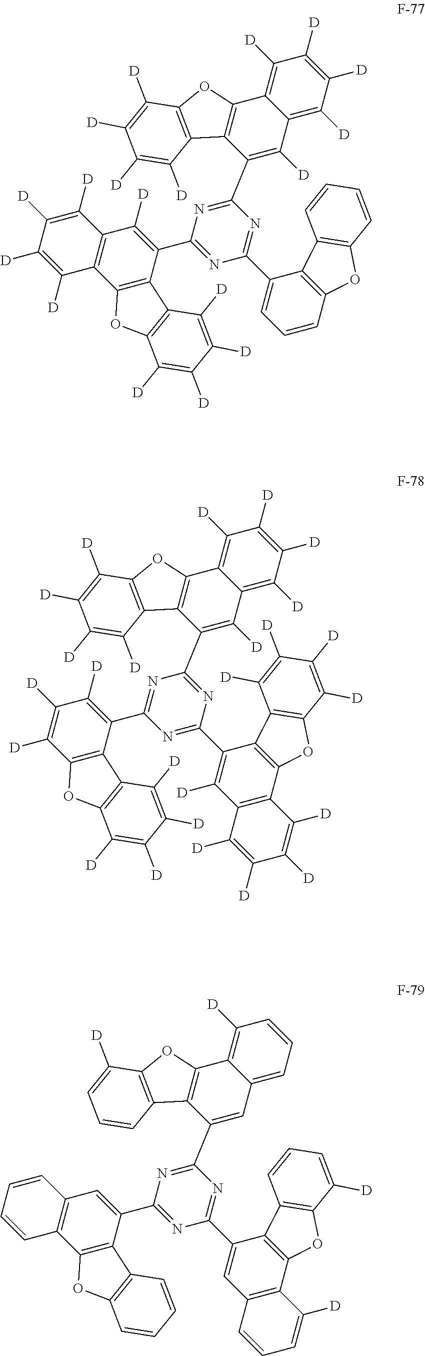

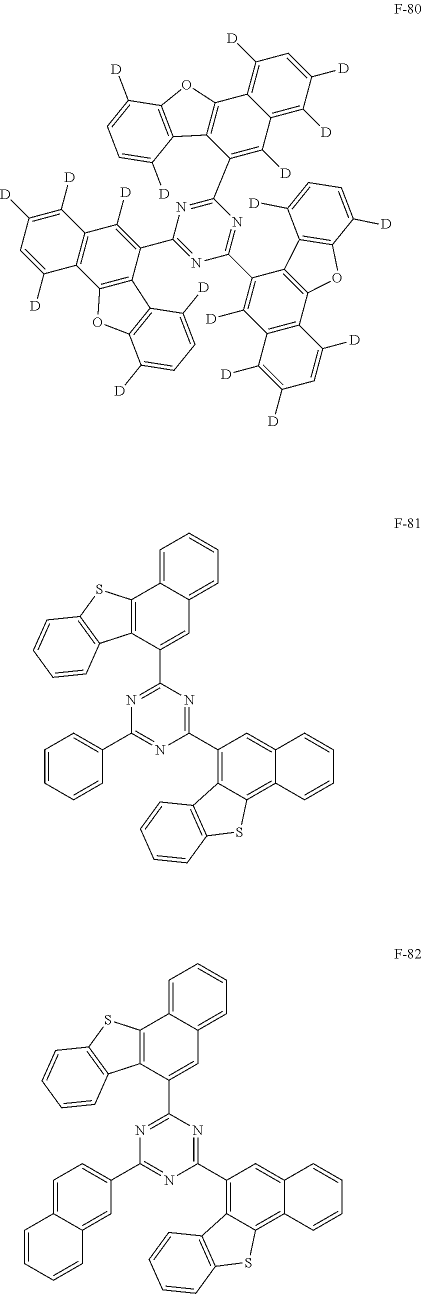 [SMILES: [2H]c1cc([2H])c2c(c1[2H])c([2H])c(-c1nc(-c3c([2H])c4c([2H])c([2H])cc([2H])c4c4oc5c([2H])ccc([2H])c5c34)nc(-c3c([2H])c4c([2H])c([2H])cc([2H])c4c4oc5c([2H])ccc([2H])c5c34)n1)c1c2oc2c([2H])ccc([2H])c21 ; c1ccc(-c2nc(-c3cc4ccccc4c4sc5ccccc5c34)nc(-c3cc4ccccc4c4sc5ccccc5c34)n2)cc1 ; c1ccc2cc(-c3nc(-c4cc5ccccc5c5sc6ccccc6c45)nc(-c4cc5ccccc5c5sc6ccccc6c45)n3)ccc2c1]

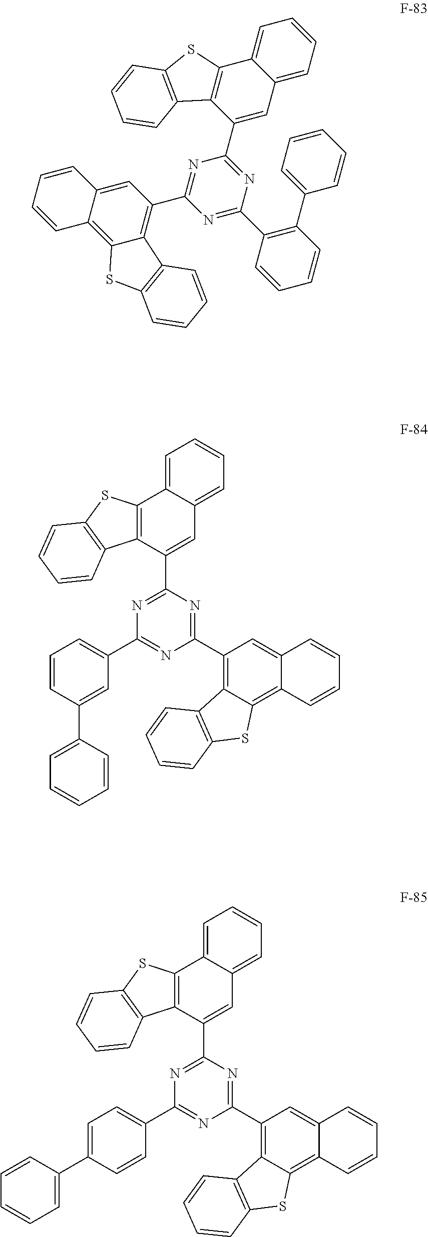 [SMILES: c1ccc(-c2ccccc2-c2nc(-c3cc4ccccc4c4sc5ccccc5c34)nc(-c3cc4ccccc4c4sc5ccccc5c34)n2)cc1 ; c1ccc(-c2cccc(-c3nc(-c4cc5ccccc5c5sc6ccccc6c45)nc(-c4cc5ccccc5c5sc6ccccc6c45)n3)c2)cc1 ; c1ccc(-c2ccc(-c3nc(-c4cc5ccccc5c5sc6ccccc6c45)nc(-c4cc5ccccc5c5sc6ccccc6c45)n3)cc2)cc1]

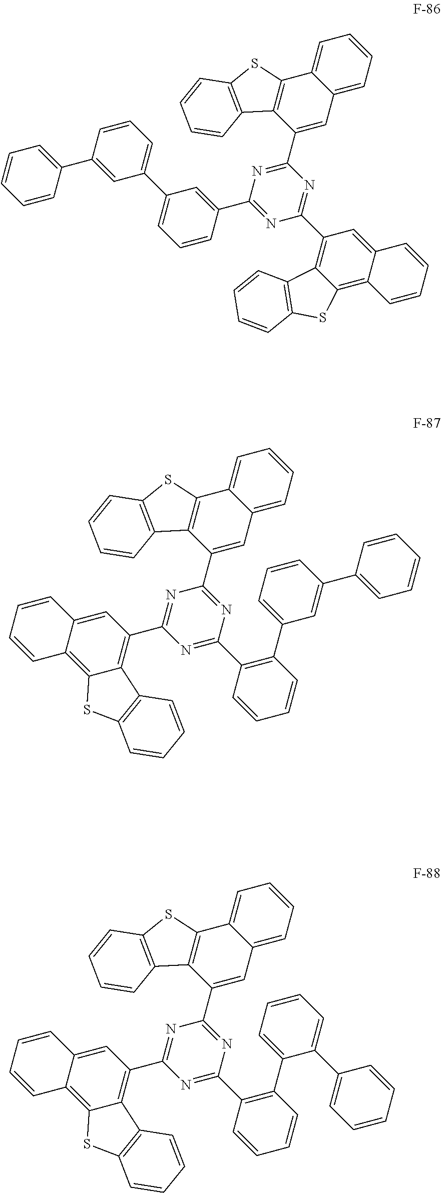 [SMILES: c1ccc(-c2cccc(-c3cccc(-c4nc(-c5cc6ccccc6c6sc7ccccc7c56)nc(-c5cc6ccccc6c6sc7ccccc7c56)n4)c3)c2)cc1 ; c1ccc(-c2cccc(-c3ccccc3-c3nc(-c4cc5ccccc5c5sc6ccccc6c45)nc(-c4cc5ccccc5c5sc6ccccc6c45)n3)c2)cc1 ; c1ccc(-c2ccccc2-c2ccccc2-c2nc(-c3cc4ccccc4c4sc5ccccc5c34)nc(-c3cc4ccccc4c4sc5ccccc5c34)n2)cc1]

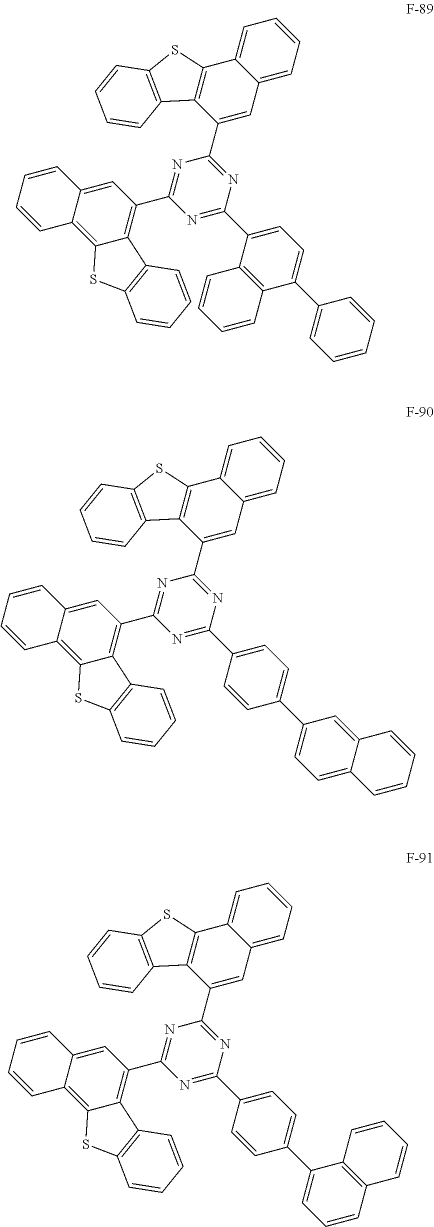 [SMILES: c1ccc(-c2ccc(-c3nc(-c4cc5ccccc5c5sc6ccccc6c45)nc(-c4cc5ccccc5c5sc6ccccc6c45)n3)c3ccccc23)cc1 ; c1ccc2cc(-c3ccc(-c4nc(-c5cc6ccccc6c6sc7ccccc7c56)nc(-c5cc6ccccc6c6sc7ccccc7c56)n4)cc3)ccc2c1 ; c1ccc2c(-c3ccc(-c4nc(-c5cc6ccccc6c6sc7ccccc7c56)nc(-c5cc6ccccc6c6sc7ccccc7c56)n4)cc3)cccc2c1]

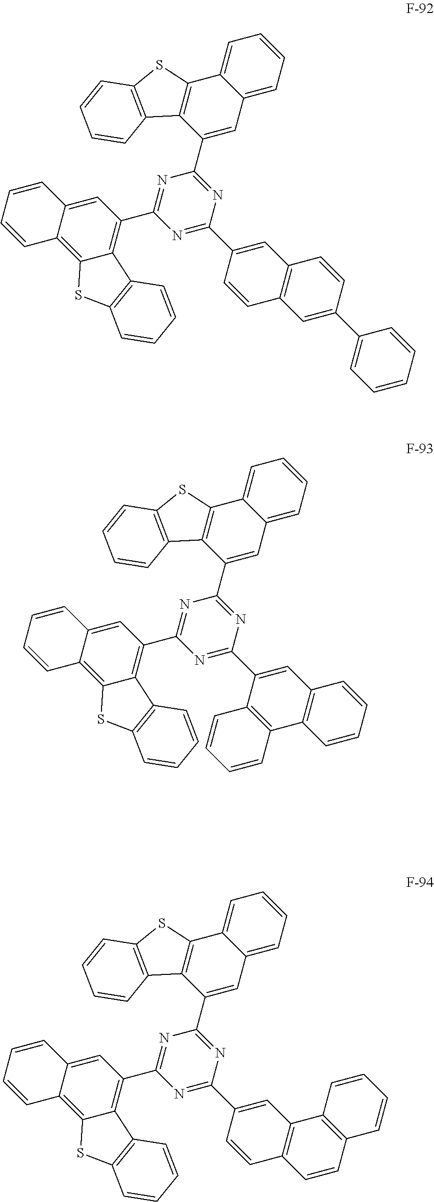 [SMILES: c1ccc(-c2ccc3cc(-c4nc(-c5cc6ccccc6c6sc7ccccc7c56)nc(-c5cc6ccccc6c6sc7ccccc7c56)n4)ccc3c2)cc1 ; c1ccc2c(c1)cc(-c1nc(-c3cc4ccccc4c4sc5ccccc5c34)nc(-c3cc4ccccc4c4sc5ccccc5c34)n1)c1ccccc12 ; c1ccc2c(c1)ccc1ccc(-c3nc(-c4cc5ccccc5c5sc6ccccc6c45)nc(-c4cc5ccccc5c5sc6ccccc6c45)n3)cc12]

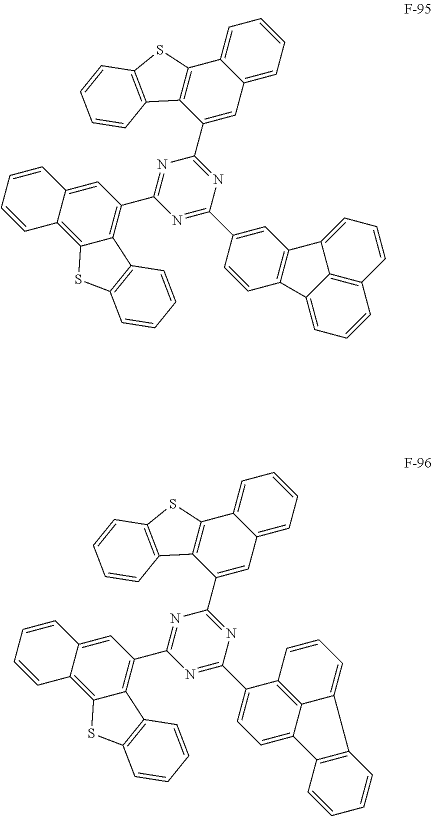 [SMILES: c1cc2c3c(cccc3c1)-c1cc(-c3nc(-c4cc5ccccc5c5sc6ccccc6c45)nc(-c4cc5ccccc5c5sc6ccccc6c45)n3)ccc1-2 ; c1ccc2c(c1)-c1cccc3c(-c4nc(-c5cc6ccccc6c6sc7ccccc7c56)nc(-c5cc6ccccc6c6sc7ccccc7c56)n4)ccc-2c13]

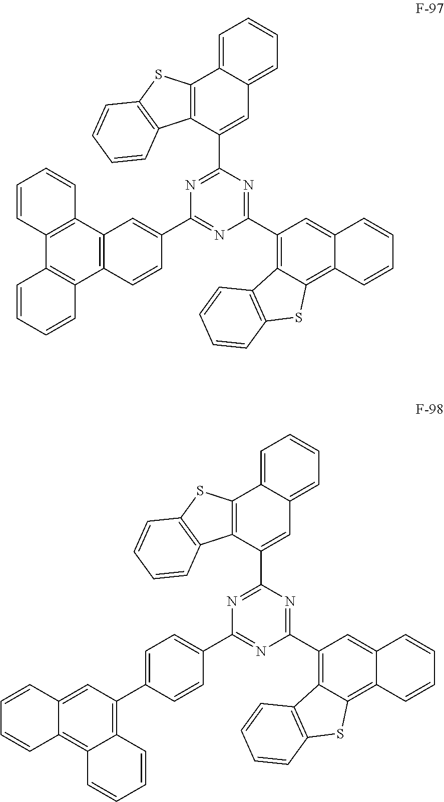 [SMILES: c1ccc2c(c1)cc(-c1nc(-c3ccc4c5ccccc5c5ccccc5c4c3)nc(-c3cc4ccccc4c4sc5ccccc5c34)n1)c1c3ccccc3sc21 ; c1ccc2c(c1)cc(-c1ccc(-c3nc(-c4cc5ccccc5c5sc6ccccc6c45)nc(-c4cc5ccccc5c5sc6ccccc6c45)n3)cc1)c1ccccc12]

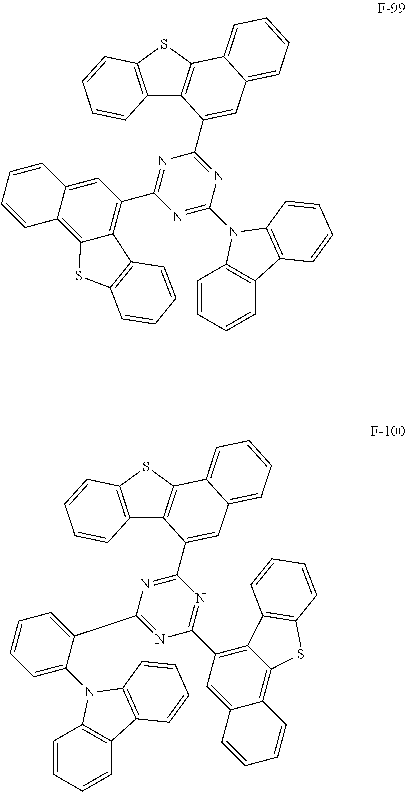 [SMILES: c1ccc2c(c1)cc(-c1nc(-c3cc4ccccc4c4sc5ccccc5c34)nc(-n3c4ccccc4c4ccccc43)n1)c1c3ccccc3sc21 ; c1ccc(-n2c3ccccc3c3ccccc32)c(-c2nc(-c3cc4ccccc4c4sc5ccccc5c34)nc(-c3cc4ccccc4c4sc5ccccc5c34)n2)c1]

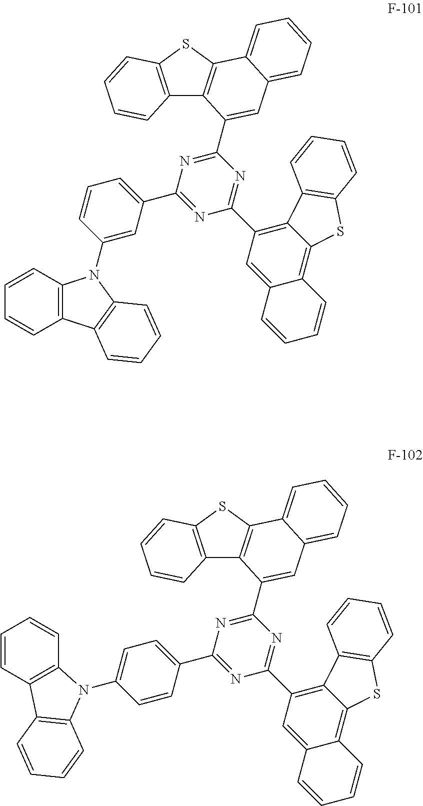 [SMILES: c1cc(-c2nc(-c3cc4ccccc4c4sc5ccccc5c34)nc(-c3cc4ccccc4c4sc5ccccc5c34)n2)cc(-n2c3ccccc3c3ccccc32)c1 ; c1ccc2c(c1)cc(-c1nc(-c3ccc(-n4c5ccccc5c5ccccc54)cc3)nc(-c3cc4ccccc4c4sc5ccccc5c34)n1)c1c3ccccc3sc21]

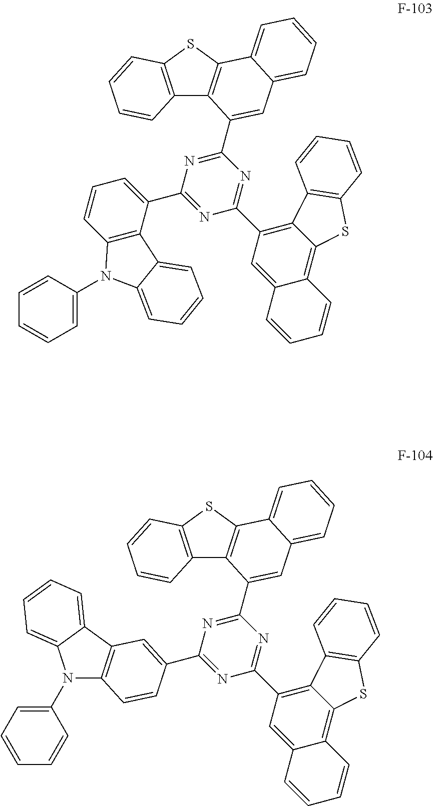 [SMILES: c1ccc(-n2c3ccccc3c3c(-c4nc(-c5cc6ccccc6c6sc7ccccc7c56)nc(-c5cc6ccccc6c6sc7ccccc7c56)n4)cccc32)cc1 ; c1ccc(-n2c3ccccc3c3cc(-c4nc(-c5cc6ccccc6c6sc7ccccc7c56)nc(-c5cc6ccccc6c6sc7ccccc7c56)n4)ccc32)cc1]

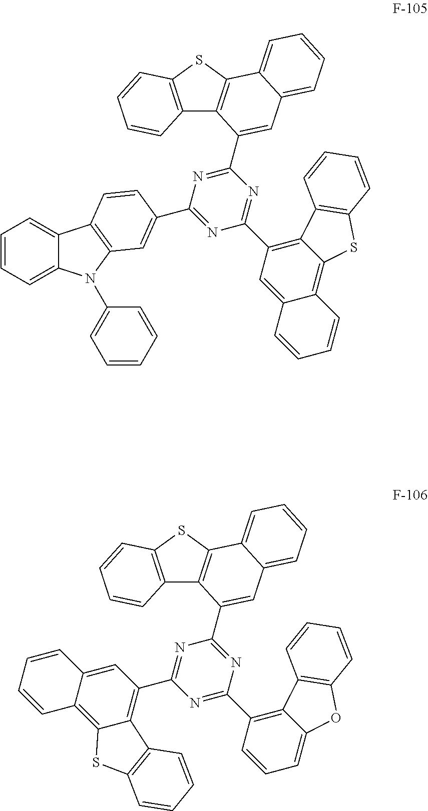 [SMILES: c1ccc(-n2c3ccccc3c3ccc(-c4nc(-c5cc6ccccc6c6sc7ccccc7c56)nc(-c5cc6ccccc6c6sc7ccccc7c56)n4)cc32)cc1 ; c1ccc2c(c1)cc(-c1nc(-c3cccc4oc5ccccc5c34)nc(-c3cc4ccccc4c4sc5ccccc5c34)n1)c1c3ccccc3sc21]

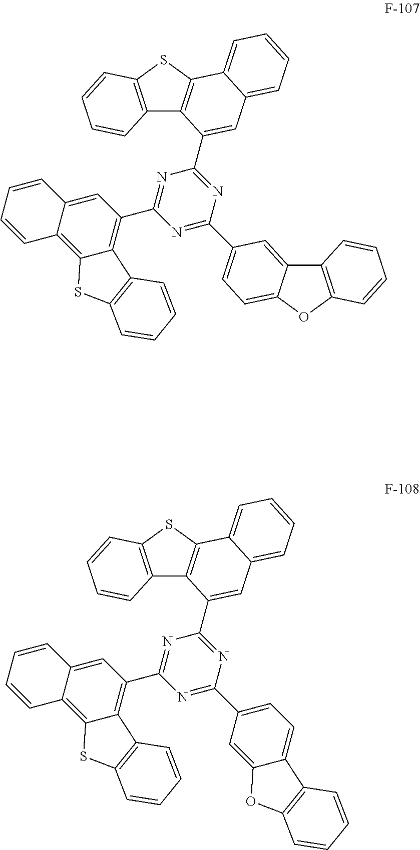 [SMILES: c1ccc2c(c1)cc(-c1nc(-c3ccc4oc5ccccc5c4c3)nc(-c3cc4ccccc4c4sc5ccccc5c34)n1)c1c3ccccc3sc21 ; c1ccc2c(c1)cc(-c1nc(-c3ccc4c(c3)oc3ccccc34)nc(-c3cc4ccccc4c4sc5ccccc5c34)n1)c1c3ccccc3sc21]

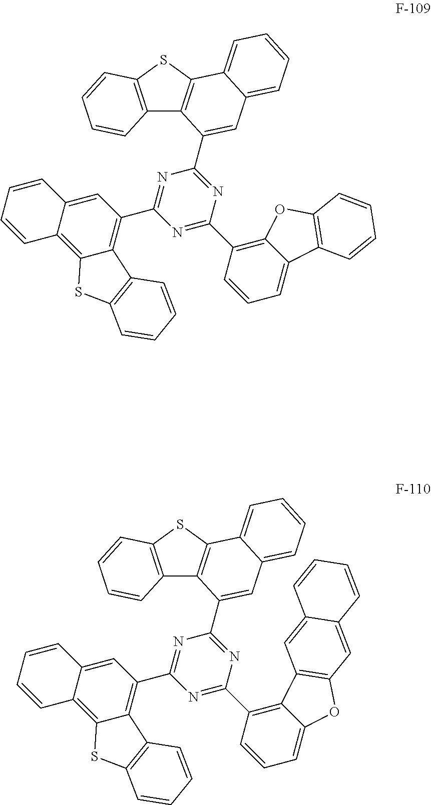 [SMILES: c1ccc2c(c1)cc(-c1nc(-c3cccc4c3oc3ccccc34)nc(-c3cc4ccccc4c4sc5ccccc5c34)n1)c1c3ccccc3sc21 ; c1ccc2cc3c(cc2c1)oc1cccc(-c2nc(-c4cc5ccccc5c5sc6ccccc6c45)nc(-c4cc5ccccc5c5sc6ccccc6c45)n2)c13]

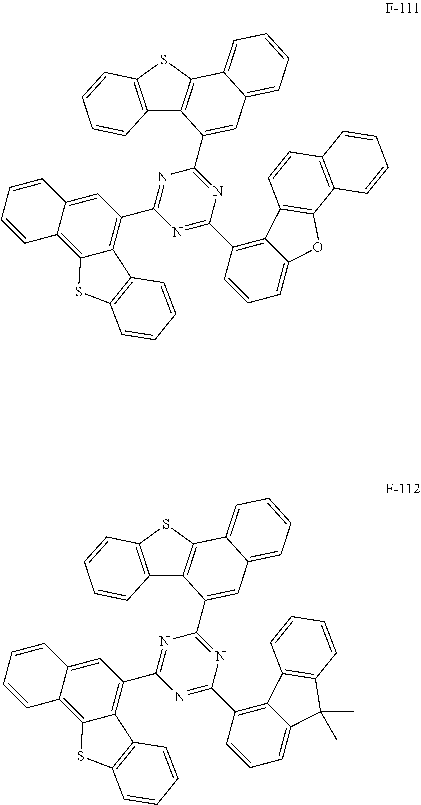 [SMILES: c1ccc2c(c1)ccc1c2oc2cccc(-c3nc(-c4cc5ccccc5c5sc6ccccc6c45)nc(-c4cc5ccccc5c5sc6ccccc6c45)n3)c21 ; CC1(C)c2ccccc2-c2c(-c3nc(-c4cc5ccccc5c5sc6ccccc6c45)nc(-c4cc5ccccc5c5sc6ccccc6c45)n3)cccc21]

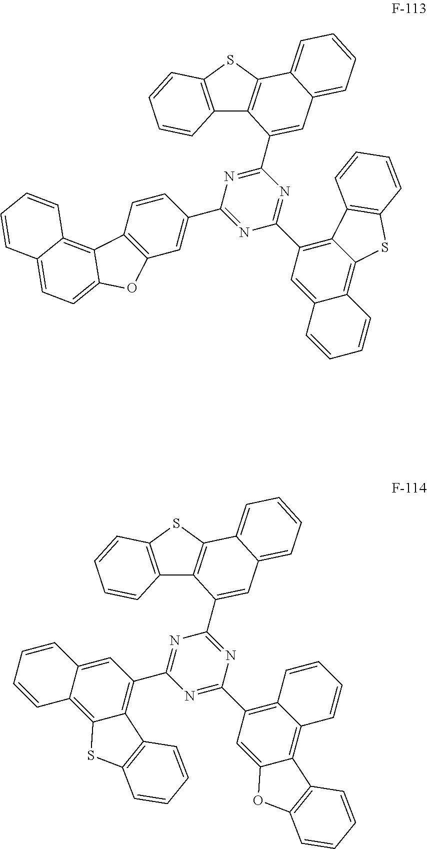 [SMILES: c1ccc2c(c1)cc(-c1nc(-c3ccc4c(c3)oc3ccc5ccccc5c34)nc(-c3cc4ccccc4c4sc5ccccc5c34)n1)c1c3ccccc3sc21 ; c1ccc2c(c1)cc(-c1nc(-c3cc4oc5ccccc5c4c4ccccc34)nc(-c3cc4ccccc4c4sc5ccccc5c34)n1)c1c3ccccc3sc21]

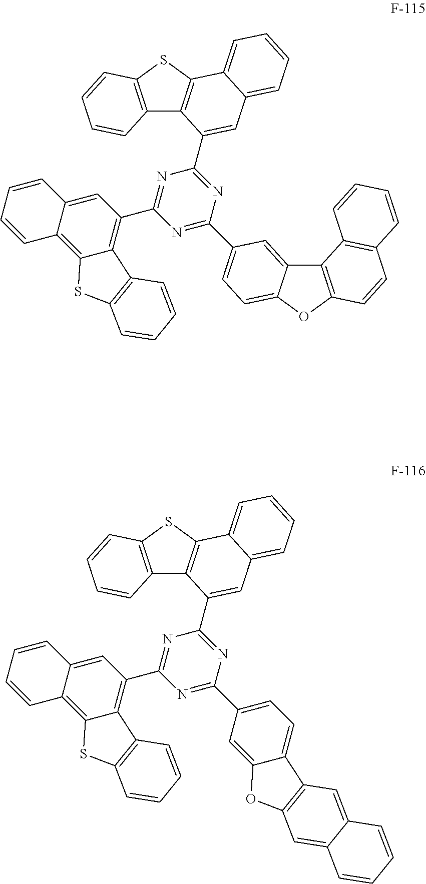 [SMILES: c1ccc2c(c1)cc(-c1nc(-c3ccc4oc5ccc6ccccc6c5c4c3)nc(-c3cc4ccccc4c4sc5ccccc5c34)n1)c1c3ccccc3sc21 ; c1ccc2cc3c(cc2c1)oc1cc(-c2nc(-c4cc5ccccc5c5sc6ccccc6c45)nc(-c4cc5ccccc5c5sc6ccccc6c45)n2)ccc13]

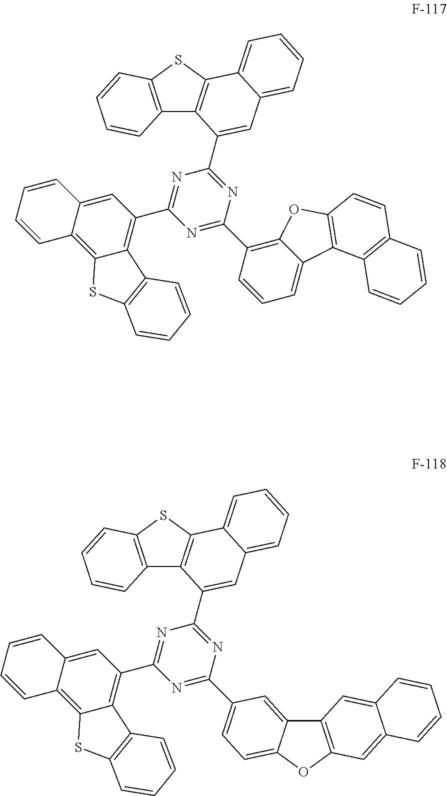 [SMILES: c1ccc2c(c1)cc(-c1nc(-c3cccc4c3oc3ccc5ccccc5c34)nc(-c3cc4ccccc4c4sc5ccccc5c34)n1)c1c3ccccc3sc21 ; c1ccc2cc3c(cc2c1)oc1ccc(-c2nc(-c4cc5ccccc5c5sc6ccccc6c45)nc(-c4cc5ccccc5c5sc6ccccc6c45)n2)cc13]

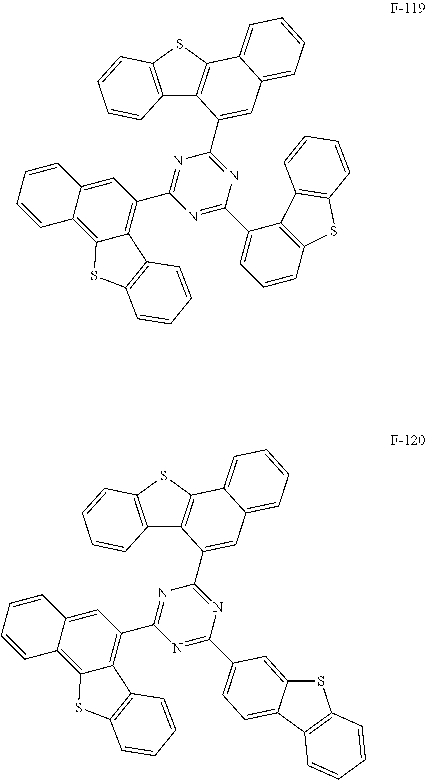 [SMILES: c1ccc2c(c1)cc(-c1nc(-c3cccc4sc5ccccc5c34)nc(-c3cc4ccccc4c4sc5ccccc5c34)n1)c1c3ccccc3sc21 ; c1ccc2c(c1)cc(-c1nc(-c3ccc4c(c3)sc3ccccc34)nc(-c3cc4ccccc4c4sc5ccccc5c34)n1)c1c3ccccc3sc21]

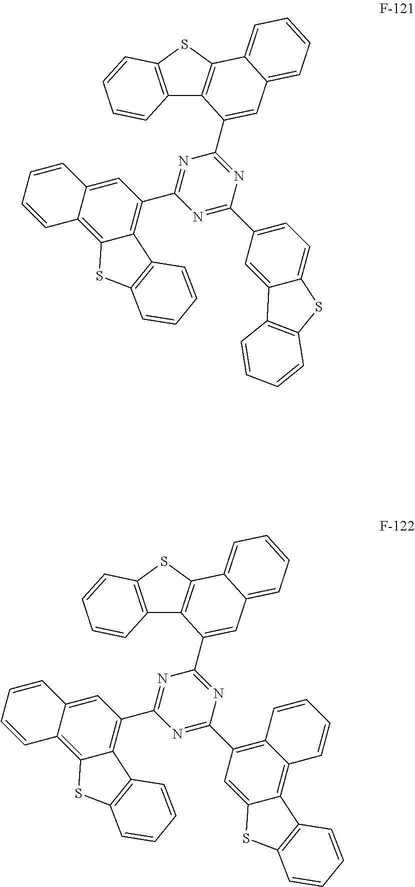 [SMILES: c1ccc2c(c1)cc(-c1nc(-c3ccc4sc5ccccc5c4c3)nc(-c3cc4ccccc4c4sc5ccccc5c34)n1)c1c3ccccc3sc21 ; c1ccc2c(c1)cc(-c1nc(-c3cc4sc5ccccc5c4c4ccccc34)nc(-c3cc4ccccc4c4sc5ccccc5c34)n1)c1c3ccccc3sc21]

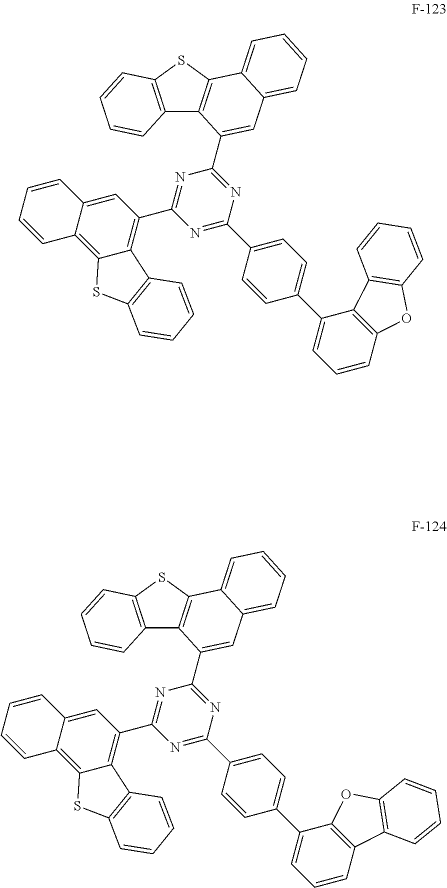 [SMILES: c1ccc2c(c1)cc(-c1nc(-c3ccc(-c4cccc5oc6ccccc6c45)cc3)nc(-c3cc4ccccc4c4sc5ccccc5c34)n1)c1c3ccccc3sc21 ; c1ccc2c(c1)cc(-c1nc(-c3ccc(-c4cccc5c4oc4ccccc45)cc3)nc(-c3cc4ccccc4c4sc5ccccc5c34)n1)c1c3ccccc3sc21]

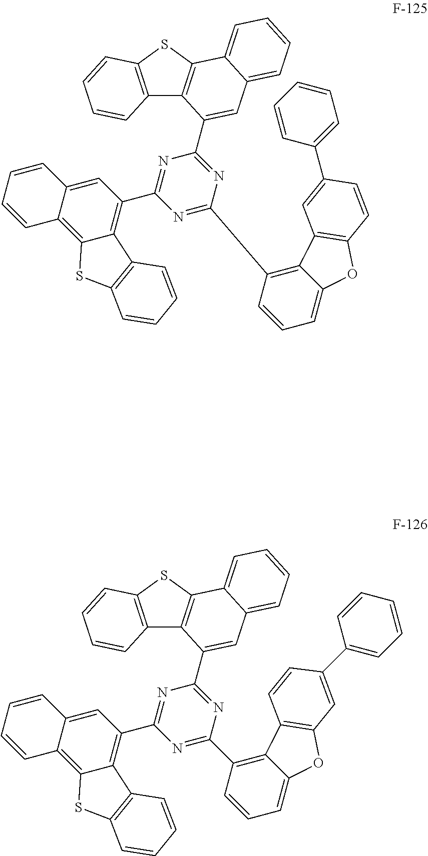 [SMILES: c1ccc(-c2ccc3oc4cccc(-c5nc(-c6cc7ccccc7c7sc8ccccc8c67)nc(-c6cc7ccccc7c7sc8ccccc8c67)n5)c4c3c2)cc1 ; c1ccc(-c2ccc3c(c2)oc2cccc(-c4nc(-c5cc6ccccc6c6sc7ccccc7c56)nc(-c5cc6ccccc6c6sc7ccccc7c56)n4)c23)cc1]

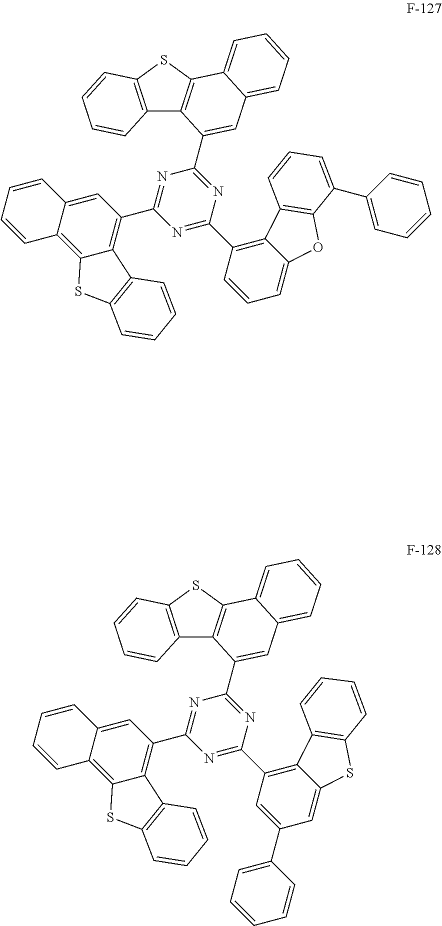 [SMILES: c1ccc(-c2cccc3c2oc2cccc(-c4nc(-c5cc6ccccc6c6sc7ccccc7c56)nc(-c5cc6ccccc6c6sc7ccccc7c56)n4)c23)cc1 ; c1ccc(-c2cc(-c3nc(-c4cc5ccccc5c5sc6ccccc6c45)nc(-c4cc5ccccc5c5sc6ccccc6c45)n3)c3c(c2)sc2ccccc23)cc1]

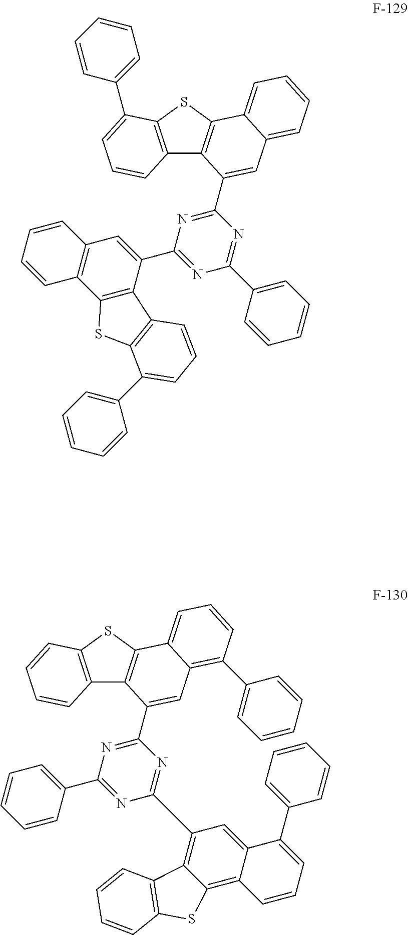 [SMILES: c1ccc(-c2nc(-c3cc4ccccc4c4sc5c(-c6ccccc6)cccc5c34)nc(-c3cc4ccccc4c4sc5c(-c6ccccc6)cccc5c34)n2)cc1 ; c1ccc(-c2nc(-c3cc4c(-c5ccccc5)cccc4c4sc5ccccc5c34)nc(-c3cc4c(-c5ccccc5)cccc4c4sc5ccccc5c34)n2)cc1]

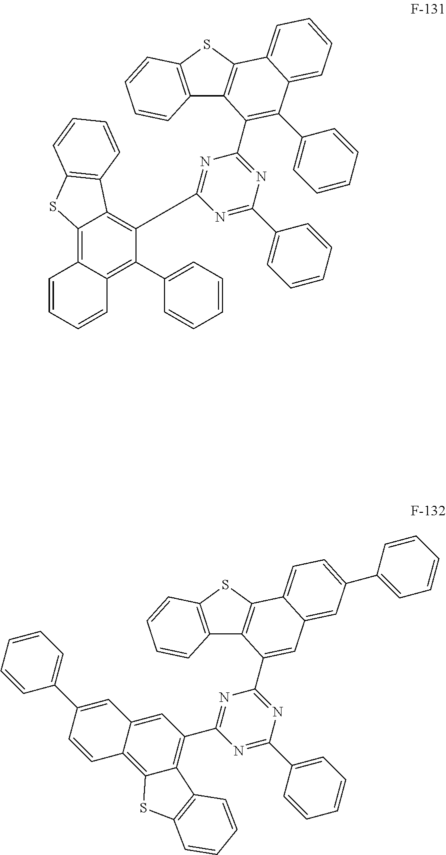 [SMILES: c1ccc(-c2nc(-c3c(-c4ccccc4)c4ccccc4c4sc5ccccc5c34)nc(-c3c(-c4ccccc4)c4ccccc4c4sc5ccccc5c34)n2)cc1 ; c1ccc(-c2ccc3c(c2)cc(-c2nc(-c4ccccc4)nc(-c4cc5cc(-c6ccccc6)ccc5c5sc6ccccc6c45)n2)c2c4ccccc4sc32)cc1]

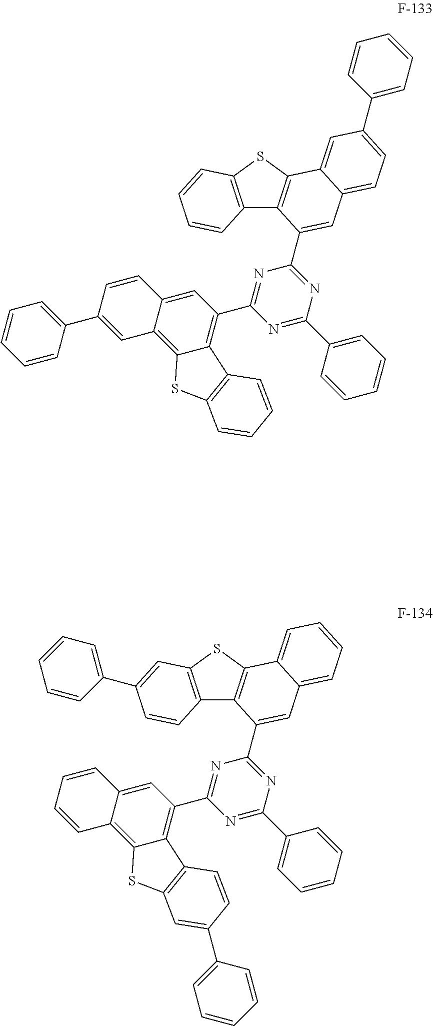 [SMILES: c1ccc(-c2ccc3cc(-c4nc(-c5ccccc5)nc(-c5cc6ccc(-c7ccccc7)cc6c6sc7ccccc7c56)n4)c4c5ccccc5sc4c3c2)cc1 ; c1ccc(-c2ccc3c(c2)sc2c4ccccc4cc(-c4nc(-c5ccccc5)nc(-c5cc6ccccc6c6sc7cc(-c8ccccc8)ccc7c56)n4)c32)cc1]

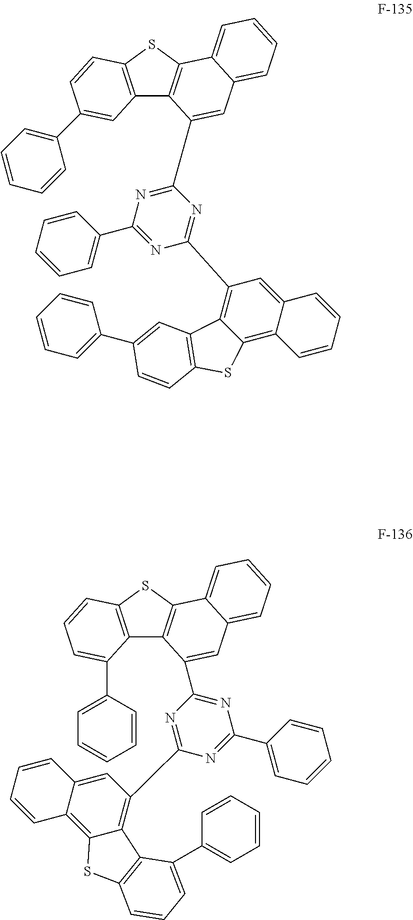 [SMILES: c1ccc(-c2ccc3sc4c5ccccc5cc(-c5nc(-c6ccccc6)nc(-c6cc7ccccc7c7sc8ccc(-c9ccccc9)cc8c67)n5)c4c3c2)cc1 ; c1ccc(-c2nc(-c3cc4ccccc4c4sc5cccc(-c6ccccc6)c5c34)nc(-c3cc4ccccc4c4sc5cccc(-c6ccccc6)c5c34)n2)cc1]

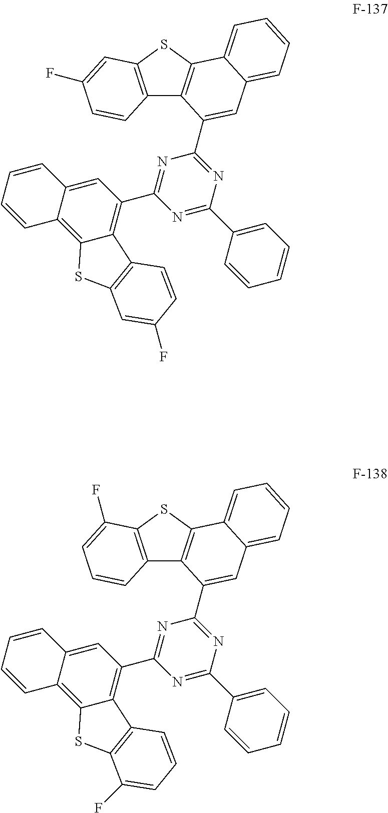 [SMILES: Fc1ccc2c(c1)sc1c3ccccc3cc(-c3nc(-c4ccccc4)nc(-c4cc5ccccc5c5sc6cc(F)ccc6c45)n3)c21 ; Fc1cccc2c1sc1c3ccccc3cc(-c3nc(-c4ccccc4)nc(-c4cc5ccccc5c5sc6c(F)cccc6c45)n3)c21]

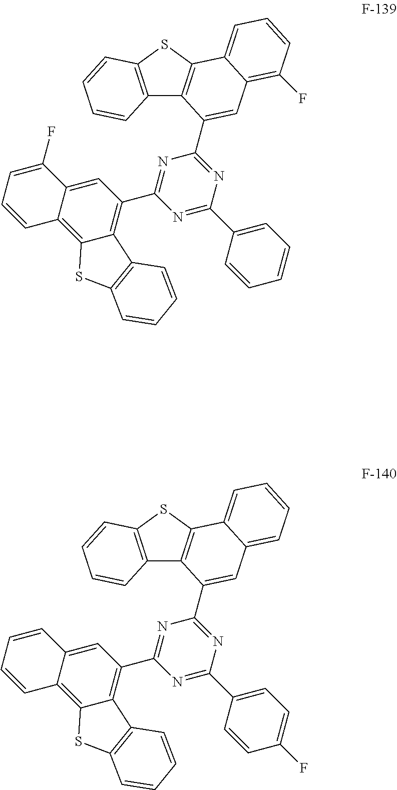 [SMILES: Fc1cccc2c1cc(-c1nc(-c3ccccc3)nc(-c3cc4c(F)cccc4c4sc5ccccc5c34)n1)c1c3ccccc3sc21 ; Fc1ccc(-c2nc(-c3cc4ccccc4c4sc5ccccc5c34)nc(-c3cc4ccccc4c4sc5ccccc5c34)n2)cc1]

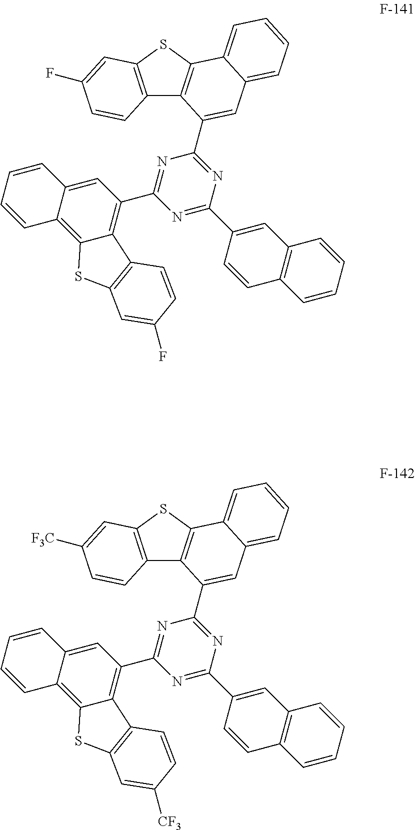 [SMILES: Fc1ccc2c(c1)sc1c3ccccc3cc(-c3nc(-c4ccc5ccccc5c4)nc(-c4cc5ccccc5c5sc6cc(F)ccc6c45)n3)c21 ; FC(F)(F)c1ccc2c(c1)sc1c3ccccc3cc(-c3nc(-c4ccc5ccccc5c4)nc(-c4cc5ccccc5c5sc6cc(C(F)(F)F)ccc6c45)n3)c21]

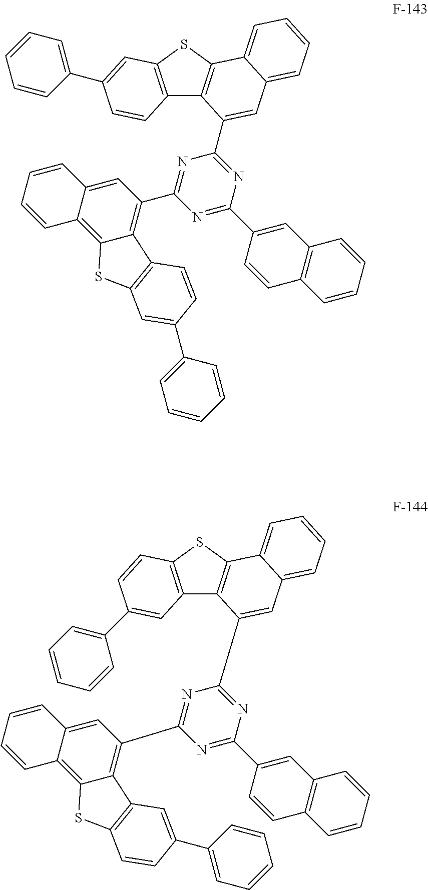 [SMILES: c1ccc(-c2ccc3c(c2)sc2c4ccccc4cc(-c4nc(-c5ccc6ccccc6c5)nc(-c5cc6ccccc6c6sc7cc(-c8ccccc8)ccc7c56)n4)c32)cc1 ; c1ccc(-c2ccc3sc4c5ccccc5cc(-c5nc(-c6ccc7ccccc7c6)nc(-c6cc7ccccc7c7sc8ccc(-c9ccccc9)cc8c67)n5)c4c3c2)cc1]

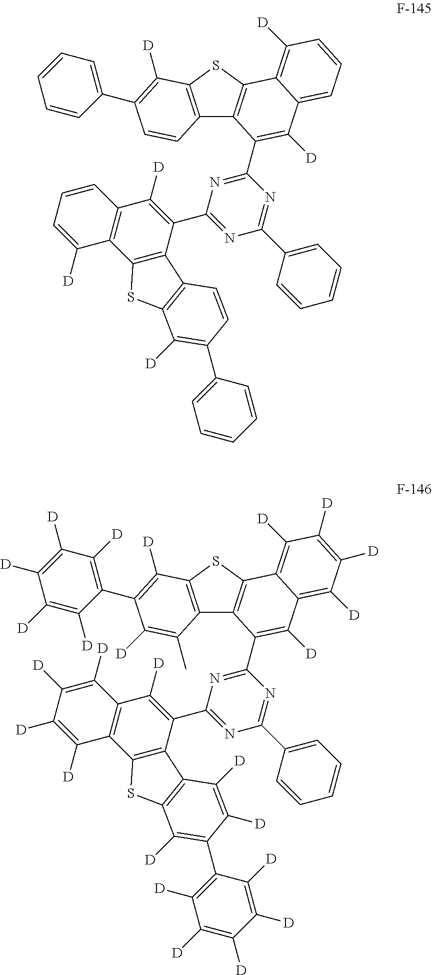 [SMILES: [2H]c1c(-c2nc(-c3ccccc3)nc(-c3c([2H])c4cccc([2H])c4c4sc5c([2H])c(-c6ccccc6)ccc5c34)n2)c2c3ccc(-c4ccccc4)c([2H])c3sc2c2c([2H])cccc12 ; [2H]c1c([2H])c([2H])c(-c2c([2H])c([2H])c3c(sc4c5c([2H])c([2H])c([2H])c([2H])c5c([2H])c(-c5nc(-c6ccccc6)nc(-c6c([2H])c7c([2H])c([2H])c([2H])c([2H])c7c7sc8c([2H])c(-c9c([2H])c([2H])c([2H])c([2H])c9[2H])c([2H])c(C)c8c67)n5)c43)c2[2H])c([2H])c1[2H]]

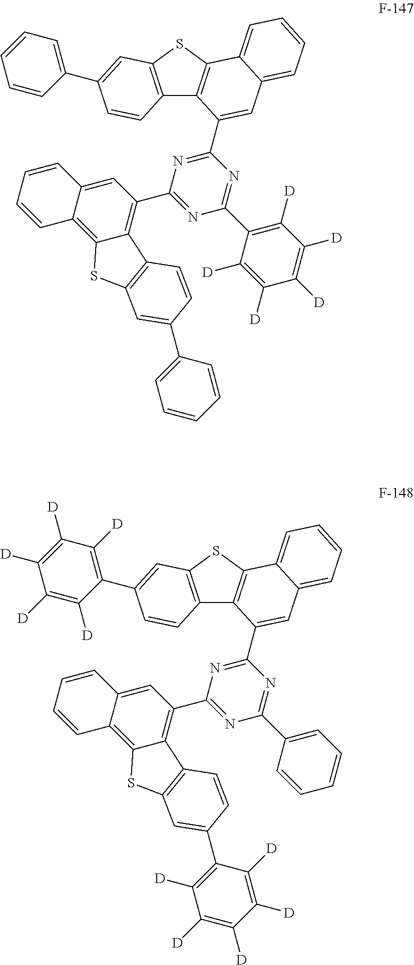 [SMILES: [2H]c1c([2H])c([2H])c(-c2nc(-c3cc4ccccc4c4sc5cc(-c6ccccc6)ccc5c34)nc(-c3cc4ccccc4c4sc5cc(-c6ccccc6)ccc5c34)n2)c([2H])c1[2H] ; [2H]c1c([2H])c([2H])c(-c2ccc3c(c2)sc2c4ccccc4cc(-c4nc(-c5ccccc5)nc(-c5cc6ccccc6c6sc7cc(-c8c([2H])c([2H])c([2H])c([2H])c8[2H])ccc7c56)n4)c32)c([2H])c1[2H]]

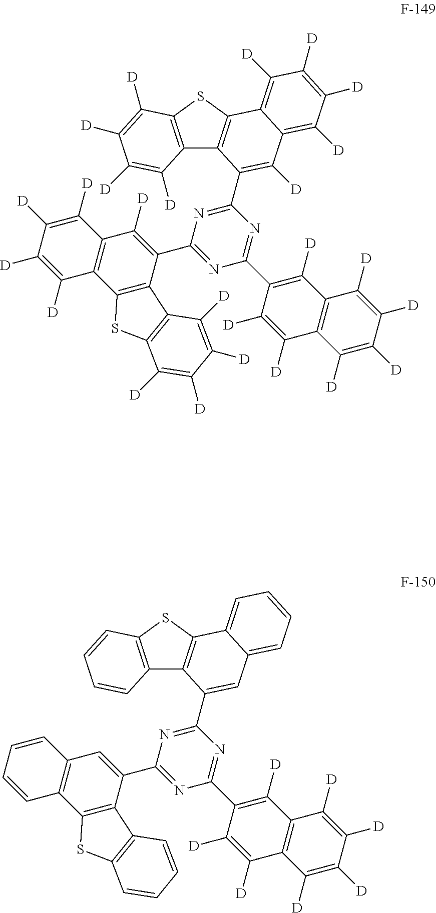 [SMILES: [2H]c1c([2H])c([2H])c2c(sc3c4c([2H])c([2H])c([2H])c([2H])c4c([2H])c(-c4nc(-c5c([2H])c([2H])c6c([2H])c([2H])c([2H])c([2H])c6c5[2H])nc(-c5c([2H])c6c([2H])c([2H])c([2H])c([2H])c6c6sc7c([2H])c([2H])c([2H])c([2H])c7c56)n4)c32)c1[2H] ; [2H]c1c([2H])c([2H])c2c([2H])c(-c3nc(-c4cc5ccccc5c5sc6ccccc6c45)nc(-c4cc5ccccc5c5sc6ccccc6c45)n3)c([2H])c([2H])c2c1[2H]]

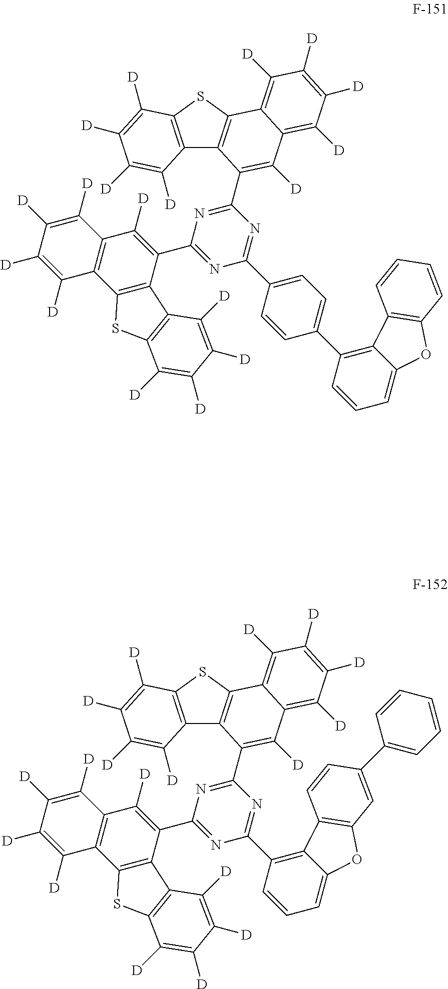 [SMILES: [2H]c1c([2H])c([2H])c2c(sc3c4c([2H])c([2H])c([2H])c([2H])c4c([2H])c(-c4nc(-c5ccc(-c6cccc7oc8ccccc8c67)cc5)nc(-c5c([2H])c6c([2H])c([2H])c([2H])c([2H])c6c6sc7c([2H])c([2H])c([2H])c([2H])c7c56)n4)c32)c1[2H] ; [2H]c1c([2H])c([2H])c2c(sc3c4c([2H])c([2H])c([2H])c([2H])c4c([2H])c(-c4nc(-c5cccc6oc7cc(-c8ccccc8)ccc7c56)nc(-c5c([2H])c6c([2H])c([2H])c([2H])c([2H])c6c6sc7c([2H])c([2H])c([2H])c([2H])c7c56)n4)c32)c1[2H]]

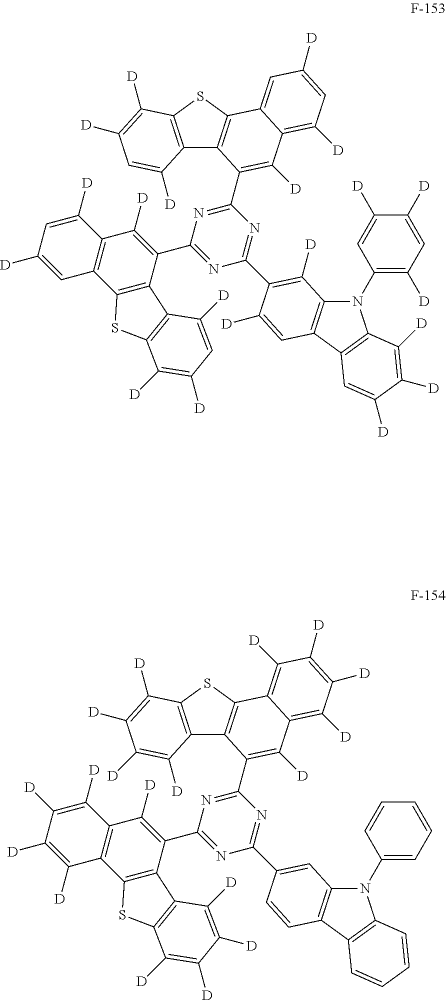 [SMILES: [2H]c1cc([2H])c2c([2H])c(-c3nc(-c4c([2H])cc5c6cc([2H])c([2H])c([2H])c6n(-c6cc([2H])c([2H])cc6[2H])c5c4[2H])nc(-c4c([2H])c5c([2H])cc([2H])cc5c5sc6c([2H])c([2H])cc([2H])c6c45)n3)c3c(sc4c([2H])c([2H])cc([2H])c43)c2c1 ; [2H]c1c([2H])c([2H])c2c(sc3c4c([2H])c([2H])c([2H])c([2H])c4c([2H])c(-c4nc(-c5ccc6c7ccccc7n(-c7ccccc7)c6c5)nc(-c5c([2H])c6c([2H])c([2H])c([2H])c([2H])c6c6sc7c([2H])c([2H])c([2H])c([2H])c7c56)n4)c32)c1[2H]]

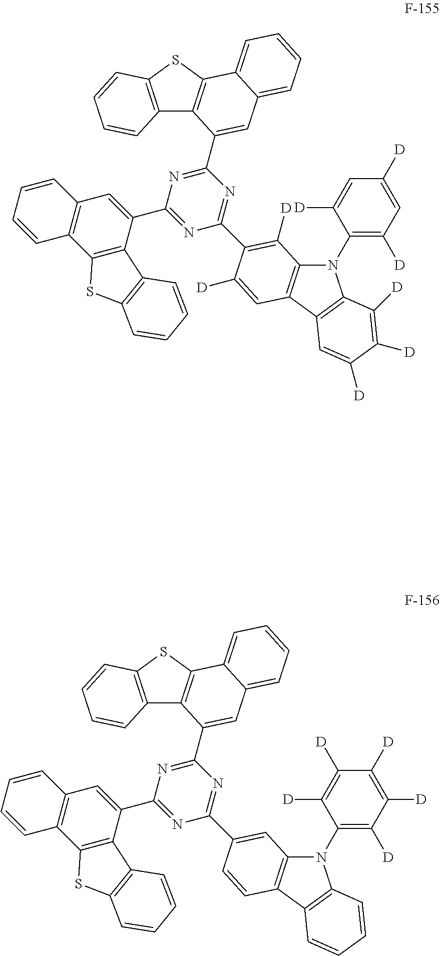 [SMILES: [2H]c1cc([2H])c(-n2c3c([2H])c([2H])c([2H])cc3c3cc([2H])c(-c4nc(-c5cc6ccccc6c6sc7ccccc7c56)nc(-c5cc6ccccc6c6sc7ccccc7c56)n4)c([2H])c32)c([2H])c1 ; [2H]c1c([2H])c([2H])c(-n2c3ccccc3c3ccc(-c4nc(-c5cc6ccccc6c6sc7ccccc7c56)nc(-c5cc6ccccc6c6sc7ccccc7c56)n4)cc32)c([2H])c1[2H]]

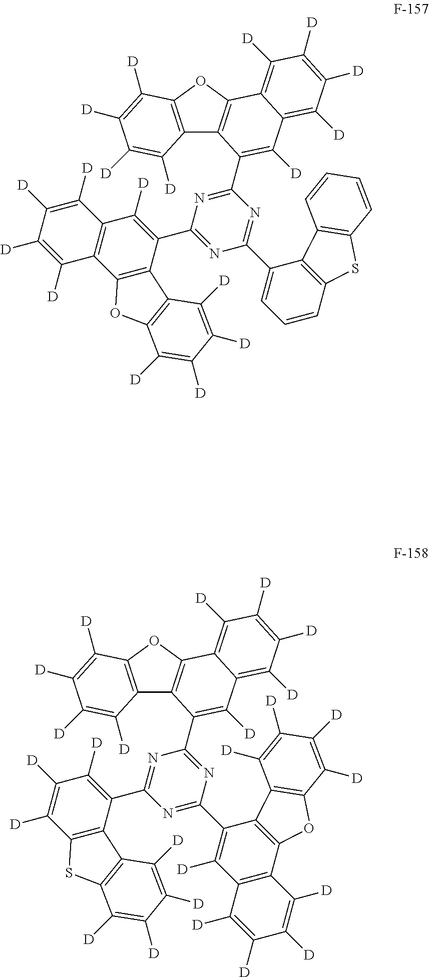 [SMILES: [2H]c1c([2H])c([2H])c2c(oc3c4c([2H])c([2H])c([2H])c([2H])c4c([2H])c(-c4nc(-c5cccc6sc7ccccc7c56)nc(-c5c([2H])c6c([2H])c([2H])c([2H])c([2H])c6c6oc7c([2H])c([2H])c([2H])c([2H])c7c56)n4)c32)c1[2H] ; [2H]c1c([2H])c([2H])c2c(oc3c4c([2H])c([2H])c([2H])c([2H])c4c([2H])c(-c4nc(-c5c([2H])c([2H])c([2H])c6sc7c([2H])c([2H])c([2H])c([2H])c7c56)nc(-c5c([2H])c6c([2H])c([2H])c([2H])c([2H])c6c6oc7c([2H])c([2H])c([2H])c([2H])c7c56)n4)c32)c1[2H]]

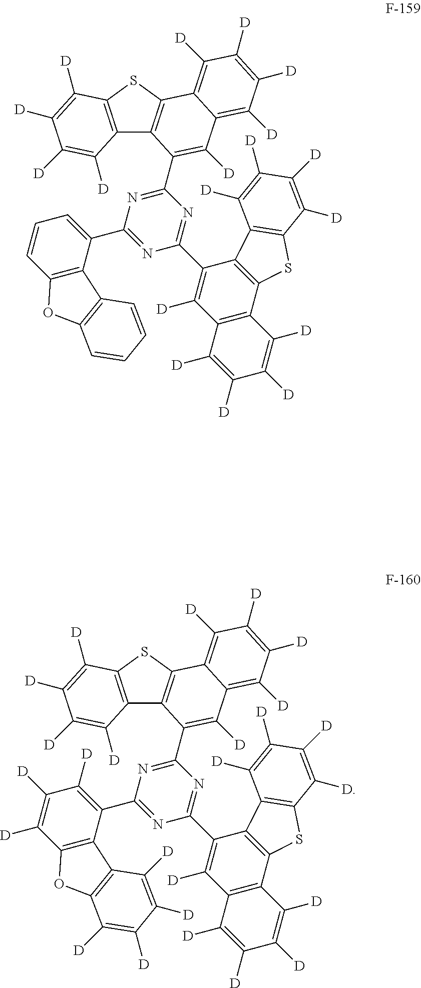

7. An organic light emitting device comprising:
a first electrode;
a second electrode; and
an organic material layer having one or more layers provided between the first electrode and the second electrode,
wherein one or more layers of the organic material layer comprise one or more of the heterocyclic compounds of claim 1.

8. The organic light emitting device of claim 7, wherein the organic material layer comprises a light emitting layer, and the light emitting layer comprises one or more of the heterocyclic compounds.

9. The organic light emitting device of claim 8, wherein the light emitting layer comprises a host, and the host comprises one or more of the heterocyclic compounds.

10. The organic light emitting device of claim 7, wherein the organic material layer comprising the heterocyclic compound further comprises a compound of the following Chemical Formula 2: 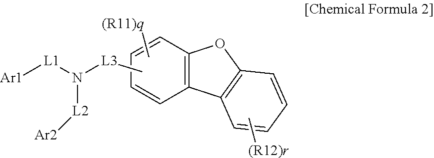
in Chemical Formula 2,
L1 to L3 are each independently a direct bond; a substituted or unsubstituted C6 to C60 arylene group; or a substituted or unsubstituted C2 to C60 heteroarylene group,
Ar1 and Ar2 are each independently a substituted or unsubstituted C6 to C60 aryl group; or a substituted or unsubstituted C2 to C60 heteroaryl group,
R11 and R12 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or are bonded to an adjacent group to form a substituted or unsubstituted C6 to C60 aromatic ring or a substituted or unsubstituted C2 to C60 hetero ring,
q is an integer from 1 to 3,
r is an integer of 1 to 4, and
when q and r are each 2 or higher, substituents in the parenthesis are the same as or different from each other.

11. The organic light emitting device of claim 10, wherein Chemical Formula 2 is selected from the following compounds:

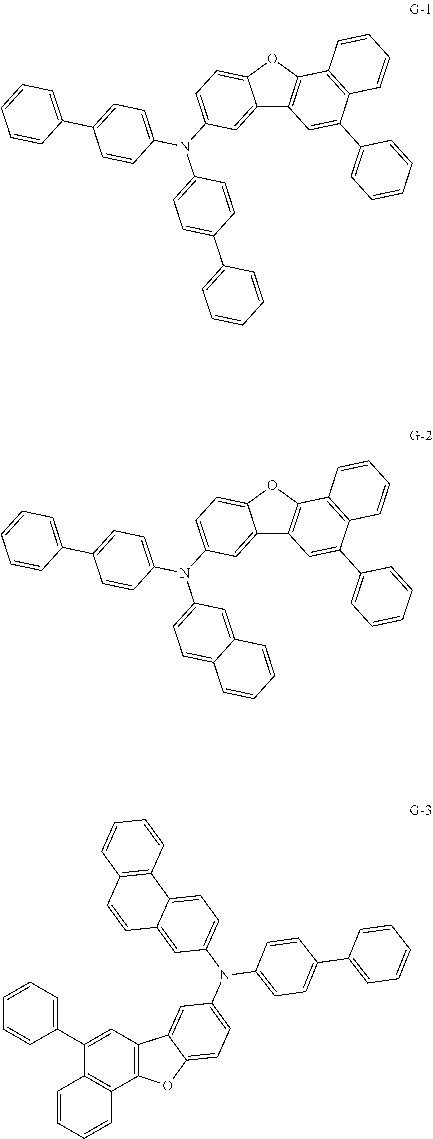 [SMILES: c1ccc(-c2ccc(N(c3ccc(-c4ccccc4)cc3)c3ccc4oc5c6ccccc6c(-c6ccccc6)cc5c4c3)cc2)cc1 ; c1ccc(-c2ccc(N(c3ccc4ccccc4c3)c3ccc4oc5c6ccccc6c(-c6ccccc6)cc5c4c3)cc2)cc1 ; c1ccc(-c2ccc(N(c3ccc4c(ccc5ccccc54)c3)c3ccc4oc5c6ccccc6c(-c6ccccc6)cc5c4c3)cc2)cc1]

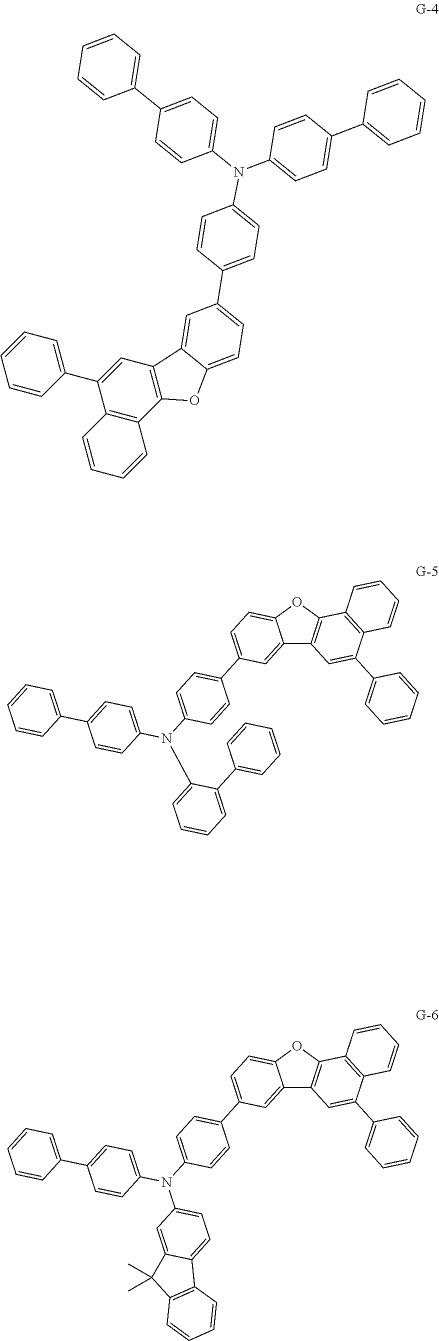 [SMILES: c1ccc(-c2ccc(N(c3ccc(-c4ccccc4)cc3)c3ccc(-c4ccc5oc6c7ccccc7c(-c7ccccc7)cc6c5c4)cc3)cc2)cc1 ; c1ccc(-c2ccc(N(c3ccc(-c4ccc5oc6c7ccccc7c(-c7ccccc7)cc6c5c4)cc3)c3ccccc3-c3ccccc3)cc2)cc1 ; CC1(C)c2ccccc2-c2ccc(N(c3ccc(-c4ccccc4)cc3)c3ccc(-c4ccc5oc6c7ccccc7c(-c7ccccc7)cc6c5c4)cc3)cc21]

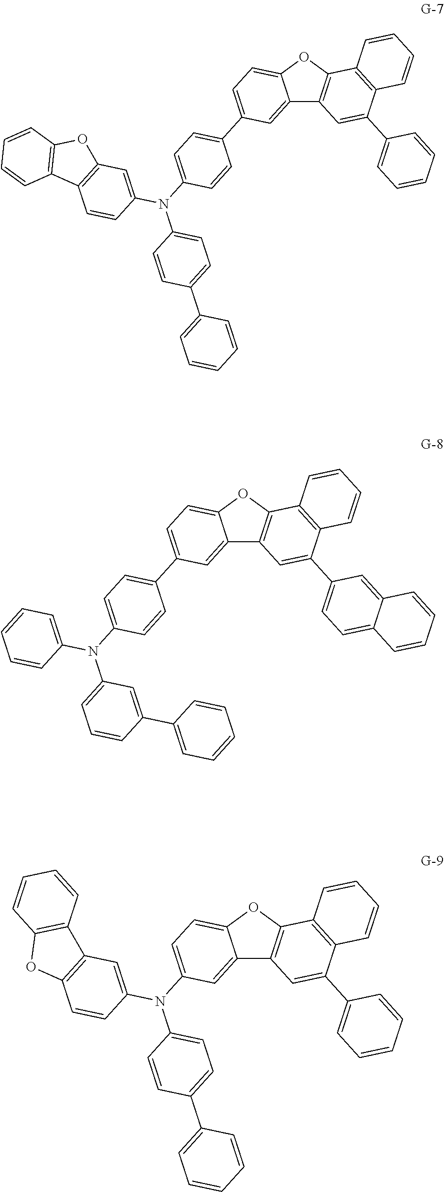 [SMILES: c1ccc(-c2ccc(N(c3ccc(-c4ccc5oc6c7ccccc7c(-c7ccccc7)cc6c5c4)cc3)c3ccc4c(c3)oc3ccccc34)cc2)cc1 ; c1ccc(-c2cccc(N(c3ccccc3)c3ccc(-c4ccc5oc6c7ccccc7c(-c7ccc8ccccc8c7)cc6c5c4)cc3)c2)cc1 ; c1ccc(-c2ccc(N(c3ccc4oc5ccccc5c4c3)c3ccc4oc5c6ccccc6c(-c6ccccc6)cc5c4c3)cc2)cc1]

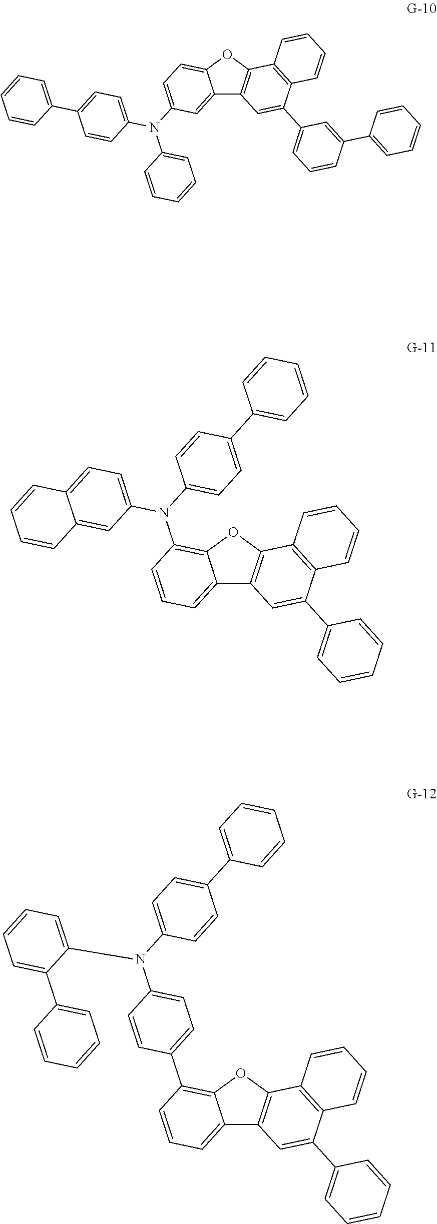 [SMILES: c1ccc(-c2ccc(N(c3ccccc3)c3ccc4oc5c6ccccc6c(-c6cccc(-c7ccccc7)c6)cc5c4c3)cc2)cc1 ; c1ccc(-c2ccc(N(c3ccc4ccccc4c3)c3cccc4c3oc3c5ccccc5c(-c5ccccc5)cc43)cc2)cc1 ; c1ccc(-c2ccc(N(c3ccc(-c4cccc5c4oc4c6ccccc6c(-c6ccccc6)cc54)cc3)c3ccccc3-c3ccccc3)cc2)cc1]

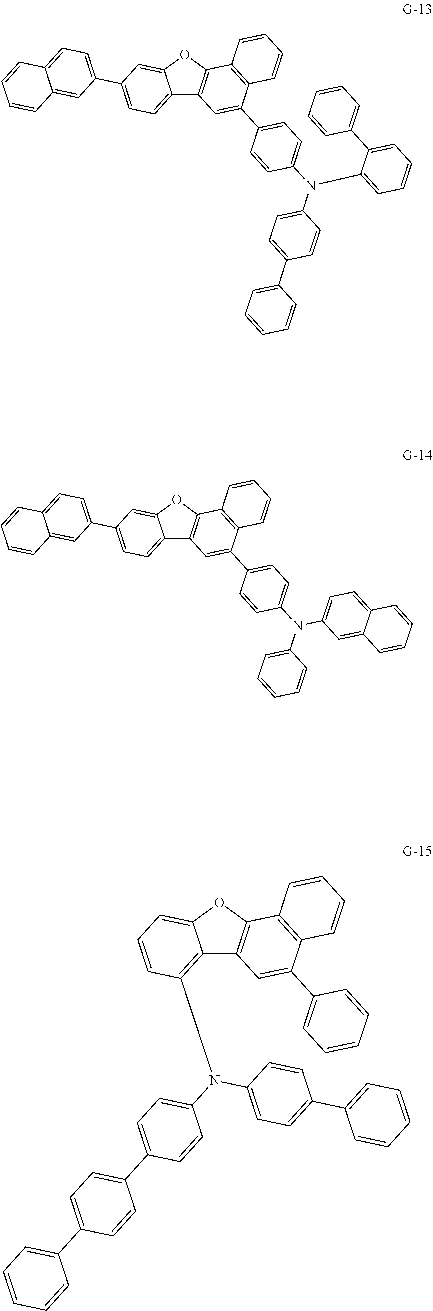 [SMILES: c1ccc(-c2ccc(N(c3ccc(-c4cc5c6ccc(-c7ccc8ccccc8c7)cc6oc5c5ccccc45)cc3)c3ccccc3-c3ccccc3)cc2)cc1 ; c1ccc(N(c2ccc(-c3cc4c5ccc(-c6ccc7ccccc7c6)cc5oc4c4ccccc34)cc2)c2ccc3ccccc3c2)cc1 ; c1ccc(-c2ccc(-c3ccc(N(c4ccc(-c5ccccc5)cc4)c4cccc5oc6c7ccccc7c(-c7ccccc7)cc6c45)cc3)cc2)cc1]

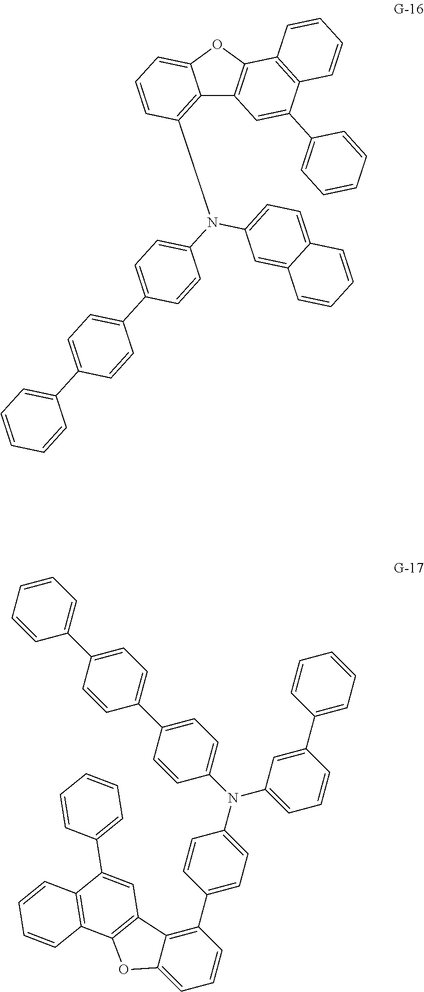 [SMILES: c1ccc(-c2ccc(-c3ccc(N(c4ccc5ccccc5c4)c4cccc5oc6c7ccccc7c(-c7ccccc7)cc6c45)cc3)cc2)cc1 ; c1ccc(-c2ccc(-c3ccc(N(c4ccc(-c5cccc6oc7c8ccccc8c(-c8ccccc8)cc7c56)cc4)c4cccc(-c5ccccc5)c4)cc3)cc2)cc1]

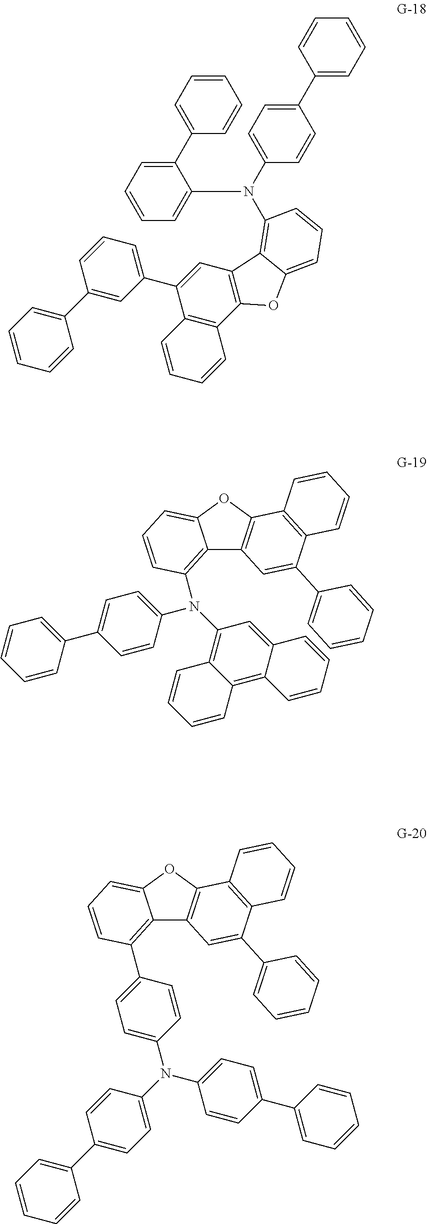 [SMILES: c1ccc(-c2ccc(N(c3ccccc3-c3ccccc3)c3cccc4oc5c6ccccc6c(-c6cccc(-c7ccccc7)c6)cc5c34)cc2)cc1 ; c1ccc(-c2ccc(N(c3cc4ccccc4c4ccccc34)c3cccc4oc5c6ccccc6c(-c6ccccc6)cc5c34)cc2)cc1 ; c1ccc(-c2ccc(N(c3ccc(-c4ccccc4)cc3)c3ccc(-c4cccc5oc6c7ccccc7c(-c7ccccc7)cc6c45)cc3)cc2)cc1]

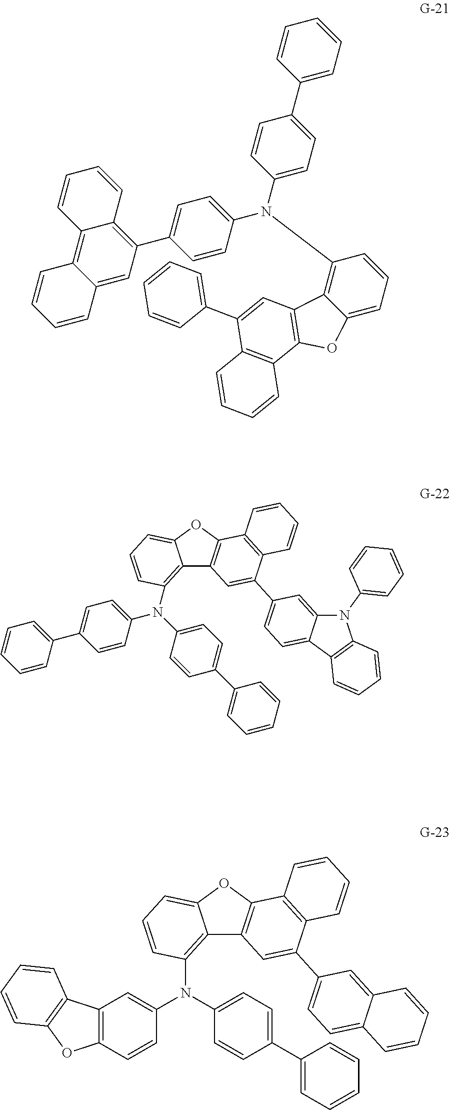 [SMILES: c1ccc(-c2ccc(N(c3ccc(-c4cc5ccccc5c5ccccc45)cc3)c3cccc4oc5c6ccccc6c(-c6ccccc6)cc5c34)cc2)cc1 ; c1ccc(-c2ccc(N(c3ccc(-c4ccccc4)cc3)c3cccc4oc5c6ccccc6c(-c6ccc7c8ccccc8n(-c8ccccc8)c7c6)cc5c34)cc2)cc1 ; c1ccc(-c2ccc(N(c3ccc4oc5ccccc5c4c3)c3cccc4oc5c6ccccc6c(-c6ccc7ccccc7c6)cc5c34)cc2)cc1]

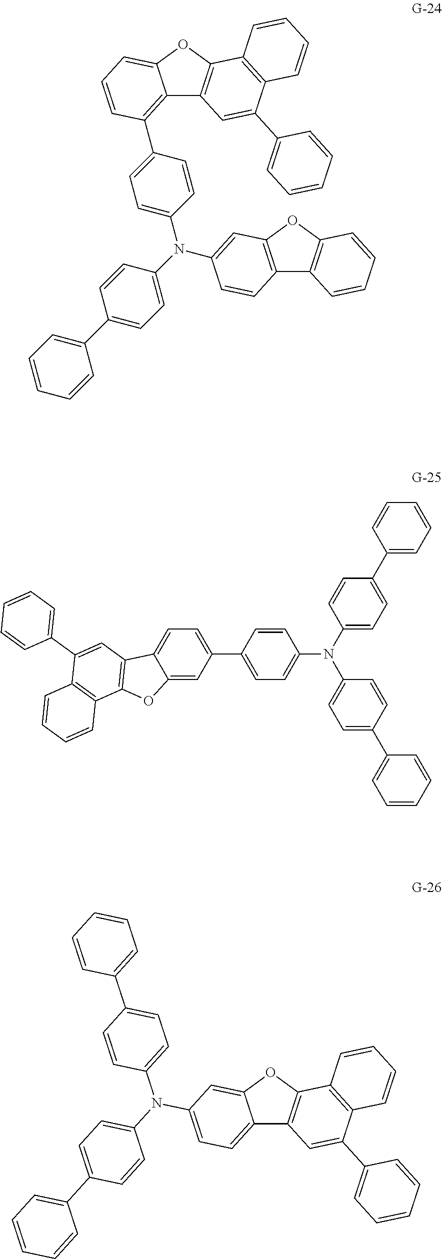 [SMILES: c1ccc(-c2ccc(N(c3ccc(-c4cccc5oc6c7ccccc7c(-c7ccccc7)cc6c45)cc3)c3ccc4c(c3)oc3ccccc34)cc2)cc1 ; c1ccc(-c2ccc(N(c3ccc(-c4ccccc4)cc3)c3ccc(-c4ccc5c(c4)oc4c6ccccc6c(-c6ccccc6)cc54)cc3)cc2)cc1 ; c1ccc(-c2ccc(N(c3ccc(-c4ccccc4)cc3)c3ccc4c(c3)oc3c5ccccc5c(-c5ccccc5)cc43)cc2)cc1]

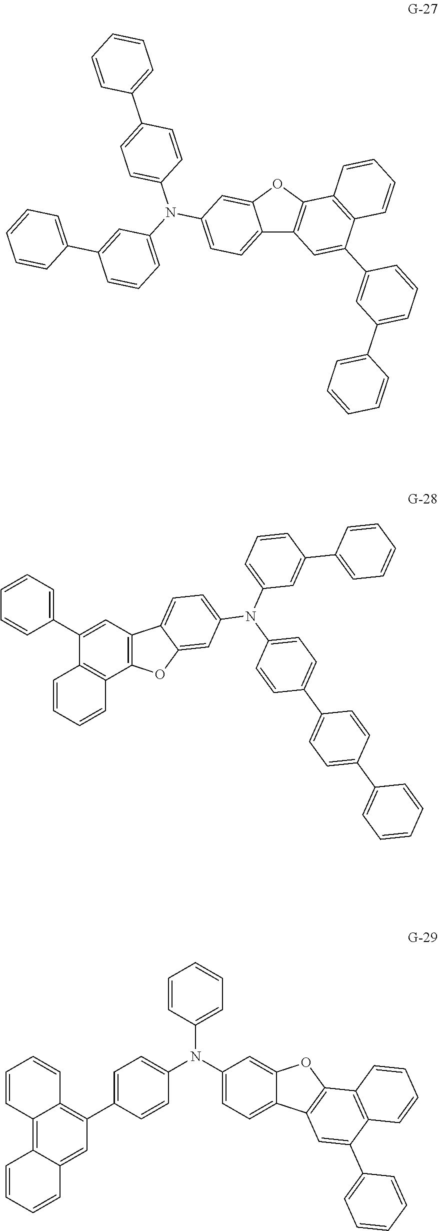 [SMILES: c1ccc(-c2ccc(N(c3cccc(-c4ccccc4)c3)c3ccc4c(c3)oc3c5ccccc5c(-c5cccc(-c6ccccc6)c5)cc43)cc2)cc1 ; c1ccc(-c2ccc(-c3ccc(N(c4cccc(-c5ccccc5)c4)c4ccc5c(c4)oc4c6ccccc6c(-c6ccccc6)cc54)cc3)cc2)cc1 ; c1ccc(-c2cc3c4ccc(N(c5ccccc5)c5ccc(-c6cc7ccccc7c7ccccc67)cc5)cc4oc3c3ccccc23)cc1]

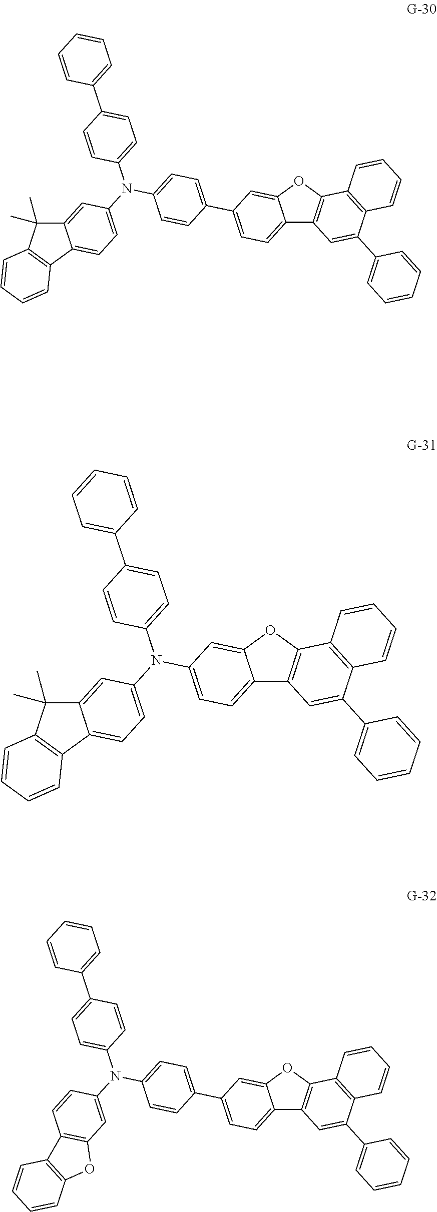 [SMILES: CC1(C)c2ccccc2-c2ccc(N(c3ccc(-c4ccccc4)cc3)c3ccc(-c4ccc5c(c4)oc4c6ccccc6c(-c6ccccc6)cc54)cc3)cc21 ; CC1(C)c2ccccc2-c2ccc(N(c3ccc(-c4ccccc4)cc3)c3ccc4c(c3)oc3c5ccccc5c(-c5ccccc5)cc43)cc21 ; c1ccc(-c2ccc(N(c3ccc(-c4ccc5c(c4)oc4c6ccccc6c(-c6ccccc6)cc54)cc3)c3ccc4c(c3)oc3ccccc34)cc2)cc1]

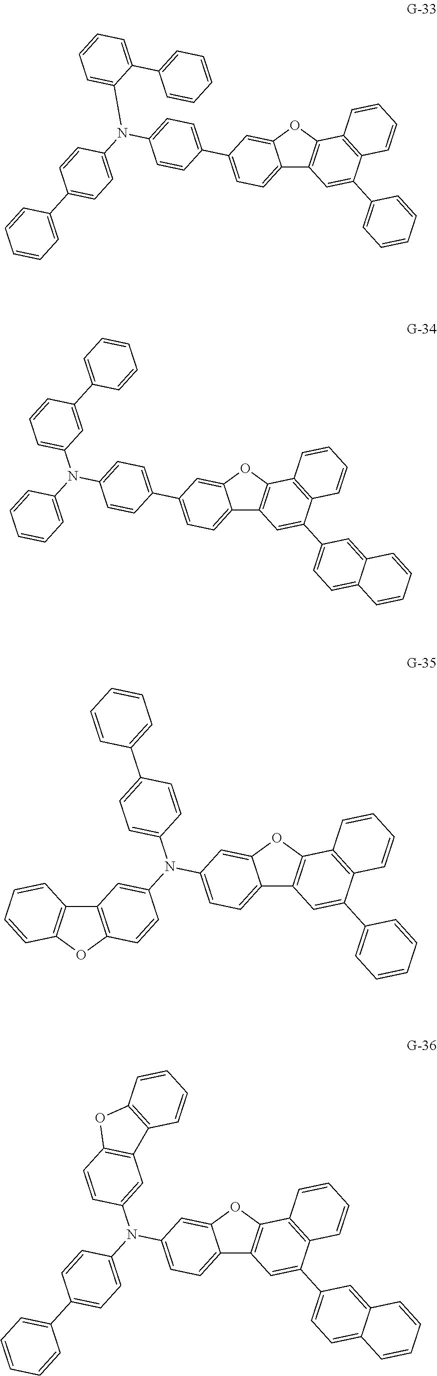 [SMILES: c1ccc(-c2ccc(N(c3ccc(-c4ccc5c(c4)oc4c6ccccc6c(-c6ccccc6)cc54)cc3)c3ccccc3-c3ccccc3)cc2)cc1 ; c1ccc(-c2cccc(N(c3ccccc3)c3ccc(-c4ccc5c(c4)oc4c6ccccc6c(-c6ccc7ccccc7c6)cc54)cc3)c2)cc1 ; c1ccc(-c2ccc(N(c3ccc4c(c3)oc3c5ccccc5c(-c5ccccc5)cc43)c3ccc4oc5ccccc5c4c3)cc2)cc1 ; c1ccc(-c2ccc(N(c3ccc4c(c3)oc3c5ccccc5c(-c5ccc6ccccc6c5)cc43)c3ccc4oc5ccccc5c4c3)cc2)cc1]

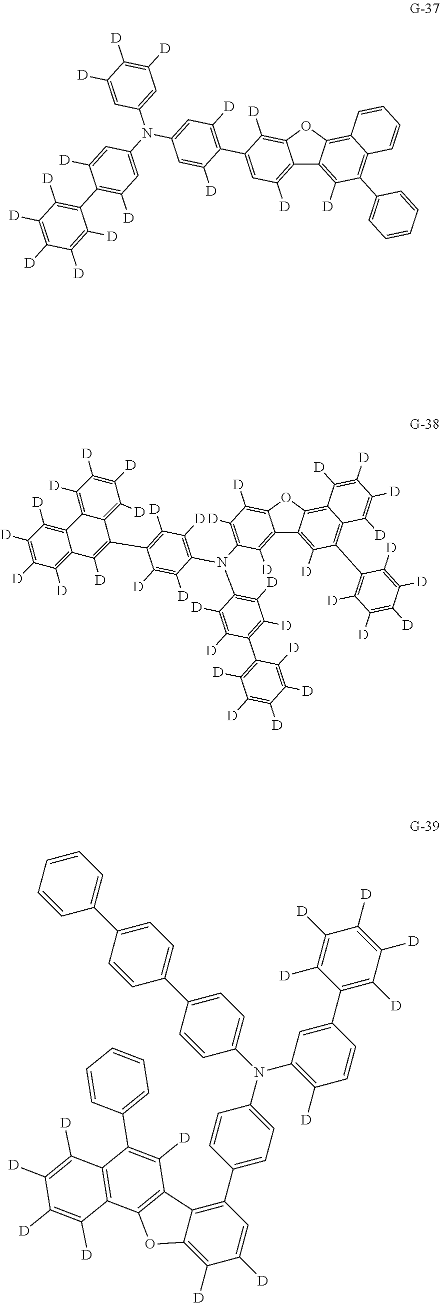

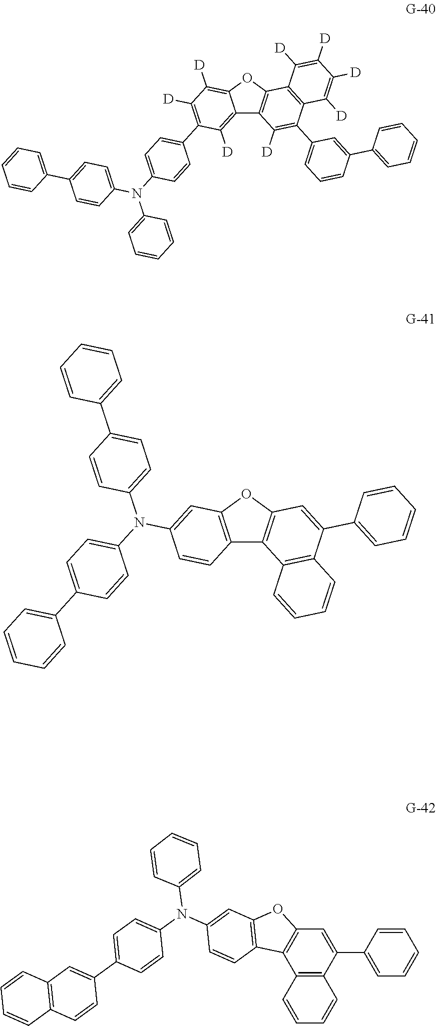 [SMILES: [2H]c1c(-c2ccc(N(c3ccccc3)c3ccc(-c4ccccc4)cc3)cc2)c([2H])c2c(oc3c4c([2H])c([2H])c([2H])c([2H])c4c(-c4cccc(-c5ccccc5)c4)c([2H])c32)c1[2H] ; c1ccc(-c2ccc(N(c3ccc(-c4ccccc4)cc3)c3ccc4c(c3)oc3cc(-c5ccccc5)c5ccccc5c34)cc2)cc1 ; c1ccc(-c2cc3oc4cc(N(c5ccccc5)c5ccc(-c6ccc7ccccc7c6)cc5)ccc4c3c3ccccc23)cc1]

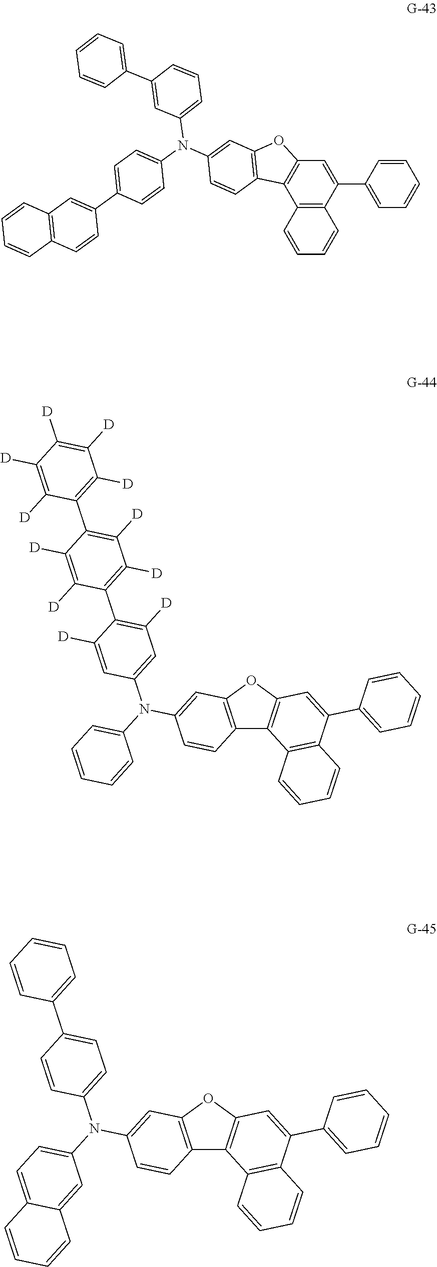 [SMILES: c1ccc(-c2cccc(N(c3ccc(-c4ccc5ccccc5c4)cc3)c3ccc4c(c3)oc3cc(-c5ccccc5)c5ccccc5c34)c2)cc1 ; [2H]c1cc(N(c2ccccc2)c2ccc3c(c2)oc2cc(-c4ccccc4)c4ccccc4c23)cc([2H])c1-c1c([2H])c([2H])c(-c2c([2H])c([2H])c([2H])c([2H])c2[2H])c([2H])c1[2H] ; c1ccc(-c2ccc(N(c3ccc4ccccc4c3)c3ccc4c(c3)oc3cc(-c5ccccc5)c5ccccc5c34)cc2)cc1]

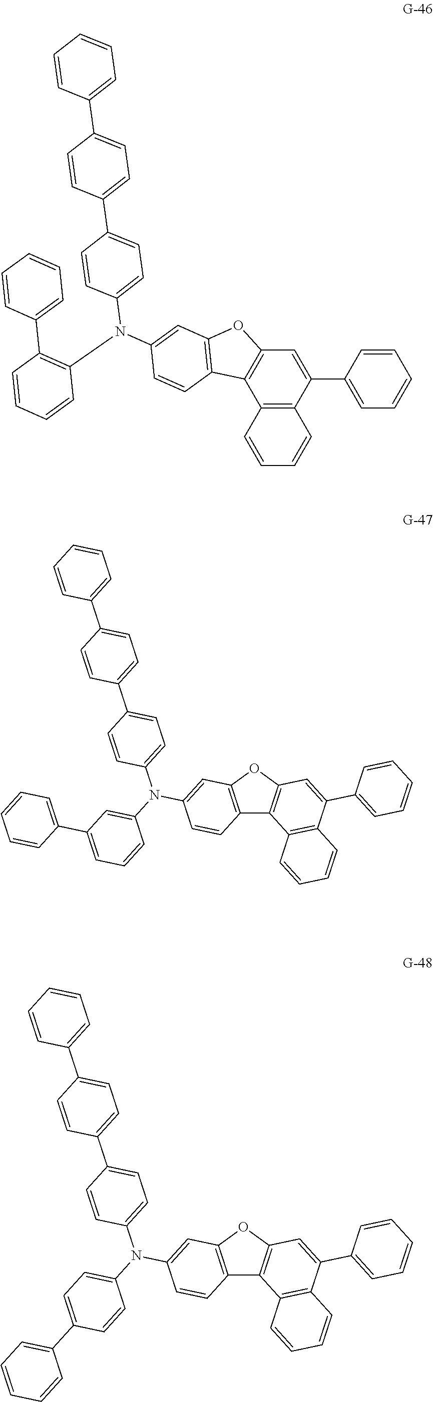 [SMILES: c1ccc(-c2ccc(-c3ccc(N(c4ccc5c(c4)oc4cc(-c6ccccc6)c6ccccc6c45)c4ccccc4-c4ccccc4)cc3)cc2)cc1 ; c1ccc(-c2ccc(-c3ccc(N(c4cccc(-c5ccccc5)c4)c4ccc5c(c4)oc4cc(-c6ccccc6)c6ccccc6c45)cc3)cc2)cc1 ; c1ccc(-c2ccc(-c3ccc(N(c4ccc(-c5ccccc5)cc4)c4ccc5c(c4)oc4cc(-c6ccccc6)c6ccccc6c45)cc3)cc2)cc1]

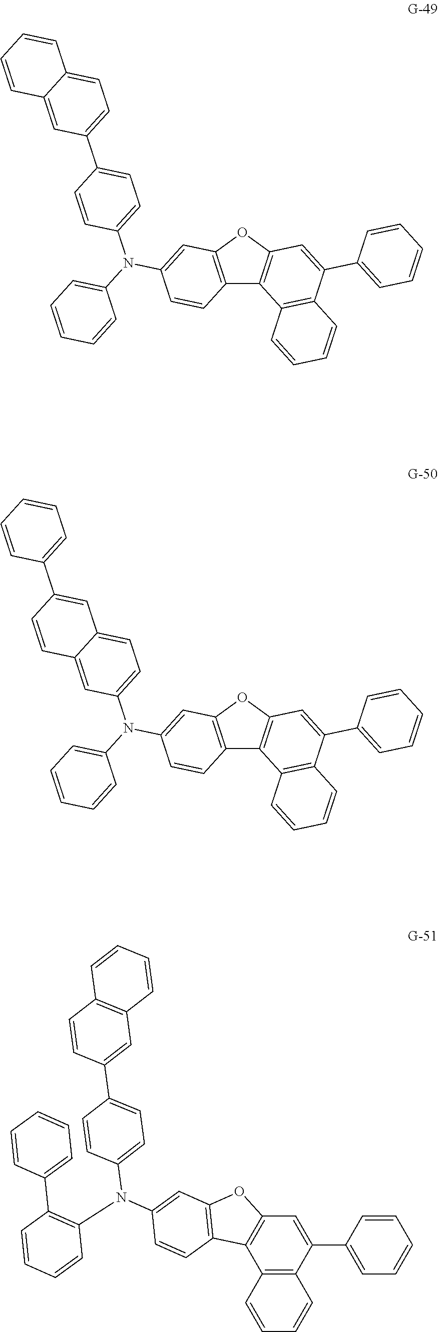 [SMILES: c1ccc(-c2cc3oc4cc(N(c5ccccc5)c5ccc(-c6ccc7ccccc7c6)cc5)ccc4c3c3ccccc23)cc1 ; c1ccc(-c2ccc3cc(N(c4ccccc4)c4ccc5c(c4)oc4cc(-c6ccccc6)c6ccccc6c45)ccc3c2)cc1 ; c1ccc(-c2ccccc2N(c2ccc(-c3ccc4ccccc4c3)cc2)c2ccc3c(c2)oc2cc(-c4ccccc4)c4ccccc4c23)cc1]

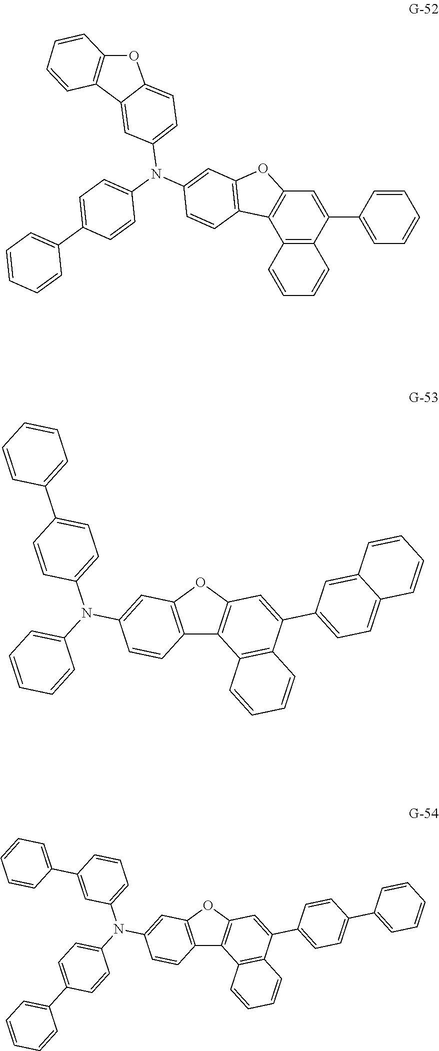 [SMILES: c1ccc(-c2ccc(N(c3ccc4c(c3)oc3cc(-c5ccccc5)c5ccccc5c34)c3ccc4oc5ccccc5c4c3)cc2)cc1 ; c1ccc(-c2ccc(N(c3ccccc3)c3ccc4c(c3)oc3cc(-c5ccc6ccccc6c5)c5ccccc5c34)cc2)cc1 ; c1ccc(-c2ccc(-c3cc4oc5cc(N(c6ccc(-c7ccccc7)cc6)c6cccc(-c7ccccc7)c6)ccc5c4c4ccccc34)cc2)cc1]

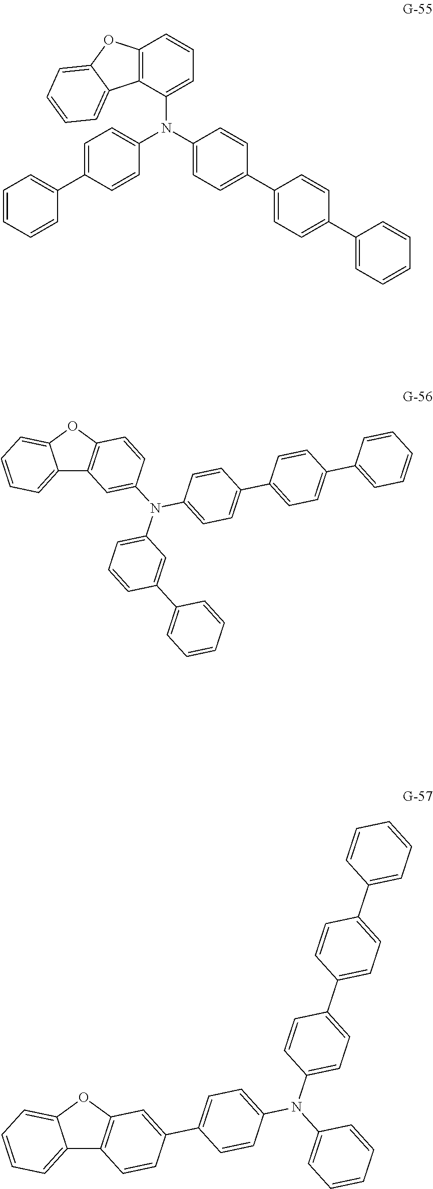 [SMILES: c1ccc(-c2ccc(-c3ccc(N(c4ccc(-c5ccccc5)cc4)c4cccc5oc6ccccc6c45)cc3)cc2)cc1 ; c1ccc(-c2ccc(-c3ccc(N(c4cccc(-c5ccccc5)c4)c4ccc5oc6ccccc6c5c4)cc3)cc2)cc1 ; c1ccc(-c2ccc(-c3ccc(N(c4ccccc4)c4ccc(-c5ccc6c(c5)oc5ccccc56)cc4)cc3)cc2)cc1]

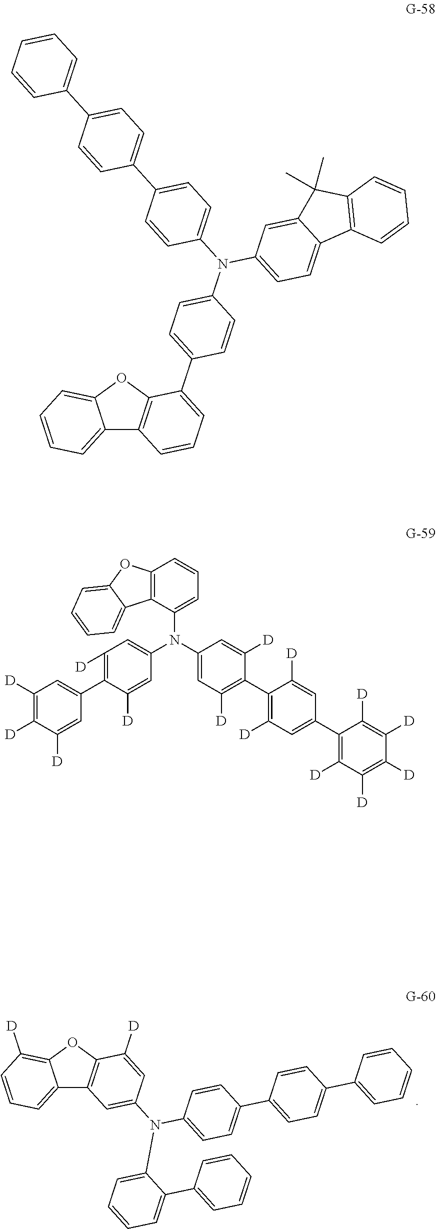 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; [2H]c1cc(-c2c([2H])cc(N(c3cc([2H])c(-c4c([2H])cc(-c5c([2H])c([2H])c([2H])c([2H])c5[2H])cc4[2H])c([2H])c3)c3cccc4oc5ccccc5c34)cc2[2H])cc([2H])c1[2H] ; [2H]c1cccc2c1oc1c([2H])cc(N(c3ccc(-c4ccc(-c5ccccc5)cc4)cc3)c3ccccc3-c3ccccc3)cc12]

12. A composition for an organic material layer of an organic light emitting device, the composition comprising the heterocyclic compound of claim 1.

13. The composition of claim 12, further comprising a compound of the following Chemical Formula 2: 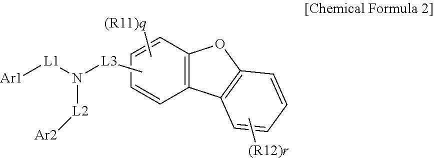
in Chemical Formula 2,
L1 to L3 are each independently a direct bond; a substituted or unsubstituted C6 to C60 arylene group; or a substituted or unsubstituted C2 to C60 heteroarylene group,
Ar1 and Ar2 are each independently a substituted or unsubstituted C6 to C60 aryl group; or a substituted or unsubstituted C2 to C60 heteroaryl group,
R11 and R12 are each independently hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, or are bonded to an adjacent group to form a substituted or unsubstituted C6 to C60 aromatic ring or a substituted or unsubstituted C2 to C60 hetero ring,
q is an integer from 1 to 3,
r is an integer of 1 to 4, and
when q and r are each 2 or higher, substituents in the parenthesis are the same as or different from each other.
